(19) United States
(12) Patent Application Publication
(10) Pub. No.: US 2022/0328185 A1
(43) Pub. Date: Oct. 13, 2022

(54) METHOD AND SYSTEM FOR PREDICTING GESTATIONAL DIABETES

(71) Applicant: Yeda Research and Development Co. Ltd., Rehovot (IL)

(72) Inventors: Eran SEGAL, Ramat-HaSharon (IL); Smadar SHILO, Rehovot (IL); Nitzan ARTZI, Rehovot (IL)

(73) Assignee: Yeda Research and Development Co. Ltd., Rehovot (IL)

(21) Appl. No.: 17/614,024

(22) PCT Filed: May 24, 2020
(86) PCT No.: PCT/IL2020/050570

§ 371 Date: Nov. 24, 2021

Related U.S. Application Data
(60) Provisional application No. 62/852,317, filed on May 24, 2019.

(51) Int. Cl. G16H 50/20; G16H 50/30; G16H 10/60; G16H 50/70; G16H 10/20

(52) U.S. Cl. CPC G16H 50/20; G16H 50/30; G16H 10/60; G16H 50/70; G16H 10/20; G01N 2800/50; G01N 2800/042

(57) ABSTRACT

A method of predicting likelihood for gestational diabetes, comprises: obtaining a plurality of parameters characterizing a female subject, accessing a computer readable medium storing a machine learning procedure trained for predicting likelihoods for gestational diabetes, feeding the procedure with the plurality of parameters, and receiving from the procedure an output indicative of a likelihood that the subject has, or expected to develop, gestational diabetes, wherein the output indicative is related non-linearly to the parameters.

### RELATED APPLICATION

This application claims the benefit of priority of U.S. Provisional Patent Application No. 62/852,317 filed on May 24, 2019, the contents of which are incorporated herein by reference in their entirety.

### FIELD AND BACKGROUND OF THE INVENTION

The present invention, in some embodiments thereof, relates to medicine and, more particularly, but not exclusively, to a method and system for predicting gestational diabetes.

Gestational diabetes mellitus (GDM) is a common medical complication of pregnancy, in which pregnant women without previous diagnosis of diabetes develop glucose intolerance, posing increased risk of short and long term complications for both mother and offspring. If undiagnosed, GDM may cause complication of pregnancy that can increase the risk of a number of maternal-fetal disorders, including macrosomia, shoulder dystocia or birth injury, premature delivery, and preeclampsia. In addition to the increased risk of complications associated with gestation and delivery, there are also serious postnatal complications associated with GDM. For instance, women with GDM may have diabetes immediately after pregnancy, and women who have had GDM have a higher chance of developing diabetes within the next years. Children of mothers with GDM have a higher risk of developing type-2 diabetes in later life. Thus, untreated GDM contributes to the overall diabetic population in both the short and long term. Currently, GDM is typically diagnosed 24-28 weeks of gestation.

### SUMMARY OF THE INVENTION

According to an aspect of some embodiments of the present invention there is provided a method of predicting likelihood for gestational diabetes. The method comprises: obtaining a plurality of parameters characterizing a female subject; accessing a computer readable medium storing a machine learning procedure trained for predicting likelihoods for gestational diabetes; feeding the procedure with the plurality of parameters; and receiving from the procedure an output indicative of a likelihood that the subject has, or expected to develop, gestational diabetes, wherein the output indicative is related non-linearly to the parameters.

According to some embodiments of the invention the plurality of parameters comprises at least one parameter extracted from an electronic health record associated with the subject.

According to some embodiments of the invention the method comprises presenting to the subject, by a user interface, a questionnaire and a set of questionnaire controls, receiving a set of response parameters entered by the subject using the questionnaire controls, wherein the plurality of parameters comprises the response parameters.

According to some embodiments of the invention the plurality of parameters comprises at least one parameter extracted from a body liquid test applied to the subject.

According to some embodiments of the invention the plurality of parameters comprises at least one parameter extracted from a diagnosis previously recorded for the subject.

According to some embodiments of the invention the plurality of parameters comprises at least one parameter indicative of a pharmaceutical prescribed for the subject.

According to some embodiments of the invention the female subject is pregnant.

According to some embodiments of the invention the method wherein the pregnant subject is at less than 12 weeks or less than 11 weeks or less than 10 weeks or less than 9 weeks or less than 8 weeks or less than 7 weeks or less than 6 weeks or less than 5 weeks gestation According to some embodiments of the invention the pregnant subject is at less than 8 weeks gestation.

According to some embodiments of the invention the pregnant subject is at less than 6 weeks gestation.

According to some embodiments of the invention the plurality of parameters comprises a result of a blood glucose test applied to the subject.

According to some embodiments of the invention the plurality of parameters comprises an absolute neutrophil count (NEUT.abs) obtained from a blood of the subject.

According to some embodiments of the invention the plurality of parameters comprises white blood cells count WBC, obtained from a blood of the subject.

According to some embodiments of the invention the plurality of parameters comprises a result of a blood glucose test applied to the subject within about 1 year before the pregnancy.

According to some embodiments of the invention the female subject is not pregnant.

According to some embodiments of the invention the female subject is undergoing an assisted reproduction treatment.

According to some embodiments of the invention the assisted reproduction treatment is selected from the group consisting of in vitro fertilization (IVF), Gamete Intrafallopian Transfer Procedure (GIFT), Zygote Intrafallopian Transfer Procedure (ZIFT), Intracytoplasmic Sperm Injection (ICSI), Intrauterine Insemination (IUI), and Therapeutic Donor Insemination (TDI).

According to some embodiments of the invention the plurality of parameters comprises at least one parameter indicative of a glucose tolerance test applied to the subject.

According to some embodiments of the invention the subject has been previously pregnant, and wherein the plurality of parameters comprises a result of a glucose tolerance test applied to the subject during a previous pregnancy.

According to some embodiments of the invention the subject has been previously pregnant, and wherein the plurality of parameters comprises a result of a blood glucose test applied to the subject during a previous pregnancy.

According to some embodiments of the invention the previous pregnancy is a most recent previous pregnancy.

According to some embodiments of the invention the previous pregnancy is a next-to-most recent previous pregnancy.

According to some embodiments of the invention the plurality of parameters comprises at least 10 or at least 20 or at least 30 or at least 40 or at least 50 or at least 100 or at least 200 or at least 300 or at least 400 or at least 500 or at least 1,000 or at least 1,500 or more of the parameters listed in Table 6.1.

According to some embodiments of the invention the plurality of parameters comprises at least 10 or at least 12 or at least 14 or at least 16 of the parameters that are listed at lines 1-40 more preferably lines 1-30 more preferably lines 1-20 of Table 6.1.

According to some embodiments of the invention the plurality of parameters comprises at least 20 or at least 22 or at least 24 or at least 26 or at least 30 or at least 32 or at least 34 or at least 36 of the parameters that are listed at lines 1-50 more preferably lines 1-45 more preferably lines 1-40 of Table 6.1.

According to some embodiments of the invention the plurality of parameters comprises least 50 or at least 60 or at least 70 or at least 80 or at least 90 of the parameters that are listed at lines 1-300 more preferably lines 1-200 more preferably lines 1-100 of Table 6.1.

According to an aspect of some embodiments of the present invention there is provided a method of predicting likelihood for gestational diabetes. The method comprises: presenting on a user interface a questionnaire and a set of questionnaire controls, and receiving from the user interface a set of response parameters entered using the questionnaire controls, wherein the set of response parameters characterizes a female subject; accessing a computer readable medium storing a machine learning procedure trained for predicting likelihoods for gestational diabetes; feeding the procedure with the set of parameters; and receiving from the procedure an output indicative of a likelihood that the female subject has, or expected to develop, gestational diabetes, wherein the output indicative is related non-linearly to the parameters.

According to an aspect of some embodiments of the present invention there is provided a method of determining whether to apply a glucose tolerance test (GTT) to a female subject that has been previously pregnant. The method comprises: obtaining a plurality of parameters characterizing a female subject, wherein the plurality of parameters comprises a result of a GTT applied to the subject during a previous pregnancy; accessing a computer readable medium storing a machine learning procedure trained for predicting likelihoods for gestational diabetes; feeding the procedure with the plurality of parameters; receiving from the procedure an output indicative of a likelihood that the subject has, or expected to develop, gestational diabetes, wherein the output indicative is related non-linearly to the parameters; and when the likelihood is below a predetermined threshold, generating an output recommending not to apply the GTT to the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for predicting likelihood for gestational diabetes, according to various exemplary embodiments of the present invention.

FIG. 2 is a schematic illustration of a client-server configuration which can be used according to some embodiments of the present invention for predicting likelihood for gestational diabetes, according to some embodiments of the present invention.

FIGS. 3A-B show data and cohort characteristics, obtained in experiments performed according to some embodiments of the present invention. FIG. 3A shows cohort selection. Pregnancies were first identified by the offspring's birth date. Second, women with pre-existing diabetes and pregnancies without a record of glucose tolerance test (GTT) were excluded. Finally, the cohort was divided into training and validation sets. FIG. 3B shows feature availability distribution. Pies are divided according to the sum of datapoints in each feature set. The majority of data originates from lab tests results during current or previous pregnancies.

FIGS. 4A-E show predictive model evaluation, obtained in experiments performed according to some embodiments of the present invention. FIG. 4A shows Receiver Operating Characteristic (ROC) curve, comparing the model of the present embodiments (solid) and the Baseline Risk Score (dashed). Lighter colored lines are ROC curves of stratified partition of the test set; bracketed values are 95% confidence intervals calculated through a normal fit of those curves. FIG. 4B shows precision-Recall (PR) curve, with the same properties as in FIG. 4A. FIG. 4C shows calibration curve, showing fraction of GDM-positive samples over binned prediction scores versus mean prediction score. FIG. 4D shows predictions on different subsets of the cohort. auPR is shown for each subset, for the model of the present embodiments (blue, “model prediction”) and the baseline score (orange, “baseline”). Predictions women with a higher baseline risk score (>2) have the greatest deviation from the baseline risk score model. The subset of patients who undergo two routine blood tests during pregnancy has a higher prevalence of GDM and a higher prediction accuracy. Error bars show 95% confidence intervals, and dark blue lines show the prevalence in each subset. FIG. 4E shows time-dependent analysis. Every point is the evaluation score of a model built only with features available at this time point (dashed lines are, from top to bottom: Previous GCT, Entire cohort, First pregnancy).

FIGS. 5A-F show Shapley values based interpretation of the model of the present embodiments, as obtained in the performed experiments. FIG. 5A shows feature importance of the top 20 contributing features. Bar colors indicate direction of influence, based on the dependence plot of this feature. FIG. 5B shows analysis of contributing feature category. Shapley values were summed for each feature set, and the mean of their absolute sum was computed across all samples, producing a feature importance score for sets of features. FIGS. 5C-E show three examples of dependence plots, showing predicted relative risk versus feature value for BMI before pregnancy (FIG. 5C), number of first-degree relatives with diagnosed DM (FIG. 5D) and pregestation HbA1C % blood test (FIG. 5E). Bands represent s.d. of the population per bin, which is related to interactions between input features. Regions of larger vertical bands represent x-axis values in which other features modified the risk attributed to the x-axis feature (n=71,952 for FIG. 5C and n=82,678 for FIGS. 5D and 5E). FIG. 5F shows combined Shapley value for all GCT/OGTT results during the previous pregnancy is plotted against GCT value during the current pregnancy. Every point is a sample and is colored according to GDM status during the previous pregnancy. While GDM in a previous pregnancy predicts that in the current pregnancy, GCT provides a continuous prediction for all women who took the test previously. Additionally, GCT in the previous pregnancy also indicates women whose GDM risk is as low as that of 20% of the population (n=45,807).

FIGS. 6A-D show a questionnaire-based prediction according to some embodiments of the present invention. FIGS. 6A-B show validation results of a questionnaire-based predictor, using 9 simple questions. Both ROC (FIG. 6A) and PR (FIG. 6B) curves of the model are shown (n=71,952). FIG. 6C shows a user interface displaying a list of questions that assemble the predictor of these embodiments. FIG. 6D shows the model of the present embodiments as a tool for GDM screening. In this scenario, the trade-off of not testing low-risk patients while retaining the current system of GCT/OGTT for all others is presented. The ratio of GCT avoidance is plotted against the predictor miss rate. That is, the percentage of GDM positive patients that would not be diagnosed following this approach (n=82,678). PCOS abbreviates polycystic ovary syndrome.

FIGS. 7A-E show evaluation of the model of the present embodiments on a geographical validation sets. FIG. 7A shows ROC curve, comparing the model of the present embodiments (solid) and the Baseline Risk Score (dashed). Lighter colored lines are ROC curves of stratified partition of the validation set (not shown in ROC); bracketed values are 95% confidence intervals calculated through a normal fit of those curves. FIG. 7B shows PR curve, with the same properties as in FIG. 7A. FIG. 7C shows the fraction of GDM-positive samples in every decile of the predicted probability. FIG. 7D shows predictions on different subsets of the cohort. auPR is shown for each subset, for the model of the present embodiments (blue) and the baseline score (orange). Error bars show 95% confidence intervals, and dark blue lines show the prevalence in each subset. Shaded area is the distribution of the relevant score. FIG. 7E shows performance by gestational age at prediction. Every point is the evaluation score of a model built only with features available at this time point. (n=46,002 for FIGS. 7A-C. Subset sample sizes are listed in FIG. 7D). Dashed lines are, from top to bottom: First pregnancy, Entire cohort, Has Prior GCT.

FIGS. 8A-D show baseline prediction, based on Baseline Risk Score. FIG. 8A shows odds ratio for the risk score composing parameters. Adjusted odds ratios were derived from a logistic regression model, both values are presented on the training set. For each pair of bars the top bar (blue in the color version) corresponds to univariate, and the bottom (orange in the color version) corresponds to adjusted. FIG. 8B shows prevalence among women grouped by risk score. Error bars represent 90% confidence intervals on the train set. FIG. 8C shows histogram of risk scores in the training set. In each bar, the top part (orange in the color version) corresponds to GDM Positive, and the bottom part (blue in the color version) corresponds to GDM Negative). FIG. 8D shows ROC curve for NIH Risk Score and for a logistic regression model trained on its constructing parameters. Results are reported on the test set. Logistic regression model does not suppress the Naive summation in the risk score.

FIG. 9 is a histogram of lab tests during pregnancy, showing time-window F0, F1 and F2, defined in experiments performed according to some embodiments of the present invention. The peaks showing are weekly, and represent the fact that patients tend to see a physician on the same day of the week.

FIGS. 10A-E includes dependence plots of 20 most significant features, ranked according to the mean absolute Shapley value, as obtained in experiments performed according to some embodiments of the present invention.

FIGS. 11A-E show evaluation of the model of the present embodiments on a geo-temporal validation set. FIG. 11A shows ROC curve, comparing the model of the present embodiments (solid) and the Baseline Risk Score (dashed). Symbols and colors are as in FIG. 7A. FIG. 11B shows PR curve, with the same properties as in FIG. 11A. FIG. 11C shows the fraction of GDM-positive samples in every decile of the predicted probability. FIG. 11D shows predictions on different subsets of the cohort. Symbols and colors are as in FIG. 7D. FIG. 11E shows performance by gestational age at prediction. Every point is the evaluation score of a model built only with features available at this time point. (n=8,540 for FIGS. 11A-C. Subset sample sizes are listed in FIG. 11D). Dashed lines are, from top to bottom: Has Prior GCT, Entire cohort, First pregnancy.

FIG. 12A is calibration curve showing a fraction of positive samples per bin versus the mean predicted probability of the bin. Blue and red bars represent the ratio of negative/positive samples in the bin, respectively.

FIG. 12B is a decision curve showing the net benefit versus the threshold probability, for both predictor and baseline. The predictor outperforms the baseline at all thresholds. (n=82,678 for all panels).

### DESCRIPTION OF SPECIFIC EMBODIMENTS OF THE INVENTION

The present invention, in some embodiments thereof, relates to medicine and, more particularly, but not exclusively, to a method and system for predicting gestational diabetes.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

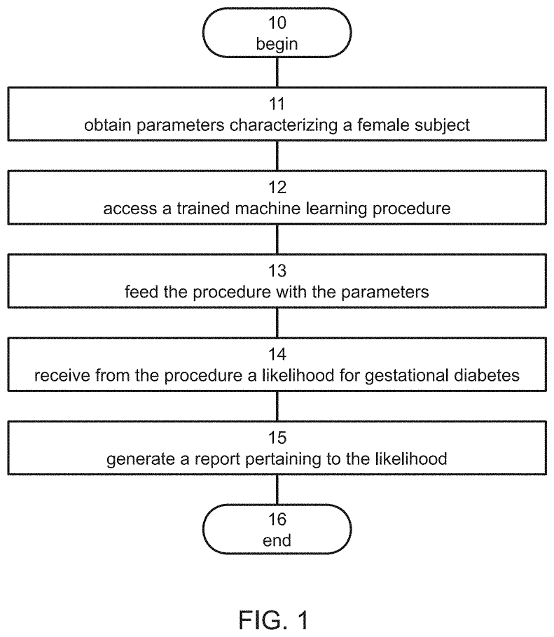

FIG. 1 is a flowchart diagram of a method suitable for predicting likelihood for gestational diabetes, according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The processing operations of the present embodiments can be embodied in many forms. For example, they can be embodied in on a tangible medium such as a computer for performing the operations. They can be embodied on a computer readable medium, comprising computer readable instructions for carrying out the method operations. They can also be embodied in electronic device having digital computer capabilities arranged to run the computer program on the tangible medium or execute the instruction on a computer readable medium.

Computer programs implementing the method according to some embodiments of this invention can commonly be distributed to users on a distribution medium such as, but not limited to, CD-ROM, flash memory devices, flash drives, or, in some embodiments, drives accessible by means of network communication, over the internet (e.g., within a cloud environment), or over a cellular network. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. Computer programs implementing the method according to some embodiments of this invention can also be executed by one or more data processors that belong to a cloud computing environment. All these operations are well-known to those skilled in the art of computer systems. Data used and/or provided by the method of the present embodiments can be transmitted by means of network communication, over the internet, over a cellular network or over any type of network, suitable for data transmission.

The method begins at 10 and continues to 11 at which a plurality of parameters characterizing a female subject is obtained. The inventors discovered that the likelihood for gestational diabetes can be predicting both during the pregnancy and before the pregnancy.

Thus, in some embodiments of the present invention the female subject is pregnant. Preferably, the pregnant subject is at less than 12 weeks or less than 11 weeks or less than 10 weeks or less than 9 weeks or less than 8 weeks or less than 7 weeks or less than 6 weeks or less than 5 weeks gestation.

Alternatively, the female subject is not pregnant, for example, the female subject can be a female subject that desires to be pregnant, or that is expected to be pregnant. In some embodiments of the present invention the female subject is undergoing an assisted reproduction treatment, such as, but not limited to, in vitro fertilization (IVF), Gamete Intrafallopian Transfer Procedure (GIFT), Zygote Intrafallopian Transfer Procedure (ZIFT), Intracytoplasmic Sperm Injection (ICSI), Intrauterine Insemination (IUI), or Therapeutic Donor Insemination (TDI).

At least one of the parameters that are obtained at 11, more preferably more than one of these parameters, more preferably at least 10 or at least 20 or at least 30 or at least 40 or at least 50 or at least 100 or at least 200 or at least 300 or at least 400 or at least 500 or at least 1,000 or at least 1,500 or more of the parameters are extracted from an electronic health record associated with the subject. Parameters extracted from an electronic health record can include, but are not limited to, anthropometric parameters (e.g., height, weight, body mass index), blood pressure measurements, blood and urine laboratory tests, diagnoses recorded by physicians, and/or pharmaceuticals prescribed to the subject.

A list of parameters from which the parameters can be selected is provided in Table 6.1 of the Examples section that follows. In some embodiments of the present invention at least 10 or at least 20 or at least 30 or at least 40 or at least 50 or at least 100 or at least 200 or at least 300 or at least 400 or at least 500 or at least 1,000 or at least 1,500 or more of the parameters are selected from the parameters listed in Table 6.1. Preferably, but not necessarily, at least 10 or at least 12 or at least 14 or at least 16 of the parameters are selected from the parameters that are listed at lines 1-40 more preferably lines 1-30 more preferably lines 1-20 of Table 6.1. In some embodiments, at least 20 or at least 22 or at least 24 or at least 26 or at least 30 or at least 32 or at least 34 or at least 36 of the parameters are selected from the parameters that are listed at lines 1-50 more preferably lines 1-45 more preferably lines 1-40 of Table 6.1. In some embodiments, at least 50 or at least 60 or at least 70 or at least 80 or at least 90 of the parameters are selected from the parameters that are listed at lines 1-300 more preferably lines 1-200 more preferably lines 1-100 of Table 6.1.

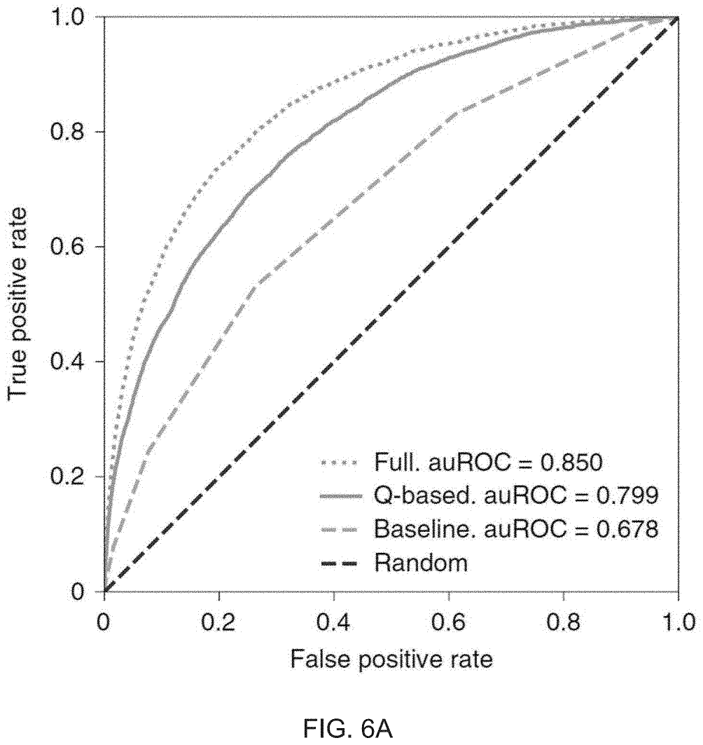

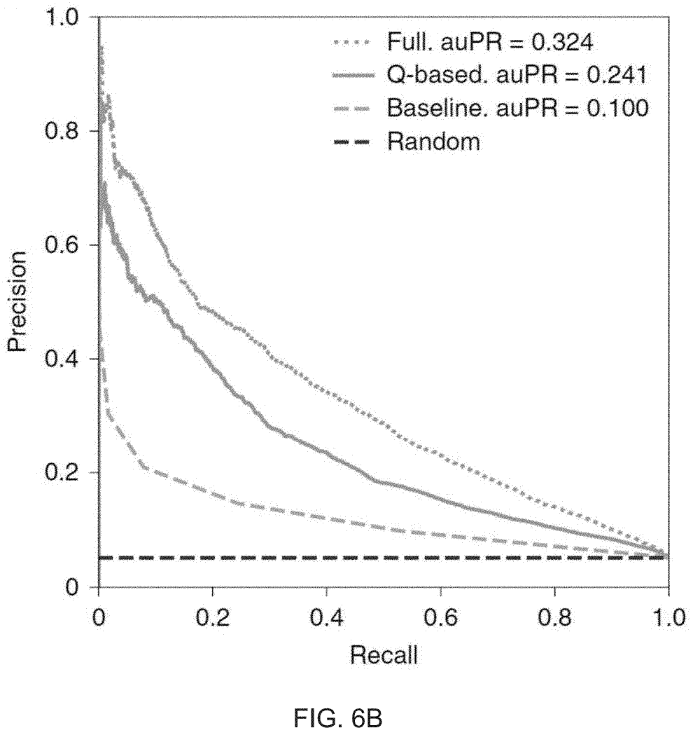

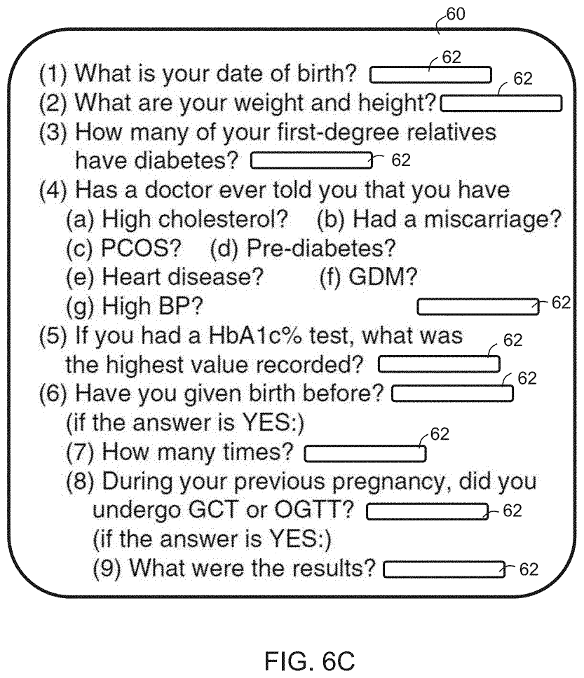

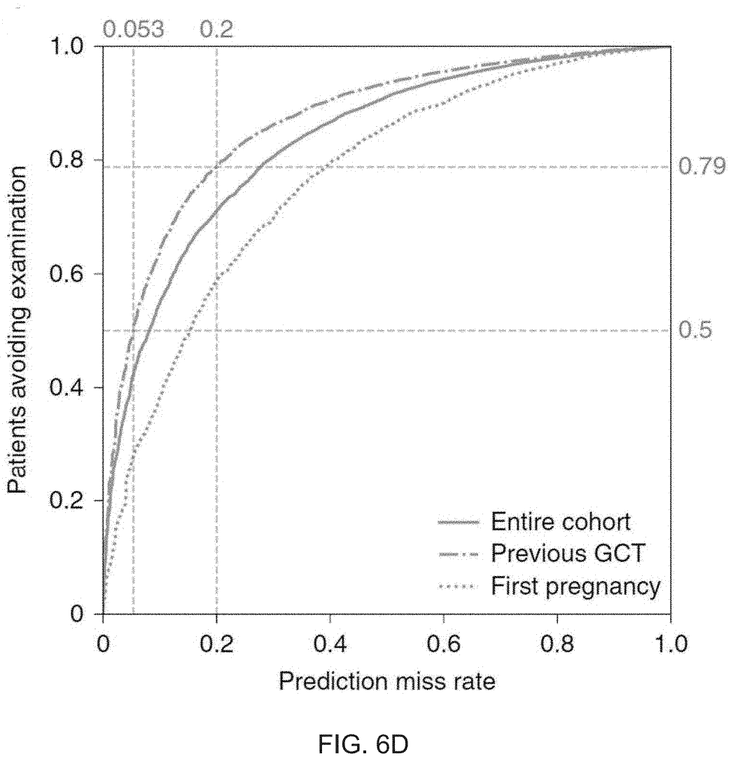

Also contemplated are embodiments in which the parameters are selected from a set of response parameters that are provided by the subject, or on behalf of the subject, by responding to a questionnaire presented to the subject, or to someone on behalf of the subject. These parameters can include anthropometric parameters (e.g., height, weight, body mass index), a parameter indicative of the age of the subject, one or more parameters indicative of history of diabetes for the subject and for close (e.g., first-degree) relatives of the subject, one or more parameters indicative of diagnoses the subject is aware of (e.g., high cholesterol, heart disease, polycystic ovary syndrome, GDM, high blood pressure), one or more parameters indicative of blood test results the subject is aware of (e.g., Hemoglobin A1c test), pregnancy history, and results of GTT (if taken) during previous pregnancies. A representative example of a questionnaire that can be presented to the subject is shown in FIG. 6C.

In some embodiments of the present invention the parameters include only parameters extracted from an electronic health record associated with the subject, in some embodiments of the present invention the parameters include only response parameters that are provided by the subject, or on her behalf, and in some embodiments of the present invention the parameters include both parameters extracted from an electronic health record associated with the subject, and response parameters that are provided by the subject or on her behalf.

The number of parameters that are extracted from an electronic health record associated with the subject is preferably at least 10 or at least 20 or at least 30 or at least 40 or at least 50 or at least 100 or at least 200 or at least 300 or at least 400 or at least 500 or at least 1,000 or at least 1,500 or more. The number of response parameters that are provided by the subject or on her behalf is preferably 20 or less, or 15 or less, or 10 or less. The advantage of this embodiment is that a relative small number of parameter allows the subject to manually respond to the questionnaire at a relatively short time.

When the parameters include both parameters extracted from an electronic health record associated with the subject, and response parameters that are provided by the subject or on her behalf, the number of parameters that are extracted from an electronic health record is optionally and preferably significantly larger (e.g., at least 2 or at least 4 or at least 6 or at least 8 or at least 10 or at least 20 or at least 40 times larger) than the number of response parameters that are provided by the subject or on her behalf.

In some embodiments of the present invention at least one of the parameters is extracted from a body liquid test applied to the subject. Representative examples of body liquid tests from which a parameter can extracted according to some embodiments of the present invention include, without limitation, 17-OH-PROGESTERONE, A.B2 GLYCOPROTEI IgG, A.B2 GLYCOPROTEI IgM, ALBUMIN, ALK. PHOSPHATASE, ALY %, ALY, AMYLASE, ANISO-F, ANTI BODY SCREEN I, ANTI CARDIOLIPIN IgG, ANTI CARDIOLIPIN IgM, ANTI THYROID PEROXID, ANTINUCLEAR Ab_(ANA), ANTITHROMBIN-III, APTT-R, APTT-sec, BASO %, BASO abs, BILIRUBIN INDIRECT, BILIRUBIN TOTAL, BILIRUBIN-U STRIP, BILIRUBIN-DIRECT, BLAST-F, BLOOD TYPE, C-REACTIVE PROTEIN, CALCIUM, CH, CHLORIDE, CHOLESTEROL, CHOLESTEROL-HDL, CHOLESTEROL-LDL calc, CHOLESTEROL/HDL, CK-CREAT.KINASE(CPK), CMV AVIDITY, CMV IgG (Add VIDAS), CMV IgM(Add.-VIDAS), CMV IgM, COMPLEMENT C3, COMPLEMENT C4, CONTROL PT, CONTROL PTT, CREATININE, CREATININE-U SAMPLE, DHEA SULPHATE, DNA (ds) Ab, DNA (ds) Ab, DNA (ds) Ab, EBV IgG-EBNA, EBV IgG-EBNA, EBV IgG-EBNA, EBV VCA IgM, EBV VCA IgM, EBV VCA IgM, EBV VCA_IgG, EBV VCA_IgG, EBV VCA_IgG, EOS %, EOS.abs, EPITHELIAL-SED, ERYTHROCYTES-SED, ERYTHROCYTES-U STRIP, ESR, ESTRADIOL (E-2), FERRITIN, FIBRINOGEN CALCU, FIBRINOGEN, FOLIC ACID, FREE ANDROGEN INDEX, FSH, First isolate, GGT, GLOBULIN, GLOM.FILTR.RATE, GLUCOSE—U STRIP, GLUCOSE, GPT (ALT), HB—other, HB, HCT, HCT/HGB Ratio, HDW, HEMOGLOBIN A, HEMOGLOBIN A1C %, HEMOGLOBIN C, HEMOGLOBIN H, HEMOGLOBIN O, HEMOGLOBIN S, HEMOLYTIC, HEPATITIS Bs Ab, HEPATITIS Bs Ag, HEPATITIS C Ab, HYPER %, HbA, HbA2, HbF, ICTERIC, IRON, KETONES-U STRIP, LDH, LEFT SHIFT, LEUCOCYTES—U STRIP, LEUCOCYTES-SED, LH, LI, LI, LIC %, LIC, LIPASE, LIPEMIC, LUC abs, LUC %, LYM %, LYMP.abs, MACRO %, MAGNESIUM, MCH, MCHC, MCV, MICRO %, MICRO %/HYPO %, MICRO-F, MICROALBUMIN-U SAMP, MICROALBUMIN/CREAT, MONO %, MONO.abs, MPXI, NEUT %, NEUT.abs, NITRITE-U STRIP, NON-HDL_CHOLESTEROL, NORMOBLAST. %, NORMOBLAST.abs, PCT, PDW, PH-U STRIP, PHOSPHORUS, PLATLATE CLUMPS, PLT, POTASSIUM, PROGESTERONE, PROLACTIN, PROT-S ANTIGEN (FREE, PROTEIN C ACTIVITY, PROTEIN-U SAMPLE, PROTEIN-URINE 24 h, PROTEIN-TOTAL, PT %, PT-INR, PT-SEC, RAPID PL.REAGIN-VDRL, RBC, RDW, RDW-CV, RDW-SD, RETICUL. COUNT abs, RETICULOCYTES COUNT %, RHEUMATOID FACTOR, RUBELLA Ab IgG, SHBG, SODIUM, SPECIFIC GRAV-U STRI, T3-FREE, TESTOSTERONE-TOTAL, THYROGLOBULIN Ab, TOXOPLASMA IgG, TOXOPLASMA IgM, TRANSFERRIN, TRIGLYCERIDES, TSH, UREA, URIC ACID, UROBILINOGEN-U STRIP, Urine culture, VARICELLA ZOSTER IgG, VARICELLA ZOSTER IgM, VITAMIN B12, VITAMIN D (25-OH), WBC, wherein the tests are applied during any of the time windows F0, F1, F2, M1, M2, M3, M4, M5, P1, P2, and P3 defined in the Examples section that follows.

In some embodiments of the present invention one or more of the parameters is a result of a blood glucose test applied to said subject.

In some embodiments of the present invention one or more of the parameters is an absolute neutrophil count (NEUT.abs) obtained from a blood of the subject.

In some embodiments of the present invention one or more of the parameters is white blood cells count WBC, obtained from a blood of the subject.

In some embodiments of the present invention one or more of the parameters is a result of a blood glucose test applied to the subject within about 1 year before the pregnancy.

In some embodiments of the present invention the parameters comprise at least one parameter extracted from a clinical or hospital diagnosis previously recorded for the subject. Representative examples of clinical and hospital diagnoses which can be used as parameters according to some embodiments of the present invention include, without limitation, ABDOMINAL PAIN, ABORTION INDUCED BY MEDICATION, ABSENCE OF MENSTRUATION, ACCIDENT/INJURY, ACNE, ACQUIRED HYPOTHYROIDISM, ACUTE APPENDICITIS WITHOUT MENTION OF PERITONITIS, ACUTE BRONCHITIS, ACUTE CONJUNCTIVITIS, ACUTE NASOPHARYNGITIS, ACUTE NONSUPPURATIVE OTITIS MEDIA, ACUTE PHARYNGITIS, ACUTE SINUSITIS, ACUTE TONSILLITIS, ACUTE UPPER RESPIRATORY INFECTIONS OF MULTIPLE OR UNSP.SITES, ACUTE UPPER RESPIRATORY INFECTIONS OF UNSPECIFIED SITE, AFTERCARE CHECK-UP NO DISEASE, ALLERGIC RHINITIS, ALLERGY, ALOPECIA, ANAL FISSURE, ANEMIA, ANEMIA COMPLICATING PREGNANCY, ANEMIA OTHER/UNSPECIFIED, ANKLE SPRAIN, ANTEPARTUM ANEMIA, ANTEPARTUM INFECTIONS OF GENITOURINARY TRACT, ANTEPARTUM THYROID DYSFUNCTION, ANXIETY STATES, ARTIFICIAL INSEMINATION, ASPIRATION CURETTAGE FOLLOWING DELIVERY OR ABORTION, ASPIRATION OF OVARY, ASTHMA, ASYMPTOMATIC BACTERIURIA IN PREGNANCY, BACK SYMPTOMS/COMPLAINTS, BACKACHE, BENIGN NEOPLASM OF SKIN, BLEPHARITIS, BREAST PAIN, BREAST SCREENING FOR MALIGNANT NEOPLASMS, CALCUL.OF GALLBLADDER WITHOUT CHOLECYSTITIS, CALCULUS OF GALLBLADDER WITHOUT MENTION OF CHOLECYSTITIS, CANDIDIASIS OF VULVA AND VAGINA, CARPAL TUNNEL SYNDROME, CELLULITIS AND ABSCESS OF UNSPECIFIED SITES, CERCLAGE-MC DONALD, CERCLAGE-UNSPECIFIED, CERVICAL INCOMPETENCE, CERVICAL SHORTENING COMPLICATING PREGNANCY, CERVICALGIA, CESAREAN SECTION AND REMOVAL OF FETUS, CHALAZION, CHEST PAIN, CHEST SYMPTOMS/COMPLAINTS, CHRONIC LYMPHOCYTIC THYROIDITIS, COAGULATION DEFECTS, COLITIS, CONSTIPATION, CONSULTATION, CONTACT DERMATITIS AND OTHER ECZEMA, CONTRACEPTIVE MANAGEMENT, CONTUSION OF BACK, CONTUSION OF UNSPECIFIED SITE, CORPUS LUTEUM CYST OR HEMATOMA, COUGH, CYSTITIS, DEBILITY, DERMATOPHYTOSIS OF FOOT, DERMATOPHYTOSIS OF NAIL, DIAGNOSTIC ULTRASOUND, DIAGNOSTIC ULTRASOUND OF ABDOMEN AND RETROPERITONEUM, DIAGNOSTIC ULTRASOUND OF GRAVID UTERUS, DIAGNOSTIC ULTRASOUND OF URINARY SYSTEM, DIARRHEA, DILATION AND CURETTAGE FOLLOWING DELIVERY OR ABORTION, DISEASES AND CONDITIONS OF THE TEETH AND SUPPORT.STRUCTURES, DISTURBANCE OF SKIN SENSATION, DIZZINESS AND GIDDINESS, DYSCHROMIA, DYSPEPSIA AND OTHER SPECIFIED DISORDERS OF FUNCTION OF STOMACH, DYSPLASIA OF CERVIX, DYSPNEA AND RESPIRATORY ABNORMALITIES, DYSURIA, E. T., ENCOUNTER FOR ASSISTED REPRODUCTIVE FERTILITY PROCEDURE CYCLE, ENCOUNTER FOR OTHER SPECIFIED PROCREATIVE MANAGEMENT, ENCOUNTERS FOR ADMINISTRATIVE PURPOSES, ENCOUNTERS FOR DISABILITY EXAMINATION, ENLARGEMENT OF LYMPH NODES, EPILEPSY, EPISTAXIS, EROSION AND ECTROPION OF CERVIX, ESOPHAGITIS, ESSENTIAL HYPERTENSION, EXCESSIVE VOMITING IN PREGNANCY, FAMILY HISTORY OF DIABETES MELLITUS, FAMILY HISTORY OF MALIGNANT NEOPLASM OF BREAST, FEVER, FILL OUT FORMS, FLANK SYMPTOMS/COMPLAINTS, FOLLICULAR CYST OF OVARY, FOLLOW-UP EXAMINATION, GENETIC COUNSELING ON PROCREATIVE MANAGEMENT, GYNECOLOGICAL EXAMINATION, HABITUAL ABORTER, HEADACHE, HEARTBURN, HEMATURIA, HEMORRHAGE OF RECTUM AND ANUS, HEMORRHOIDS, HERPES SIMPLEX WITHOUT MENTION OF COMPLICATION, HORDEOLUM AND OTHER DEEP INFLAMMATION OF EYELID, HYDRONEPHROSIS, HYPEREMESIS GRAVIDARUM WITH METABOLIC DISTURBANCE, HYSTEROSCOPY, I. V. F., IMPACTED CERUMEN, IN VITRO FERTILIZATION OR TEST TUBE PREGNANCY, INFECTIONS OF GENITOURINARY TRACT IN PREGNANCY, INFECTIOUS DIARRHEA, INFECTIVE OTITIS EXTERNA, INFERTILITY, INFERTILITY/SUBFERTILITY, INFLUENZA, INGROWING NAIL, INJECT.OR INFUSION OF OTHER THERAPEUTIC OR PROPHYLACTIC SUBS., INJECTION OF ANTIBIOTIC, INJECTION OF RH IMMUNE GLOBULIN, INJURIES, INSECT BITE, IRON DEFICIENCY ANEMIA, IRREGULAR MENSTRUAL CYCLE, ISSUE OF MEDICAL CERTIFICATE, ISSUE OF REPEAT PRESCRIPTION, KNEE SYMPTOMS/COMPLAINTS, LABORATORY EXAMINATION, LAPAROSCOPIC APPENDECTOMY, LAPAROSCOPIC CHOLECYSTECTOMY 1992, LAPAROSCOPY, LATE EFFECT OF COMPLICATION OF PREGNANCY, LATE EFFECT OF INJURY TO CRANIAL NERVE, LEG.INDUCED ABORTION, LEUKORRHEA, LOW BACK COMPLT.W/O RADIATION, LUMBAGO, MANUAL EXAMINATION OF BREAST, MIGRAINE, MILD HYPEREMESIS GRAVIDARUM, MISSED ABORTION, MOTHER WITH SINGLE LIVEBORN, MYALGIA AND MYOSITIS, MYOPIA, NAUSEA, NAUSEA AND VOMITING, NECK SPRAIN, NO DISEASE, NONSPECIFIC ELEVATION OF LEVELS OF TRANSAMINASE OR LDH, NONSPECIFIC FINDINGS ON EXAMINATION OF BLOOD, NORMAL PREGNANCY, NOTHING ABNORMAL FOUND, OBESITY, OBSERVATION—PATIENT UNDER OBSERVATION, OBSERVATION AND EVALUATION FOR SUSPECTED CONDITIONS, OBSERVATION FOLLOWING OTHER ACCIDENT, OBSERVATION FOR UNSPECIFIED SUSPECTED CONDITION, ORAL APHTHAE, ORAL CONTRACEPTION COUNSELING, ORAL CONTRACEPTION PRESCRIPTION, OTALGIA, OTHER, OTHER ABNORMAL PRODUCTS OF CONCEPTION, OTHER ACNE, OTHER ADVANCED MATERNAL AGE, OTHER AND UNSPEC.NONINFECTIOUS GASTROENTERITIS AND COLITIS, OTHER AND UNSPECIFIED ESCHERICHIA COLI, OTHER AND UNSPECIFIED INJURY TO UNSPECIFIED SITE, OTHER AND UNSPECIFIED OVARIAN CYST, OTHER ATOPIC DERMATITIS AND RELATED CONDITIONS, OTHER B-COMPLEX DEFICIENCIES, OTHER DIAGNOSTIC ULTRASOUND, OTHER DISORDERS OF LACRIMAL GLAND, OTHER DYSPNEA AND RESPIRATORY ABNORMALITY, OTHER FETAL CONDITIONS, OTHER GENITOURINARY INSTILLATION, OTHER ILL-DEFINED AND UNKNOWN CAUSES OF MORBIDITY AND MORTALITY, OTHER ILL-DEFINED CONDITIONS, OTHER MALAISE AND FATIGUE, OTHER NONTHROMBOCYTOPENIC PURPURAS, OTHER OVARIAN HYPERFUNCTION, OTHER SPECIFIED DISEASES OF HAIR AND HAIR FOLLICLES, OTHER SPECIFIED NONINFLAMMATORY DISORDERS OF VAGINA, OTHER SPECIFIED VIRAL WARTS, OTHER THALASSEMIA, PAIN IN JOINT, PAIN IN LIMB, PALPITATIONS, PERSONAL HISTORY OF ALLERGY TO ANALGESIC AGENT, PERSONAL HISTORY OF ALLERGY TO OTHER ANTIBIOTIC AGENT, PERSONAL HISTORY OF ALLERGY TO PENICILLIN, PERSONAL HISTORY OF ALLERGY TO SPECIFIED MEDICINAL AGENTS, PITYRIASIS VERSICOLOR, POLYCYSTIC OVARIES, POSTCOITAL BLEEDING, PREGNANCY EXAMINATION OR TEST, PREGNANCY HIGH RISK, PREGNANCY WITH HISTORY OF PRE-TERM LABOR, PREGNANCY: CONFIRMED, PREGNANT STATE, PROTEINURIA, PRURITUS OF GENITAL ORGANS, PYELONEPHRITIS, RASH AND OTHER NONSPECIFIC SKIN ERUPTION, REGIONAL ENTERITIS OF UNSPECIFIED SITE, RELEASE OF TORSION OF OVARY, RENAL COLIC, ROUTINE GENERAL MEDICAL EXAMINATION AT A HEALTH CARE FACILITY, SCANTY OR INFREQUENT MENSTRUATION, SCIATICA, SCREENING FOR OTHER SPECIFIED CONDITIONS, SEBACEOUS CYST, SEBORRHEIC DERMATITIS, SHOULDER SYMPTOMS/COMPLAINTS, SIGNS AND SYMPTOMS IN BREAST, SPONTANEOUS ABORTION, SUPERVISION OF HIGH RISK PREGNANCY RESULTING FROM ASSISTED REPRODUCTIVE TECHNOLOGY, SUPERVISION OF HIGH-RISK PREGN.WITH POOR OBSTETRIC HISTORY, SUPERVISION OF HIGH-RISK PREGNANCY, SUPERVISION OF HIGH-RISK PREGNANCY WITH HISTORY OF ABORTION, SUPERVISION OF NORMAL FIRST PREGNANCY, SUPERVISION OF OTHER HIGH-RISK PREGNANCY, SUPERVISION OF OTHER NORMAL PREGNANCY, SYNCOPE AND COLLAPSE, SYNOVITIS AND TENOSYNOVITIS, TEMPOROMANDIBULAR JOINT DISORDERS, THREATENED ABORTION, THREATENED PREMATURE LABOR, THYROTOXICOSIS WITH OR WITHOUT GOITER, TOBACCO USE DISORDER, TORSION OF OVARY, TWIN PREGNANCY, U.R.I., UNKNOWN CATEGORY, UNKNOWN OR UNSPECIFIED CAUSE OF MORBIDITY, UNSP.VIRAL INFECT.IN CONDITIONS CLASSIF.ELSEWHERE, UNSPECIFIED ABORTION, UNSPECIFIED ACQUIRED HYPOTHYROIDISM, UNSPECIFIED ANTEPARTUM HEMORRHAGE, UNSPECIFIED HEMORRHAGE IN EARLY PREGNANCY, UNSPECIFIED HYPERTROPHIC AND ATROPHIC CONDITIONS OF SKIN, UNSPECIFIED VOMITING OF PREGNANCY, URINARY TRACT INFECTION, URTICARIA, UTERINE LEIOMYOMA, UTERINE SCAR FROM PREV.SURGERY, VAGINAL HEMATOMA, VAGINITIS AND VULVOVAGINITIS, VARICOSE VEINS OF LOWER EXTREMITIES, VIRAL WARTS, VITAMIN D DEFICIENCY, VOICE DISTURBANCE, VOLUME DEPLETION DISORDER, VOMITING, VOMITING/NAUSEA OF PREGNANCY, wherein the diagnoses correspond to any of the time windows F0, F1, F2, M1, M2, M3, M4, M5, P1, P2, and P3 defined in the Examples section that follows.

In some embodiments of the present invention the parameters comprise at least one parameter indicative of a pharmaceutical prescribed for said subject. Representative examples of prescribed pharmaceuticals which can be used as parameters according to some embodiments of the present invention include, without limitation, ACAMOL CPL 500 MG 21, ACAMOLI FRUIT S/F SYR 125 mg/5 mL 100 mL, ACAMOLI STRAW. S/F SYR 125 mg/5 mL 100 mL, ACAMOLI SUP 150 MG 12, ACIDOPHILUS PROBIOTIC CAP BOX 30, ADVIL LIQUI-GELS CAP 200 MG 16, ADVIL LIQUI-GELS CAP 200 MG 40, AEROVENT SOL 0.25 mg/1 mL 20 mL, AFLUMYCIN CR CF 20 GM, AGISTEN 1 VAGINAL TAB 500 MG, AGISTEN ALOEVERA CR 1% 20 GM, AGISTEN V VAG. TAB 200 MG 3, AGISTEN VAG TAB 200 mg 3, AGISTEN VAGINAL CR 2% 20 GM, AKTIFERRIN SOFT CAP 34 mg 30, AKTIFERRIN-F SOFT CAP 30, ALCOHOL SPT 70% 100 ML, ALCOTINT SPT 70% 100 ML, ALLERGYX TAB 10 MG 10, AMOXICLAV TEVA TAB 875 mg 14, ANAESTHETIC AUR 10 mL, AUGMENTIN BID TAB 875 MG 14, AUGMENTIN TAB 500 MG 20, AVAMYS AQ. NASAL 120 INH SPR 27.5 MCG, BABY D3 DRP 2001U 10 ML, BACTROBAN OIN 2% 15 g, BEDODEKA INJ 1000 MCG/1 ML 100, BETACORTEN CR 0.1% 15 GM, BEVITEX SUBLINGUAL TAB 1000 MCG 30, CEFOVIT FORTE CAP 500 mg 20, CETROTIDE VIA 0.25 mg 7, CIPRALEX, CLARINASE REPETABS TAB 14, CLEXANE INJ 40 mg/0.4 mL 2×0.4 mL, CLEXANE SAFETY LOCK INJ 40 MG/0.4 ML 2, CLEXANE SAFETY LOCK INJ 60 MG/0.6 ML 2, CLOTREE-TEVA VAG OVL 200 MG 3, CLOTRIMAZOLE, COLCHICINE TAB 0.5 MG 30, COMAGIS CR CF 15 GM, CRINONE VAG. GEL 8% 15U, DALACIN VAGINAL OVL 100 MG 3, D-DROPS DRP 200 UNIT 10 mL, DECAPEPTYL INJ 0.1 mg/1 mL 7, DERMACOMBIN CR CF 15 GM, DESOREN AUR CF BOT 5 ML, DETHAMYCIN EYE, DEXAMOL COLD 30D+20N CPL, DEXAMOL CPL 500 MG 20, DEX-OTIC AUR CF BOT 5 ML, DICLECTIN DR TAB 10 mg/10 mg 100, DOXYLIN TAB 100 MG 10, D-TABS TAB 400 IU 90, DUPHASTON TAB 10 MG BOX 20, ELASTAN TREAT CR 75 ML, ELOCOM CR 0.1% 15 GM, ELTROXIN TAB 100 MCG 100, ELTROXIN TAB 50 MCG 100, EMLA CR 5% 30 GM, ENDOMETRIN VAGINAL TAB 100 MG BOX 30, ESTROFEM TAB 2 MG 28, EUTHYROX TAB 100 mcg 100, EUTHYROX TAB 50 mcg 100, FAMOTIDINE-TEVA TAB 20 MG 30, FEMINA SOAP LIQ 330 ML, FENISTIL GEL 0.1% 30 GM, FERRIFOL TAB CF BOX 30, FERRIPEL-3 SYR 50 MG/5 ML 110 ML, FERROGRAD FOLIC TAB CF BOX 30, FLIXONASE NASAL SPR 0.05%, FOLEX 400 TAB CF BOX 30, FOLI 5, FOLIC ACID, FORIC PREGNANCY TAB CF BOX 30, FOROL TAB 30, GENTLE IRON CAP 25 MG BOX 90, GESTON INJ 50 mg/1 mL 1 mL, GLYCERIN, GONAL-F-PEN INJ 3001U 0.5 ML, GONAL-F-PEN INJ 4501U 0.75 ML, GONAL-F-PEN INJ 900 IU 1.5 ML, GYNO-DAKTARIN VAG. CAP 400 mg 3, GYNO-PEVARYL VAG TAB 150 MG 3, HYDROAGISTEN CR CF 15 GM, IKACLOMIN TAB 50 MG 10, IRON BIS-GLYCINATE DH CAP 90, IRON PLUS FEMINA, KAMRHO-D I.M. INJ 300 mcg 2 mL, LANTON CAP 30 MG BOX 28, LEMOCIN CHERRY SUGAR FREE LOZ CF 24, LEMOCIN LEMON SUGAR FREE LOZ CF 24, LORATADINE, MAALOX SUS CF BOT 355 ML, MAARAZ 9M:OMEGA-3+PRENATA, MACRODANTIN 29/M CAP 100 MG 30, MAXITROL OCC CF TUB 3.5 GM, MELIANE TAB 21, MENOGON < >< > INJ 751U/751U 10, MENOPUR MULTIDOSE INJ 1200 UNIT, MENOPUR MULTIDOSE INJ 600 UNIT, MENOPUR VIA 751U 10, MICROPIRIN TAB 100 MG 28, MICROPIRIN TAB 75 MG 28, MICROPIRIN TAB 75 mg 30, MOXYPEN CAP 500 MG 10, MOXYPEN CAP 500 mg 20, MOXYVIT CAP 500 MG 20, NASOCORT AQUA NASAL SPR 64 MCG, NORMALAX PWD 240 GM, NUROFEN LIQUID CAP 200 MG BOX 20, NUROFEN LIQUID CAP 200 MG BOX 40, NUROFEN ORANGE CHILD SUS 100 MG/5 ML 100 ML, NUROFEN ORANGE CHILD SUS 100 MG/5 ML 150 ML, NUROFEN STRAWB.CHILD SUS 100 MG/5 ML 150 ML, NUROFEN STRAWB.PED. SUS 100 mg/5 mL 100 mL, NUSSIDEX TAB CF 20, OMEGA D3 9 MONTHS, OMEGA-3 9 MONTHS, OMEPRADEX CPL 20 mg 30, OPTALGIN CPL 500 MG 21, OPTALGIN CPL 500 mg 42, OPTALGIN DRP 500 MG/1 ML 10 ML, OPTALGIN TAB 500 MG 20, ORACORT E PAS TUB 5 g, ORGALUTRAN SYRI.< >< > INJ 0.25 MG 0.5 ML, OTHER, OTIDIN AUR CF 10 GM, OTRIMER SPR 0.9% 50 ML, OTRIVIN MENTHOL M.D SPR 0.1% 10 ML, OTRIVIN NASAL M.D. SPR 0.1% 10 ML, OTRIVONIM BABY WIP PKG 2×36U, OVITRELLE PREFILLED INJ 250 mcg, PAPAVERINE TAB 80 MG 30, PARO TOOTHBRUSH MEDIC NO. 726, PEN-RAFA VK CPL 500 mg 40, PHENIMYXIN COL 8 ML, POLYCUTAN CR 15 GM, PRAMIN INJ 10 MG/2 ML 5, PRAMIN SUP 20 MG 6, PRAMIN TAB 10 MG 30, PREDNISONE TAB 20 MG 30, PREDNISONE TAB 5 MG 30, PRE-GENTLY BADATZ, PRENATAL 9 MONTHS, PRENATAL MULTIVITAMINS D.H TAB 100, PRENATAL MULTIVITAMINS D.H TAB 30, PRENATAL PLUS CF TAB CF BOX 30, PRIMOLUT NOR TAB 5 MG 20, PROCTO-GLYVENOL CR CF TUB 30 GM, PROGESTERON RETARD 29/M INJ 500 MG/2 ML 3, PROGYNOVA TAB 2 MG 28, PUREGON INJ 300 IU 0.36 ML, PUREGON INJ 900 IU, REOLIN EFFERV. TAB 200 MG 30, SEDURAL TAB 100 MG 30, SERETIDE DISKUS INH 50/250 MCG U, STREP A TEST-QUICKVUE IN-LINE, STREPSILS HONEY-LEMON LOZ CF 24, SYMBICORT TURBUHALER INH 160 mcg/4.5 mcg, SYNTHOMYCINE OCC 5% 3.6 GM, TEEJEL GEL CF 10 GM, TELFAST TAB 180 MG 15, TEVACUTAN CR 15 g, TEVADERM CR CF 15 GM, THYMI SYR BOT 100 ML, THYMOLI SYR BOT 100 ML, TIPTIPOT FERRIPEL-3 DRP 50 mg/1 mL 15 mL, TIPTIPOT NOVIMOL SUS 100 mg/1 mL 15 mL, TRIBEMIN TAB 20, TRIDERM CR CF 15 GM, UTROGESTAN ORAL/VAG CAP 100 mg 30, UTROGESTAN ORAL/VAG CAP 200 mg 15, VAXIGRIP VAC, VENTOLIN CFC FREE INH 0.1 MG/INH, VENTOLIN RESPIR. SOL 5 mg/1 mL 20 mL, VIT B1, VIT B12, VIT.B12+FOLIC A.SUBLING. TAB CF 60, VITA-CAL+D, VITAMIDYNE D DRP 200 IU 10 ML, VITAMIN D3 D.H DRP 400 UNIT 15 ML, VOLTAREN EMULGEL GEL 1% 100 GM, YES OR NO DIRECT KIT 1, ZINNAT TAB 250 MG 10, ZINNAT TAB 500 MG 10, ZOFRAN TAB 4 MG 10, ZOFRAN TAB 8 MG 10, ZOVIRAX CR 5% 2 GM, wherein the pharmaceutical are prescribed during any of the time windows F0, F1, F2, M1, M2, M3, M4, M5, P1, P2, and P3 defined in the Examples section that follows.

In some embodiments of the present invention one or more of the parameters is indicative of a GTT applied to the subject. When the subject has been previously pregnant, one of the parameters is a result of a GTT applied to said subject during a previous pregnancy, e.g., the most recent previous pregnancy or the next-to-most recent previous pregnancy.

A list of parameters that relate to GTT and which are contemplated according to some embodiments of the present invention, includes, without limitation, 100 g GTT 0 minutes at the first previous pregnancy, 100 g GTT 0 minutes at the 2nd previous pregnancy, 100 g GTT 0 minutes at 3rd previous pregnancy, 100 g GTT 120 minutes at the 1st previous pregnancy, 100 g GTT 120 minutes at the 2nd previous pregnancy, 100 g GTT 120 minutes at the 3rd previous pregnancy, 100 g GTT 180 minutes at the 1st previous pregnancy, 100 g GTT 180 minutes at the 2nd previous pregnancy, 100 g GTT 180 minutes at the 3rd previous pregnancy, 100 g GTT 60 minutes at the 1st previous pregnancy, 100 g GTT 60 minutes at the 2nd previous pregnancy, 100 g GTT 60 minutes at the 3rd previous pregnancy, 50 g GTT at the 1st previous pregnancy, 50 g GTT at the 2nd previous pregnancy, 50 g GTT at the 3rd previous pregnancy.

In some embodiments of the present invention one or more of the parameters is the Body Mass Index (BMI) of the subject, as measured during any of the time windows F0, F1, F2, M1, M2, M3, M4, M5, P1, P2, and P3 defined in the Examples section that follows.

In some embodiments of the present invention one of the parameters is the number of previous births delivered by the subject.

In some embodiments of the present invention one or more of the parameters is the diastolic blood pressure sampled during any of the time windows F0, F1, F2, M1, M2, M3, M4, M5, P1, P2, and P3 defined in the Examples section that follows, and in some embodiments of the present invention one or more of the parameters is the systolic blood pressure sampled during any of the time windows F0, F1, F2, M1, M2, M3, M4, M5, P1, P2, and P3 defined in the Examples section that follows.

Referring again to FIG. 1, the method proceeds to 12 at which a computer readable medium storing a machine learning procedure is accessed. The machine learning procedure is trained for predicting likelihoods for gestational diabetes.

As used herein the term “machine learning” refers to a procedure embodied as a computer program configured to induce patterns, regularities, or rules from previously collected data to develop an appropriate response to future data, or describe the data in some meaningful way.

Representative examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

Following is an overview of some machine learning procedures suitable for the present embodiments.

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

An advantage of a support vector machine is that once the support vectors have been identified, the remaining observations can be removed from the calculations, thus greatly reducing the computational complexity of the problem. An SVM typically operates in two phases: a training phase and a testing phase. During the training phase, a set of support vectors is generated for use in executing the decision rule. During the testing phase, decisions are made using the decision rule. A support vector algorithm is a method for training an SVM. By execution of the algorithm, a training set of parameters is generated, including the support vectors that characterize the SVM. A representative example of a support vector algorithm suitable for the present embodiments includes, without limitation, sequential minimal optimization.

In KNN analysis, the affinity or closeness of objects is determined. The affinity is also known as distance in a feature space between objects. Based on the determined distances, the objects are clustered and an outlier is detected. Thus, the KNN analysis is a technique to find distance-based outliers based on the distance of an object from its kth-nearest neighbors in the feature space. Specifically, each object is ranked on the basis of its distance to its kth-nearest neighbors. The farthest away object is declared the outlier. In some cases the farthest objects are declared outliers. That is, an object is an outlier with respect to parameters, such as, a k number of neighbors and a specified distance, if no more than k objects are at the specified distance or less from the object. The KNN analysis is a classification technique that uses supervised learning. An item is presented and compared to a training set with two or more classes. The item is assigned to the class that is most common amongst its k-nearest neighbors. That is, compute the distance to all the items in the training set to find the k nearest, and extract the majority class from the k and assign to item.

Association rule algorithm is a technique for extracting meaningful association patterns among features.

The term “association”, in the context of machine learning, refers to any interrelation among features, not just ones that predict a particular class or numeric value. Association includes, but it is not limited to, finding association rules, finding patterns, performing feature evaluation, performing feature subset selection, developing predictive models, and understanding interactions between features.

The term “association rules” refers to elements that co-occur frequently within the datasets. It includes, but is not limited to association patterns, discriminative patterns, frequent patterns, closed patterns, and colossal patterns.

A usual primary step of association rule algorithm is to find a set of items or features that are most frequent among all the observations. Once the list is obtained, rules can be extracted from them.

The aforementioned self-organizing map is an unsupervised learning technique often used for visualization and analysis of high-dimensional data. Typical applications are focused on the visualization of the central dependencies within the data on the map. The map generated by the algorithm can be used to speed up the identification of association rules by other algorithms. The algorithm typically includes a grid of processing units, referred to as “neurons”. Each neuron is associated with a feature vector referred to as observation. The map attempts to represent all the available observations with optimal accuracy using a restricted set of models. At the same time the models become ordered on the grid so that similar models are close to each other and dissimilar models far from each other. This procedure enables the identification as well as the visualization of dependencies or associations between the features in the data.

Feature evaluation algorithms are directed to the ranking of features or to the ranking followed by the selection of features based on their impact.

Information gain is one of the machine learning methods suitable for feature evaluation. The definition of information gain requires the definition of entropy, which is a measure of impurity in a collection of training instances. The reduction in entropy of the target feature that occurs by knowing the values of a certain feature is called information gain. Information gain may be used as a parameter to determine the effectiveness of a feature in explaining the response to the treatment. Symmetrical uncertainty is an algorithm that can be used by a feature selection algorithm, according to some embodiments of the present invention. Symmetrical uncertainty compensates for information gain's bias towards features with more values by normalizing features to a [0,1] range.

Subset selection algorithms rely on a combination of an evaluation algorithm and a search algorithm. Similarly to feature evaluation algorithms, subset selection algorithms rank subsets of features. Unlike feature evaluation algorithms, however, a subset selection algorithm suitable for the present embodiments aims at selecting the subset of features with the highest impact on predicting likelihood for gestational diabetes, while accounting for the degree of redundancy between the features included in the subset. The benefits from feature subset selection include facilitating data visualization and understanding, reducing measurement and storage requirements, reducing training and utilization times, and eliminating distracting features to improve classification.

Two basic approaches to subset selection algorithms are the process of adding features to a working subset (forward selection) and deleting from the current subset of features (backward elimination). In machine learning, forward selection is done differently than the statistical procedure with the same name. The feature to be added to the current subset in machine learning is found by evaluating the performance of the current subset augmented by one new feature using cross-validation. In forward selection, subsets are built up by adding each remaining feature in turn to the current subset while evaluating the expected performance of each new subset using cross-validation. The feature that leads to the best performance when added to the current subset is retained and the process continues. The search ends when none of the remaining available features improves the predictive ability of the current subset. This process finds a local optimum set of features.

Backward elimination is implemented in a similar fashion. With backward elimination, the search ends when further reduction in the feature set does not improve the predictive ability of the subset. The present embodiments contemplate search algorithms that search forward, backward or in both directions. Representative examples of search algorithms suitable for the present embodiments include, without limitation, exhaustive search, greedy hill-climbing, random perturbations of subsets, wrapper algorithms, probabilistic race search, schemata search, rank race search, and Bayesian classifier.

A decision tree is a decision support algorithm that forms a logical pathway of steps involved in considering the input to make a decision.

The term “decision tree” refers to any type of tree-based learning algorithms, including, but not limited to, model trees, classification trees, and regression trees.

A decision tree can be used to classify the datasets or their relation hierarchically. The decision tree has tree structure that includes branch nodes and leaf nodes. Each branch node specifies an attribute (splitting attribute) and a test (splitting test) to be carried out on the value of the splitting attribute, and branches out to other nodes for all possible outcomes of the splitting test. The branch node that is the root of the decision tree is called the root node. Each leaf node can represent a classification (e.g., whether a particular parameter influences on the likelihood for gestational diabetes) or a value (e.g., the predicted likelihood for gestational diabetes). The leaf nodes can also contain additional information about the represented classification such as a confidence score that measures a confidence level in the represented classification (i.e., the accuracy of the prediction).

Regression techniques which may be used in accordance with some embodiments the present invention include, but are not limited to linear Regression, Multiple Regression, logistic regression, probit regression, ordinal logistic regression ordinal Probit-Regression, Poisson Regression, negative binomial Regression, multinomial logistic Regression (MLR) and truncated regression.

A logistic regression or log it regression is a type of regression analysis used for predicting the outcome of a categorical dependent variable (a dependent variable that can take on a limited number of values, whose magnitudes are not meaningful but whose ordering of magnitudes may or may not be meaningful) based on one or more predictor variables. Logistic regression may also predict the probability of occurrence for each data point. Logistic regressions also include a multinomial variant. The multinomial logistic regression model is a regression model which generalizes logistic regression by allowing more than two discrete outcomes. That is, it is a model that is used to predict the probabilities of the different possible outcomes of a categorically distributed dependent variable, given a set of independent variables (which may be real-valued, binary-valued, categorical-valued, etc.). For binary-valued variables, a cutoff between the 0 and 1 associations is typically determined using the Yuden Index.

A Bayesian network is a model that represents variables and conditional interdependencies between variables. In a Bayesian network variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some embodiments, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future predictions of the likelihood for gestational diabetes. An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Instance-based techniques generate a new model for each instance, instead of basing predictions on trees or networks generated (once) from a training set.

The term “instance”, in the context of machine learning, refers to an example from a dataset.

Instance-based techniques typically store the entire dataset in memory and build a model from a set of records similar to those being tested. This similarity can be evaluated, for example, through nearest-neighbor or locally weighted methods, e.g., using Euclidian distances. Once a set of records is selected, the final model may be built using several different techniques, such as the naive Bayes.

Neural networks are a class of algorithms based on a concept of inter-connected “neurons.” In a typical neural network, neurons contain data values, each of which affects the value of a connected neuron according to connections with pre-defined strengths, and whether the sum of connections to each particular neuron meets a pre-defined threshold. By determining proper connection strengths and threshold values (a process also referred to as training), a neural network can achieve efficient recognition of images and characters. Oftentimes, these neurons are grouped into layers in order to make connections between groups more obvious and to each computation of values. Each layer of the network may have differing numbers of neurons, and these may or may not be related to particular qualities of the input data.

In one implementation, called a fully-connected neural network, each of the neurons in a particular layer is connected to and provides input value to those in the next layer. These input values are then summed and this sum compared to a bias, or threshold. If the value exceeds the threshold for a particular neuron, that neuron then holds a positive value which can be used as input to neurons in the next layer of neurons. This computation continues through the various layers of the neural network, until it reaches a final layer. At this point, the output of the neural network routine can be read from the values in the final layer. Unlike fully-connected neural networks, convolutional neural networks operate by associating an array of values with each neuron, rather than a single value. The transformation of a neuron value for the subsequent layer is generalized from multiplication to convolution.

The machine learning procedure used according to some embodiments of the present invention is a trained machine learning procedure, which provides output that is related non-linearly to the parameters with which it is fed.

A machine learning procedure can be trained according to some embodiments of the present invention by feeding a machine learning training program with parameters that characterizes each of a cohort of female subjects that has been diagnosed as either having or not having gestational diabetes. Once the data are fed, the machine learning training program generates a trained machine learning procedure which can then be used without the need to re-train it.

For example, when it is desired to employ decision trees, machine learning training program learns the structure of each tree in a plurality of decision trees (e.g., how many nodes there are in each tree, and how these are connected to one another), and also selects the decision rules for split nodes of each tree. At least a portion of the decision rules relate to one or more of the parameters that characterize the female subject. A simple decision rule may be a threshold for the value of a particular parameter, but more complex rules, relating to more than one parameters are also contemplated. The machine learning training program also accumulates data at the leaves of the trees. The structures of the trees, the decision rules for the split nodes, and the data at the leaves are all selected by the machine learning training program, automatically and typically without user intervention, such that the parameters at the root of the trees provide the likelihood for gestational diabetes at the leaves of the trees. The final result of the machine learning training program in this case is a set of trees, where the structures, the decision rules for split nodes, and leaf data for each trees are defined by the machine learning training program.

The method proceeds to 13 at which the trained machine learning procedure is fed with the parameters, and to 14 at which an output indicative of the likelihood that the subject has, or expected to develop, gestational diabetes, is received from the procedure. In some embodiments of the present invention the method proceeds to 15 at which a report predating to the likelihood is generated. The report can be displayed on a display device or transmitted to a computer readable medium.

The method ends at 16.

The inventors found that selected operations of the method can also be used for screening. For example, the method can be used for determining whether to apply a GTT to a female subject that has been previously pregnant. In these embodiments, the parameters that are obtained at 11 comprise a result of a GTT applied to the subject during a previous pregnancy, and the likelihood that is received from the procedure is used for determining whether or not to apply the GTT to the subject. Specifically, when the likelihood is below a predetermined threshold (e.g., below 0.6 or below 0.5 or below 0.4 or below 0.3 or below 0.2 or below 0.1), the method can generate an output recommending not to apply the GTT to the subject, and when the likelihood is above the predetermined threshold, the method can generate an output recommending to apply the GTT to the subject. As demonstrated in the Example section that follows, it was unexpectedly found by the inventors that a GTT, e.g., a 1 h 50 g GTT, performed in previous pregnancies is far more predictive than, for example, a history of GDM. The advantage of using GTT in previous pregnancies as a predictor for the likelihood is that the method of the present embodiments is more cost-effective and efficient than the GTT, and can therefore be used as a selective screening method. The inventors found that avoiding 50% of the GTTs of patients who previously did a GTT would result in only 5% miss rate when diagnosing GDM according to the traditional guidelines. Accurate selective screening is advantageous since it can both reduces costs and physical inconvenience for women at low risk for GDM development.

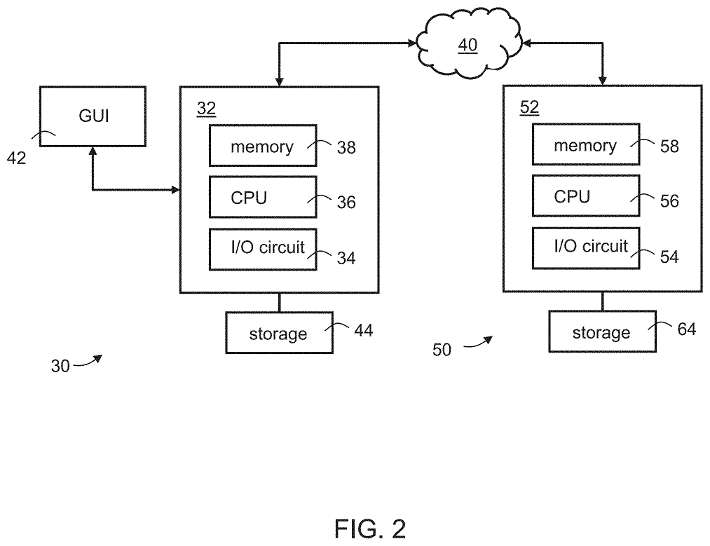

The prediction of likelihood for gestational diabetes can be executed according to some embodiments of the present invention by a server-client configuration, as will now be explained with reference to FIG. 2.

FIG. 2 illustrates a client computer 30 having a hardware processor 32, which typically comprises an input/output (I/O) circuit 34, a hardware central processing unit (CPU) 36 (e.g., a hardware microprocessor), and a hardware memory 38 which typically includes both volatile memory and non-volatile memory. CPU 36 is in communication with I/O circuit 34 and memory 38. Client computer 30 preferably comprises a user interface, e.g., a graphical user interface (GUI), 42 in communication with processor 32. I/O circuit 34 preferably communicates information in appropriately structured form to and from GUI 42. Also shown is a server computer 50 which can similarly include a hardware processor 52, an I/O circuit 54, a hardware CPU 56, a hardware memory 58. I/O circuits 34 and 54 of client 30 and server 50 computers preferable operate as transceivers that communicate information with each other via a wired or wireless communication. For example, client 30 and server 50 computers can communicate via a network 40, such as a local area network (LAN), a wide area network (WAN) or the Internet. Server computer 50 can be in some embodiments be a part of a cloud computing resource of a cloud computing facility in communication with client computer 30 over the network 40.

GUI 42 and processor 32 can be integrated together within the same housing or they can be separate units communicating with each other. GUI 42 can optionally and preferably be part of a system including a dedicated CPU and I/O circuits (not shown) to allow GUI 42 to communicate with processor 32. Processor 32 issues to GUI 42 graphical and textual output generated by CPU 36. Processor 32 also receives from GUI 42 signals pertaining to control commands generated by GUI 42 in response to user input. GUI 42 can be of any type known in the art, such as, but not limited to, a keyboard and a display, a touch screen, and the like. In preferred embodiments, GUI 42 is a GUI of a mobile device such as a smartphone, a tablet, a smartwatch and the like. When GUI 42 is a GUI of a mobile device, the CPU circuit of the mobile device can serve as processor 32 and can execute the method optionally and preferably by executing code instructions.

Client 30 and server 50 computers can further comprise one or more computer-readable storage media 44, 64, respectively. Media 44 and 64 are preferably non-transitory storage media storing computer code instructions for executing the method of the present embodiments, and processors 32 and 52 execute these code instructions. The code instructions can be run by loading the respective code instructions into the respective execution memories 38 and 58 of the respective processors 32 and 52. Storage media 64 preferably also store one or more databases including a database of psychologically annotated olfactory perception signatures as further detailed hereinabove.

In operation, processor 32 of client computer 30 displays on GUI 42 a questionnaire and a set of questionnaire controls, such as, but not limited to, a slider, a dropdown menu, a combo box, a text box and the like. A representative example of a displayed questionnaire 60 and a set of controls 62 is shown in FIG. 6C. A female subject can enter response parameters using the questionnaire controls displayed on GUI 42.

Processor 32 receives the response parameters from GUI 42 and typically transmits these parameters to server computer 50 over network 40. Media 64 can store a machine learning procedure trained for predicting likelihoods for gestational diabetes. Server computer 50 can access media 64, feed the stored procedure with the parameters received from client computer 30, and receive from the procedure an output indicative of the likelihood that the female subject that is characterized by the parameters has, or is expected to develop, gestational diabetes. Server computer 50 can also transmit to client computer 30 the obtained likelihood, and client computer 30 can display this information on GUI 42.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

GDM is defined as glucose intolerance that is first recognized in pregnancy. GDM is a common complication of pregnancy, occurring in 3%-9% of pregnancies [1], typically diagnosed between 24-28 weeks of gestation [2]. GDM is associated with short and long term clinical outcomes, affecting both mothers and infants. Mothers with GDM have a higher chance for an operative delivery and are more likely to develop type 2 diabetes [3]. Offsprings of diabetic mothers are predisposed to fetal macrosomia, respiratory difficulties and metabolic complications in the neonatal period and have a higher risk for future obesity and alteration in glucose metabolism [4-6].

The rising prevalence of GDM, reflective of the prevalence of type 2 diabetes, warrant the development of new prevention strategies [7]. Although results from randomized controlled trials aimed at prevention of GDM with nutritional and lifestyle interventions are conflicting [8], some studies demonstrated that a major reduction of the risk is possible, especially when interventions initiate early in pregnancy, during the first or early second trimester [9-10]. Identification of women at a high risk for GDM development at an early stage of pregnancy will therefore enable the implementation of early intervention strategies, which may prevent or reduce GDM prevalence and its associated comorbidities.

In recent years, several studies have utilized electronic health records (EHRs) to construct prediction models for mortality [11,12] and disease onset [13-15]. Despite numerous research studies on risk factors for GDM development [16], no predictive model has been established in clinical practice to date. In this Example, a model for GDM prediction is constructed based on nationwide EHR data. The performance of the model is evaluated from pregnancy initiation up to 20 weeks of gestation.

### Methods

### Data

Data were extracted from the database of Clalit Health Service, the largest health care provider in Israel. Nearly five million individuals, representing over 50% of Israel's adults population, are currently enrolled in Clalit [17], a non-governmental, non-profit organization included in the national health insurance law in Israel. Dating back to 2002, the database contains EHRs of over 11 million patients, with over 5.4 billion numerical and categorical entries. The data analyzed included anthropometrics (height and weight), blood pressure measurements, blood and urine lab tests, diagnoses recorded by physicians, and pharmaceuticals prescribed. Most of the data originates from community clinics records, but records from Clalit's hospitals were also included in the analysis.

### Study Population and Target Definition

In Israel, GDM is defined by a two-step oral glucose tolerance test (GTT) which is performed routinely to all pregnant women during 24-28 weeks of pregnancy according to National Institutes of Health (NIH) guidelines [18]. In the first step, a 50 g, 1 hour GTT is performed; women with glucose levels higher than 200 mg/dL receive a GDM diagnosis. Women with glucose values above 140 mg/dL are referred to the second step, in which an additional 100 g, 3 hours GTT is performed. Women with two glucose measurements above the thresholds of 95, 180, 155 and 140 mg/dL under fasting conditions, one, two and three hours after glucose intake, respectively, also receive a GDM diagnosis [2,19].

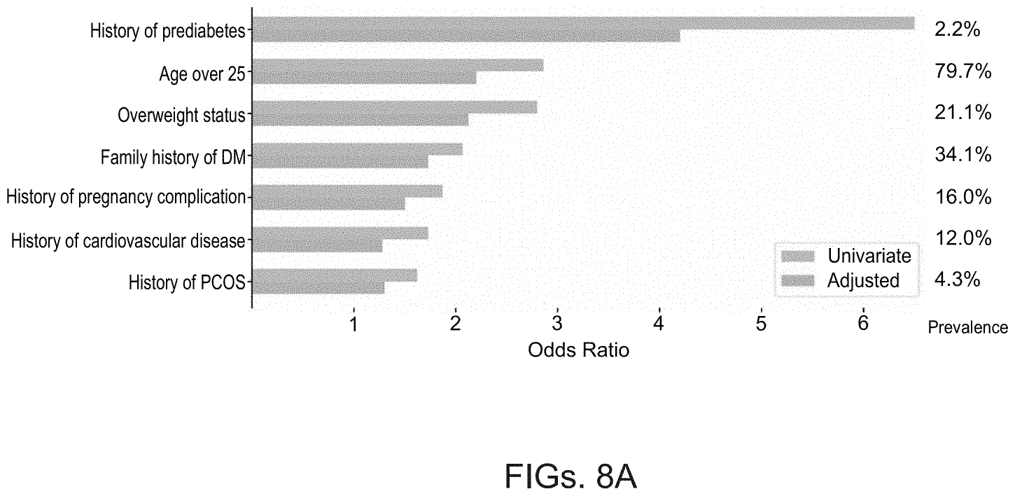

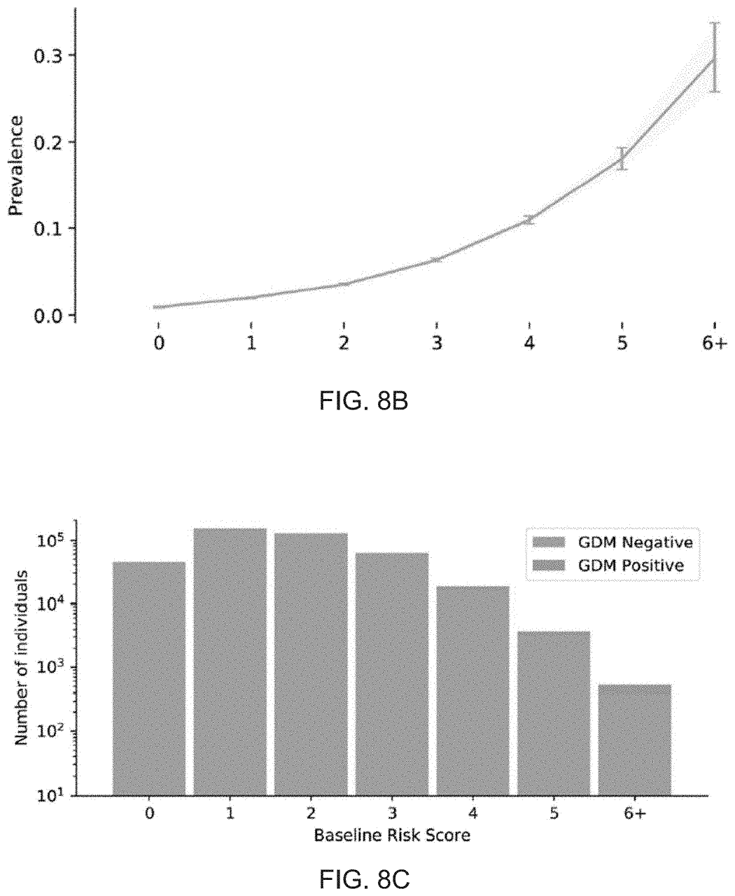

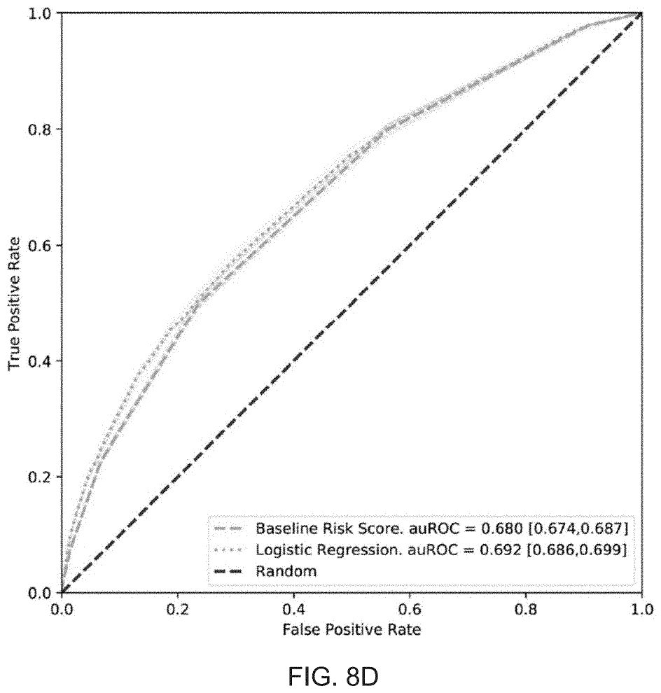

Accordingly, GDM status was defined based on the GTT results. In cases in which more than one GTT was available, a GDM diagnosis was defined if at least one of the tests were positive. Women who were supposed to undergo a 100 g GTT due to a high result on the 50 g GTT, but had no record of the test results, were excluded. Women with pre-pregnancy record of diabetes determined by a pre-pregnancy Hemoglobin A1c (HbA1c) blood test above 6.4% or a diabetes diagnosis were also excluded. In total, 588,744 pregnancies of 368,381 women were included in the cohort (see FIGS. 8A-D, below).

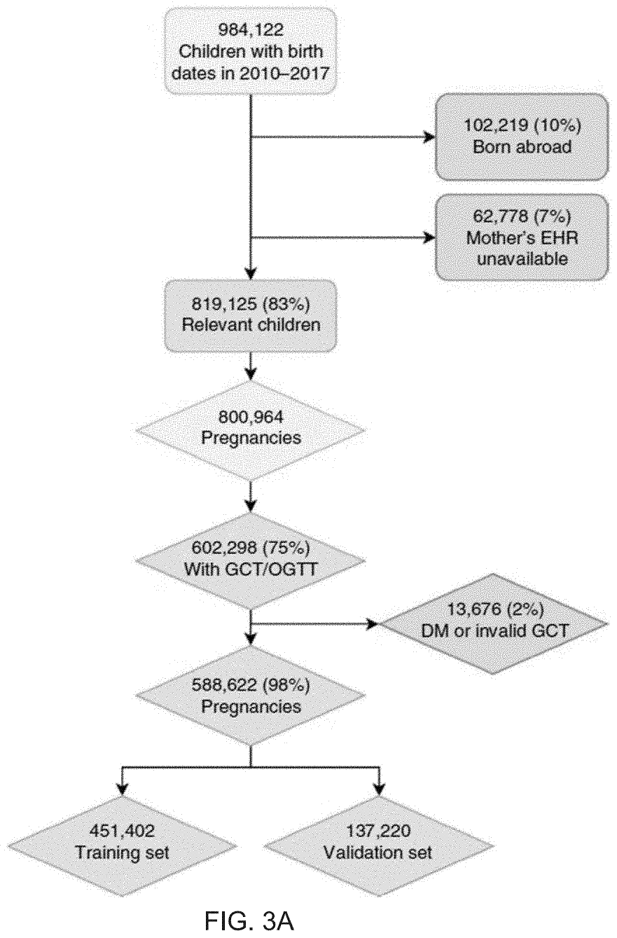

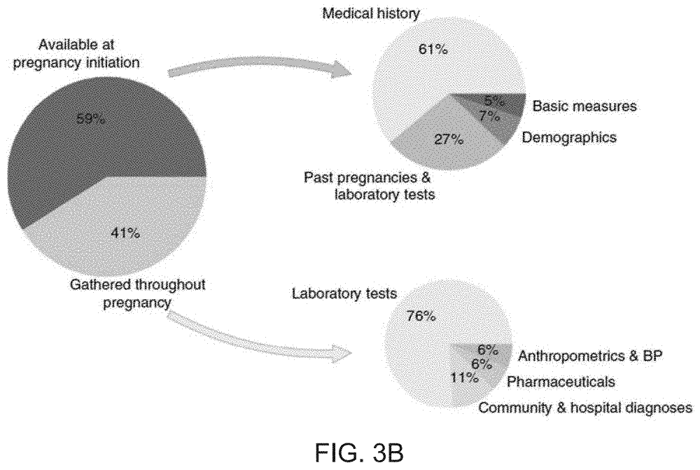

Prior to any analysis of the data, the study population was split to a training set and a test set. To emulate practical use, the test set was defined according to a temporal validation scheme [20]. Pregnancies that ended during 2017 or 2018 composed the test set, and pregnancies that ended before Dec. 31, 2016 composed the training set. This choice thus represents a setting in which the model may be implemented in practice. Throughout this Example, all results are reported on the test set, unless stated otherwise. Data and cohort characteristics are shown in FIG. 3A and Table 1.1. Table 1.1 lists numbers of data items (laboratory tests, diagnoses and so on) before patients underwent GCT during pregnancy are presented BP, blood pressure.

TABLE 1.1
Basic Characteristics of the Cohort Data

| | | | Validation sets | | |
|---|---|---|---|---|---|
| | | | | | Geo- |
| | | Training set | Future | Geographical | temporal |
| Cohort | Pregnancies in | 451,402 | 82,678 | 46,002 | 8,540 |
| | cohort (n) | | | | |
| | GCT result | 108 ± 28 | 112 ± 29 | 103 ± 26 | 108 ± 27 |
| | (mg dl⁻¹; | | | | |
| | mean + s.d.) | | | | |
| | GDM prevalence | 3.6 | 4.9 | 2.4 | 3.9 |
| | (%) | | | | |
| Patients | Unique patients (n) | 305,554 | 82,380 | 32,028 | 8,509 |
| | Unique patients for | 152,927 | 26,407 | 14,205 | 2,432 |
| | which this is their | | | | |
| | first pregnancy | | | | |
| | Age at pregnancy | 29.8 ± 5.3 | 30.4 ± 5.4 | 28.6 ± 5.4 | 29.1 ± 5.5 |
| | initiation (years) | | | | |
| | (mean ± s.d.) | | | | |
| | BMI at pregnancy | 23.3 ± 4.6 | 23.1 ± 4.5 | 23.6 ± 4.5 | 23.5 ± 4.4 |
| | initiation (kg m⁻²) | | | | |
| | (mean ± s.d.) | | | | |
| Data | Laboratory tests (n) | 184,823,814 | 58,272,694 | 16,348,392 | 5,185,971 |
| Available | Height, weight or | 2,862,363 | 1,144,582 | 316,611 | 131,466 |
| | BP recorded (n) | | | | |
| | Diagnoses recorded | 34,153,464 | 11,396,483 | 3,350,785 | 1,093,094 |
| | (n) | | | | |
| | Pharmaceuticals | 25,098,401 | 7,996,519 | 2,317,888 | 741,957 |
| | dispensed (n) | | | | |

### Baseline Risk Score

Currently there are no validated GDM prediction tools that are employed in clinical practice. The National Institute of Health (NIH) recommends women to rank themselves by counting the number of risk factors they possess according to a short questionnaire [21]. To create a baseline model, a close proxy to this score was calculated for every woman in the cohort, denoted here as their “Baseline Risk Score”. An analysis of the features and their predictive power is described in FIGS. 8A-D.

### Features and Predictions

2355 features were constructed from the dataset. 295 of them are available at the initiation of pregnancy, and the rest 2060 are generated from data gathered throughout the pregnancy. The features available at the initiation of pregnancy include (i) demographics (e.g., ethnicity), (ii) basic measures (e.g., age, weight, height), and medical history gathered prior to the current pregnancy, including data on (iii) previous pregnancies and (iv) data from non-pregnancy periods. Features gathered throughout the current pregnancy include (i) blood and urine lab tests, (ii) clinic and hospital diagnoses, (iii) anthropometrics and blood-pressure measurements, and (iv) pharmaceuticals prescribed and collected. A complete list of the features, including methods for feature generation are available in Appendix 3, below. The percentage of feature availability per category is presented in FIG. 3B.

Predictions were generated using a Gradient Boosting Machine (GBM) model [22], built with decision-tree base-learners. Cross-validation among the training set was used to set hyperparameters. Cross-validation results and exact hyperparameters values are described in Appendix 4, below.

### Model Interpretations

To understand how single features relate to the model's output Shapley values [26] were used as they are suited for complex models such as artificial neural networks and gradient boosting machines. Shapley values partition the prediction result of every sample into the contribution of each constituent feature value, by estimating the difference between models with subsets of the feature space. By averaging over all samples, Shapley values estimate the contribution of each feature to the overall model predictions.

### Results

### GDM Prediction Model

A baseline, termed Baseline Risk Score, was defined as the summation of seven binary variables that the NIH recommends to use as GDM risk factors. Odds ratios for these seven parameters are presented in FIG. 8A. Odds ratios for all the parameters are greater than one (1.28-3.92), consistent with their classification as risk factors. The risk score is predictive of GDM status (FIGS. 8B-D). However, the highest risk for GDM development that can be predicted by this model is no more than 30%.

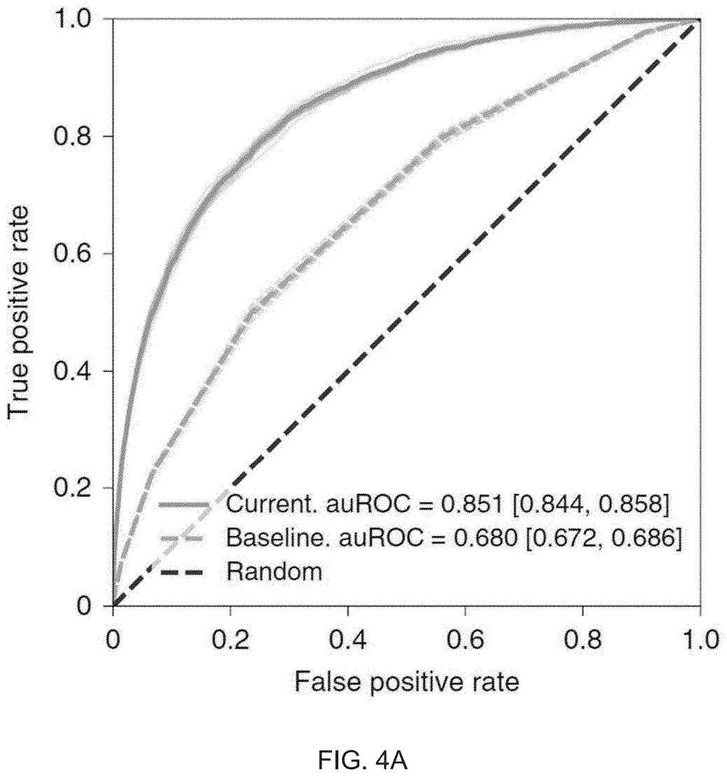

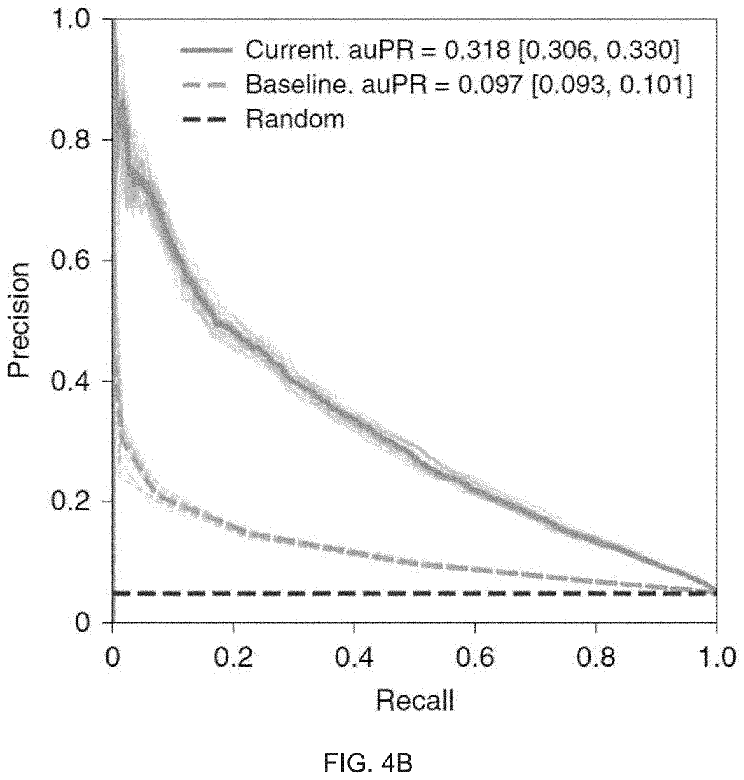

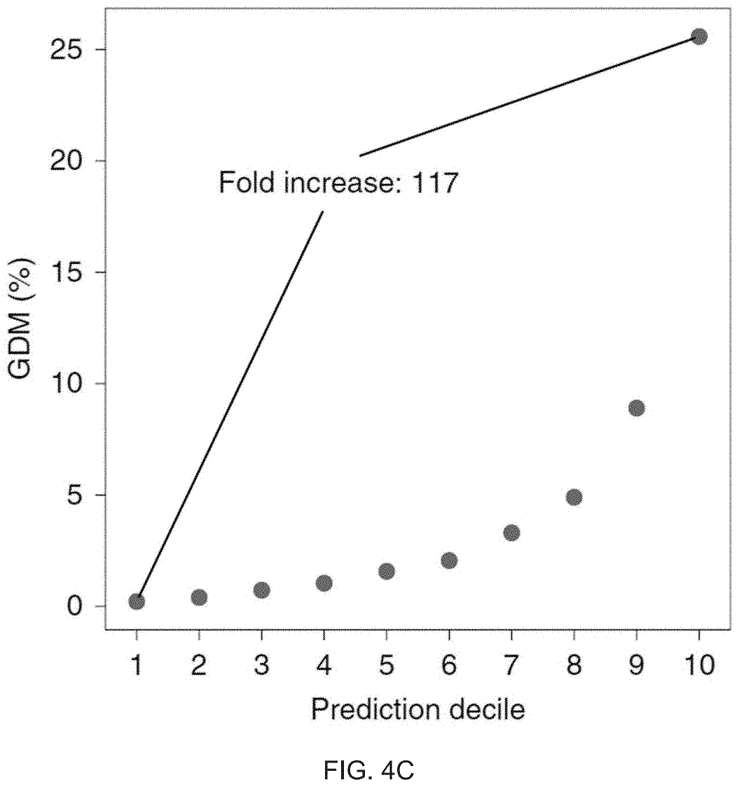

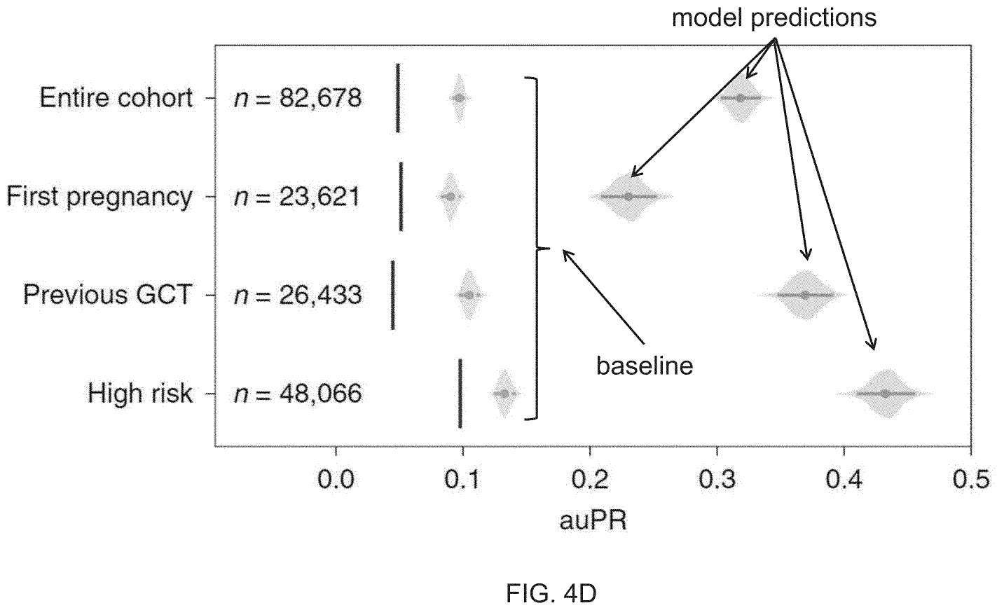

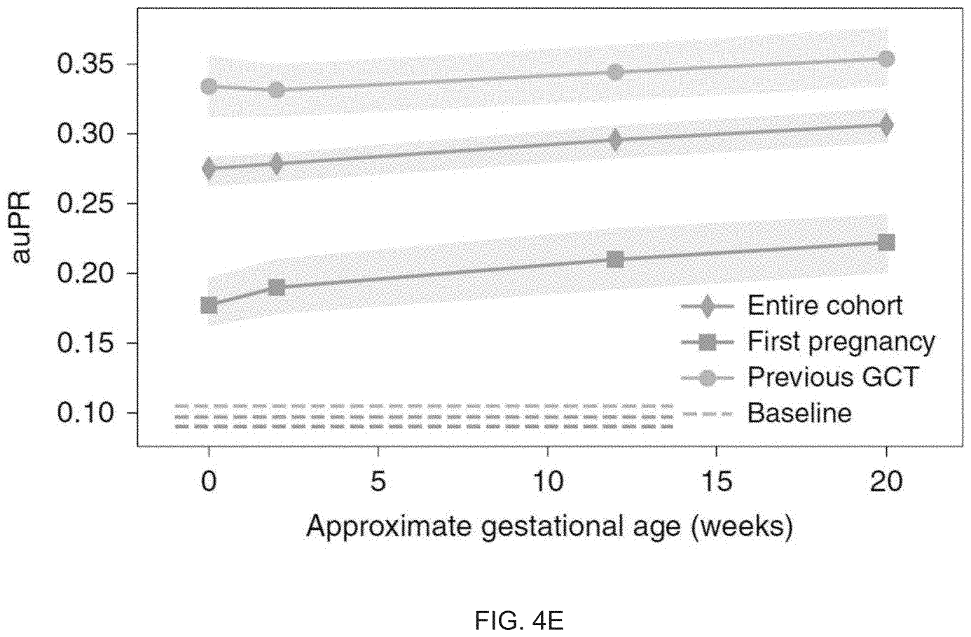

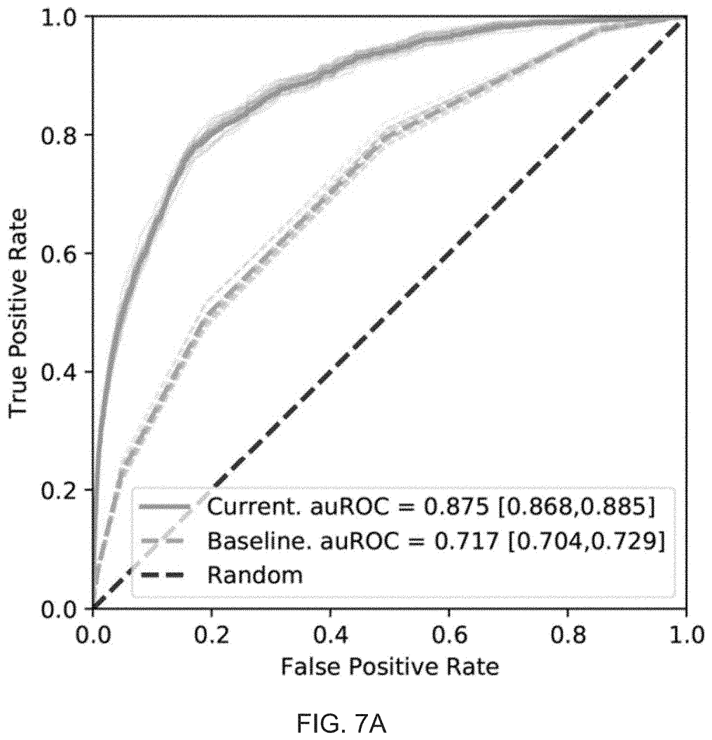

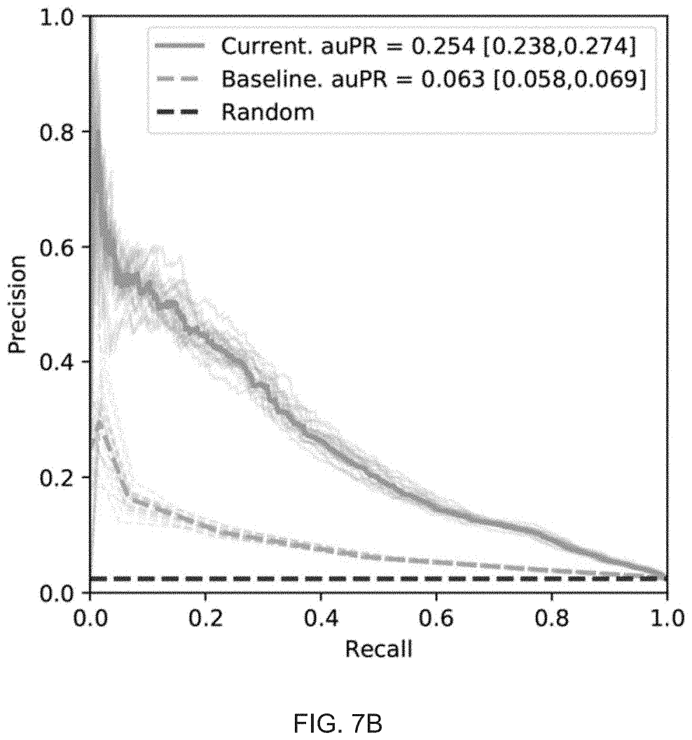

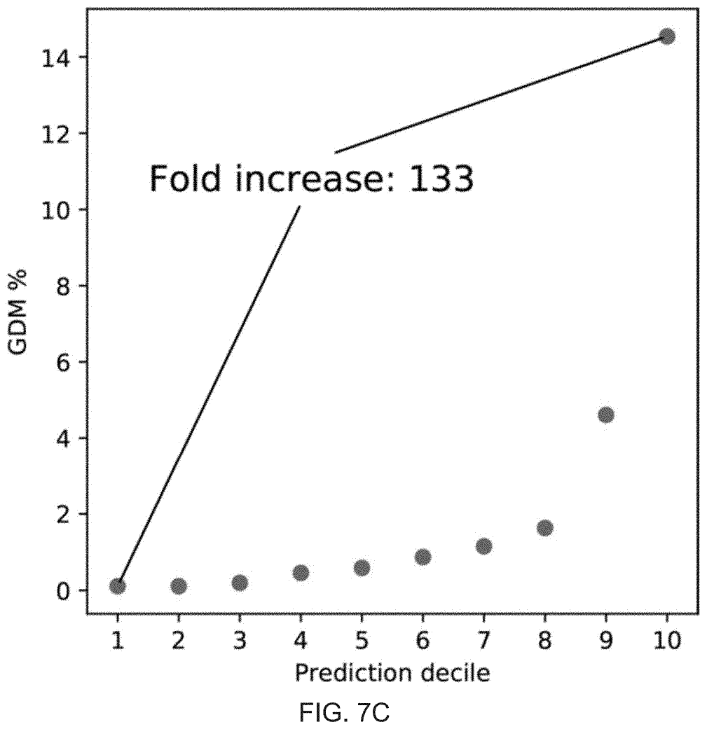

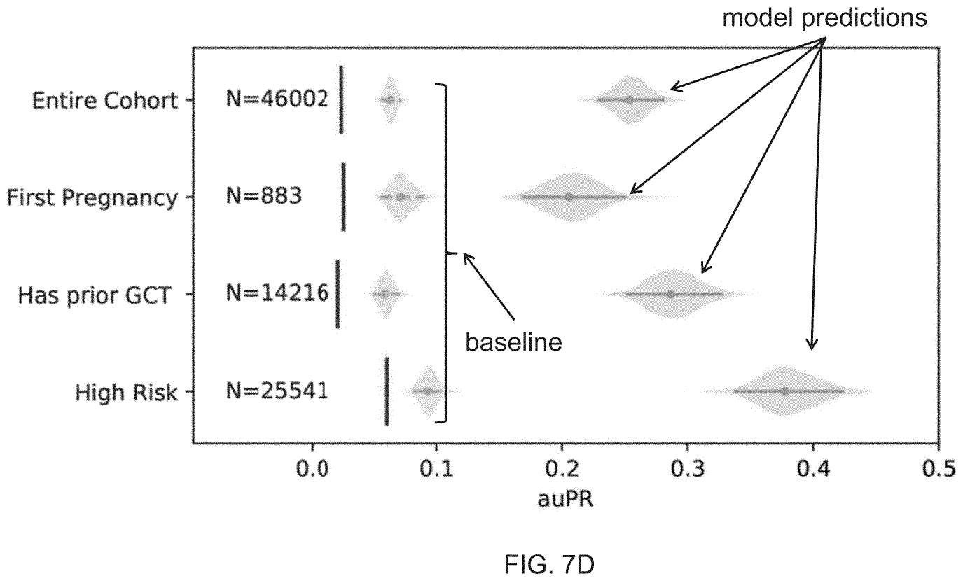

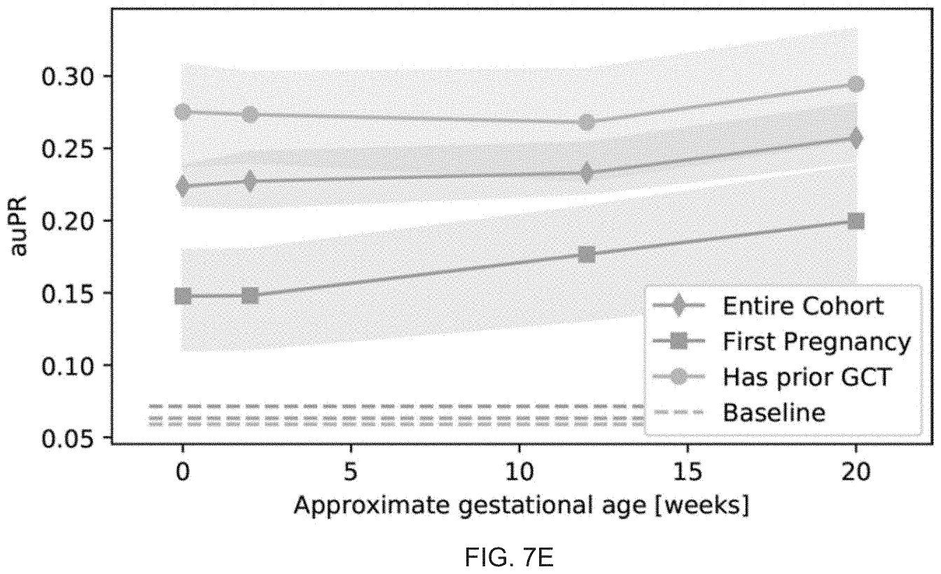

To evaluate whether EHR-derived information may improve GDM predictions, a set of 2355 features was compiled. These features were then used to train a gradient boosting model to predict the probability of each held-out individual (not part of the training set) to develop GDM. The predictive model evaluation is shown in FIGS. 4A-E, (see also FIGS. 7A-E and 11A-E for evaluation on the geographical validation set and the geo-temporal validation set, respectively).

The EHR-based model of the present embodiments achieved an area under the receiver operating characteristic curve (auROC) of 0.854 and Precision-Recall (auPR) of 0.318, compared to a auROC of 0.682 and auPR of 0.097 by the baseline model (FIGS. 4A-B). It was ensured that the model predictions are well-calibrated [28], namely that its predictions reflect the actual expected risk of an individual (FIG. 4C).

The ability to predict GDM at different weeks of gestation was evaluated by constructing models based only on data collected prior to that week. The results of this analysis show that although the prediction improves by incorporating features gathered during pregnancy, predictions at pregnancy initiation still outperform the baseline model by 2-3 folds of auPR. This effect is stronger for women in their 2nd pregnancy onwards (FIG. 4E).

The Inventors next examined whether the predictions differ in accuracy for different subsets of the population, consisting of (1) Exact gestational age: the subset of women with their gestational age logged, who underwent the GTT in the recommended period; (2) First pregnancy: women with no previous record of pregnancy; (3) Has GTT: women who have a record of a GTT from a previous pregnancy; (4) Two blood tests available: women with two separate records of a fasting glucose blood test in different trimesters in pregnancy; and (5) High risk: woman with Baseline Risk Score greater than or equal to 3. Across all subgroups, the EHR-based model of the present embodiments had higher auROC and auPR values as compare to the baseline model (FIG. 4D).

Table 1.2 summarizes the evaluation results in the geographical and temporal validation sets.

TABLE 1.2

| | | | Geographical | | | |
|---|---|---|---|---|---|---|
| | Future validation | | validation | | Temporal validation | |
| | (n = 82,678) | | (n = 46,002) | | (n = 8,540) | |
| | Full | Baseline | Full | Baseline | Full | Baseline |
| Set | Model | RS | Model | RS | Model | RS |
| auROC | 0.851 | 0.680 | 0.875 | 0.717 | 0.864 | 0.706 |
| | (0.847-0.855) | (0.675-0.686) | (0.866-0.885) | (0.708-0.728) | (0.856-0.880) | (0.683-0.732) |
| auPR | 0.318 | 0.097 | 0.254 | 0.063 | 0.287 | 0.094 |
| | (0.307-0.329) | (0.095-0.100) | (0.241-0.270) | (0.061-0.068) | (0.248-0.341) | (0.08-0111) |
| % GDM is | 0.22% | | 0.11% | | 0% | |
| lowest | | | | | | |
| decile | | | | | | |
| % GDM is | 25.6% | | 14.6% | | 22.2% | |
| highest | | | | | | |
| decile | | | | | | |
| PPV at | 62.5% | | 53.3% | | 60.7% | |
| TPR = 10% | | | | | | |

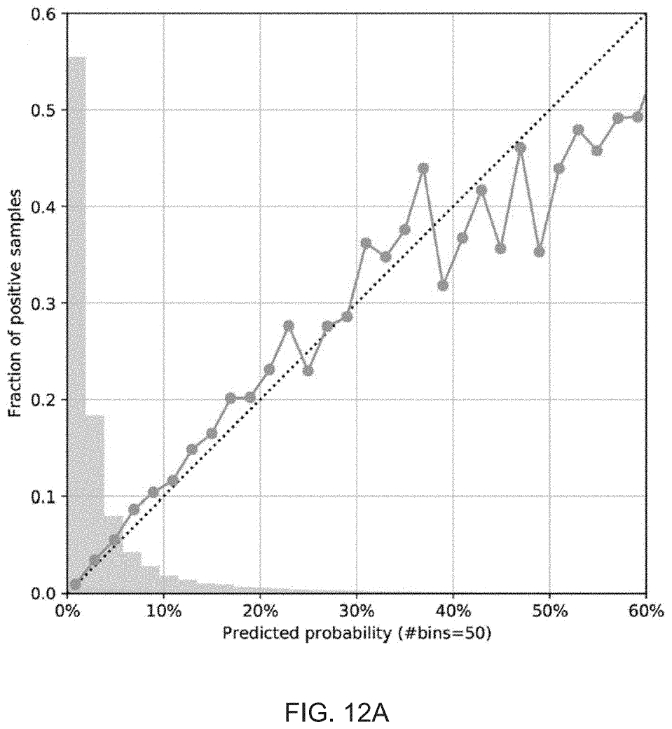

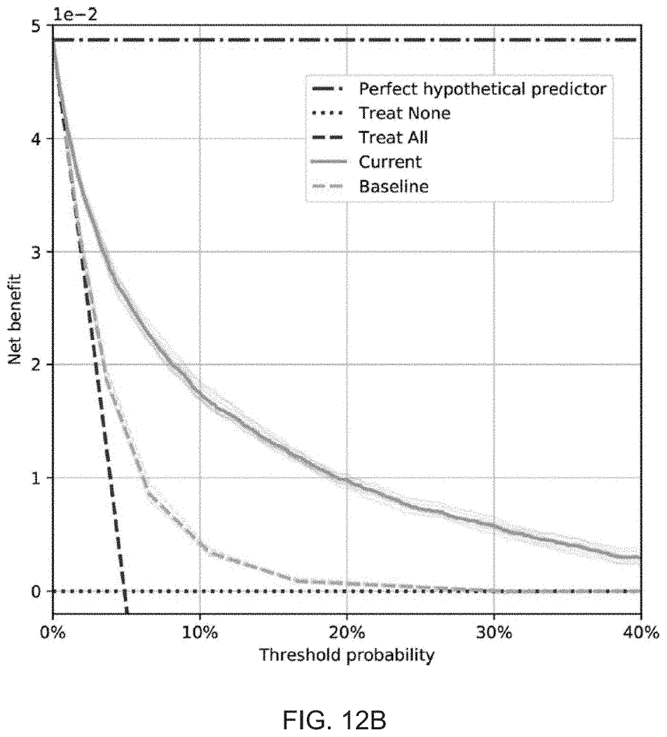

The study ensured that the model predictions reflect the actual expected risk of an individual and, furthermore, demonstrate the utility of the predictor by considering its decision curve (FIGS. 12A and 12B).

### Model Interpretation

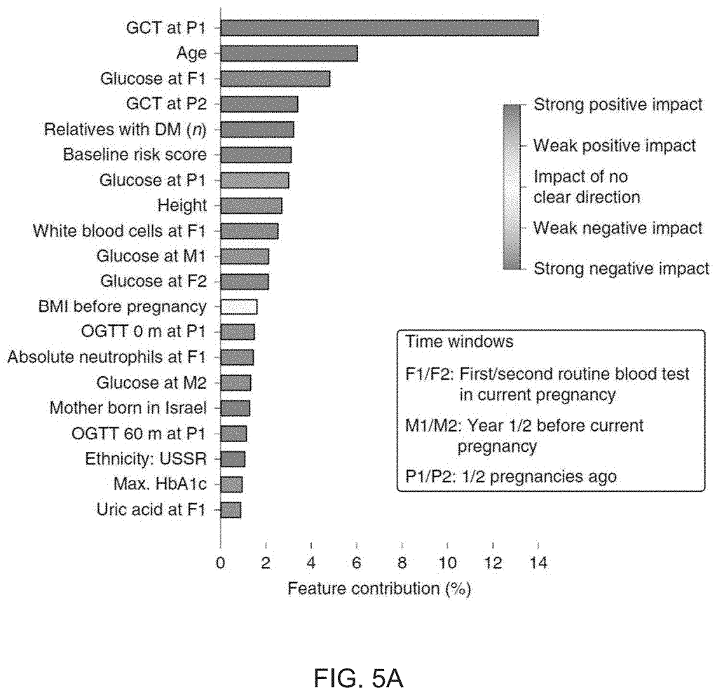

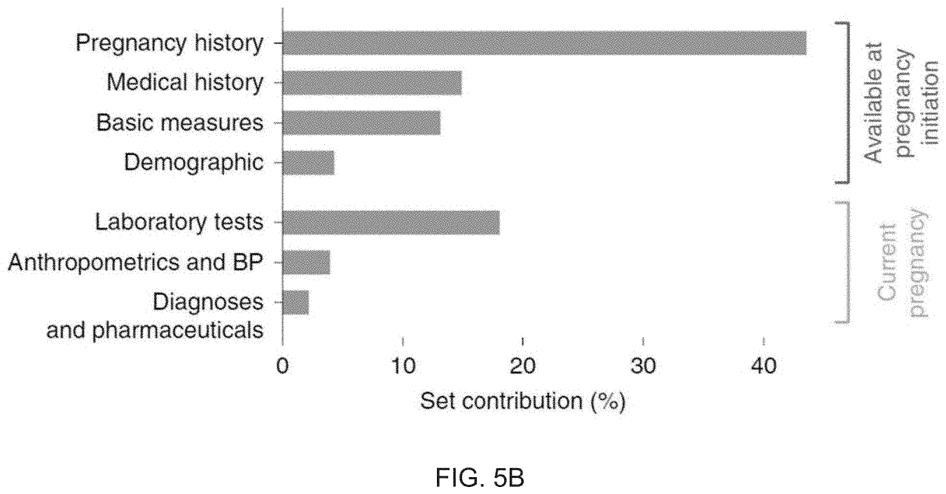

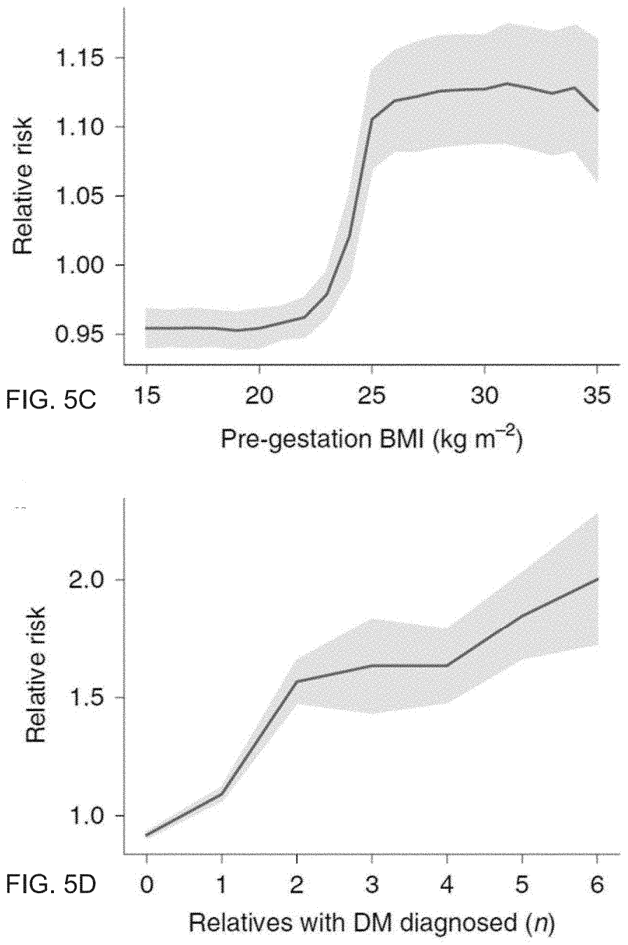

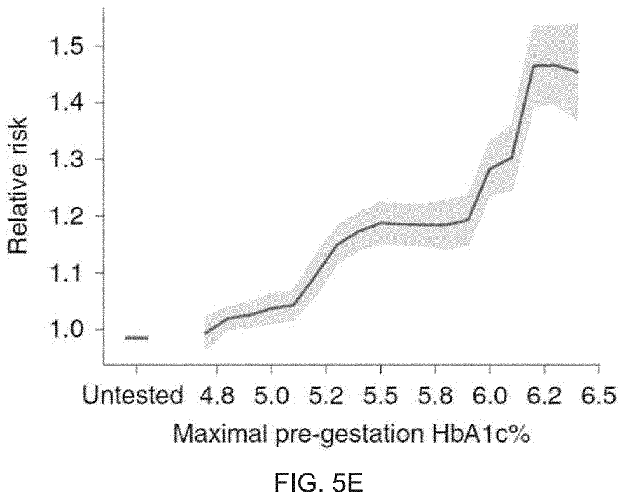

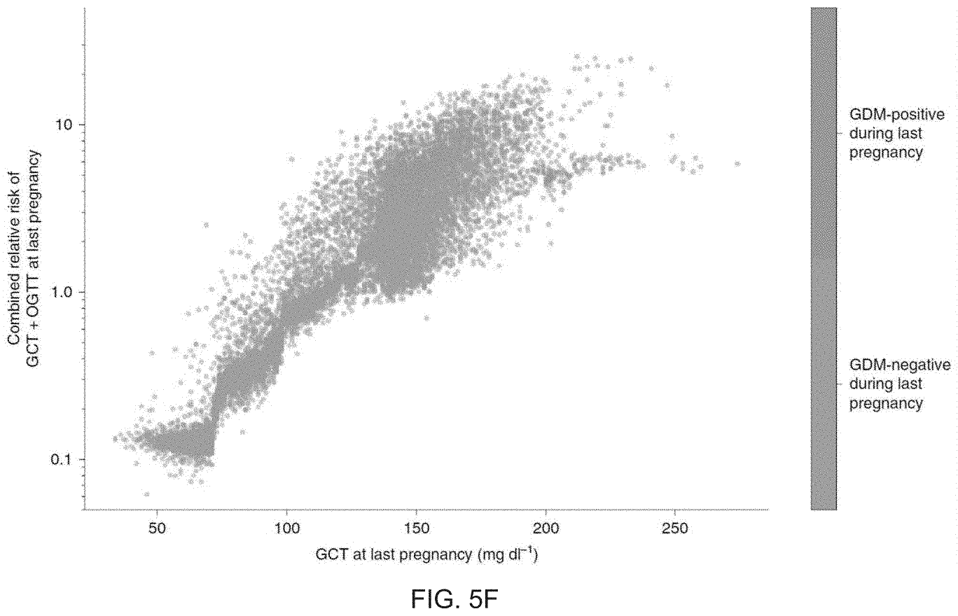

FIGS. 5A-E show Shapley values based interpretation of the model of the present embodiments. The Shapley analysis identified the most predictive feature for GDM diagnosis to be the 50 g GTT result in the previous pregnancy, followed by maternal age and fasting blood glucose in the first trimester (FIG. 5A).

The additive nature of the Shapley values allows construction of a feature importance score for feature sets, by summing of Shapley values per set. The results of this analysis (for the sets defined in Methods, above) are presented in FIG. 5B.

The Shapley values were further used to build Dependence Plots that capture the non-linear associations of every feature. Dependence plots show the Shapley value of a specific feature, representing its predicted contribution, in the form of relative risk (RR), against the feature's value (see Appendix 5, below). In this Example, the dependence plots for two well known risk factors for GDM were examined: pre-pregnancy maternal BMI [29], and the number of relatives diagnosed with diabetes mellitus (DM) [30]. For prepregnancy BMI, the RR for GDM starts to increase above 21, reaches above 1 in BMI values above 24, and plateaus at 1.2 in BMI above 30 (FIG. 5C). The RR for GDM increases as the number of the first degree family members with GDM increases, reaching a RR of 1.8 in women with 6 relatives diagnosed with DM (FIG. 5D). Analysis of pre-gestation HbA1C revealed an increase of the RR for GDM with an increase in the pregestational HbA1C, even in values that are considered to be normal range (less than 5.7%). A steeper increase in the RR occurs in values of HbA1C>5.9% (FIG. 5E).

To further explore the importance of GCT in the previous pregnancy, a further analysis was conducted: for every patient the combined Shapley value was plotted for all glucose tests (GCT and OGTT, if applicable) during previous pregnancy versus the value of GCT in the previous pregnancy (FIG. 5F). This analysis revealed that the GCT result in the previous pregnancy is more predictive than GDM diagnosis in the previous pregnancy. For example, a patient with GCT of 180 mg dl⁻¹ will be at a higher risk of GDM in her next pregnancy irrespective of whether she is diagnosed with GDM after 100-g OGTT. On the other hand, a patient with GCT of <75 mg dl⁻¹ will have a GDM risk in her next pregnancy as low as 20% of the population prevalence.

### A Simpler Prediction Model

A simpler prediction model was establish based on a reduced number of features as opposed to a model based on many EHR features. To this end, the performance of a model based on 9 questions that a patient can answer herself. This predictor achieves an auROC of 0.801 and auPR of 0.238, compared to 0.680 and 0.100, respectively, for the baseline model (FIGS. 6A-C).

This study also emulated usage of the predictive model of the present embodiments as a screening tool to identify women who are less likely to develop GDM, rather than subjecting those who fall below a certain risk threshold to the usual two-step GCT plus OGTT (GCT/OGTT) diagnostic process. The trade-off of missing diagnoses when implementing such screening across varying risk group thresholds was assessed by analyzing the proportion of women who could avoid testing versus the predictor miss rate. That is, the percentage of GDM-positive women not accurately diagnosed by this approach (FIG. 6D). Indeed, the results demonstrate that a large proportion of the population could avoid taking the test. For example, if one permits 20% of diagnoses to be missed, which is on a par with the misdiagnosis rate of GCT, then 79% of all women with a GCT result in their previous pregnancy can avoid the test in their next pregnancy.

### APPENDIX 1

### Target and Cohort Definitions

The version of Clalit Health database that was used does not include exact delivery dates for all women, however it has approximate (±1 month) birth date of every child. As such, the cohort was defined by collecting all birth dates of children Clalit-insured mothers, and looking for GTTs in the relevant period prior to the delivery, namely 32 weeks before the logged date of birth to 7 weeks after the logged date of birth. The fact that 50 g GTTs are only used in pregnancy in Israel, and the fact that pregnancy period we looked for to begin with, mean the GTTs of the data are all pregnancy-related.

GTTs appear in the lab tests data under five distinct tests: one for 1 hour 50 g result, and four for fasting, 1 h, 2 h and 3 h 100 g results. GDM was defined in accordance to practice, regardless of the order of the tests, and without consideration of whether a relevant diagnosis was recorded. In case more than one GTT was conducted, a positive result in a single GTT we considered to be positive. A small portion of pregnancies (n=8228 in the training set and n=1,525 in the test set, 1.6%) was excluded for women that had a 50 g result of 140 mg/dL or higher, but do not have a record of a 100 g GTT.

The cohort was defined our according to the relevant date of delivery. Main cohort included pregnancies that ended between Jan. 1, 2010 to Dec. 31, 2016, and test cohort included pregnancies that ended between Jan. 1, 2017 to Dec. 31, 2017.

The sole exclusion criteria was pre-pregnancy notion of (non-gestational) diabetes. Normally women with DM does not take a GTT during pregnancy, but it appears that some (<0.2%) do. To address that, patients who had one of the following markers prior to pregnancy start were excluded: (1) a recorded diagnosis of DM, defined as any of the ICD9 codes in 250.x or 357.2, or (2) a recorded non-pregnancy HbA1c % blood test of 6.5 or higher. Note that although fasting glucose could also be used to diagnose diabetes, this metric is less accurate in the data as some non-fasting patients still take the test, and was therefore not used.

### APPENDIX 2

### Baseline Risk Score Definition

The original Risk Score suggested by the NIH [21] includes eight parameters, of which all except for ethnicity are relevant for Israeli population. Seven of these parameters were therefore included in the score, defined according to the following binary variables enumerated as (1) through (7).

Binary variable (1): Overweight status. This binary variable was set to be true if non pregnancy BMI is higher than 25 kg/m², and false otherwise. If there is no record of BMI prior to the pregnancy, this binary variable was set to be false.

Binary variable (2): Family history of diabetes. This binary variable was set to be true if a first degree relative (parent or sibling) has at least one diagnosis of DM, defined as any of the ICD9 codes in 250.x or 357.2, and false otherwise. Only diagnoses available at pregnancy initiation are considered.

Binary variable (3): Age. This binary variable was set to be true if the patient was 25 or more years of age at pregnancy start, and false otherwise.

Binary variable (4): History of pregnancy complication. This binary variable was set to be the logic OR operation of the following markers: (a) History of GDM according to GTTs, defined similar to the target; (b) History of miscarriage or stillbirth, seen in a form of a diagnosis with ICD9 632, 634.x, 635.x or 637.x; and (c) History of a liveborn baby with birth weight higher than 4 kg. Note that birth weight is only logged for deliveries done in Clalit owned hospitals (about 30% of the deliveries)

Binary variable (5): History of PCOS. This binary variable was set to be true if the patient has at least one diagnosis of PCOS, ICD9 code 256.4, and false otherwise. Only diagnoses available at pregnancy start were considered.

Binary variable (6): Problems with insulin or blood sugar. This binary variable was set to be true if the patient has at least one diagnosis of prediabetes, either according to ICD9 codes 790.2x or by a HbA1c test in the range 5.7% to 6.4%, and false otherwise. Only diagnoses and tests available at pregnancy initiation were considered.

Binary variable (7): High blood pressure, high cholesterol, and/or heart disease. This binary variable was set to be the logic OR operation of the following markers: (a) History of high BP, defined as two or more BP tests with systolic BP over 140 or diastolic BP over 90, (blood pressure measurements taken during pregnancies are not included in this analysis); and (b) Recorded relevant ICD9 of 401.x, 272.x 390.x-449.x.

The final Baseline Risk Score is, then, the number of “true” entries in Binary variables (1) through (7), and therefore ranges from 0 to 7. An analysis of the odds ratio of the constructing variables, as well as a comparison to a logistic regression model from the above binary variables is presented in FIGS. 8A-D. It is noted that the logistic regression model does not significantly improve performance.

### APPENDIX 3

### Feature Generation Mechanism

2355 features were constructed from the dataset. The following list describes the generation mechanism for each.

(A): Features that are available at pregnancy initiation (295 features).

(A)(1): Demographics (41 features).

(A)(1)(i): Was the patient born in Israel (True/False)

(A)(1)(ii): Features describing ethnicity. 15 features breaking down the origin of the patient's ancestors, as logged in their country of origin. World's countries were clustered into 14 categories, corresponding to Israel's major ethnic groups. North Africa, Iraq, Iran, Yemen, East Europe, West Europe, ex-USSR, North America, Latin America, Arab, Mediterranean, Ethiopia, Asia and Africa. Another feature logs the percentage of unknown origin.
(A)(1)(iii): Socio-economic data of the locality the patient attended most clinic visits. Although personalized socio-economic data were not available, we generated some estimates using the data available by Israel's Central Bureau of Statistics [40]. Features include locality type (length 20, 1-hot vector) and locality religion breakdown (length 5, summing to 1 vector).

(A)(2): Personal characteristics (7 features).

(A)(2)(i): Age at pregnancy initiation.

(A)(2)(ii): Weight, height and BMI. Only samples available before the current pregnancy and outside past pregnancies were considered, median for all samples with age 18 and up was calculated.

(A)(2)(iii): Systolic and Diastolic blood pressure. Only samples available before the current pregnancy and outside past pregnancies were considered, median for all samples with age 18 and up was calculated.

(A)(2)(iv): Number of children born in current pregnancy—1 for single born, 2 for twins etc.

(A)(3): Pregnancy history (103 features).

(A)(3)(i): History of GDM.

(A)(3)(i)(a): Any history of GDM according to past pregnancies' GTTs (True/False).

(A)(3)(i)(b): GDM status in each of the last 3 pregnancies.

(A)(3)(ii): History of miscarriage. seen in a form of a diagnosis with ICD9 632, 634.x, 635.x or 637.x

(A)(3)(iii): Largest baby weight. maximal birth weight recorded. Note that birth weight is only available for 25% of the cohort.

(A)(3)(iv): Number of previous births. number of children born before current pregnancy.

(A)(3)(v): Lab tests during last 3 pregnancies.

(A)(3)(v)(a): Median values during each pregnancy of the following tests, the 25 most common lab tests. HB, HCT, RBC, MCV, MCH, MCHC, WBC, PLT, LYM %, NEUT %, MONO %, EOS %, BASO %, LYMP (abs), NEUT (abs), MONO (abs), EOS (abs), BASO (abs), MPV, RDW, Urine culture, MICRO %, MACRO %, HYPO %, HYPER % (75 features).

(A)(3)(v)(b): Median values during each pregnancy of fasting glucose and HbA1c % (6 features).

(A)(3)(v)(c): GTT results (both 50 g and 100 g), if available (15 features).

(A)(4): Medical history outside of pregnancy (144 features).

(A)(4)(i): Number of first degree relative (parent or sibling) with at least one diagnosis of DM, defined as any of the ICD9 codes in 250.x or 357.2. Only diagnoses available at pregnancy initiation are considered.

(A)(4)(ii): History of PCOS, according to ICD9 code 256.4. Only diagnoses available at pregnancy start are considered.

(A)(4)(iii): History of prediabetes.

(A)(4)(iii)(a): Diagnoses. true if the patient has at least one diagnosis of prediabetes according to ICD9 codes 790.2x.

(A)(4)(iii)(b): Maximal HbA1c % logged.

(A)(4)(iii)(c): Joint prediabetes definition. either according to diagnosis or by a HbA1c test in the range 5.7% to 6.4%. Only diagnoses and tests available at pregnancy initiation are considered.

(A)(4)(iv): Features related to high blood pressure, high cholesterol, and/or heart disease.

(A)(4)(iv)(a): Number of high BP tests with systolic BP over 140 or diastolic BP over 90. Blood pressure measurements taken during pregnancies are not included in this analysis.

(A)(4)(iv)(b): Recorded relevant ICD9 of 401.x (hypertension), 272.x (high cholesterol) and 390.x-449.x (heart diseases) (3 True/False features).

(A)(4)(v): Baseline Risk Score value (Appendix 1).

(A)(4)(vi): Lab tests during last 5 years (132 features). logging the median value in every window M1-M5 (see Time Windows ahead). We considered the top 25 most common tests. HB, HCT, RBC, MCV, MCH, MCHC, WBC, PLT, LYM %, NEUT %, MONO %, EOS %, BASO %, LYMP (abs), NEUT (abs), MONO (abs), EOS (abs), BASO (abs), MPV, RDW, Urine culture, MICRO %, MACRO %, HYPO % and HYPER %, plus Glucose and HbA1c %. We only considered data gathered outside of pregnancy periods for these features.

(A)(4)(vii): Two coefficients of a linear regression for fasting glucose vs. time (only if 3 or more measurements were available).

(B): Features gathered throughout current pregnancy (2060 features).

(B)(1): Lab tests (524 features). Median values of 250 most common lab tests during F0-F2 (see Time Windows ahead).

(B)(2): Clinic and hospital diagnoses (906 features). Counts of 300 most common diagnoses in community clinics and 100 most common diagnoses in hospitals, plus “other” count for all the non-top diagnoses, for each window in F0-F2 (see Time Windows ahead).

(B)(3): Anthropometrics and blood-pressure measurements (27 features).

(B)(3)(i): Medians of weight, height, BMI, systolic and diastolic BP and the time between the measurement to the GTT, for each window in F0-F2 (see Time Windows ahead).

(B)(3)(ii): Two coefficients of a linear regression for weight vs. time, for 10-20 weeks of gestation (only if 3 or more measurements were available).

(B)(3)(iii): 2×2 coefficients of a linear regression for systolic/diastolic blood pressure vs. time, for 0-20 weeks of gestation (only if 3 or more measurements were available).

(B)(4): Pharmaceuticals (603 features). Counts of 300 most common medications in, plus “other” count for all the non-top medications, for each window in F0-F2 (see Time Windows ahead).

A complete list of all the 2355 features used in this study is provided in Table 6.1 below (see Appendix 6).

### Time Windows

Windows during pregnancy were defined according to the medical examination pregnancy schedule for Israeli women [19]. Three relative-time windows within the pregnancy period were defined. These time-windows are named F0, F1 and F2, and are defined as follows.

Time-window F0: from 30 to 22 weeks before the GTT, representing −4 to 4 weeks of gestation.

Time-window F1: from 22 to 12 weeks before the GTT, representing 4 to 14 weeks of gestation. This window includes the period in which women attend the first blood test during pregnancy, which is recommended during 6-12 weeks of gestation.

Time-window F2: from 12 to 4 weeks before the GTT, representing 14 to 22 weeks of gestation. This window includes the period in which women attend the second blood test during pregnancy (triple test), which is recommended during 16-18 weeks of gestation.

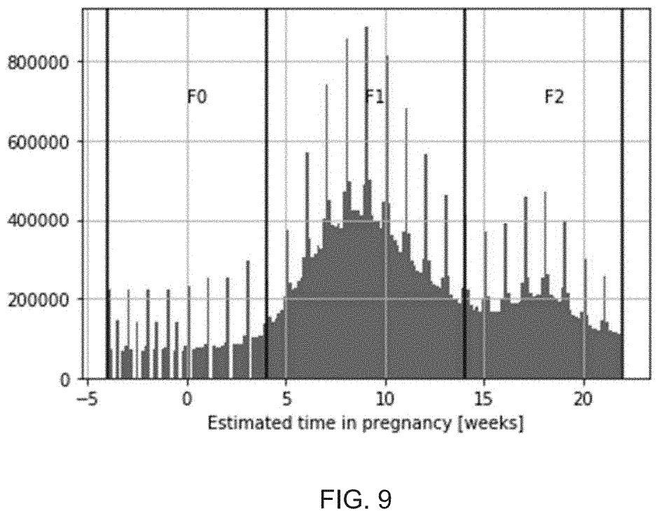

This choice is backed by the test population in the data, as shown in FIG. 9.

For medical history outside pregnancy periods, five one-year time-windows were defined, to covering the five years prior to the date of approximate gestation. These one-year time-windows are named M1 (last year before pregnancy) to M5 (5 to 4 years before pregnancy).

Additional time windows were defined for past pregnancy periods. These are denoted by P1 (last pregnancy), P2 (two pregnancies ago) and P3 (three pregnancies ago). Pregnancies were located according to the birth date of a child, and pregnancy period was defined as 40 weeks before that date plus 2.5 months in each direction to cover randomization of birth dates.

### APPENDIX 4

### Prediction Details

A gradient boosting predictor trained with the LightGBM [41] python package was used. Hyperparameters were selected following a cross-validated random search, with the following settings selected: num_boost_round=603, num_leaves=20, learning_rate=0.05, feature_fraction=0.2, bagging_fraction=0.8, bagging_freq=5, min_data_in_leaf=4.

### APPENDIX 5

### Dependence Plots

To draw the dependence plots, the resulting Shapley value were converted to Relative Risk (RR). In Shapley analysis, the log-likelihood (LL) of the predicted probability is calculated according to

LL=ϕ₀+ϕ₁+ . . . +ϕ_{d}

where ϕ₀ is the base Shapley value (the log it of the population prevalence P₀), and ϕᵢ for i∈{1, . . . , d} are the Shapley values related to features 1, . . . ,d. The predicted probability based on a single feature is then

$${P}_{i} = S ( {ϕ}_{0} + {ϕ}_{i} )$$

$$where$$

$$S ( x ) = \frac{1}{1 + {e}^{- x}}$$

is the Sigmoid function, the inverse of the log it function. The relative risk related to a single feature and sample was therefore defined as:

$$R {R}_{i} = \frac{{P}_{i}}{{P}_{0}} = \frac{S ( {ϕ}_{0} + {ϕ}_{i} )}{S ( {ϕ}_{0} )}$$

For a set D={i, j, . . . } of features, this definition extends to

$$R {R}_{i} = \frac{{P}_{i}}{{P}_{0}} = \frac{S ( {ϕ}_{0} + {\sum }_{i ∈ D} {ϕ}_{i} )}{S ( {ϕ}_{0} )}$$

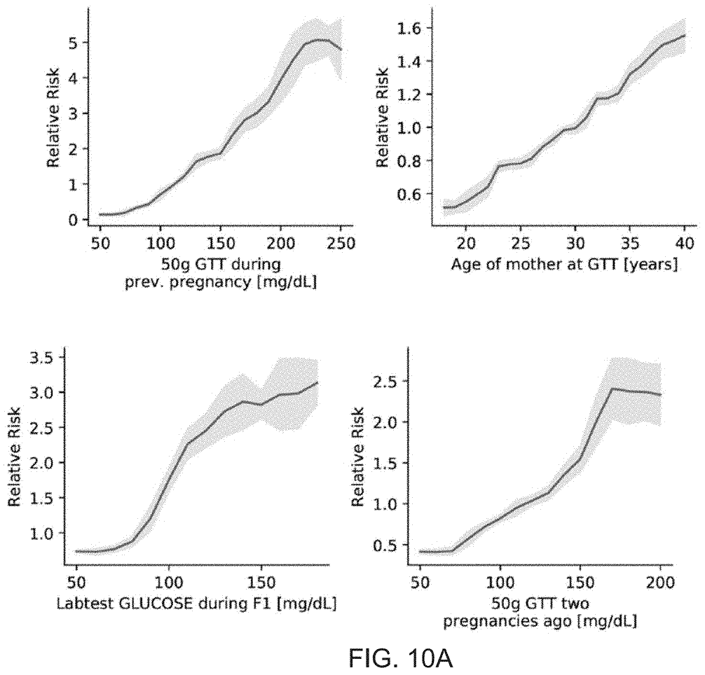

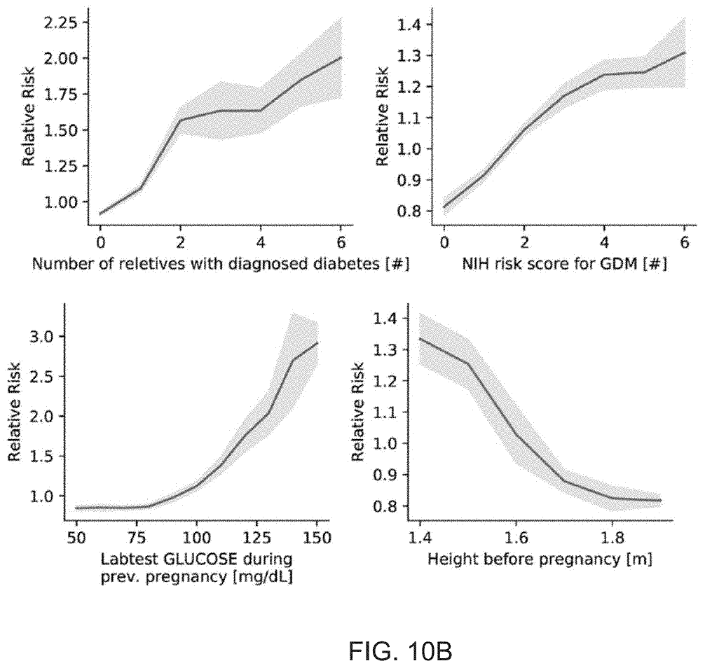

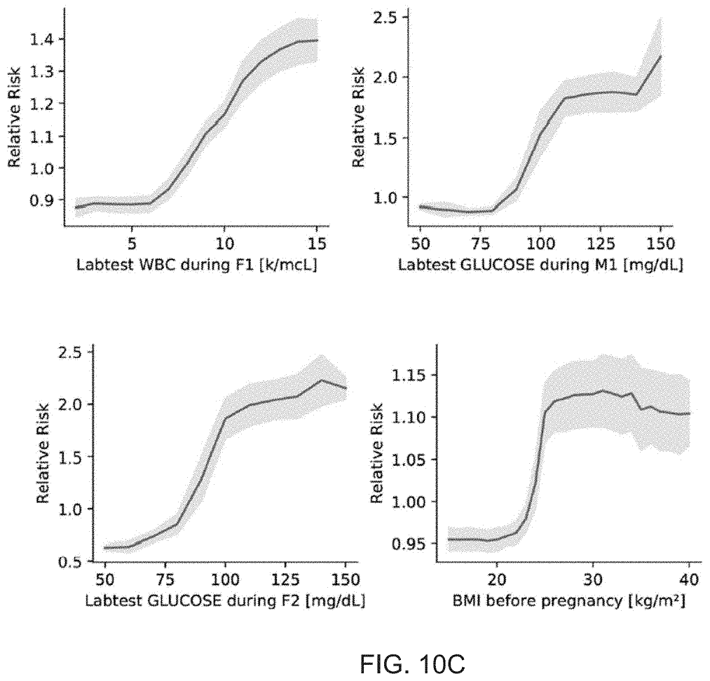

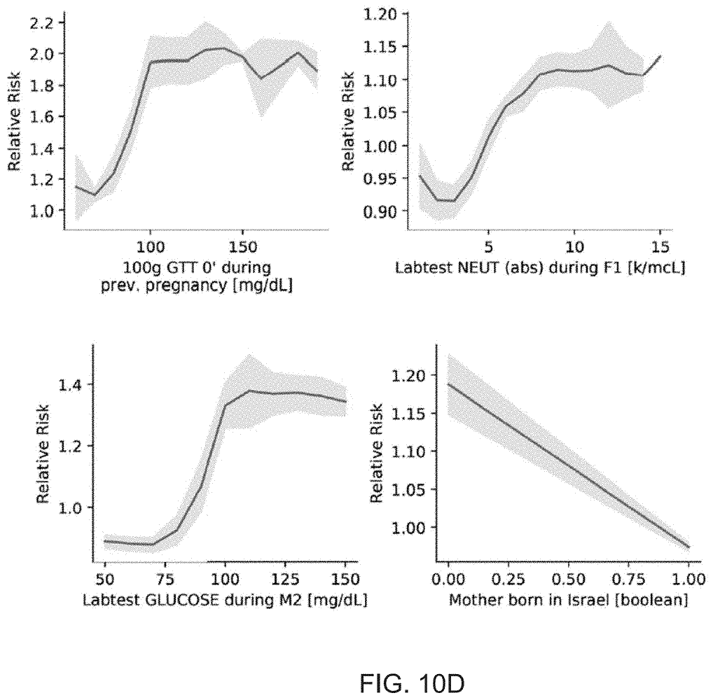

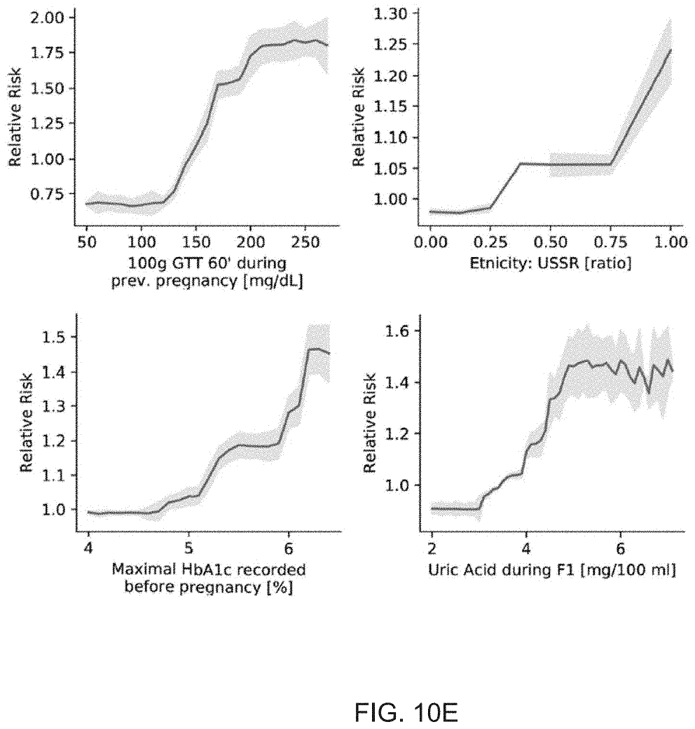

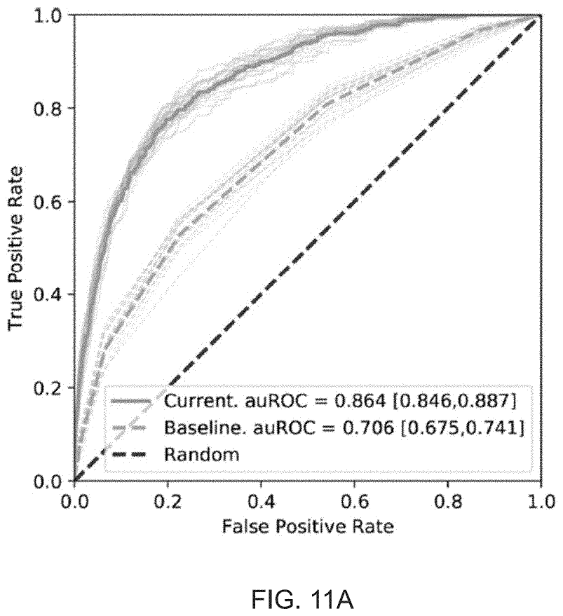

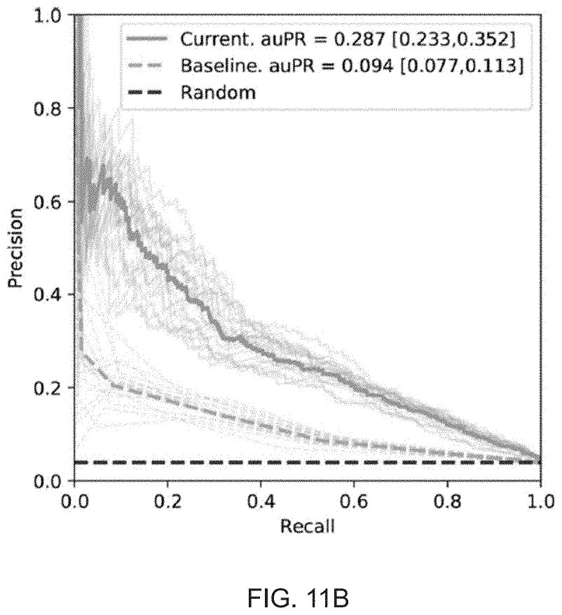

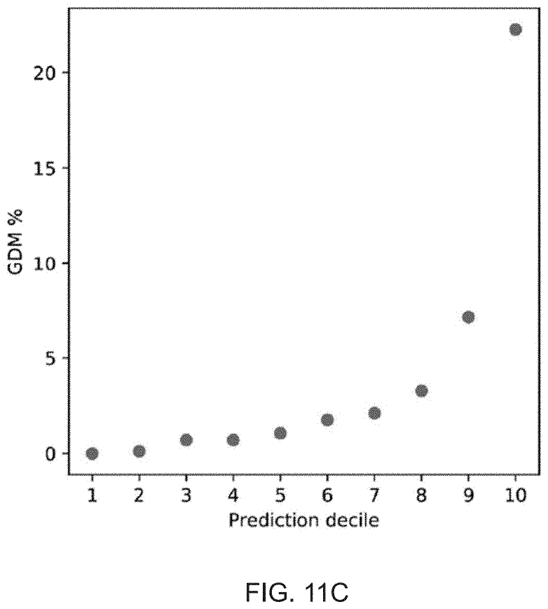

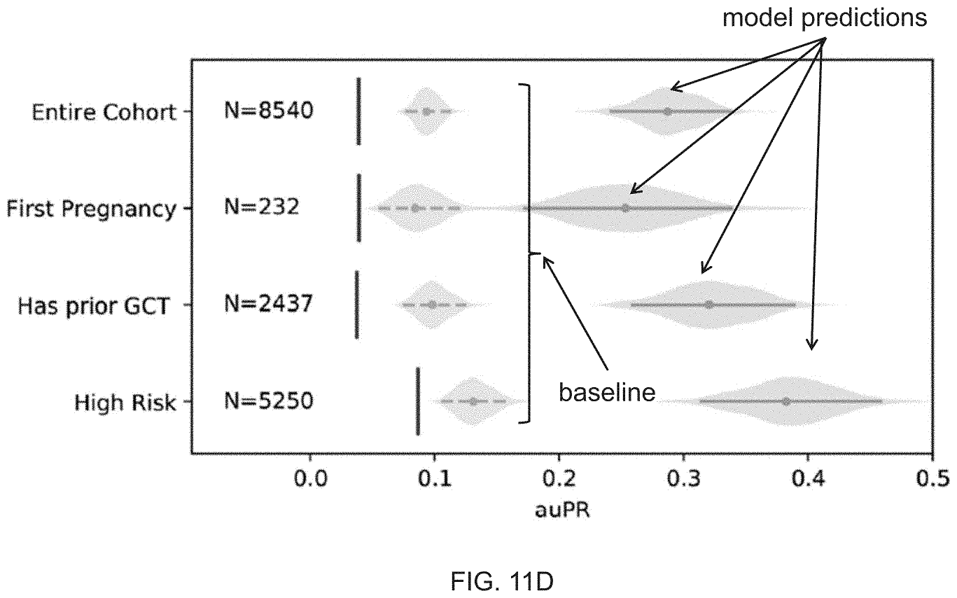

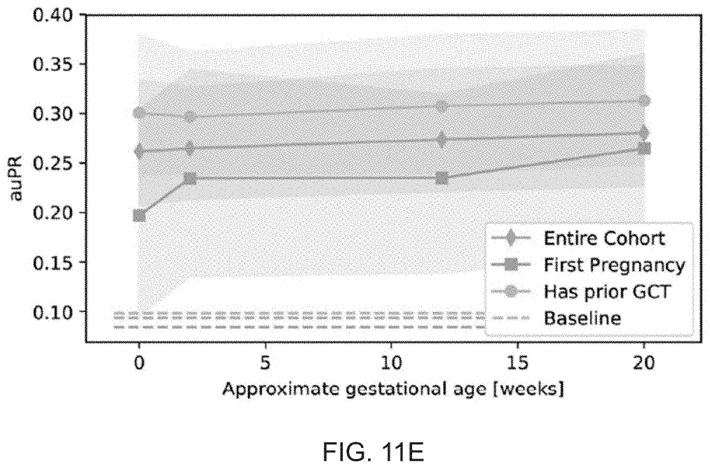

To plot the dependence plot, mean and standard deviations of the RR were calculated for each bin of feature value. This resembles a standard dependence plot, only with RR instead of Shapley values presented. FIGS. 10A-E include plots of top 20 features (ordered left to right, top to bottom). Each predicted relative risk is plotted versus feature value. Bands represent SD area of the population per bin, which is connected to interactions between input features.

### APPENDIX 6

### Complete List of Features

Table 6.1 lists all the 2355 features used in this study. The list is sorted according the significance of the respective feature for predicting the likelihood for GDM, in descending order, so that from the standpoint of prediction accuracy it is more preferred to select a parameter that is listed higher in Table 6.1, than a parameter that is listed lower in Table 6.1. For example, when N parameters are used, it is preferred to select those parameters from lines 1 through M of Table 6.1, where N≤M≤2355.

TABLE 6.1

| # | feature_name | description |
|---|---|---|
| 1 | prev_pregnancy_1_GTT_50g | 50 g GTT at 1st previous pregnancy |
| 2 | age | Age of mother at GTT |
| 3 | F1_GLUCOSE | Labtest GLUCOSE, sampled during F1: first blood test (weeks 6-12) |
| 4 | NIH_risk_score | NIH risk score for GDM |
| 5 | prev_pregnancy_2_GTT_50g | 50 g GTT at 2nd previous pregnancy |
| 6 | n_DM_reletives | Number of reletives with diagnosed diabetes |
| 7 | prev_pregnancy_1_GLUCOSE | Labtest GLUCOSE, sampled during 1st previous pregnancy |
| 8 | baseline_BMI | Baseline BMI |
| 9 | baseline_height | Baseline height |
| 10 | F1_NEUT_abs | Labtest NEUT.abs, sampled during F1: first blood test (weeks 6-12) |
| 11 | F1_WBC | Labtest WBC, sampled during F1: first blood test (weeks 6-12) |
| 12 | F2_GLUCOSE | Labtest GLUCOSE, sampled during F2: second blood test (weeks 16-19) |
| 13 | M1_GLUCOSE | Labtest GLUCOSE, sampled during M1: year 1 before pregnancy |
| 14 | mother_born_in_israel | Mother born in Israel |
| 15 | M2_GLUCOSE | Labtest GLUCOSE, sampled during M2: year 2 before pregnancy |
| 16 | max_HbA1c | Maximal HbA1c % recorded before pregnancy |
| 17 | prev_pregnancy_1_GTT_100g_120min | 100 g GTT 120 minutes at 1st previous pregnancy |
| 18 | prev_pregnancy_1_GTT_100g_180min | 100 g GTT 180 minutes at 1st previous pregnancy |
| 19 | prev_pregnancy_1_GTT_100g_60min | 100 g GTT 60 minutes at 1st previous pregnancy |
| 20 | F2_systolic_BP | Systolic blood pressure sampled during F2: second blood test |
| | | (weeks 16-19) |
| 21 | ethnicity_ussr | Etnicity: USSR |
| 22 | F1_URIC_ACID | Labtest URIC ACID, sampled during F1: first blood test (weeks 6-12) |
| 23 | F1_BMI | BMI sampled during F1: first blood test (weeks 6-12) |
| 24 | M3_GLUCOSE | Labtest GLUCOSE, sampled during M3: year 3 before pregnancy |
| 25 | M4_GLUCOSE | Labtest GLUCOSE, sampled during M4: year 4 before pregnancy |
| 26 | F2_NEUT_abs | Labtest NEUT.abs, sampled during F2: second blood test (weeks 16-19) |
| 27 | prev_pregnancy_1_RBC | Labtest RBC, sampled during 1st previous pregnancy |
| 28 | F1_MICRO_perc_or_HYPO_perc | Labtest MICRO %/HYPO %, sampled during F1: first blood test |
| | | (weeks 6-12) |
| 29 | F1_TRIGLYCERIDES | Labtest TRIGLYCERIDES, sampled during F1: first blood test |
| | | (weeks 6-12) |
| 30 | n_prev_births | Number of mother's previous births |
| 31 | prev_pregnancy_1_NEUT_perc | Labtest NEUT %, sampled during 1st previous pregnancy |
| 32 | F1_GPT_ALT | Labtest GPT (ALT), sampled during F1: first blood test (weeks 6-12) |
| 33 | prev_pregnancy_1_MPV | Labtest MPV, sampled during 1st previous pregnancy |
| 34 | F2_diastolic_BP | Diastolic blood pressure sampled during F2: second blood test |
| | | (weeks 16-19) |
| 35 | baseline_weight | Baseline weight |
| 36 | F1_CREATININE | Labtest CREATININE, sampled during F1: first blood test |
| | | (weeks 6-12) |
| 37 | F2_WBC | Labtest WBC, sampled during F2: second blood test (weeks 16-19) |
| 38 | prev_pregnancy_2_GLUCOSE | Labtest GLUCOSE, sampled during 2nd previous pregnancy |
| 39 | F0_GLUCOSE | Labtest GLUCOSE, sampled during F0: before and early pregnancy |
| 40 | largest_baby_weight | Highest birth weight of baby recorded |
| 41 | prev_pregnancy_1_HCT | Labtest HCT, sampled during 1st previous pregnancy |
| 42 | F1_LYMP_abs | Labtest LYMP.abs, sampled during F1: first blood test (weeks 6-12) |
| 43 | prev_pregnancy_3_GTT_50g | 50 g GTT at 3rd previous pregnancy |
| 44 | prev_pregnancy_1_MCHC | Labtest MCHC, sampled during 1st previous pregnancy |
| 45 | prediabetes_history | History of prediabetes |
| 46 | F2_MICRO_perc_or_HYPO_perc | Labtest MICRO %/HYPO %, sampled during F2: second blood test |
| | | (weeks 16-19) |
| 47 | F1_TSH | Labtest TSH, sampled during F1: first blood test (weeks 6-12) |
| 48 | F1_HCT | Labtest HCT, sampled during F1: first blood test (weeks 6-12) |
| 49 | prev_pregnancy_1_GTT_100g_0min | 100 g GTT 0 minutes at 1st previous pregnancy |
| 50 | F0_GGT | Labtest GGT, sampled during F0: before and early pregnancy |
| 51 | F2_MONO_abs | Labtest MONO.abs, sampled during F2: second blood test |
| | | (weeks 16-19) |
| 52 | prev_pregnancy_1_is_GDM | GDM positive at 1st previous pregnancy |
| 53 | F1_CH | Labtest CH, sampled during F1: first blood test (weeks 6-12) |
| 54 | baseline_systolic_BP | Baseline systolic blood pressure |
| 55 | prev_pregnancy_1_EOS_perc | Labtest EOS %, sampled during 1st previous pregnancy |
| 56 | M1_WBC | Labtest WBC, sampled during Ml: year 1 before pregnancy |
| 57 | multiple_gestation_n | Multiple gestation: number of born children |
| 58 | F1_HYPO_perc | Labtest HYPO %, sampled during F1: first blood test (weeks 6-12) |
| 59 | F1_RUBELLA_Ab_IgG | Labtest RUBELLA Ab IgG, sampled during F1: first blood test |
| | | (weeks 6-12) |
| 60 | F2_MCHC | Labtest MCHC, sampled during F2: second blood test (weeks 16-19) |
| 61 | M1_HB | Labtest HB, sampled during M1: year 1 before pregnancy |
| 62 | prev_pregnancy_1_MONO_perc | Labtest MONO %, sampled during 1st previous pregnancy |
| 63 | prev_pregnancy_1_WBC | Labtest WBC, sampled during 1st previous pregnancy |
| 64 | M2_HB | Labtest HB, sampled during M2: year 2 before pregnancy |
| 65 | F2_HYPO_perc | Labtest HYPO %, sampled during F2: second blood test (weeks 16-19) |
| 66 | M5_GLUCOSE | Labtest GLUCOSE, sampled during M5: year 5 before pregnancy |
| 67 | prev_pregnancy_1_LYMP_abs | Labtest LYMP.abs, sampled during 1st previous pregnancy |
| 68 | F1_PLT | Labtest PLT, sampled during F1: first blood test (weeks 6-12) |
| 69 | F1_GGT | Labtest GGT, sampled during F1: first blood test (weeks 6-12) |
| 70 | F1_systolic_BP | Systolic blood pressure sampled during F1: first blood test |
| | | (weeks 6-12) |
| 71 | prev_pregnancy_1_HB | Labtest HB, sampled during 1st previous pregnancy |
| 72 | M1_NEUT_abs | Labtest NEUT.abs, sampled during M1: year 1 before pregnancy |
| 73 | F1_diastolic_BP | Diastolic blood pressure sampled during F1: first blood test |
| | | (weeks 6-12) |
| 74 | F2_clinic_V22_NORMAL_PREGNANCY | Clinic diagnosis NORMAL PREGNANCY (V22), count during |
| | | F2: second blood test (weeks 16-19) |
| 75 | F0_PROGESTERONE | Labtest PROGESTERONE, sampled during F0: before and early |
| | | pregnancy |
| 76 | F0_ESTRADIOL_E_2 | Labtest ESTRADIOL (E-2), sampled during F0: before and early |
| | | pregnancy |
| 77 | F1_RBC | Labtest RBC, sampled during F1: first blood test (weeks 6-12) |
| 78 | prev_pregnancy_1_EOS_abs | Labtest EOS.abs, sampled during 1st previous pregnancy |
| 79 | M2_HCT | Labtest HCT, sampled during M2: year 2 before pregnancy |
| 80 | baseline_diastolic_BP | Baseline diastolic blood pressure |
| 81 | F1_clinic_V68_ENCOUNTERS_{—} | Clinic diagnosis ENCOUNTERS FOR ADMINISTRATIVE |
| | FOR_ADMINISTRATIVE_{—} | PURPOSES (V68), count during F1: first blood test (weeks 6-12) |
| | PURPOSES | |
| 82 | F0_clinic_V68_ENCOUNTERS_{—} | Clinic diagnosis ENCOUNTERS FOR ADMINISTRATIVE |
| | FOR_ADMINISTRATIVE_{—} | PURPOSES (V68), count during F0: before and early pregnancy |
| | PURPOSES | |
| 83 | prev_pregnancy_2_GTT_100g_60min | 100 g GTT 60 minutes at 2nd previous pregnancy |
| 84 | prev_pregnancy_1_PLT | Labtest PLT, sampled during 1st previous pregnancy |
| 85 | F0_TRIGLYCERIDES | Labtest TRIGLYCERIDES, sampled during F0: before and early |
| | | pregnancy |
| 86 | F2_BMI | BMI sampled during F2: second blood test (weeks 16-19) |
| 87 | M2_BASO_abs | Labtest BASO abs, sampled during M2: year 2 before pregnancy |
| 88 | F1_MONO_abs | Labtest MONO.abs, sampled during F1: first blood test (weeks 6-12) |
| 89 | prev_pregnancy_2_MPV | Labtest MPV, sampled during 2nd previous pregnancy |
| 90 | prev_pregnancy_1_{—} | Labtest HEMOGLOBIN A1C %, sampled during 1st previous |
| | HEMOGLOBIN_A1C_perc | pregnancy |
| 91 | prev_pregnancy_1_NEUT_abs | Labtest NEUT.abs, sampled during 1st previous pregnancy |
| 92 | F1_CHOLESTEROL | Labtest CHOLESTEROL, sampled during F1: first blood test |
| | | (weeks 6-12) |
| 93 | F1_PDW | Labtest PDW, sampled during F1: first blood test (weeks 6-12) |
| 94 | BP_dia_change_coef_b | Estimated diastolic blood pressure at 26 weeks |
| 95 | F1_HDW | Labtest HDW, sampled during F1: first blood test (weeks 6-12) |
| 96 | F0_NON_HDL_CHOLESTEROL | Labtest NON-HDL_CHOLESTEROL, sampled during F0: before |
| | | and early pregnancy |
| 97 | F1_HEMOGLOBIN_A1C_perc | Labtest HEMOGLOBIN A1C %, sampled during F1: first blood |
| | | test (weeks 6-12) |
| 98 | M4_HCT | Labtest HCT, sampled during M4: year 4 before pregnancy |
| 99 | prev_pregnancy_2_GTT_100g_120min | 100 g GTT 120 minutes at 2nd previous pregnancy |
| 100 | Ml_EOS_perc | Labtest EOS %, sampled during M1: year 1 before pregnancy |
| 101 | Ml_LYMP_abs | Labtest LYMP.abs, sampled during M1: year 1 before pregnancy |
| 102 | prev_pregnancy_1_MCV | Labtest MCV, sampled during 1st previous pregnancy |
| 103 | prev_pregnancy_1_MONO_abs | Labtest MONO.abs, sampled during 1st previous pregnancy |
| 104 | F0_pharma_G03GA08_OVITRELLE_{—} | Prescription of OVITRELLE PREFILLED INJ 250 mcg |
| | PREFILLED_INJ_250mcg | (G03GA08), count during F0: before and early pregnancy |
| 105 | F1_BILIRUBIN_DIRECT | Labtest BILIRUBIN-DIRECT, sampled during F1: first blood test |
| | | (weeks 6-12) |
| 106 | F2_clinic_V68_ENCOUNTERS_{—} | Clinic diagnosis ENCOUNTERS FOR ADMINISTRATIVE |
| | FOR_ADMINISTRATIVE_{—} | PURPOSES (V68), count during F2: second blood test (weeks 16-19) |
| | PURPOSES | |
| 107 | F1_T4_FREE | Labtest T4- FREE, sampled during F1: first blood test (weeks 6-12) |
| 108 | M1_HCT | Labtest HCT, sampled during M1: year 1 before pregnancy |
| 109 | F0_GPT_ALT | Labtest GPT (ALT), sampled during F0: before and early pregnancy |
| 110 | M4_MONO_abs | Labtest MONO.abs, sampled during M4: year 4 before pregnancy |
| 111 | F1_clinic_6430_MILD_{—} | Clinic diagnosis MILD HYPEREMESIS GRAVIDARUM (6430), |
| | HYPEREMESIS_GRAVIDARUM | count during F1: first blood test (weeks 6-12) |
| 112 | prev_pregnancy_1_RDW | Labtest RDW, sampled during 1st previous pregnancy |
| 113 | F1_ALK_PHOSPHATASE | Labtest ALK. PHOSPHATASE, sampled during F1: first blood |
| | | test (weeks 6-12) |
| 114 | ethnicity_yemen | Etnicity: Yemen |
| 115 | F1_HCT_or_HGB_Ratio | Labtest HCT/HGB Ratio, sampled during F1: first blood test |
| | | (weeks 6-12) |
| 116 | F2_LYMP_abs | Labtest LYMP.abs, sampled during F2: second blood test |
| | | (weeks 16-19) |
| 117 | GDM_history | Recorded history of GDM according to GTTs |
| 118 | F1_BILIRUBIN_INDIRECT | Labtest BILIRUBIN INDIRECT, sampled during F1: first blood |
| | | test (weeks 6-12) |
| 119 | F1_BILIRUBIN_TOTAL | Labtest BILIRUBIN TOTAL, sampled during F1: first blood test |
| | | (weeks 6-12) |
| 120 | M5_HB | Labtest HB, sampled during M5: year 5 before pregnancy |
| 121 | M2_MPV | Labtest MPV, sampled during M2: year 2 before pregnancy |
| 122 | M3_BASO_abs | Labtest BASO abs, sampled during M3: year 3 before pregnancy |
| 123 | F1_CHOLESTEROL_LDL_calc | Labtest CHOLESTEROL-LDL calc, sampled during F1: first |
| | | blood test (weeks 6-12) |
| 124 | prev_pregnancy_1_MICRO_perc | Labtest MICRO %, sampled during 1st previous pregnancy |
| 125 | F1_pharma_A03FA01_{—} | Prescription of PRAMIN TAB 10 MG 30 (A03FA01), count |
| | PRAMIN_TAB_10MG_30 | during F1: first blood test (weeks 6-12) |
| 126 | F2_GLUCOSE_U_STRIP | Labtest GLUCOSE - U STRIP, sampled during F2: second blood |
| | | test (weeks 16-19) |
| 127 | M4_WBC | Labtest WBC, sampled during M4: year 4 before pregnancy |
| 128 | M4_PLT | Labtest PLT, sampled during M4: year 4 before pregnancy |
| 129 | F2_URIC_ACID | Labtest URIC ACID, sampled during F2: second blood test (weeks 16-19) |
| 130 | M3_EOS_perc | Labtest EOS %, sampled during M3: year 3 before pregnancy |
| 131 | F1_weight_time_to_GTT | Time to GTT of weight sampled during F1: first blood test (weeks 6-12) |
| 132 | M4_EOS_perc | Labtest EOS %, sampled during M4: year 4 before pregnancy |
| 133 | M3_HCT | Labtest HCT, sampled during M3: year 3 before pregnancy |
| 134 | F1_MPXI | Labtest MPXI, sampled during F1: first blood test (weeks 6-12) |
| 135 | F1_TRANSFERRIN | Labtest TRANSFERRIN, sampled during F1: first blood test |
| | | (weeks 6-12) |
| 136 | F1_EOS_perc | Labtest EOS %, sampled during F1: first blood test (weeks 6-12) |
| 137 | F1_LUC_abs | Labtest LUC abs, sampled during F1: first blood test (weeks 6-12) |
| 138 | F2_LUC_abs | Labtest LUC abs, sampled during F2: second blood test (weeks 16-19) |
| 139 | BP_sys_change_coef_b | Estimated systolic blood pressure at 26 weeks |
| 140 | prev_pregnancy_2_EOS_perc | Labtest EOS %, sampled during 2nd previous pregnancy |
| 141 | prev_pregnancy_2_HCT | Labtest HCT, sampled during 2nd previous pregnancy |
| 142 | M3_NEUT_perc | Labtest NEUT %, sampled during M3: year 3 before pregnancy |
| 143 | F2_GPT_ALT | Labtest GPT (ALT), sampled during F2: second blood test (weeks 16-19) |
| 144 | F2_NEUT_perc | Labtest NEUT %, sampled during F2: second blood test (weeks 16-19) |
| 145 | prev_pregnancy_2_HB | Labtest HB, sampled during 2nd previous pregnancy |
| 146 | F0_BMI | BMI sampled during F0: before and early pregnancy |
| 147 | prev_pregnancy_2_NEUT_abs | Labtest NEUT.abs, sampled during 2nd previous pregnancy |
| 148 | M3_RDW | Labtest RDW, sampled during M3: year 3 before pregnancy |
| 149 | prev_pregnancy_2_WBC | Labtest WBC, sampled during 2nd previous pregnancy |
| 150 | M3_HB | Labtest HB, sampled during M3: year 3 before pregnancy |
| 151 | F2_TRIGLYCERIDES | Labtest TRIGLYCERIDES, sampled during F2: second blood test |
| | | (weeks 16-19) |
| 152 | F2_HEMOGLOBIN_A1C_perc | Labtest HEMOGLOBIN A1C %, sampled during F2: second |
| | | blood test (weeks 16-19) |
| 153 | prev_pregnancy_2_LYMP_abs | Labtest LYMP.abs, sampled during 2nd previous pregnancy |
| 154 | F2_PH_U_STRIP | Labtest PH- U STRIP, sampled during F2: second blood test |
| | | (weeks 16-19) |
| 155 | F1_height | Height sampled during F1: first blood test (weeks 6-12) |
| 156 | M2_MACRO_perc | Labtest MACRO %, sampled during M2: year 2 before pregnancy |
| 157 | F0_URIC_ACID | Labtest URIC ACID, sampled during F0: before and early |
| | | pregnancy |
| 158 | F2_pharma_Unknown_{—} | Prescription of OTHER (Unknown_ATC), count during F2: |
| | ATC_OTHER | second blood test (weeks 16-19) |
| 159 | F2_POTASSIUM_v1 | Labtest POTASSIUM, sampled during F2: second blood test |
| | | (weeks 16-19) |
| 160 | prev_pregnancy_2_PLT | Labtest PLT, sampled during 2nd previous pregnancy |
| 161 | F1_POTASSIUM_v1 | Labtest POTASSIUM, sampled during F1: first blood test |
| | | (weeks 6-12) |
| 162 | M5_NEUT_perc | Labtest NEUT %, sampled during M5: year 5 before pregnancy |
| 163 | F2_weight | Weight sampled during F2: second blood test (weeks 16-19) |
| 164 | M5_HCT | Labtest HCT, sampled during M5: year 5 before pregnancy |
| 165 | prev_pregnancy_2_LYM_perc | Labtest LYM %, sampled during 2nd previous pregnancy |
| 166 | F2_EOS_perc | Labtest EOS %, sampled during F2: second blood test |
| | | (weeks 16-19) |
| 167 | F2_HCT | Labtest HCT, sampled during F2: second blood test (weeks 16-19) |
| 168 | F1_BASO_perc | Labtest BASO %, sampled during F1: first blood test |
| | | (weeks 6-12) |
| 169 | M3_MCH | Labtest MCH, sampled during M3: year 3 before pregnancy |
| 170 | M1_MCHC | Labtest MCHC, sampled during M1: year 1 before pregnancy |
| 171 | F1_PROGESTERONE | Labtest PROGESTERONE, sampled during F1: first blood test |
| | | (weeks 6-12) |
| 172 | prev_pregnancy_2_NEUT_perc | Labtest NEUT %, sampled during 2nd previous pregnancy |
| 173 | ethnicity_unknown | Etnicity: Unknown |
| 174 | M2_RDW | Labtest RDW, sampled during M2: year 2 before pregnancy |
| 175 | F1_UROBILINOGEN_U_STRIP | Labtest UROBILINOGEN-U STRIP, sampled during F1: first |
| | | blood test (weeks 6-12) |
| 176 | F1_NON_HDL_CHOLESTEROL | Labtest NON-HDL_CHOLESTEROL, sampled during F1: first |
| | | blood test (weeks 6-12) |
| 177 | F1_MCHC | Labtest MCHC, sampled during F1: first blood test (weeks 6-12) |
| 178 | M1_RBC | Labtest RBC, sampled during M1: year 1 before pregnancy |
| 179 | BP_sys_change_coef_a | Estimated change in systolic blood pressure by 26 weeks |
| 180 | F2_TSH | Labtest TSH, sampled during F2: second blood test (weeks 16-19) |
| 181 | prev_pregnancy_2_is_GDM | GDM positive at 2nd previous pregnancy |
| 182 | F1_UREA | Labtest UREA, sampled during F1: first blood test (weeks 6-12) |
| 183 | F1_PT_INR | Labtest PT-INR, sampled during F1: first blood test (weeks 6-12) |
| 184 | M3_MCV | Labtest MCV, sampled during M3: year 3 before pregnancy |
| 185 | ethnicity_asia | Etnicity: Asian |
| 186 | M5_MONO_abs | Labtest MONO.abs, sampled during M5: year 5 before pregnancy |
| 187 | F2_MICRO_perc | Labtest MICRO %, sampled during F2: second blood test |
| | | (weeks 16-19) |
| 188 | prev_pregnancy_1_HYPER_{—} | Labtest HYPER %, sampled during 1st previous pregnancy |
| | perc | |
| 189 | prev_pregnancy_3_RBC | Labtest RBC, sampled during 3rd previous pregnancy |
| 190 | M2_LYM_perc | Labtest LYM %, sampled during M2: year 2 before pregnancy |
| 191 | F1_BASO_abs | Labtest BASO abs, sampled during F1: first blood test |
| | | (weeks 6-12) |
| 192 | M2_EOS_abs | Labtest EOS.abs, sampled during M2: year 2 before pregnancy |
| 193 | M2_NEUT_abs | Labtest NEUT.abs, sampled during M2: year 2 before pregnancy |
| 194 | M3_RBC | Labtest RBC, sampled during M3: year 3 before pregnancy |
| 195 | locality_type_Non_Jewish_{—} | Lives in Non-Jewish Locality 20,000-49,999 residents |
| | Locality_20000_to_49999_{—} | |
| | residents | |
| 196 | F0_clinic_628_{—} | Clinic diagnosis INFERTILITY, FEMALE (628), count during |
| | INFERTILITY_FEMALE | F0: before and early pregnancy |
| 197 | F1_HbA | Labtest HbA, sampled during F1: first blood test (weeks 6-12) |
| 198 | Ml_MONO_abs | Labtest MONO.abs, sampled during M1: year 1 before pregnancy |
| 199 | F1_HB | Labtest HB, sampled during F1: first blood test (weeks 6-12) |
| 200 | M2_MONO_perc | Labtest MONO %, sampled during M2: year 2 before pregnancy |
| 201 | F0_systolic_BP | Systolic blood pressure sampled during F0: before and early |
| | | pregnancy |
| 202 | F2_LYM_perc | Labtest LYM %, sampled during F2: second blood test |
| | | (weeks 16-19) |
| 203 | M4_MICRO_perc | Labtest MICRO %, sampled during M4: year 4 before pregnancy |
| 204 | F1_FOLIC_ACID | Labtest FOLIC ACID, sampled during F1: first blood test |
| | | (weeks 6-12) |
| 205 | F1_RDW | Labtest RDW, sampled during F1: first blood test (weeks 6-12) |
| 206 | M3_NEUT_abs | Labtest NEUT.abs, sampled during M3: year 3 before pregnancy |
| 207 | F1_VITAMIN_B12 | Labtest VITAMIN B12, sampled during F1: first blood test |
| | | (weeks 6-12) |
| 208 | prev_pregnancy_2_GTT_100g_180min | 100 g GTT 180 minutes at 2nd previous pregnancy |
| 209 | F1_MICRO_perc | Labtest MICRO %, sampled during F1: first blood test |
| | | (weeks 6-12) |
| 210 | M2_LYMP_abs | Labtest LYMP.abs, sampled during M2: year 2 before pregnancy |
| 211 | F2_RDW_SD | Labtest RDW-SD, sampled during F2: second blood test |
| | | (weeks 16-19) |
| 212 | F2_T4_FREE | Labtest T4- FREE, sampled during F2: second blood test |
| | | (weeks 16-19) |
| 213 | F0_CHOLESTEROL | Labtest CHOLESTEROL, sampled during F0: before and early |
| | | pregnancy |
| 214 | F2_MCH | Labtest MCH, sampled during F2: second blood test (weeks 16- |
| | | 19) |
| 215 | F0_FERRITIN | Labtest FERRITIN, sampled during F0: before and early |
| | | pregnancy |
| 216 | F2_CHLORIDE | Labtest CHLORIDE, sampled during F2: second blood test |
| | | (weeks 16-19) |
| 217 | F0_BASO_abs | Labtest BASO abs, sampled during F0: before and early |
| | | pregnancy |
| 218 | F2_VITAMIN_B12 | Labtest VITAMIN B12, sampled during F2: second blood test |
| | | (weeks 16-19) |
| 219 | M3_MCHC | Labtest MCHC, sampled during M3: year 3 before pregnancy |
| 220 | M2_RBC | Labtest RBC, sampled during M2: year 2 before pregnancy |
| 221 | M4_HB | Labtest HB, sampled during M4: year 4 before pregnancy |
| 222 | F2_HbA | Labtest HbA, sampled during F2: second blood test (weeks 16-19) |
| 223 | M3_MPV | Labtest MPV, sampled during M3: year 3 before pregnancy |
| 224 | M1_MICRO_perc | Labtest MICRO %, sampled during M1: year 1 before pregnancy |
| 225 | F2_BILIRUBIN_TOTAL | Labtest BILIRUBIN TOTAL, sampled during F2: second blood |
| | | test (weeks 16-19) |
| 226 | M5_RDW | Labtest RDW, sampled during M5: year 5 before pregnancy |
| 227 | M5_MCHC | Labtest MCHC, sampled during M5: year 5 before pregnancy |
| 228 | M4_MPV | Labtest MPV, sampled during M4: year 4 before pregnancy |
| 229 | M2_MICRO_perc | Labtest MICRO %, sampled during M2: year 2 before pregnancy |
| 230 | M4_MONO_perc | Labtest MONO %, sampled during M4: year 4 before pregnancy |
| 231 | prev_pregnancy_1_BASO_perc | Labtest BASO %, sampled during 1st previous pregnancy |
| 232 | M2_MCV | Labtest MCV, sampled during M2: year 2 before pregnancy |
| 233 | prev_pregnancy_1_MCH | Labtest MCH, sampled during 1st previous pregnancy |
| 234 | glucose_change_coef_a | Estimated glucose change rate in 5 years prior to pregnancy |
| 235 | high_cholesterol_history | Recorded history of high cholesterol |
| 236 | F2_LDH | Labtest LDH, sampled during F2: second blood test |
| | | (weeks 16-19) |
| 237 | prev_pregnancy_3_RDW | Labtest RDW, sampled during 3rd previous pregnancy |
| 238 | F1_clinic_7_OBSERVATION_{—} | Clinic diagnosis OBSERVATION - PATIENT UNDER |
| | PATIENT_UNDER_{—} | OBSERVATION (7), count during F1: first blood test |
| | OBSERVATION | (weeks 6-12) |
| 239 | F2_clinic_7245_BACKACHE_{—} | Clinic diagnosis BACKACHE, UNSPECIFIED (7245), count |
| | UNSPECIFIED | during F2: second blood test (weeks 16-19) |
| 240 | prev_pregnancy_3_NEUT_abs | Labtest NEUT.abs, sampled during 3rd previous pregnancy |
| 241 | prev_pregnancy_3_MCV | Labtest MCV, sampled during 3rd previous pregnancy |
| 242 | F2_T3_FREE | Labtest T3- FREE, sampled during F2: second blood test (weeks |
| | | 16-19) |
| 243 | prev_pregnancy_2_GTT_{—} | 100 g GTT 0 minutes at 2nd previous pregnancy |
| | 100g_0min | |
| 244 | F2_ESTRADIOL_E_2 | Labtest ESTRADIOL (E-2), sampled during F2: second blood test |
| | | (weeks 16-19) |
| 245 | prev_pregnancy_2_HYPO_perc | Labtest HYPO %, sampled during 2nd previous pregnancy |
| 246 | F0_HbA | Labtest HbA, sampled during F0: before and early pregnancy |
| 247 | F0_MONO_abs | Labtest MONO.abs, sampled during F0: before and early |
| | | pregnancy |
| 248 | F0_FREE_ANDROGEN_{—} | Labtest FREE ANDROGEN INDEX, sampled during F0: before |
| | INDEX | and early pregnancy |
| 249 | F2_EOS_abs | Labtest EOS.abs, sampled during F2: second blood test |
| | | (weeks 16-19) |
| 250 | F2_BILIRUBIN_INDIRECT | Labtest BILIRUBIN INDIRECT, sampled during F2: second |
| | | blood test (weeks 16-19) |
| 251 | F2_GGT | Labtest GGT, sampled during F2: second blood test |
| | | (weeks 16-19) |
| 252 | M4_NEUT_abs | Labtest NEUT.abs, sampled during M4: year 4 before pregnancy |
| 253 | prev_pregnancy_3_HB | Labtest HB, sampled during 3rd previous pregnancy |
| 254 | prev_pregnancy_2_MICRO_perc | Labtest MICRO %, sampled during 2nd previous pregnancy |
| 255 | F0_CREATININE | Labtest CREATININE, sampled during F0: before and early |
| | | pregnancy |
| 256 | F1_weight | Weight sampled during F1: first blood test (weeks 6-12) |
| 257 | locality_type_Jewish_Locality_{—} | Lives in Jewish Locality 500,000 and more residents |
| | 500000_and_more_residents | |
| 258 | F0_LUC_abs | Labtest LUC abs, sampled during F0: before and early pregnancy |
| 259 | prev_pregnancy_1_HYPO_perc | Labtest HYPO %, sampled during 1st previous pregnancy |
| 260 | F0_LH | Labtest LH, sampled during F0: before and early pregnancy |
| 261 | F0_NEUT_abs | Labtest NEUT.abs, sampled during F0: before and early |
| | | pregnancy |
| 262 | M2_WBC | Labtest WBC, sampled during M2: year 2 before pregnancy |
| 263 | M3_BASO_perc | Labtest BASO %, sampled during M3: year 3 before pregnancy |
| 264 | F0_HDW | Labtest HDW, sampled during F0: before and early pregnancy |
| 265 | F1_ERYTHROCYTES_U_{—} | Labtest ERYTHROCYTES-U STRIP, sampled during F1: first |
| | STRIP | blood test (weeks 6-12) |
| 266 | F1_DHEA_SULPHATE | Labtest DHEA SULPHATE, sampled during F1: first blood test |
| | | (weeks 6-12) |
| 267 | F2_UREA | Labtest UREA, sampled during F2: second blood test |
| | | (weeks 16-19) |
| 268 | F2_PROGESTERONE | Labtest PROGESTERONE, sampled during F2: second blood test |
| | | (weeks 16-19) |
| 269 | M4_LYMP_abs | Labtest LYMP.abs, sampled during M4: year 4 before pregnancy |
| 270 | M1_RDW | Labtest RDW, sampled during M1: year 1 before pregnancy |
| 271 | M5_HYPO_perc | Labtest HYPO %, sampled during M5: year 5 before pregnancy |
| 272 | M1_PLT | Labtest PLT, sampled during M1: year 1 before pregnancy |
| 273 | F1_LDH | Labtest LDH, sampled during F1: first blood test (weeks 6-12) |
| 274 | F1_pharma_N02BB02_{—} | Prescription of OPTALGIN CPL 500 mg 42 (N02BB02), count |
| | OPTALGIN_CPL_{—} | during F1: first blood test (weeks 6-12) |
| | 500mg_42 | |
| 275 | M5_EOS_perc | Labtest EOS %, sampled during M5: year 5 before pregnancy |
| 276 | F2_HYPER_perc | Labtest HYPER %, sampled during F2: second blood test |
| | | (weeks 16-19) |
| 277 | F0_pharma_B03AD03_{—} | Prescription of AKTIFERRIN-F SOFT CAP 30 (B03AD03), |
| | AKTIFERRIN_F_SOFT_{—} | count during F0: before and early pregnancy |
| | CAP_30 | |
| 278 | F2_RBC | Labtest RBC, sampled during F2: second blood test (weeks 16-19) |
| 279 | F0_CH | Labtest CH, sampled during F0: before and early pregnancy |
| 280 | F0_LYM_perc | Labtest LYM %, sampled during F0: before and early pregnancy |
| 281 | F2_pharma_A11AA06_{—} | Prescription of PRENATAL 9 MONTHS (ALTMAN) CPL 60 |
| | PRENATAL_9_MONTHS_{—} | (A11AA06), count during F2: second blood test (weeks 16-19) |
| | ALTMAN_CPL_60 | |
| 282 | F2_HDW | Labtest HDW, sampled during F2: second blood test |
| | | (weeks 16-19) |
| 283 | M4_MCH | Labtest MCH, sampled during M4: year 4 before pregnancy |
| 284 | BP_dia_change_coef_a | Estimated change in diastolic blood pressure by 26 weeks |
| 285 | F1_MONO_perc | Labtest MONO %, sampled during F1: first blood test |
| | | (weeks 6-12) |
| 286 | weight_change_coef_b | Estimated weight at 26 weeks |
| 287 | F0_CHOLESTEROL_HDL | Labtest CHOLESTEROL- HDL, sampled during F0: before and |
| | | early pregnancy |
| 288 | F1_HYPER_perc | Labtest HYPER %, sampled during F1: first blood test |
| | | (weeks 6-12) |
| 289 | F2_MCV | Labtest MCV, sampled during F2: second blood test |
| | | (weeks 16-19) |
| 290 | F1_HbA2 | Labtest HbA2, sampled during F1: first blood test (weeks 6-12) |
| 291 | M4_BASO_perc | Labtest BASO %, sampled during M4: year 4 before pregnancy |
| 292 | M4_MCV | Labtest MCV, sampled during M4: year 4 before pregnancy |
| 293 | prev_pregnancy_3_EOS_perc | Labtest EOS %, sampled during 3rd previous pregnancy |
| 294 | locality_type_Jewish_Locality_{—} | Lives in Jewish Locality 20,000-49,999 residents |
| | 20000_to_49999_residents | |
| 295 | F2_weight_time_to_GTT | Time to GTT of weight sampled during F2: second blood test |
| | | (weeks 16-19) |
| 296 | F1_T3_FREE | Labtest T3- FREE, sampled during F1: first blood test |
| | | (weeks 6-12) |
| 297 | F2_height | Height sampled during F2: second blood test (weeks 16-19) |
| 298 | F1_SPECIFIC_GRAV_U_STRI | Labtest SPECIFIC GRAV-U STRI, sampled during F1: first blood |
| | | test (weeks 6-12) |
| 299 | weight_change_coef_a | Estimated weight change from 10 weeks to 26 weeks |
| 300 | prev_pregnancy_2_MCV | Labtest MCV, sampled during 2nd previous pregnancy |
| 301 | F1_pharma_G03DA04_{—} | Prescription of ENDOMETRIN VAGINAL TAB 100 MG BOX |
| | ENDOMETRIN_VAGINAL_{—} | 30 (G03DA04), count during F1: first blood test (weeks 6-12) |
| | TAB_100MG_BOX_30 | |
| 302 | F2_CHOLESTEROL_HDL | Labtest CHOLESTEROL- HDL, sampled during F2: second |
| | | blood test (weeks 16-19) |
| 303 | F0_DHEA_SULPHATE | Labtest DHEA SULPHATE, sampled during F0: before and early |
| | | pregnancy |
| 304 | prev_pregnancy_2_MCHC | Labtest MCHC, sampled during 2nd previous pregnancy |
| 305 | prev_pregnancy_2_RBC | Labtest RBC, sampled during 2nd previous pregnancy |
| 306 | prev_pregnancy_2_MACRO_perc | Labtest MACRO %, sampled during 2nd previous pregnancy |
| 307 | F0_WBC | Labtest WBC, sampled during F0: before and early pregnancy |
| 308 | M4_NEUT_perc | Labtest NEUT %, sampled during M4: year 4 before pregnancy |
| 309 | F1_ESTRADIOL_E_2 | Labtest ESTRADIOL (E-2), sampled during F1: first blood test |
| | | (weeks 6-12) |
| 310 | F2_pharma_C10AX06_{—} | Prescription of MAARAZ 9M: OMEGA-3 + PRENATAL(ALT |
| | MAARAZ_9M_OMEGA_{—} | KIT 120 (C10AX06), count during F2: second blood test |
| | 3+PRENATAL_ALT_KIT_120 | (weeks 16-19) |
| 311 | M1_HYPO_perc | Labtest HYPO %, sampled during M1: year 1 before pregnancy |
| 312 | F1_KETONES_U_STRIP | Labtest KETONES- U STRIP, sampled during F1: first blood test |
| | | (weeks 6-12) |
| 313 | F2_PLT | Labtest PLT, sampled during F2: second blood test (weeks 16-19) |
| 314 | locality_type_Moshav | Lives in Moshav |
| 315 | prev_pregnancy_2_MONO_perc | Labtest MONO %, sampled during 2nd previous pregnancy |
| 316 | F2_SPECIFIC_GRAV_U_STRI | Labtest SPECIFIC GRAV-U STRI, sampled during F2: second |
| | | blood test (weeks 16-19) |
| 317 | F2_PROTEIN_U_STRIP | Labtest PROTEIN- U STRIP, sampled during F2: second blood |
| | | test (weeks 16-19) |
| 318 | F2_APTT_sec | Labtest APTT-sec, sampled during F2: second blood test |
| | | (weeks 16-19) |
| 319 | prediabetes_diag_history | Recorded diagnosis of prediabetes |
| 320 | M3_HYPO_perc | Labtest HYPO %, sampled during M3: year 3 before pregnancy |
| 321 | F1_GLUCOSE_U_STRIP | Labtest GLUCOSE - U STRIP, sampled during F1: first blood test |
| | | (weeks 6-12) |
| 322 | F2_MACRO_perc | Labtest MACRO %, sampled during F2: second blood test |
| | | (weeks 16-19) |
| 323 | F1_FIBRINOGEN_CALCU | Labtest FIBRINOGEN CALCU, sampled during F1: first blood |
| | | test (weeks 6-12) |
| 324 | F1_RDW_SD | Labtest RDW-SD, sampled during F1: first blood test |
| | | (weeks 6-12) |
| 325 | F0_IRON | Labtest IRON, sampled during F0: before and early pregnancy |
| 326 | F1_clinic_5990_URINARY_{—} | Clinic diagnosis URINARY TRACT INFECTION, SITE NOT |
| | TRACT_INFECTION_SITE_{—} | SPECIFIED (5990), count during F1: first blood test (weeks 6-12) |
| | NOT_SPECIFIED | |
| 327 | F1_LEUCOCYTES_U_STRIP | Labtest LEUCOCYTES - U STRIP, sampled during F1: first |
| | | blood test (weeks 6-12) |
| 328 | M4_RBC | Labtest RBC, sampled during M4: year 4 before pregnancy |
| 329 | prev_pregnancy_3_NEUT_perc | Labtest NEUT %, sampled during 3rd previous pregnancy |
| 330 | F2_LEUCOCYTES_U_STRIP | Labtest LEUCOCYTES - U STRIP, sampled during F2: second |
| | | blood test (weeks 16-19) |
| 331 | M1_MCV | Labtest MCV, sampled during M1: year 1 before pregnancy |
| 332 | F1_MCH | Labtest MCH, sampled during F1: first blood test (weeks 6-12) |
| 333 | prev_pregnancy_1_LYM_perc | Labtest LYM %, sampled during 1st previous pregnancy |
| 334 | F2_clinic_V6801_ENCOUNTERS_{—} | Clinic diagnosis ENCOUNTERS FOR DISABILITY |
| | FOR_DISABILITY_EXAMINATION | EXAMINATION (V6801), count during F2: second blood test |
| | | (weeks 16-19) |
| 335 | F0_KETONES_U_STRIP | Labtest KETONES- U STRIP, sampled during F0: before and |
| | | early pregnancy |
| 336 | locality_type_Non_Jewish_{—} | Lives in Non-Jewish Locality 10,000-19,999 residents |
| | Locality_10000_to_19999_{—} | |
| | residents | |
| 337 | F1_GLOBULIN | Labtest GLOBULIN, sampled during F1: first blood test |
| | | (weeks 6-12) |
| 338 | F2_ALK_PHOSPHATASE | Labtest ALK. PHOSPHATASE, sampled during F2: second blood |
| | | test (weeks 16-19) |
| 339 | F2_BP_time_to_GTT | Time to GTT of blood pressure sampled during F2: second blood |
| | | test (weeks 16-19) |
| 340 | F0_pharma_N06AB10_{—} | Prescription of CIPRALEX (MUSHLAM) TAB 10 MG 28 |
| | CIPRALEX_MUSHLAM_{—} | (N06AB10), count during F0: before and early pregnancy |
| | TAB_10MG_28 | |
| 341 | M5_MONO_perc | Labtest MONO %, sampled during M5: year 5 before pregnancy |
| 342 | F2_FERRITIN | Labtest FERRITIN, sampled during F2: second blood test |
| | | (weeks 16-19) |
| 343 | F2_PT_INR | Labtest PT-INR, sampled during F2: second blood test |
| | | (weeks 16-19) |
| 344 | M3_EOS_abs | Labtest EOS.abs, sampled during M3: year 3 before pregnancy |
| 345 | F1_CONTROL_PTT | Labtest CONTROL PTT, sampled during F1: first blood test |
| | | (weeks 6-12) |
| 346 | locality_type_Kibbutz | Lives in Kibbutz |
| 347 | F1_PHOSPHORUS | Labtest PHOSPHORUS, sampled during F1: first blood test |
| | | (weeks 6-12) |
| 348 | F0_COMPLEMENT_C3 | Labtest COMPLEMENT C3, sampled during F0: before and early |
| | | pregnancy |
| 349 | F2_MPXI | Labtest MPXI, sampled during F2: second blood test |
| | | (weeks 16-19) |
| 350 | F1_clinic_P027_ISSUE_OF_{—} | Clinic diagnosis ISSUE OF MEDICAL CERTIFICATE (P027), |
| | MEDICAL_CERTIFICATE | count during F1: first blood test (weeks 6-12) |
| 351 | F0_pharma_R02AA20_{—} | Prescription of LEMOCIN CHERRY SUGAR FREE LOZ CF 24 |
| | LEMOCIN_CHERRY_SUGAR_{—} | (R02AA20), count during F0: before and early pregnancy |
| | FREE_LOZ_CF_24 | |
| 352 | M3_PLT | Labtest PLT, sampled during M3: year 3 before pregnancy |
| 353 | F2_clinic_6161_VAGINITIS_{—} | Clinic diagnosis VAGINITIS AND VULVOVAGINITIS (6161), |
| | AND_VULVOVAGINITIS | count during F2: second blood test (weeks 16-19) |
| 354 | prev_pregnancy_3_GTT_{—} | 100 g GTT 180 minutes at 3rd previous pregnancy |
| | 100g_180min | |
| 355 | F2_NON_HDL_CHOLESTEROL | Labtest NON-HDL_CHOLESTEROL, sampled during F2: second |
| | | blood test (weeks 16-19) |
| 356 | prev_pregnancy_3_GTT_{—} | 100 g GTT 60 minutes at 3rd previous pregnancy |
| | 100g_60min | |
| 357 | F0_MONO_perc | Labtest MONO %, sampled during F0: before and early pregnancy |
| 358 | M5_MACRO_perc | Labtest MACRO %, sampled during M5: year 5 before pregnancy |
| 359 | F1_hospital_64303_MILD_{—} | Hospital diagnosis MILD HYPEREMESIS GRAVIDARUM, |
| | HYPEREMESIS_GRAVIDARUM_{—} | ANTEPARTUM (64303), count during F1: first blood test |
| | ANTEPARTUM | (weeks 6-12) |
| 360 | F0_RDW | Labtest RDW, sampled during F0: before and early pregnancy |
| 361 | prev_pregnancy_1_MACRO_perc | Labtest MACRO %, sampled during 1st previous pregnancy |
| 362 | F0_TSH | Labtest TSH, sampled during F0: before and early pregnancy |
| 363 | M5_EOS_abs | Labtest EOS.abs, sampled during M5: year 5 before pregnancy |
| 364 | F0_APTT_sec | Labtest APTT-sec, sampled during F0: before and early |
| | | pregnancy |
| 365 | M5_MPV | Labtest MPV, sampled during M5: year 5 before pregnancy |
| 366 | M1_HYPER_perc | Labtest HYPER %, sampled during M1: year 1 before pregnancy |
| 367 | F2_clinic_7_OBSERVATION_{—} | Clinic diagnosis OBSERVATION - PATIENT UNDER |
| | PATIENT_UNDER_{—} | OBSERVATION (7), count during F2: second blood test |
| | OBSERVATION | (weeks 16-19) |
| 368 | F1_clinic_6510_TWIN_{—} | Clinic diagnosis TWIN PREGNANCY (6510), count during F1: |
| | PREGNANCY | first blood test (weeks 6-12) |
| 369 | M5_MCH | Labtest MCH, sampled during M5: year 5 before pregnancy |
| 370 | F1_17_OH_PROGESTERONE | Labtest 17-OH-PROGESTERONE, sampled during F1: first |
| | | blood test (weeks 6-12) |
| 371 | prev_pregnancy_3_GTT_{—} | 100 g GTT 120 minutes at 3rd previous pregnancy |
| | 100g_120min | |
| 372 | prev_pregnancy_2_RDW | Labtest RDW, sampled during 2nd previous pregnancy |
| 373 | F0_pharma_N02BE01_{—} | Prescription of ACAMOL CPL 500 MG 21 (N02BE01), count |
| | ACAMOL_CPL_500MG_21 | during F0: before and early pregnancy |
| 374 | F0_PROT_S_ANTIGEN_FREE | Labtest PROT-S ANTIGEN (FREE, sampled during F0: before |
| | | and early pregnancy |
| 375 | F2_VITAMIN_D_25_OH | Labtest VITAMIN D (25-OH), sampled during F2: second blood |
| | | test (weeks 16-19) |
| 376 | M4_LYM_perc | Labtest LYM %, sampled during M4: year 4 before pregnancy |
| 377 | F1_PROLACTIN | Labtest PROLACTIN, sampled during F1: first blood test |
| | | (weeks 6-12) |
| 378 | F1_pharma_N02BE01_{—} | Prescription of TIPTIPOT NOVIMOL SUS 100 mg/1 mL 15 mL |
| | TIPTIPOT_NOVIMOL_{—} | (N02BE01), count during F1: first blood test (weeks 6-12) |
| | SUS_100mg_OR_1mL_15mL | |
| 379 | F1_clinic_7291_MYALGIA_{—} | Clinic diagnosis MYALGIA AND MYOSITIS, UNSPECIFIED |
| | AND_MYOSITIS_{—} | (7291), count during F1: first blood test (weeks 6-12) |
| | UNSPECIFIED | |
| 380 | F2_clinic_3810_ACUTE_{—} | Clinic diagnosis ACUTE NONSUPPURATIVE OTITIS MEDIA |
| | NONSUPPURATIVE_{—} | (3810), count during F2: second blood test (weeks 16-19) |
| | OTITIS_MEDIA | |
| 381 | F0_MPV | Labtest MPV, sampled during F0: before and early pregnancy |
| 382 | F1_clinic_0799_UNSP_VIR | Clinic diagnosis UNSP.VIRAL INFECT.IN CONDITIONS |
| | AL_INFECT_IN_CONDITIONS_{—} | CLASSIF.ELSEWHERE, UNSP.SITE (0799), count during F1: |
| | CLASSIF_ELSEWHERE_{—} | first blood test (weeks 6-12) |
| | UNSP_SITE | |
| 383 | F2_clinic_6929_CONTACT_{—} | Clinic diagnosis CONTACT DERMATITIS AND OTHER |
| | DERMATITIS_AND_OTHER_{—} | ECZEMA, UNSPECIFIED CAUSE (6929), count during F2: |
| | ECZEMA_UNSPECIFIED_CAUSE | second blood test (weeks 16-19) |
| 384 | F0_PH_U_STRIP | Labtest PH- U STRIP, sampled during F0: before and early |
| | | pregnancy |
| 385 | F2_HB | Labtest HB, sampled during F2: second blood test (weeks 16-19) |
| 386 | F0_MAGNESIUM | Labtest MAGNESIUM, sampled during F0: before and early |
| | | pregnancy |
| 387 | F1_pharma_B03AD03_{—} | Prescription of AKTIFERRIN-F SOFT CAP 30 (B03AD03), |
| | AKTIFERRIN_F_SOFT_{—} | count during F1: first blood test (weeks 6-12) |
| | CAP_30 | |
| 388 | F0_clinic_632_MISSED_{—} | Clinic diagnosis MISSED ABORTION (632), count during F0: |
| | ABORTION | before and early pregnancy |
| 389 | F0_CA1CIUM | Labtest CA1CIUM, sampled during F0: before and early |
| | | pregnancy |
| 390 | F1_EOS_abs | Labtest EOS.abs, sampled during F1: first blood test (weeks 6-12) |
| 391 | F2_EPITHELIAL_SED | Labtest EPITHELIAL-SED, sampled during F2: second blood test |
| | | (weeks 16-19) |
| 392 | prev_pregnancy_2_MONO_abs | Labtest MONO.abs, sampled during 2nd previous pregnancy |
| 393 | F2_clinic_677_LATE_EFFECT_{—} | Clinic diagnosis LATE EFFECT OF COMPLICATION OF |
| | OF_COMPLICATION_OF_{—} | PREGNANCY, CHILDBIRTH, PUERPERIUM (677), count |
| | PREGNANCY_CHILDBIRTH_{—} | during F2: second blood test (weeks 16-19) |
| | PUERPERIUM | |
| 394 | F0_BP_time_to_GTT | Time to GTT of blood pressure sampled during F0: before and |
| | | early pregnancy |
| 395 | F2_clinic_5640_CONSTIPATION | Clinic diagnosis CONSTIPATION (5640), count during F2: |
| | | second blood test (weeks 16-19) |
| 396 | M3_MONO_perc | Labtest MONO %, sampled during M3: year 3 before pregnancy |
| 397 | F2_clinic_2449_UNSPECIFIED_{—} | Clinic diagnosis UNSPECIFIED ACQUIRED |
| | ACQUIRED_HYPOTHYROIDISM | HYPOTHYROIDISM (2449), count during F2: second blood test |
| | | (weeks 16-19) |
| 398 | F1_MACRO_perc | Labtest MACRO %, sampled during F1: first blood test |
| | | (weeks 6-12) |
| 399 | F2_CHOLESTEROL_LDL_calc | Labtest CHOLESTEROL-LDL calc, sampled during F2: second |
| | | blood test (weeks 16-19) |
| 400 | F1_pharma_H03AA01_ELT | Prescription of ELTROXIN TAB 50 MCG 100 (H03AA01), count |
| | ROXIN_TAB_50MCG_100 | during F1: first blood test (weeks 6-12) |
| 401 | F2_pharma_U0_LABON_TIME | Prescription of LABON TIME (U0), count during F2: second |
| | | blood test (weeks 16-19) |
| 402 | F0_clinic_7_OBSERVATION_P | Clinic diagnosis OBSERVATION - PATIENT UNDER |
| | ATIENT_UNDER_{—} | OBSERVATION (7), count during F0: before and early |
| | OBSERVATION | pregnancy |
| 403 | F2_pharma_U1_CLEAR_TEST | Prescription of CLEAR TEST (U1), count during F2: second |
| | | blood test (weeks 16-19) |
| 404 | F0_BILIRUBIN_DIRECT | Labtest BILIRUBIN-DIRECT, sampled during F0: before and |
| | | early pregnancy |
| 405 | F0_CHOLESTEROL_LDL_calc | Labtest CHOLESTEROL-LDL calc, sampled during F0: before |
| | | and early pregnancy |
| 406 | M2_MONO_abs | Labtest MONO.abs, sampled during M2: year 2 before pregnancy |
| 407 | F1_CMV_AVIDITY | Labtest CMV AVIDITY, sampled during F1: first blood test |
| | | (weeks 6-12) |
| 408 | F0_TRANSFERRIN | Labtest TRANSFERRIN, sampled during F0: before and early |
| | | pregnancy |
| 409 | F2_CONTROL_PT | Labtest CONTROL PT, sampled during F2: second blood test |
| | | (weeks 16-19) |
| 410 | F2_pharma_H03AA01_{—} | Prescription of ELTROXIN TAB 50 MCG 100 (H03AA01), count |
| | ELTROXIN_TAB_50MCG_100 | during F2: second blood test (weeks 16-19) |
| 411 | prev_pregnancy_3_WBC | Labtest WBC, sampled during 3rd previous pregnancy |
| 412 | F1_ERYTHROCYTES_SED | Labtest ERYTHROCYTES-SED, sampled during F1: first blood |
| | | test (weeks 6-12) |
| 413 | F1_clinic_6264_IRREGULAR_{—} | Clinic diagnosis IRREGULAR MENSTRUAL CYCLE (6264), |
| | MENSTRUAL_CYCLE | count during F1: first blood test (weeks 6-12) |
| 414 | F1_clinic_487_INFLUENZA | Clinic diagnosis INFLUENZA (487), count during F1: first blood |
| | | test (weeks 6-12) |
| 415 | F2_LI | Labtest LI, sampled during F2: second blood test (weeks 16-19) |
| 416 | F1_clinic_Z887_DIAGNOSTIC_{—} | Clinic diagnosis DIAGNOSTIC ULTRASOUND (Z887), count |
| | ULTRASOUND | during F1: first blood test (weeks 6-12) |
| 417 | F0_RETICULOCYTES_COUNT_perc | Labtest RETICULOCYTES COUNT %, sampled during F0: |
| | | before and early pregnancy |
| 418 | M5_WBC | Labtest WBC, sampled during M5: year 5 before pregnancy |
| 419 | M2_BASO_perc | Labtest BASO %, sampled during M2: year 2 before pregnancy |
| 420 | F1_CONTROL_PT | Labtest CONTROL PT, sampled during F1: first blood test |
| | | (weeks 6-12) |
| 421 | F2_PCT | Labtest PCT, sampled during F2: second blood test (weeks 16-19) |
| 422 | F2_clinic_7847_EPISTAXIS | Clinic diagnosis EPISTAXIS (7847), count during F2: second |
| | | blood test (weeks 16-19) |
| 423 | F1_CHOLESTEROL_HDL | Labtest CHOLESTEROL- HDL, sampled during F1: first blood |
| | | test (weeks 6-12) |
| 424 | M2_HYPO_perc | Labtest HYPO %, sampled during M2: year 2 before pregnancy |
| 425 | F2_HbF | Labtest HbF, sampled during F2: second blood test (weeks 16-19) |
| 426 | F0_THYROGLOBULIN_Ab | Labtest THYROGLOBULIN Ab, sampled during F0: before and |
| | | early pregnancy |
| 427 | F1_clinic_677_LATE_EFFECT_OF_{—} | Clinic diagnosis LATE EFFECT OF COMPLICATION OF |
| | COMPLICATION_OF_PREGNANCY_{—} | PREGNANCY, CHILDBIRTH, PUERPERIUM (677), count |
| | CHILDBIRTH_PUERPERIUM | during F1: first blood test (weeks 6-12) |
| 428 | F1_pharma_G03DB01_{—} | Prescription of DUPHASTON TAB 10 MG BOX 20 (G03DB01), |
| | DUPHASTON_TAB_{—} | count during F1: first blood test (weeks 6-12) |
| | 10MG_BOX_20 | |
| 429 | F2_GLOBULIN | Labtest GLOBULIN, sampled during F2: second blood test |
| | | (weeks 16-19) |
| 430 | F0_clinic_8470_NECK_SPRAIN | Clinic diagnosis NECK SPRAIN (8470), count during F0: before |
| | | and early pregnancy |
| 431 | M5_RBC | Labtest RBC, sampled during M5: year 5 before pregnancy |
| 432 | F0_PT_perc | Labtest PT %, sampled during F0: before and early pregnancy |
| 433 | F0_MCH | Labtest MCH, sampled during F0: before and early pregnancy |
| 434 | prev_pregnancy_2_{—} | Labtest HEMOGLOBIN A1C %, sampled during 2nd previous |
| | HEMOGLOBIN_A1C_perc | pregnancy |
| 435 | F1_clinic_V71_OBSERVATION_{—} | Clinic diagnosis OBSERVATION AND EVALUATION FOR |
| | AND_EVALUATION_FOR_{—} | SUSPECTED CONDITIONS (V71), count during F1: first blood |
| | SUSPECTED_CONDITIONS | test (weeks 6-12) |
| 436 | F2_pharma_D01AC01_{—} | Prescription of CLOTRIMAZOLE (CLOTRIVIT) CR 1% 20 g |
| | CLOTRIMAZOLE_CLOTRIVIT_{—} | (D01AC01), count during F2: second blood test (weeks 16-19) |
| | CR_1%_20g | |
| 437 | F1_pharma_B03BB01_{—} | Prescription of FOLIC ACID (SAM-ON) TAB 5 MG 30 |
| | FOLIC_ACID_SAM_ON_{—} | (B03BB01), count during F1: first blood test (weeks 6-12) |
| | TAB_5MG_30 | |
| 438 | F0_MCHC | Labtest MCHC, sampled during F0: before and early pregnancy |
| 439 | M4_MCHC | Labtest MCHC, sampled during M4: year 4 before pregnancy |
| 440 | F0_clinic_216_BENIGN_{—} | Clinic diagnosis BENIGN NEOPLASM OF SKIN (216), count |
| | NEOPLASM_OF_SKIN | during F0: before and early pregnancy |
| 441 | M1_BASO_abs | Labtest BASO abs, sampled during M1: year 1 before pregnancy |
| 442 | F1_clinic_1104_{—} | Clinic diagnosis DERMATOPHYTOSIS OF FOOT (1104), count |
| | DERMATOPHYTOSIS_{—} | during F1: first blood test (weeks 6-12) |
| | OF_FOOT | |
| 443 | F0_GLOBULIN | Labtest GLOBULIN, sampled during F0: before and early |
| | | pregnancy |
| 444 | prev_pregnancy_2_MCH | Labtest MCH, sampled during 2nd previous pregnancy |
| 445 | F2_MAGNESIUM | Labtest MAGNESIUM, sampled during F2: second blood test |
| | | (weeks 16-19) |
| 446 | F2_PT_perc | Labtest PT %, sampled during F2: second blood test |
| | | (weeks 16-19) |
| 447 | F2_pharma_A11AA06_{—} | Prescription of PRENATAL PLUS CF TAB CF BOX 30 |
| | PRENATAL_PLUS_CF_{—} | (A11AA06), count during F2: second blood test (weeks 16-19) |
| | TAB_CF_BOX_30 | |
| 448 | F2_pharma_N02BE01_{—} | Prescription of ACAMOL CPL 500 MG 21 (N02BE01), count |
| | ACAMOL_CPL_500MG_21 | during F2: second blood test (weeks 16-19) |
| 449 | F1_LYM_perc | Labtest LYM %, sampled during F1: first blood test (weeks 6-12) |
| 450 | F1_IRON | Labtest IRON, sampled during F1: first blood test (weeks 6-12) |
| 451 | F1_clinic_525_DISEASES_{—} | Clinic diagnosis DISEASES AND CONDITIONS OF THE |
| | AND_CONDITIONS_OF_{—} | TEETH AND SUPPORT.STRUCTURES (525), count during F1: |
| | THE_TEETH_AND_{—} | first blood test (weeks 6-12) |
| | SUPPORT_STRUCTURES | |
| 452 | F0_GLUCOSE_U_STRIP | Labtest GLUCOSE - U STRIP, sampled during F0: before and |
| | | early pregnancy |
| 453 | F1_HbF | Labtest HbF, sampled during F1: first blood test (weeks 6-12) |
| 454 | M4_EOS_abs | Labtest EOS.abs, sampled during M4: year 4 before pregnancy |
| 455 | F2_C_REACTIVE_PROTEIN | Labtest C-REACTIVE PROTEIN, sampled during F2: second |
| | | blood test (weeks 16-19) |
| 456 | F2_pharma_D01AC63_{—} | Prescription of COMAGIS CR CF 15 GM (D01AC63), count |
| | COMAGIS_CR_CF_15GM | during F2: second blood test (weeks 16-19) |
| 457 | F2_clinic_7865_CHEST_PAIN | Clinic diagnosis CHEST PAIN (7865), count during F2: second |
| | | blood test (weeks 16-19) |
| 458 | F2_pharma_B03BB01_FOLIC_{—} | Prescription of FOLIC ACID (SAM-ON) TAB 5 MG 30 |
| | ACID_SAM_ON_TAB_5MG_30 | (B03BB01), count during F2: second blood test (weeks 16-19) |
| 459 | F1_SODIUM_v1 | Labtest SODIUM, sampled during F1: first blood test |
| | | (weeks 6-12) |
| 460 | locality_type_Non_Jewish_{—} | Lives in Non-Jewish Locality 50,000-99,999 residents |
| | Locality_50000_to_99999_{—} | |
| | residents | |
| 461 | F1_FERRITIN | Labtest FERRITIN, sampled during F1: first blood test |
| | | (weeks 6-12) |
| 462 | F0_ALK_PHOSPHATASE | Labtest ALK. PHOSPHATASE, sampled during F0: before and |
| | | early pregnancy |
| 463 | F0_A_B2_GLYCOPROTEI_{—} | Labtest A.B2 GLYCOPROTEI IgM, sampled during F0: before |
| | IgM | and early pregnancy |
| 464 | F1_PROT_S_ANTIGEN_FREE | Labtest PROT-S ANTIGEN (FREE, sampled during F1: first |
| | | blood test (weeks 6-12) |
| 465 | F2_clinic_L05_FLANK_{—} | Clinic diagnosis FLANK SYMPTOMS/COMPLAINTS (L05), |
| | SYMPTOMS_OR_{—} | count during F2: second blood test (weeks 16-19) |
| | COMPLAINTS | |
| 466 | F0_clinic_7242_LUMBAGO | Clinic diagnosis LUMBAGO (7242), count during F0: before and |
| | | early pregnancy |
| 467 | F1_pharma_H03AA01_{—} | Prescription of EUTHYROX TAB 100 mcg 100 (H03AA01), |
| | EUTHYROX_TAB_{—} | count during F1: first blood test (weeks 6-12) |
| | 100mcg_100 | |
| 468 | F1_APTT_sec | Labtest APTT-sec, sampled during F1: first blood test |
| | | (weeks 6-12) |
| 469 | F2_hospital_Z6812_{—} | Hospital diagnosis HYSTEROSCOPY (Z6812), count during F2: |
| | HYSTEROSCOPY | second blood test (weeks 16-19) |
| 470 | F0_clinic_A03_FEVER | Clinic diagnosis FEVER (A03), count during F0: before and early |
| | | pregnancy |
| 471 | F0_HYPER_perc | Labtest HYPER %, sampled during F0: before and early |
| | | pregnancy |
| 472 | F0_clinic_V22_NORMAL_{—} | Clinic diagnosis NORMAL PREGNANCY (V22), count during |
| | PREGNANCY | F0: before and early pregnancy |
| 473 | M4_RDW | Labtest RDW, sampled during M4: year 4 before pregnancy |
| 474 | M5_PLT | Labtest PLT, sampled during M5: year 5 before pregnancy |
| 475 | F0_TESTOSTERONE_TOTAL | Labtest TESTOSTERONE- TOTAL, sampled during F0: before |
| | | and early pregnancy |
| 476 | F0_pharma_U1_CLEAR_TEST | Prescription of CLEAR TEST (U1), count during F0: before and |
| | | early pregnancy |
| 477 | F2_clinic_7993_DEBILITY_{—} | Clinic diagnosis DEBILITY, UNSPECIFIED (7993), count |
| | UNSPECIFIED | during F2: second blood test (weeks 16-19) |
| 478 | F1_clinic_7245_BACKACHE_{—} | Clinic diagnosis BACKACHE, UNSPECIFIED (7245), count |
| | UNSPECIFIED | during F1: first blood test (weeks 6-12) |
| 479 | M2_NEUT_perc | Labtest NEUT %, sampled during M2: year 2 before pregnancy |
| 480 | F2_PROTEIN_TOTAL | Labtest PROTEIN-TOTAL, sampled during F2: second blood test |
| | | (weeks 16-19) |
| 481 | F2_AMYLASE | Labtest AMYLASE, sampled during F2: second blood test |
| | | (weeks 16-19) |
| 482 | F2_pharma_G01AF02_{—} | Prescription of AGISTEN VAG TAB 200 mg 3 (G01AF02), count |
| | AGISTEN_VAG_TAB_{—} | during F2: second blood test (weeks 16-19) |
| | 200mg_3 | |
| 483 | F0_HbF | Labtest HbF, sampled during F0: before and early pregnancy |
| 484 | F1_hospital_Z67591_CERC | Hospital diagnosis CERCLAGE-MC DONALD |
| | LAGE_MC_DONALD_{—} | (ENCIRCLEMENT SUTURE, CERVIX) (Z67591), count during |
| | ENCIRCLEMENT_{—} | F1: first blood test (weeks 6-12) |
| | SUTURE_CERVIX | |
| 485 | M2_MCH | Labtest MCH, sampled during M2: year 2 before pregnancy |
| 486 | F0_weight | Weight sampled during F0: before and early pregnancy |
| 487 | F0_MPXI | Labtest MPXI, sampled during F0: before and early pregnancy |
| 488 | F1_clinic_678_OTHER_{—} | Clinic diagnosis OTHER FETAL CONDITIONS (678), count |
| | FETAL_CONDITIONS | during F1: first blood test (weeks 6-12) |
| 489 | F1_hospital_632_MISSED_{—} | Hospital diagnosis MISSED ABORTION (632), count during F1: |
| | ABORTION | first blood test (weeks 6-12) |
| 490 | F0_pharma_H03AA01_{—} | Prescription of ELTROXIN TAB 100 MCG 100 (H03AA01), |
| | ELTROXIN_TAB_100MCG_100 | count during F0: before and early pregnancy |
| 491 | prev_pregnancy_3_GTT_{—} | 100 g GTT 0 minutes at 3rd previous pregnancy |
| | 100g_0min | |
| 492 | F2_clinic_462_ACUTE_{—} | Clinic diagnosis ACUTE PHARYNGITIS (462), count during F2: |
| | PHARYNGITIS | second blood test (weeks 16-19) |
| 493 | F0_COMPLEMENT_C4 | Labtest COMPLEMENT C4, sampled during F0: before and early |
| | | pregnancy |
| 494 | F1_PROTEIN_TOTAL | Labtest PROTEIN-TOTAL, sampled during F1: first blood test |
| | | (weeks 6-12) |
| 495 | F1_hospital_V220_{—} | Hospital diagnosis SUPERVISION OF NORMAL FIRST |
| | SUPERVISION_OF_{—} | PREGNANCY (V220), count during F1: first blood test |
| | NORMAL_FIRST_PREGNANCY | (weeks 6-12) |
| 496 | F1_PROTEIN_C_ACTIVITY | Labtest PROTEIN C ACTIVITY, sampled during F1: first blood |
| | | test (weeks 6-12) |
| 497 | F1_RETICUL_COUNT_abs | Labtest RETICUL. COUNT abs, sampled during F1: first blood |
| | | test (weeks 6-12) |
| 498 | F2_VARICELLA_ZOSTER_IgG | Labtest VARICELLA ZOSTER IgG, sampled during F2: second |
| | | blood test (weeks 16-19) |
| 499 | F0_BILIRUBIN_TOTAL | Labtest BILIRUBIN TOTAL, sampled during F0: before and |
| | | early pregnancy |
| 500 | F1_clinic_6918_OTHER_{—} | Clinic diagnosis OTHER ATOPIC DERMATITIS AND |
| | ATOPIC_DERMATITIS_AND_{—} | RELATED CONDITIONS (6918), count during F1: first blood |
| | RELATED_CONDITIONS | test (weeks 6-12) |
| 501 | M1_MPV | Labtest MPV, sampled during M1: year 1 before pregnancy |
| 502 | F0_diastolic_BP | Diastolic blood pressure sampled during F0: before and early |
| | | pregnancy |
| 503 | F2_PROTEIN_U_SAMPLE | Labtest PROTEIN- U SAMPLE, sampled during F2: second |
| | | blood test (weeks 16-19) |
| 504 | F0_RBC | Labtest RBC, sampled during F0: before and early pregnancy |
| 505 | F0_RUBELLA_Ab_IgG | Labtest RUBELLA Ab IgG, sampled during F0: before and early |
| | | pregnancy |
| 506 | F1_TESTOSTERONE_TOTAL | Labtest TESTOSTERONE- TOTAL, sampled during F1: first |
| | | blood test (weeks 6-12) |
| 507 | F1_CK_CREAT_KINASE_CPK | Labtest CK-CREAT.KINASE(CPK), sampled during F1: first |
| | | blood test (weeks 6-12) |
| 508 | F0_clinic_D09_NAUSEA | Clinic diagnosis NAUSEA (D09), count during F0: before and |
| | | early pregnancy |
| 509 | F0_VARICELLA_ZOSTER_IgG | Labtest VARICELLA ZOSTER IgG, sampled during F0: before |
| | | and early pregnancy |
| 510 | F2_KETONES_U_STRIP | Labtest KETONES- U STRIP, sampled during F2: second blood |
| | | test (weeks 16-19) |
| 511 | F0_clinic_V726_LABORATORY_{—} | Clinic diagnosis LABORATORY EXAMINATION (V726), |
| | EXAMINATION | count during F0: before and early pregnancy |
| 512 | F1_FIBRINOGEN | Labtest FIBRINOGEN, sampled during F1: first blood test |
| | | (weeks 6-12) |
| 513 | M5_LYMP_abs | Labtest LYMP.abs, sampled during M5: year 5 before pregnancy |
| 514 | F0_ALBUMIN | Labtest ALBUMIN, sampled during F0: before and early |
| | | pregnancy |
| 515 | F1_clinic_6981_PRURITUS_{—} | Clinic diagnosis PRURITUS OF GENITAL ORGANS (6981), |
| | OF_GENITAL_ORGANS | count during F1: first blood test (weeks 6-12) |
| 516 | F0_FSH | Labtest FSH, sampled during F0: before and early pregnancy |
| 517 | F0_hospital_V2681_{—} | Hospital diagnosis ENCOUNTER FOR ASSISTED |
| | ENCOUNTER_FOR_ASSISTED_{—} | REPRODUCTIVE FERTILITY PROCEDURE CYCLE (V2681), |
| | REPRODUCTIVE_FERTILITY_{—} | count during F0: before and early pregnancy |
| | PROCEDURE_CYCLE | |
| 518 | F1_clinic_9223_{—} | Clinic diagnosis CONTUSION OF BACK (9223), count during |
| | CONTUSION_OF_BACK | F1: first blood test (weeks 6-12) |
| 519 | F0_clinic_5589_OTHER_AND_{—} | Clinic diagnosis OTHER AND UNSPEC.NONINFECTIOUS |
| | UNSPEC_NONINFECTIOUS_{—} | GASTROENTERITIS AND COLITIS (5589), count during F0: |
| | GASTROENTERITIS_{—} | before and early pregnancy |
| | AND_COLITIS | |
| 520 | F2_clinic_8470_NECK_{—} | Clinic diagnosis NECK SPRAIN (8470), count during F2: second |
| | SPRAIN | blood test (weeks 16-19) |
| 521 | M3_HYPER_perc | Labtest HYPER %, sampled during M3: year 3 before pregnancy |
| 522 | prev_pregnancy_3_HYPER_perc | Labtest HYPER %, sampled during 3rd previous pregnancy |
| 523 | F2_clinic_0092_{—} | Clinic diagnosis INFECTIOUS DIARRHEA (0092), count during |
| | INFECTIOUS_DIARRHEA | F2: second blood test (weeks 16-19) |
| 524 | prev_pregnancy_3_MONO_abs | Labtest MONO.abs, sampled during 3rd previous pregnancy |
| 525 | F0_PROTEIN_URINE_24h | Labtest PROTEIN- URINE 24 h, sampled during F0: before and |
| | | early pregnancy |
| 526 | F0_hospital_78060_FEVER_{—} | Hospital diagnosis FEVER, UNSPECIFIED (78060), count |
| | UNSPECIFIED | during F0: before and early pregnancy |
| 527 | F2_LUC_perc | Labtest LUC %, sampled during F2: second blood test |
| | | (weeks 16-19) |
| 528 | F0_MCV | Labtest MCV, sampled during F0: before and early pregnancy |
| 529 | F2_RDW | Labtest RDW, sampled during F2: second blood test |
| | | (weeks 16-19) |
| 530 | prev_pregnancy_3_{—} | Labtest GLUCOSE, sampled during 3rd previous pregnancy |
| | GLUCOSE | |
| 531 | F1_PT_perc | Labtest PT %, sampled during F1: first blood test (weeks 6-12) |
| 532 | F1_pharma_A01AC21_{—} | Prescription of ORACORT E PAS TUB 5 g (A01AC21), count |
| | ORACORT_E_PAS_TUB_5g | during F1: first blood test (weeks 6-12) |
| 533 | F0_GLOM_FILTR_RATE | Labtest GLOM.FILTR.RATE, sampled during F0: before and |
| | | early pregnancy |
| 534 | M5_NEUT_abs | Labtest NEUT.abs, sampled during M5: year 5 before pregnancy |
| 535 | F1_pharma_C10AX06_{—} | Prescription of OMEGA-3 9 MONTHS (ALTMAN) CAP BOX |
| | OMEGA_3_9_MONTHS_{—} | 60 (C10AX06), count during F1: first blood test (weeks 6-12) |
| | ALTMAN_CAP_BOX_60 | |
| 536 | M3_MICRO_perc | Labtest MICRO %, sampled during M3: year 3 before pregnancy |
| 537 | F2_clinic_W84_PREGNANCY_{—} | Clinic diagnosis PREGNANCY HIGH RISK (W84), count during |
| | HIGH_RISK | F2: second blood test (weeks 16-19) |
| 538 | F0_HYPO_perc | Labtest HYPO %, sampled during F0: before and early pregnancy |
| 539 | F0_pharma_J01DC02_ZINNAT_{—} | Prescription of ZINNAT TAB 500 MG 10 (J01DC02), count |
| | TAB_500MG_10 | during F0: before and early pregnancy |
| 540 | F0_ERYTHROCYTES_U_STRIP | Labtest ERYTHROCYTES-U STRIP, sampled during F0: before |
| | | and early pregnancy |
| 541 | F2_CHOLESTEROL | Labtest CHOLESTEROL, sampled during F2: second blood test |
| | | (weeks 16-19) |
| 542 | F2_clinic_401_ESSENTIAL_{—} | Clinic diagnosis ESSENTIAL HYPERTENSION (401), count |
| | HYPERTENSION | during F2: second blood test (weeks 16-19) |
| 543 | F2_TESTOSTERONE_TOTAL | Labtest TESTOSTERONE- TOTAL, sampled during F2: second |
| | | blood test (weeks 16-19) |
| 544 | F1_pharma_A02BA03_{—} | Prescription of FAMOTIDINE-TEVA TAB 20 MG 30 |
| | FAMOTIDINE_TEVA_TAB_{—} | (A02BA03), count during F1: first blood test (weeks 6-12) |
| | 20MG_30 | |
| 545 | M2_PLT | Labtest PLT, sampled during M2: year 2 before pregnancy |
| 546 | F1_C_REACTIVE_PROTEIN | Labtest C-REACTIVE PROTEIN, sampled during F1: first blood |
| | | test (weeks 6-12) |
| 547 | F0_CREATININE_U_SAMPLE | Labtest CREATININE- U SAMPLE, sampled during F0: before |
| | | and early pregnancy |
| 548 | prev_pregnancy_2_EOS_abs | Labtest EOS.abs, sampled during 2nd previous pregnancy |
| 549 | M3_LYM_perc | Labtest LYM %, sampled during M3: year 3 before pregnancy |
| 550 | F1_ANTI_THYROID_PEROXID | Labtest ANTI THYROID PEROXID, sampled during F1: first |
| | | blood test (weeks 6-12) |
| 551 | F2_pharma_C10AX06_{—} | Prescription of OMEGA-3 9 MONTHS (ALTMAN) CAP BOX |
| | OMEGA_3_9_MONTHS_{—} | 60 (C10AX06), count during F2: second blood test (weeks 16-19) |
| | ALTMAN_CAP_BOX_60 | |
| 552 | F1_clinic_1101_{—} | Clinic diagnosis DERMATOPHYTOSIS OF NAIL (1101), count |
| | DERMATOPHYTOSIS_{—} | during F1: first blood test (weeks 6-12) |
| | OF_NAIL | |
| 553 | F2_clinic_7243_SCIATICA | Clinic diagnosis SCIATICA (7243), count during F2: second |
| | | blood test (weeks 16-19) |
| 554 | F1_clinic_628_INFERTILITY_{—} | Clinic diagnosis INFERTILITY, FEMALE (628), count during |
| | FEMALE | F1: first blood test (weeks 6-12) |
| 555 | F0_PT_INR | Labtest PT-INR, sampled during F0: before and early pregnancy |
| 556 | F1_clinic_7270_SYNOVITIS_{—} | Clinic diagnosis SYNOVITIS AND TENOSYNOVITIS (7270), |
| | AND_TENOSYNOVITIS | count during F1: first blood test (weeks 6-12) |
| 557 | F1_VARICELLA_ZOSTER_IgG | Labtest VARICELLA ZOSTER IgG, sampled during F1: first |
| | | blood test (weeks 6-12) |
| 558 | F2_CA1CIUM | Labtest CA1CIUM, sampled during F2: second blood test |
| | | (weeks 16-19) |
| 559 | F0_clinic_0799_UNSP_VIRAL_{—} | Clinic diagnosis UNSP.VIRAL INFECT.IN CONDITIONS |
| | INFECT_IN_CONDITIONS_{—} | CLASSIF.ELSEWHERE, UNSP.SITE (0799), count during F0: |
| | CLASSIF_ELSEWHERE_{—} | before and early pregnancy |
| | UNSP_SITE | |
| 560 | F1_clinic_346_MIGRAINE | Clinic diagnosis MIGRAINE (346), count during F1: first blood |
| | | test (weeks 6-12) |
| 561 | F2_LH | Labtest LH, sampled during F2: second blood test (weeks 16-19) |
| 562 | M1_EOS_abs | Labtest EOS.abs, sampled during M1: year 1 before pregnancy |
| 563 | F2_clinic_2452_CHRONIC_{—} | Clinic diagnosis CHRONIC LYMPHOCYTIC THYROIDITIS |
| | LYMPHOCYTIC_THYROIDITIS | (2452), count during F2: second blood test (weeks 16-19) |
| 564 | locality_type_Jewish_Locality_{—} | Lives in Jewish Locality 100,000-199,999 residents |
| | 100000_to_199999_residents | |
| 565 | F2_pharma_B03AD03_AKT | Prescription of AKTIFERRIN-F SOFT CAP 30 (B03AD03), |
| | IFERRIN_F_SOFT_CAP_30 | count during F2: second blood test (weeks 16-19) |
| 566 | F0_clinic_7802_SYNCOPE_{—} | Clinic diagnosis SYNCOPE AND COLLAPSE (7802), count |
| | AND_COLLAPSE | during F0: before and early pregnancy |
| 567 | F0_pharma_G03GA02_{—} | Prescription of MENOPUR VIA 75IU 10 (G03GA02), count |
| | MENOPUR_VIA_75IU_10 | during F0: before and early pregnancy |
| 568 | F1_MCV | Labtest MCV, sampled during F1: first blood test (weeks 6-12) |
| 569 | F1_clinic_A97_NO_DISEASE | Clinic diagnosis NO DISEASE (A97), count during F1: first |
| | | blood test (weeks 6-12) |
| 570 | F0_MICROALBUMIN_{—} | Labtest MICROALBUMIN-U SAMP, sampled during F0: before |
| | U_SAMP | and early pregnancy |
| 571 | prev_pregnancy_3_HYPO_perc | Labtest HYPO %, sampled during 3rd previous pregnancy |
| 572 | F1_clinic_07810_VIRAL_{—} | Clinic diagnosis VIRAL WARTS, UNSPECIFIED (07810), count |
| | WARTS_UNSPECIFIED | during F1: first blood test (weeks 6-12) |
| 573 | F0_pharma_G03AA10_{—} | Prescription of MELIANE TAB 21 (G03AA10), count during F0: |
| | MELIANE_TAB_21 | before and early pregnancy |
| 574 | F2_pharma_J01DC02_{—} | Prescription of ZINNAT TAB 250 MG 10 (J01DC02), count |
| | ZINNAT_TAB_250MG_10 | during F2: second blood test (weeks 16-19) |
| 575 | main_religion_Christians | Lives in locality with main religion Christians |
| 576 | F0_HCT | Labtest HCT, sampled during F0: before and early pregnancy |
| 577 | M5_HYPER_perc | Labtest HYPER %, sampled during M5: year 5 before pregnancy |
| 578 | M3_WBC | Labtest WBC, sampled during M3: year 3 before pregnancy |
| 579 | F2_clinic_3801_INFECTIVE_{—} | Clinic diagnosis INFECTIVE OTITIS EXTERNA (3801), count |
| | OTITIS_EXTERNA | during F2: second blood test (weeks 16-19) |
| 580 | F0_clinic_6235_LEUKORRHEA_{—} | Clinic diagnosis LEUKORRHEA, NOT SPECIFIED AS |
| | NOT_SPECIFIED_AS_INFECTIVE | INFECTIVE (6235), count during F0: before and early pregnancy |
| 581 | F2_pharma_H03AA01_{—} | Prescription of EUTHYROX TAB 100 mcg 100 (H03AA01), |
| | EUTHYROX_TAB_{—} | count during F2: second blood test (weeks 16-19) |
| | 100mcg_100 | |
| 582 | F1_pharma_A11CC05_D_{—} | Prescription of D-TABS TAB 400IU 90 (A11CC05), count during |
| | TABS_TAB_400IU_90 | F1: first blood test (weeks 6-12) |
| 583 | F1_hospital_Z9649_OTHER_{—} | Hospital diagnosis OTHER GENITOURINARY INSTILLATION |
| | GENITOURINARY_{—} | (Z9649), count during F1: first blood test (weeks 6-12) |
| | INSTILLATION | |
| 584 | F1_clinic_V724_PREGNANCY_{—} | Clinic diagnosis PREGNANCY EXAMINATION OR TEST, |
| | EXAMINATION_OR_TEST_{—} | PREGNANCY UNCONFIRMED (V724), count during F1: first |
| | PREGNANCY_UNCONFIRMED | blood test (weeks 6-12) |
| 585 | F0_MACRO_perc | Labtest MACRO %, sampled during F0: before and early |
| | | pregnancy |
| 586 | F2_LEUCOCYTES_SED | Labtest LEUCOCYTES-SED, sampled during F2: second blood |
| | | test (weeks 16-19) |
| 587 | F0_MICRO_perc_or_HYPO_perc | Labtest MICRO %/HYPO %, sampled during F0: before and early |
| | | pregnancy |
| 588 | prev_pregnancy_2_BASO_abs | Labtest BASO abs, sampled during 2nd previous pregnancy |
| 589 | locality_type_Jewish_Locality_{—} | Lives in Jewish Locality 2,000-4,999 residents |
| | 2000_to_4999_residents | |
| 590 | F0_PDW | Labtest PDW, sampled during F0: before and early pregnancy |
| 591 | F2_17_OH_PROGESTERONE | Labtest 17-OH-PROGESTERONE, sampled during F2: second |
| | | blood test (weeks 16-19) |
| 592 | F0_pharma_J01DC02_{—} | Prescription of ZINNAT TAB 250 MG 10 (J01DC02), count |
| | ZINNAT_TAB_250MG_10 | during F0: before and early pregnancy |
| 593 | M1_NEUT_perc | Labtest NEUT %, sampled during M1: year 1 before pregnancy |
| 594 | M5_LYM_perc | Labtest LYM %, sampled during M5: year 5 before pregnancy |
| 595 | M1_BASO_perc | Labtest BASO %, sampled during M1: year 1 before pregnancy |
| 596 | F2_pharma_R01AD08_{—} | Prescription of AVAMYS AQ. NASAL 120 INH SPR 27.5 MCG |
| | AVAMYS_AQ_NASAL_120_INH_{—} | (R01AD08), count during F2: second blood test (weeks 16-19) |
| | SPR_27_5MCG | |
| 597 | M5_BASO_abs | Labtest BASO abs, sampled during M5: year 5 before pregnancy |
| 598 | F2_BASO_perc | Labtest BASO %, sampled during F2: second blood test |
| | | (weeks 16-19) |
| 599 | F2_clinic_6792_ORAL_{—} | Clinic diagnosis ORAL CONTRACEPTION COUNSELING |
| | CONTRACEPTION_{—} | (6792), count during F2: second blood test (weeks 16-19) |
| | COUNSELING | |
| 600 | F0_HEMOGLOBIN_A | Labtest HEMOGLOBIN A, sampled during F0: before and early |
| | | pregnancy |
| 601 | F0_MICRO_perc | Labtest MICRO %, sampled during F0: before and early |
| | | pregnancy |
| 602 | F2_clinic_Z8909_{—} | Clinic diagnosis CONSULTATION, NOT OTHERWISE |
| | CONSULTATION_NOT_{—} | SPECIFIED (Z8909), count during F2: second blood test |
| | OTHERWISE_SPECIFIED | (weeks 16-19) |
| 603 | F1_clinic_7802_SYNCOPE_{—} | Clinic diagnosis SYNCOPE AND COLLAPSE (7802), count |
| | AND_COLLAPSE | during F1: first blood test (weeks 6-12) |
| 604 | F1_clinic_6929_CONTACT_{—} | Clinic diagnosis CONTACT DERMATITIS AND OTHER |
| | DERMATITIS_AND_OTHER_{—} | ECZEMA, UNSPECIFIED CAUSE (6929), count during F1: first |
| | ECZEMA_UNSPECIFIED_CAUSE | blood test (weeks 6-12) |
| 605 | F1_MPV | Labtest MPV, sampled during F1: first blood test (weeks 6-12) |
| 606 | F2_clinic_28709_OBESITY_{—} | Clinic diagnosis OBESITY (BMI >30) (28709), count during F2: |
| | BMI_>30 | second blood test (weeks 16-19) |
| 607 | F1_clinic_Y10_INFERTILITY_{—} | Clinic diagnosis INFERTILITY/SUBFERTILITY (Y10), count |
| | OR_SUBFERTILITY | during F1: first blood test (weeks 6-12) |
| 608 | F0_clinic_346_MIGRAINE | Clinic diagnosis MIGRAINE (346), count during F0: before and |
| | | early pregnancy |
| 609 | F2_CMV_IgG | Labtest CMV IgG, sampled during F2: second blood test |
| | | (weeks 16-19) |
| 610 | F1_hospital_65420_UTERINE_{—} | Hospital diagnosis UTERINE SCAR FROM |
| | SCAR_FROM_PREV_{—} | PREV.SURGERY, UNSP.EPISODE OF CARE, IN PREGN. |
| | SURGERY_UNSP_EPISODE_{—} | (65420), count during F1: first blood test (weeks 6-12) |
| | OF_CARE_IN_PREGN | |
| 611 | F0_FIBRINOGEN | Labtest FIBRINOGEN, sampled during F0: before and early |
| | | pregnancy |
| 612 | F2_THYROGLOBULIN_Ab | Labtest THYROGLOBULIN Ab, sampled during F2: second |
| | | blood test (weeks 16-19) |
| 613 | F2_pharma_G01AF02_{—} | Prescription of AGISTEN VAGINAL CR 2% 20 GM (G01AF02), |
| | AGISTEN_VAGINAL_CR_{—} | count during F2: second blood test (weeks 16-19) |
| | 2%_20GM | |
| 614 | F1_EPITHELIAL_SED | Labtest EPITHELIAL-SED, sampled during F1: first blood test |
| | | (weeks 6-12) |
| 615 | F0_BILIRUBIN_INDIRECT | Labtest BILIRUBIN INDIRECT, sampled during F0: before and |
| | | early pregnancy |
| 616 | M3_LYMP_abs | Labtest LYMP.abs, sampled during M3: year 3 before pregnancy |
| 617 | F1_pharma_B03BA01_VIT_{—} | Prescription of VIT B12 (FLORIS) SUBLING TAB 1000 MCG |
| | B12_FLORIS_SUBLING_{—} | 30 (B03BA01), count during F1: first blood test (weeks 6-12) |
| | TAB_1000MCG_30 | |
| 618 | F0_clinic_7062_SEBACEOUS_{—} | Clinic diagnosis SEBACEOUS CYST (7062), count during F0: |
| | CYST | before and early pregnancy |
| 619 | F2_RETICULOCYTES_{—} | Labtest RETICULOCYTES COUNT %, sampled during F2: |
| | COUNT_perc | second blood test (weeks 16-19) |
| 620 | F0_APTT_R | Labtest APTT-R, sampled during F0: before and early pregnancy |
| 621 | F2_clinic_0091_COLITIS_{—} | Clinic diagnosis COLITIS, ENTERITIS, GASTROENTERITIS |
| | ENTERITIS_{—} | PRESUMED INFECTIOUS ORIGIN (0091), count during F2: |
| | GASTROENTERITIS_{—} | second blood test (weeks 16-19) |
| | PRESUMED_INFECTIOUS_{—} | |
| | ORIGIN | |
| 622 | F0_clinic_3801_INFECTIV | Clinic diagnosis INFECTIVE OTITIS EXTERNA (3801), count |
| | E_OTITIS_EXTERNA | during F0: before and early pregnancy |
| 623 | F2_NORMOBLAST_abs | Labtest NORMOBLAST.abs, sampled during F2: second blood |
| | | test (weeks 16-19) |
| 624 | F0_LI | Labtest LI, sampled during F0: before and early pregnancy |
| 625 | F2_RUBELLA_Ab_IgG | Labtest RUBELLA Ab IgG, sampled during F2: second blood test |
| | | (weeks 16-19) |
| 626 | F1_ESR | Labtest ESR, sampled during F1: first blood test (weeks 6-12) |
| 627 | F0_clinic_7851_PALPITATIONS | Clinic diagnosis PALPITATIONS (7851), count during F0: before |
| | | and early pregnancy |
| 628 | F1_LI | Labtest LI, sampled during F1: first blood test (weeks 6-12) |
| 629 | F1_BLOOD_TYPE | Labtest BLOOD TYPE, sampled during F1: first blood test |
| | | (weeks 6-12) |
| 630 | F1_CA1CIUM | Labtest CA1CIUM, sampled during F1: first blood test |
| | | (weeks 6-12) |
| 631 | F2_clinic_X18_BREAST_{—} | Clinic diagnosis BREAST PAIN (X18), count during F2: second |
| | PAIN | blood test (weeks 16-19) |
| 632 | F1_clinic_V6801_ENCOUNTERS_{—} | Clinic diagnosis ENCOUNTERS FOR DISABILITY |
| | FOR_DISABILITY_{—} | EXAMINATION (V6801), count during F1: first blood test |
| | EXAMINATION | (weeks 6-12) |
| 633 | F2_CREATININE | Labtest CREATININE, sampled during F2: second blood test |
| | | (weeks 16-19) |
| 634 | F2_clinic_643_EXCESSIVE_{—} | Clinic diagnosis EXCESSIVE VOMITING IN PREGNANCY |
| | VOMITING_IN_PREGNANCY | (643), count during F2: second blood test (weeks 16-19) |
| 635 | F1_clinic_6236_VAGINAL_{—} | Clinic diagnosis VAGINAL HEMATOMA (6236), count during |
| | HEMATOMA | F1: first blood test (weeks 6-12) |
| 636 | F1_clinic_692_CONTACT_{—} | Clinic diagnosis CONTACT DERMATITIS AND OTHER |
| | DERMATITIS_AND_{—} | ECZEMA (692), count during F1: first blood test (weeks 6-12) |
| | OTHER_ECZEMA | |
| 637 | F1_AMYLASE | Labtest AMYLASE, sampled during F1: first blood test |
| | | (weeks 6-12) |
| 638 | locality_type_Non_Jewish_{—} | Lives in Non-Jewish Other Rural Locality |
| | Other_Rural_Locality | |
| 639 | F0_clinic_460_ACUTE_{—} | Clinic diagnosis ACUTE NASOPHARYNGITIS (COMMON |
| | NASOPHARYNGITIS_{—} | COLD) (460), count during F0: before and early pregnancy |
| | COMMON_COLD | |
| 640 | F2_pharma_N02BE01_{—} | Prescription of DEXAMOL CPL 500 MG 20 (N02BE01), count |
| | DEXAMOL_CPL_500MG_20 | during F2: second blood test (weeks 16-19) |
| 641 | F0_VITAMIN_D_25_OH | Labtest VITAMIN D (25-OH), sampled during F0: before and |
| | | early pregnancy |
| 642 | F0_pharma_A06AD15_{—} | Prescription of NORMALAX PWD 240 GM (A06AD15), count |
| | NORMALAX_PWD_240GM | during F0: before and early pregnancy |
| 643 | F2_ANTITHROMBIN_III | Labtest ANTITHROMBIN-III, sampled during F2: second blood |
| | | test (weeks 16-19) |
| 644 | F0_pharma_A11CC05_D_{—} | Prescription of D-DROPS DRP 200 UNIT 10 mL (A11CC05), |
| | DROPS_DRP_200UNIT_10mL | count during F0: before and early pregnancy |
| 645 | F0_clinic_Z887_DIAGNOSTIC_{—} | Clinic diagnosis DIAGNOSTIC ULTRASOUND (Z887), count |
| | ULTRASOUND | during F0: before and early pregnancy |
| 646 | F0_pharma_A02BC03_{—} | Prescription of LANTON CAP 30 MG BOX 28 (A02BC03), count |
| | LANTON_CAP_30MG_{—} | during F0: before and early pregnancy |
| | BOX_28 | |
| 647 | F2_CREATININE_U_SAMPLE | Labtest CREATININE- U SAMPLE, sampled during F2: second |
| | | blood test (weeks 16-19) |
| 648 | F1_clinic_V25_{—} | Clinic diagnosis CONTRACEPTIVE MANAGEMENT (V25), |
| | CONTRACEPTIVE_{—} | count during F1: first blood test (weeks 6-12) |
| | MANAGEMENT | |
| 649 | F0_RDW_CV | Labtest RDW-CV, sampled during F0: before and early pregnancy |
| 650 | F0_SODIUM_v1 | Labtest SODIUM, sampled during F0: before and early pregnancy |
| 651 | F1_clinic_V22_NORMAL_{—} | Clinic diagnosis NORMAL PREGNANCY (V22), count during |
| | PREGNANCY | F1: first blood test (weeks 6-12) |
| 652 | F1_clinic_7880_RENAL_COLIC | Clinic diagnosis RENAL COLIC (7880), count during F1: first |
| | | blood test (weeks 6-12) |
| 653 | F0_clinic_2662_OTHER_B_{—} | Clinic diagnosis OTHER B-COMPLEX DEFICIENCIES (2662), |
| | COMPLEX_DEFICIENCIES | count during F0: before and early pregnancy |
| 654 | F2_SODIUM_v1 | Labtest SODIUM, sampled during F2: second blood test |
| | | (weeks 16-19) |
| 655 | F1_clinic_L05_FLANK_{—} | Clinic diagnosis FLANK SYMPTOMS/COMPLAINTS (L05), |
| | SYMPTOMS_OR_{—} | count during F1: first blood test (weeks 6-12) |
| | COMPLAINTS | |
| 656 | F0_C_REACTIVE_PROTEIN | Labtest C-REACTIVE PROTEIN, sampled during F0: before and |
| | | early pregnancy |
| 657 | F2_CK_CREAT_KINASE_CPK | Labtest CK-CREAT.KINASE(CPK), sampled during F2: second |
| | | blood test (weeks 16-19) |
| 658 | M1_HEMOGLOBIN_A1C_perc | Labtest HEMOGLOBIN A1C %, sampled during M1: year 1 |
| | | before pregnancy |
| 659 | F2_clinic_V23_SUPERVISION_{—} | Clinic diagnosis SUPERVISION OF HIGH-RISK PREGNANCY |
| | OF_HIGH_RISK_PREGNANCY | (V23), count during F2: second blood test (weeks 16-19) |
| 660 | M1_LYM_perc | Labtest LYM %, sampled during M1: year 1 before pregnancy |
| 661 | F1_clinic_V828_SCREENING_{—} | Clinic diagnosis SCREENING FOR OTHER SPECIFIED |
| | FOR_OTHER_SPECIFIED_{—} | CONDITIONS (V828), count during F1: first blood test |
| | CONDITIONS | (weeks 6-12) |
| 662 | F1_pharma_J01XE01_{—} | Prescription of MACRODANTIN 29/M CAP 100 MG 30 |
| | MACRODANTIN_29_OR_{—} | (J01XE01), count during F1: first blood test (weeks 6-12) |
| | M_CAP_100MG_30 | |
| 663 | F1_clinic_595_CYSTITIS | Clinic diagnosis CYSTITIS (595), count during F1: first blood |
| | | test (weeks 6-12) |
| 664 | M1_MONO_perc | Labtest MONO %, sampled during M1: year 1 before pregnancy |
| 665 | locality_type_Jewish_Locality_{—} | Lives in Jewish Locality 200,000-499,999 residents |
| | 200000_to_499999_residents | |
| 666 | prev_pregnancy_1_BASO_abs | Labtest BASO abs, sampled during 1st previous pregnancy |
| 667 | F0_clinic_W84_{—} | Clinic diagnosis PREGNANCY HIGH RISK (W84), count during |
| | PREGNANCY_HIGH_RISK | F0: before and early pregnancy |
| 668 | F0_EOS_perc | Labtest EOS %, sampled during F0: before and early pregnancy |
| 669 | F2_clinic_286_{—} | Clinic diagnosis COAGULATION DEFECTS (286), count during |
| | COAGULATION_DEFECTS | F2: second blood test (weeks 16-19) |
| 670 | F1_pharma_C10AX06_{—} | Prescription of OMEGA D3 9 MONTHS (SUPH) S.GEL CAP 60 |
| | OMEGA_D3_9_MONTHS_{—} | (C10AX06), count during F1: first blood test (weeks 6-12) |
| | SUPH_S_GEL_CAP_60 | |
| 671 | F0_BASO_perc | Labtest BASO %, sampled during F0: before and early pregnancy |
| 672 | F1_ANTI_CARDIOLIPIN_IgG | Labtest ANTI CARDIOLIPIN IgG, sampled during F1: first blood |
| | | test (weeks 6-12) |
| 673 | M2_HYPER_perc | Labtest HYPER %, sampled during M2: year 2 before pregnancy |
| 674 | F0_ESR | Labtest ESR, sampled during F0: before and early pregnancy |
| 675 | F1_pharma_H02AB07_{—} | Prescription of PREDNISONE TAB 5 MG 30 (H02AB07), count |
| | PREDNISONE_TAB_{—} | during F1: first blood test (weeks 6-12) |
| | 5MG_30 | |
| 676 | F1_clinic_V263_GENETIC_{—} | Clinic diagnosis GENETIC COUNSELING ON PROCREATIVE |
| | COUNSELING_ON_{—} | MANAGEMENT (V263), count during F1: first blood test |
| | PROCREATIVE_{—} | (weeks 6-12) |
| | MANAGEMENT | |
| 677 | F1_NEUT_perc | Labtest NEUT %, sampled during F1: first blood test (weeks 6-12) |
| 678 | F0_PCT | Labtest PCT, sampled during F0: before and early pregnancy |
| 679 | F2_hospital_V234_{—} | Hospital diagnosis SUPERVISION OF HIGH-RISK |
| | SUPERVISION_OF_HIGH_{—} | PREGN.WITH POOR OBSTETRIC HISTORY (V234), count |
| | RISK_PREGN_WITH_POOR_{—} | during F2: second blood test (weeks 16-19) |
| | OBSTETRIC_HISTORY | |
| 680 | F2_pharma_G03DA04_{—} | Prescription of CRINONE VAG. GEL 8% 15U (G03DA04), |
| | CRINONE_VAG_GEL_8%_15U | count during F2: second blood test (weeks 16-19) |
| 681 | F2_MPV | Labtest MPV, sampled during F2: second blood test |
| | | (weeks 16-19) |
| 682 | locality_type_Jewish_Locality_{—} | Lives in Jewish Locality 10,000-19,999 residents |
| | 10000_to_19999_residents | |
| 683 | F0_ANTITHROMBIN_III | Labtest ANTITHROMBIN-III, sampled during F0: before and |
| | | early pregnancy |
| 684 | locality_type_Jewish_Other_{—} | Lives in Jewish Other Rural Locality |
| | Rural_Locality | |
| 685 | F1_clinic_0_OTHER | Clinic diagnosis OTHER (0), count during F1: first blood test |
| | | (weeks 6-12) |
| 686 | F0_clinic_5368_DYSPEPSIA_{—} | Clinic diagnosis DYSPEPSIA AND OTHER SPECIFIED |
| | AND_OTHER_SPECIFIED_{—} | DISORDERS OF FUNCTION OF STOMACH (5368), count |
| | DISORDERS_OF_{—} | during F0: before and early pregnancy |
| | FUNCTION_OF_STOMACH | |
| 687 | F2_clinic_6466_INFECTIONS_{—} | Clinic diagnosis INFECTIONS OF GENITOURINARY TRACT |
| | OF_GENITOURINARY_{—} | IN PREGNANCY (6466), count during F2: second blood test |
| | TRACT_IN_PREGNANCY | (weeks 16-19) |
| 688 | F0_clinic_1101_{—} | Clinic diagnosis DERMATOPHYTOSIS OF NAIL (1101), count |
| | DERMATOPHYTOSIS_{—} | during F0: before and early pregnancy |
| | OF_NAIL | |
| 689 | F2_clinic_L15_KNEE_{—} | Clinic diagnosis KNEE SYMPTOMS/COMPLAINTS (L15), |
| | SYMPTOMS_OR_{—} | count during F2: second blood test (weeks 16-19) |
| | COMPLAINTS | |
| 690 | F2_RDW_CV | Labtest RDW-CV, sampled during F2: second blood test |
| | | (weeks 16-19) |
| 691 | F2_clinic_7802_SYNCOPE_{—} | Clinic diagnosis SYNCOPE AND COLLAPSE (7802), count |
| | AND_COLLAPSE | during F2: second blood test (weeks 16-19) |
| 692 | F0_pharma_G03DA04_{—} | Prescription of GESTON INJ 50 mg/1 mL 1 mL (G03DA04), count |
| | GESTON_INJ_50mg_OR_1mL_1mL | during F0: before and early pregnancy |
| 693 | F0_ANTI_CARDIOLIPIN_IgG | Labtest ANTI CARDIOLIPIN IgG, sampled during F0: before |
| | | and early pregnancy |
| 694 | F0_clinic_L15_KNEE_{—} | Clinic diagnosis KNEE SYMPTOMS/COMPLAINTS (L15), |
| | SYMPTOMS_OR_{—} | count during F0: before and early pregnancy |
| | COMPLAINTS | |
| 695 | F0_HEMOGLOBIN_A1C_perc | Labtest HEMOGLOBIN A1C %, sampled during F0: before and |
| | | early pregnancy |
| 696 | F2_clinic_7231_CERVICALGIA | Clinic diagnosis CERVICALGIA (7231), count during F2: second |
| | | blood test (weeks 16-19) |
| 697 | F1_THYROGLOBULIN_Ab | Labtest THYROGLOBULIN Ab, sampled during F1: first blood |
| | | test (weeks 6-12) |
| 698 | F0_LYMP_abs | Labtest LYMP.abs, sampled during F0: before and early |
| | | pregnancy |
| 699 | F1_LUC_perc | Labtest LUC %, sampled during F1: first blood test (weeks 6-12) |
| 700 | F1_clinic_242_{—} | Clinic diagnosis THYROTOXICOSIS WITH OR WITHOUT |
| | THYROTOXICOSIS_WITH_{—} | GOITER (242), count during F1: first blood test (weeks 6-12) |
| | OR_WITHOUT_GOITER | |
| 701 | F0_pharma_A11AA06_{—} | Prescription of PRENATAL MULTIVITAMINS D.H TAB 30 |
| | PRENATAL_MULTIVITAMINS_{—} | (A11AA06), count during F0: before and early pregnancy |
| | D_H_TAB_30 | |
| 702 | F0_T4_FREE | Labtest T4- FREE, sampled during F0: before and early |
| | | pregnancy |
| 703 | prev_pregnancy_3_HCT | Labtest HCT, sampled during 3rd previous pregnancy |
| 704 | F2_GOT_AST | Labtest GOT (AST), sampled during F2: second blood test |
| | | (weeks 16-19) |
| 705 | F2_pharma_B01AB05_{—} | Prescription of CLEXANE SAFETY LOCK INJ 40 MG/0.4 ML 2 |
| | CLEXANE_SAFETY_LOCK_{—} | (B01AB05), count during F2: second blood test (weeks 16-19) |
| | INJ_40MG_OR_0_4ML_2 | |
| 706 | M4_MACRO_perc | Labtest MACRO %, sampled during M4: year 4 before pregnancy |
| 707 | prev_pregnancy_3_MONO_perc | Labtest MONO %, sampled during 3rd previous pregnancy |
| 708 | F0_CHLORIDE | Labtest CHLORIDE, sampled during F0: before and early |
| | | pregnancy |
| 709 | F0_clinic_678_OTHER_{—} | Clinic diagnosis OTHER FETAL CONDITIONS (678), count |
| | FETAL_CONDITIONS | during F0: before and early pregnancy |
| 710 | F1_pharma_{——}STREP_A_TEST_{—} | Prescription of STREP A TEST- QUICKVUE IN-LINE ( ), count |
| | QUICKVUE_IN_LINE | during F1: first blood test (weeks 6-12) |
| 711 | F0_CHOLESTEROL_or_HDL | Labtest CHOLESTEROL/ HDL, sampled during F0: before and |
| | | early pregnancy |
| 712 | F1_pharma_R05CA20_{—} | Prescription of THYMOLI SYR BOT 100 ML (R05CA20), count |
| | THYMOLI_SYR_BOT_100ML | during F1: first blood test (weeks 6-12) |
| 713 | F0_SHBG | Labtest SHBG, sampled during F0: before and early pregnancy |
| 714 | F1_pharma_R06AX13_{—} | Prescription of LORATADINE (LORATADIM) TAB 10 mg 10 |
| | LORATADINE_{—} | (R06AX13), count during F1: first blood test (weeks 6-12) |
| | LORATADIM_TAB_{—} | |
| | 10mg_10 | |
| 715 | F1_HEMOGLOBIN_S | Labtest HEMOGLOBIN S, sampled during F1: first blood test |
| | | (weeks 6-12) |
| 716 | F1_dinic_7030_INGROWING_{—} | Clinic diagnosis INGROWING NAIL (7030), count during F1: |
| | NAIL | first blood test (weeks 6-12) |
| 717 | F1_pharma_A03FA01_{—} | Prescription of PRAMIN INJ 10 MG/2 ML 5 (A03FA01), count |
| | PRAMIN_INJ_10MG_OR_{—} | during F1: first blood test (weeks 6-12) |
| | 2ML_5 | |
| 718 | F0_clinic_V25_{—} | Clinic diagnosis CONTRACEPTIVE MANAGEMENT (V25), |
| | CONTRACEPTIVE_{—} | count during F0: before and early pregnancy |
| | MANAGEMENT | |
| 719 | F0_clinic_401_ESSENTIAL_{—} | Clinic diagnosis ESSENTIAL HYPERTENSION (401), count |
| | HYPERTENSION | during F0: before and early pregnancy |
| 720 | F2_clinic_V268_IN_VITRO_{—} | Clinic diagnosis IN VITRO FERTILIZATION OR TEST TUBE |
| | FERTILIZATION_OR_{—} | PREGNANCY (V268), count during F2: second blood test |
| | TEST_TUBE_PREGNANCY | (weeks 16-19) |
| 721 | F0_pharma_G03DA04_{—} | Prescription of CRINONE VAG. GEL 8% 15U (G03DA04), |
| | CRINONE_VAG_GEL_8%_15U | count during F0: before and early pregnancy |
| 722 | F0_pharma_N02BB02_{—} | Prescription of OPTALGIN CPL 500 MG 21 (N02BB02), count |
| | OPTALGIN_CPL_500MG_21 | during F0: before and early pregnancy |
| 723 | F2_pharma_S02CA06_{—} | Prescription of DESOREN AUR CF BOT 5 ML (S02CA06), count |
| | DESOREN_AUR_CF_{—} | during F2: second blood test (weeks 16-19) |
| | BOT_5ML | |
| 724 | F2_hospital_V146_PERSONAL_{—} | Hospital diagnosis PERSONAL HISTORY OF ALLERGY TO |
| | HISTORY_OF_ALLERGY_{—} | ANALGESIC AGENT (V146), count during F2: second blood |
| | TO_ANALGESIC_AGENT | test (weeks 16-19) |
| 725 | F2_RETICUL_COUNT_abs | Labtest RETICUL. COUNT abs, sampled during F2: second |
| | | blood test (weeks 16-19) |
| 726 | F0_ERYTHROCYTES_SED | Labtest ERYTHROCYTES-SED, sampled during F0: before and |
| | | early pregnancy |
| 727 | F1_A_B2_GLYCOPROTEI_IgG | Labtest A.B2 GLYCOPROTEI IgG, sampled during F1: first |
| | | blood test (weeks 6-12) |
| 728 | F1_pharma_H01CC01_{—} | Prescription of ORGALUTRAN SYRI.<><> INJ 0.25 MG 0.5 ML |
| | ORGALUTRAN_SYRI_<><>_{—} | (H01CC01), count during F1: first blood test (weeks 6-12) |
| | INJ_0_25MG_0_5ML | |
| 729 | F0_pharma_Z07_AFTER_{—} | Prescription of AFTER 10 STICK KIT (Z07), count during F0: |
| | 10_STICK_KIT | before and early pregnancy |
| 730 | prev_pregnancy_3_MICRO_perc | Labtest MICRO %, sampled during 3rd previous pregnancy |
| 731 | F1_COMPLEMENT_C4 | Labtest COMPLEMENT C4, sampled during F1: first blood test |
| | | (weeks 6-12) |
| 732 | F0_HB | Labtest HB, sampled during F0: before and early pregnancy |
| 733 | F0_clinic_2452_CHRONIC_{—} | Clinic diagnosis CHRONIC LYMPHOCYTIC THYROIDITIS |
| | LYMPHOCYTIC_{—} | (2452), count during F0: before and early pregnancy |
| | THYROIDITIS | |
| 734 | F1_pharma_J01DB01_{—} | Prescription of CEFOVIT FORTE CAP 500 mg 20 (J01DB01), |
| | CEFOVIT_FORTE_CAP_{—} | count during F1: first blood test (weeks 6-12) |
| | 500mg_20 | |
| 735 | F2_clinic_6236_VAGINAL_{—} | Clinic diagnosis VAGINAL HEMATOMA (6236), count during |
| | HEMATOMA | F2: second blood test (weeks 16-19) |
| 736 | F0_pharma_D01AC24_{—} | Prescription of TEVACUTAN CR 15 g (D01AC24), count during |
| | TEVACUTAN_CR_15g | F0: before and early pregnancy |
| 737 | F2_MICROALBUMIN_or_{—} | Labtest MICROALBUMIN/CREAT, sampled during F2: second |
| | CREAT | blood test (weeks 16-19) |
| 738 | F1_clinic_78791_DIARRHEA | Clinic diagnosis DIARRHEA (78791), count during F1: first |
| | | blood test (weeks 6-12) |
| 739 | F2_pharma_J01DC02_{—} | Prescription of ZINNAT TAB 500 MG 10 (J01DC02), count |
| | ZINNAT_TAB_500MG_10 | during F2: second blood test (weeks 16-19) |
| 740 | F1_clinic_643_EXCESSIVE_{—} | Clinic diagnosis EXCESSIVE VOMITING IN PREGNANCY |
| | VOMITING_IN_PREGNANCY | (643), count during F1: first blood test (weeks 6-12) |
| 741 | F0_PT_SEC | Labtest PT-SEC, sampled during F0: before and early pregnancy |
| 742 | F1_BP_time_to_GTT | Time to GTT of blood pressure sampled during F1: first blood test |
| | | (weeks 6-12) |
| 743 | F1_pharma_A11AA06_{—} | Prescription of PRENATAL 9 MONTHS (ALTMAN) CPL 60 |
| | PRENATAL_9_MONTHS_{—} | (A11AA06), count during F1: first blood test (weeks 6-12) |
| | ALTMAN_CPL_60 | |
| 744 | F0_clinic_487_INFLUENZA | Clinic diagnosis INFLUENZA (487), count during F0: before and |
| | | early pregnancy |
| 745 | F2_A_B2_GLYCOPROTEI_IgG | Labtest A.B2 GLYCOPROTEI IgG, sampled during F2: second |
| | | blood test (weeks 16-19) |
| 746 | M5_MCV | Labtest MCV, sampled during M5: year 5 before pregnancy |
| 747 | F2_pharma_A06AD15_NOR | Prescription of NORMALAX PWD 240 GM (A06AD15), count |
| | MALAX_PWD_240GM | during F2: second blood test (weeks 16-19) |
| 748 | F1_PROTEIN_U_SAMPLE | Labtest PROTEIN- U SAMPLE, sampled during F1: first blood |
| | | test (weeks 6-12) |
| 749 | F0_hospital_Z9921_INJECTION_{—} | Hospital diagnosis INJECTION OF ANTIBIOTIC (Z9921), count |
| | OF_ANTIBIOTIC | during F0: before and early pregnancy |
| 750 | F0_pharma_{——}ELASTAN_{—} | Prescription of ELASTAN TREAT CR 75 ML ( ), count during |
| | TREAT_CR_75ML | F0: before and early pregnancy |
| 751 | F0_pharma_B03BB01_FOLIC_{—} | Prescription of FOLIC ACID (NEW) D.H TAB 400 mcg 100 |
| | ACID_NEW_D_H_TAB_{—} | (B03BB01), count during F0: before and early pregnancy |
| | 400mcg_100 | |
| 752 | F2_pharma_{——}STREP_A_TEST_{—} | Prescription of STREP A TEST- QUICKVUE IN-LINE ( ), count |
| | QUICKVUE_IN_LINE | during F2: second blood test (weeks 16-19) |
| 753 | F1_pharma_Unknown_ATC_{—} | Prescription of OTHER (Unknown_ATC), count during F1: first |
| | OTHER | blood test (weeks 6-12) |
| 754 | F1_clinic_461_ACUTE_{—} | Clinic diagnosis ACUTE SINUSITIS (461), count during F1: first |
| | SINUSITIS | blood test (weeks 6-12) |
| 755 | F1_clinic_6221_DYSPLASIA_{—} | Clinic diagnosis DYSPLASIA OF CERVIX (UTERI) (6221), |
| | OF_CERVIX_UTERI | count during F1: first blood test (weeks 6-12) |
| 756 | F0_clinic_9249_CONTUSION_{—} | Clinic diagnosis CONTUSION OF UNSPECIFIED SITE (9249), |
| | OF_UNSPECIFIED_SITE | count during F0: before and early pregnancy |
| 757 | F0_pharma_R03AC02_{—} | Prescription of VENTOLIN CFC FREE INH 0.1 MG/INH |
| | VENTOLIN_CFC_FREE_INH_{—} | (R03AC02), count during F0: before and early pregnancy |
| | 0_1MG_OR_INH | |
| 758 | F1_clinic_7910_PROTEINURIA | Clinic diagnosis PROTEINURIA (7910), count during F1: first |
| | | blood test (weeks 6-12) |
| 759 | F1_PH_U_STRIP | Labtest PH- U STRIP, sampled during F1: first blood test |
| | | (weeks 6-12) |
| 760 | F1_clinic_7062_SEBACEOUS_{—} | Clinic diagnosis SEBACEOUS CYST (7062), count during F1: |
| | CYST | first blood test (weeks 6-12) |
| 761 | F1_COMPLEMENT_C3 | Labtest COMPLEMENT C3, sampled during F1: first blood test |
| | | (weeks 6-12) |
| 762 | F2_PDW | Labtest PDW, sampled during F2: second blood test |
| | | (weeks 16-19) |
| 763 | F0_EBV_VCA_IgM | Labtest EBV VCA IgM, sampled during F0: before and early |
| | | pregnancy |
| 764 | F1_pharma_G01AF02_{—} | Prescription of AGISTEN 1 VAGINAL TAB 500 MG |
| | AGISTEN_1_VAGINAL_{—} | (G01AF02), count during F1: first blood test (weeks 6-12) |
| | TAB_500MG | |
| 765 | F2_pharma_G01AF02_{—} | Prescription of CLOTREE-TEVA VAG OVL 200 MG 3 |
| | CLOTREE_TEVA_VAG_{—} | (G01AF02), count during F2: second blood test (weeks 16-19) |
| | OVL_200MG_3 | |
| 766 | F2_clinic_0_UNKNOWN_{—} | Clinic diagnosis UNKNOWN CATEGORY (0), count during F2: |
| | CATEGORY | second blood test (weeks 16-19) |
| 767 | F2_clinic_7030_INGROWING_{—} | Clinic diagnosis INGROWING NAIL (7030), count during F2: |
| | NAIL | second blood test (weeks 16-19) |
| 768 | F0_hospital_V146_PERSONAL_{—} | Hospital diagnosis PERSONAL HISTORY OF ALLERGY TO |
| | HISTORY_OF_ALLERGY_TO_{—} | ANALGESIC AGENT (V146), count during F0: before and early |
| | ANALGESIC_AGENT | pregnancy |
| 769 | F2_GLOM_FILTR_RATE | Labtest GLOM.FILTR.RATE, sampled during F2: second blood |
| | | test (weeks 16-19) |
| 770 | F0_clinic_3720_ACUTE_{—} | Clinic diagnosis ACUTE CONJUNCTIVITIS (3720), count |
| | CONJUNCTIVITIS | during F0: before and early pregnancy |
| 771 | locality_type_Communal_{—} | Lives in Communal Locality |
| | Locality | |
| 772 | F0_POTASSIUM_v1 | Labtest POTASSIUM, sampled during F0: before and early |
| | | pregnancy |
| 773 | ethnicity_iran | Etnicity: Iran |
| 774 | F0_GOT_AST | Labtest GOT (AST), sampled during F0: before and early |
| | | pregnancy |
| 775 | F2_pharma_A12AX01_{—} | Prescription of VITA-CAL + D (NEW) TAB CF BOX 60 |
| | VITA_CAL+D_NEW_{—} | (A12AX01), count during F2: second blood test (weeks 16-19) |
| | TAB_CF_BOX_60 | |
| 776 | F0_clinic_1_FILL_OUT_{—} | Clinic diagnosis FILL OUT FORMS (1), count during F0: before |
| | FORMS | and early pregnancy |
| 777 | F2_ESR | Labtest ESR, sampled during F2: second blood test (weeks 16-19) |
| 778 | F2_hospital_64193_{—} | Hospital diagnosis UNSPECIFIED ANTEPARTUM |
| | UNSPECIFIED_{—} | HEMORRHAGE (64193), count during F2: second blood test |
| | ANTEPARTUM_{—} | (weeks 16-19) |
| | HEMORRHAGE | |
| 779 | F0_clinic_R74_U_R_I_{—} | Clinic diagnosis U.R.I. (HEAD COLD) (R74), count during F0: |
| | HEAD_COLD | before and early pregnancy |
| 780 | F2_pharma_R01AA07_{—} | Prescription of OTRIVIN NASAL M.D. SPR 0.1% 10 ML |
| | OTRIVIN_NASAL_M_D_{—} | (R01AA07), count during F2: second blood test (weeks 16-19) |
| | SPR_0_1%_10ML | |
| 781 | F1_pharma_M01AE01_{—} | Prescription of NUROFEN LIQUID CAP 200 MG BOX 20 |
| | NUROFEN_LIQUID_CAP_{—} | (M01AE01), count during F1: first blood test (weeks 6-12) |
| | 200MG_BOX_20 | |
| 782 | F2_ANTI_THYROID_PEROXID | Labtest ANTI THYROID PEROXID, sampled during F2: second |
| | | blood test (weeks 16-19) |
| 783 | F2_clinic_7844_VOICE_{—} | Clinic diagnosis VOICE DISTURBANCE (7844), count during |
| | DISTURBANCE | F2: second blood test (weeks 16-19) |
| 784 | F2_clinic_Z887_DIAGNOSTIC_{—} | Clinic diagnosis DIAGNOSTIC ULTRASOUND (Z887), count |
| | ULTRASOUND | during F2: second blood test (weeks 16-19) |
| 785 | M1_MCH | Labtest MCH, sampled during M1: year 1 before pregnancy |
| 786 | F0_pharma_B03BB01_FOLIC_{—} | Prescription of FOLIC ACID (SAM-ON) TAB 5 MG 30 |
| | ACID_SAM_ON_TAB_5MG_30 | (B03BB01), count during F0: before and early pregnancy |
| 787 | F0_SPECIFIC_GRAV_U_STRI | Labtest SPECIFIC GRAV-U STRI, sampled during F0: before |
| | | and early pregnancy |
| 788 | F2_pharma_B01AC06_{—} | Prescription of MICROPIRIN TAB 75 MG 28 (B01AC06), count |
| | MICROPIRIN_TAB_75MG_28 | during F2: second blood test (weeks 16-19) |
| 789 | F1_pharma_J01DC02_{—} | Prescription of ZINN AT TAB 250 MG 10 (J01DC02), count |
| | ZINNAT_TAB_250MG_10 | during F1: first blood test (weeks 6-12) |
| 790 | F2_pharma_D01AC24_{—} | Prescription of TEVACUTAN CR 15 g (D01AC24), count during |
| | TEVACUTAN_CR_15g | F2: second blood test (weeks 16-19) |
| 791 | F1_clinic_3_AFTERCARE_{—} | Clinic diagnosis AFTERCARE CHECK-UP NO DISEASE (3), |
| | CHECK_UP_NO_DISEASE | count during F1: first blood test (weeks 6-12) |
| 792 | prev_pregnancy_3_LYM_perc | Labtest LYM %, sampled during 3rd previous pregnancy |
| 793 | F1_clinic_7019_UNSPECIFIED_{—} | Clinic diagnosis UNSPECIFIED HYPERTROPHIC AND |
| | HYPERTROPHIC_AND_{—} | ATROPHIC CONDITIONS OF SKIN (7019), count during F1: |
| | ATROPHIC_CONDITIONS_OF_SKIN | first blood test (weeks 6-12) |
| 794 | F1_VITAMIN_D_25_OH | Labtest VITAMIN D (25-OH), sampled during F1: first blood test |
| | | (weeks 6-12) |
| 795 | F0_clinic_1121_CANDIDIASIS_{—} | Clinic diagnosis CANDIDIASIS OF VULVA AND VAGINA |
| | OF_VULVA_AND_VAGINA | (1121), count during F0: before and early pregnancy |
| 796 | F2_hospital_V2385_{—} | Hospital diagnosis SUPERVISION OF HIGH RISK |
| | SUPERVISION_OF_HIGH_{—} | PREGNANCY RESULTING FROM ASSISTED |
| | RISK_PREGNANCY_{—} | REPRODUCTIVE TECHNOLOGY (V2385), count during F2: second |
| | RESULTING_FROM_{—} | blood test (weeks 16-19) |
| | ASSISTED_REPROD_{—} | |
| | UCTIVE_TECHNOLOGY | |
| 797 | ethnicity_north_africa | Etnicity: North Africa |
| 798 | F0_weight_time_to_GTT | Time to GTT of weight sampled during F0: before and early |
| | | pregnancy |
| 799 | F1_pharma_G03CA03_{—} | Prescription of ESTROFEM TAB 2 MG 28 (G03CA03), count |
| | ESTROFEM_TAB_2MG_28 | during F1: first blood test (weeks 6-12) |
| 800 | F0_LEUCOCYTES_U_STRIP | Labtest LEUCOCYTES - U STRIP, sampled during F0: before |
| | | and early pregnancy |
| 801 | F0_CONTROL_PT | Labtest CONTROL PT, sampled during F0: before and early |
| | | pregnancy |
| 802 | F0_clinic_708_URTICARIA | Clinic diagnosis URTICARIA (708), count during F0: before and |
| | | early pregnancy |
| 803 | F1_pharma_G03DA04_{—} | Prescription of CRINONE VAG. GEL 8% 15U (G03DA04), |
| | CRINONE_VAG_GEL_8%_15U | count during F1: first blood test (weeks 6-12) |
| 804 | F1_hospital_Z6992_{—} | Hospital diagnosis ARTIFICIAL INSEMINATION (Z6992), |
| | ARTIFICIAL_INSEMINATION | count during F1: first blood test (weeks 6-12) |
| 805 | F1_pharma_G01AF02_AGI | Prescription of AGISTEN VAG TAB 200 mg 3 (G01AF02), count |
| | STEN_VAG_TAB_200mg_3 | during F1: first blood test (weeks 6-12) |
| 806 | F2_pharma_B01AC06_{—} | Prescription of MICROPIRIN TAB 75 mg 30 (B01AC06), count |
| | MICROPIRIN_TAB_75mg_30 | during F2: second blood test (weeks 16-19) |
| 807 | F2_A_B2_GLYCOPROTEI_IgM | Labtest A.B2 GLYCOPROTEI IgM, sampled during F2: second |
| | | blood test (weeks 16-19) |
| 808 | prev_pregnancy_3_BASO_perc | Labtest BASO %, sampled during 3rd previous pregnancy |
| 809 | F1_clinic_0_UNKNOWN_{—} | Clinic diagnosis UNKNOWN CATEGORY (0), count during F1: |
| | CATEGORY | first blood test (weeks 6-12) |
| 810 | F2_PROTEIN_URINE_24h | Labtest PROTEIN- URINE 24 h, sampled during F2: second blood |
| | | test (weeks 16-19) |
| 811 | F1_pharma_R05DA21_{—} | Prescription of DEXAMOL COLD 30D + 20N CPL (R05DA21), |
| | DEXAMOL_COLD_30D+20N_CPL | count during F1: first blood test (weeks 6-12) |
| 812 | F1_pharma_A11DB01_{—} | Prescription of TRIBEMIN TAB 20 (A11DB01), count during F1: |
| | TRIBEMIN_TAB_20 | first blood test (weeks 6-12) |
| 813 | F2_clinic_2872_OTHER_{—} | Clinic diagnosis OTHER NONTHROMBOCYTOPENIC |
| | NONTHROMBOCYTOPENIC_{—} | PURPURAS (2872), count during F2: second blood test |
| | PURPURAS | (weeks 16-19) |
| 814 | F1_clinic_4_NOTHING_{—} | Clinic diagnosis NOTHING ABNORMAL FOUND (4), count |
| | ABNORMAL_FOUND | during F1: first blood test (weeks 6-12) |
| 815 | F1_clinic_708_URTICARIA | Clinic diagnosis URTICARIA (708), count during F1: first blood |
| | | test (weeks 6-12) |
| 816 | F0_LUC_perc | Labtest LUC %, sampled during F0: before and early pregnancy |
| 817 | prev_pregnancy_3_MPV | Labtest MPV, sampled during 3rd previous pregnancy |
| 818 | F1_pharma_G01AF02_{—} | Prescription of AGISTEN VAGINAL CR 2% 20 GM (G01AF02), |
| | AGISTEN_VAGINAL_CR_{—} | count during F1: first blood test (weeks 6-12) |
| | 2%_20GM | |
| 819 | F2_clinic_78079_OTHER_{—} | Clinic diagnosis OTHER MALAISE AND FATIGUE (78079), |
| | MALAISE_AND_FATIGUE | count during F2: second blood test (weeks 16-19) |
| 820 | F0_clinic_69010_{—} | Clinic diagnosis SEBORRHEIC DERMATITIS, UNSPECIFIED |
| | SEBORRHEIC_DERMATITIS_{—} | (69010), count during F0: before and early pregnancy |
| | UNSPECIFIED | |
| 821 | F2_clinic_6829_CELLULITIS_{—} | Clinic diagnosis CELLULITIS AND ABSCESS OF |
| | AND_ABSCESS_OF_{—} | UNSPECIFIED SITES (6829), count during F2: second blood test |
| | UNSPECIFIED_SITES | (weeks 16-19) |
| 822 | F0_VITAMIN_B12 | Labtest VITAMIN B12, sampled during F0: before and early |
| | | pregnancy |
| 823 | F2_clinic_6482_ANEMIA_{—} | Clinic diagnosis ANEMIA COMPLICATING PREGNANCY, |
| | COMPLICATING_PREGNANCY_{—} | CHILDBIRTH, OR THE PUERPERIUM (6482), count during |
| | CHILDBIRTH_OR_THE_{—} | F2: second blood test (weeks 16-19) |
| | PUERPERIUM | |
| 824 | F2_hospital_78903_{—} | Hospital diagnosis ABDOMINAL PAIN, RT. LOWER |
| | ABDOMINAL_PAIN_RT_{—} | QUADRANT (78903), count during F2: second blood test |
| | LOWER_QUADRANT | (weeks 16-19) |
| 825 | F2_APTT_R | Labtest APTT-R, sampled during F2: second blood test |
| | | (weeks 16-19) |
| 826 | F2_pharma_A02BC03_{—} | Prescription of LANTON CAP 30 MG BOX 28 (A02BC03), count |
| | LANTON_CAP_30MG_BOX_28 | during F2: second blood test (weeks 16-19) |
| 827 | M4_HYPER_perc | Labtest HYPER %, sampled during M4: year 4 before pregnancy |
| 828 | F2_pharma_B03AD03_{—} | Prescription of FERROGRAD FOLIC TAB CF BOX 30 |
| | FERROGRAD_FOLIC_TAB_{—} | (B03AD03), count during F2: second blood test (weeks 16-19) |
| | CF_BOX_30 | |
| 829 | F0_clinic_8450_ANKLE_SPRAIN | Clinic diagnosis ANKLE SPRAIN (8450), count during F0: |
| | | before and early pregnancy |
| 830 | F1_clinic_U80_INJURIES | Clinic diagnosis INJURIES (U80), count during F1: first blood |
| | | test (weeks 6-12) |
| 831 | F2_COMPLEMENT_C3 | Labtest COMPLEMENT C3, sampled during F2: second blood |
| | | test (weeks 16-19) |
| 832 | F0_pharma_S02DA30_{—} | Prescription of OTIDIN AUR CF 10 GM (S02DA30), count |
| | OTIDIN_AUR_CF_10GM | during F0: before and early pregnancy |
| 833 | F2_clinic_7998_OTHER_ILL_{—} | Clinic diagnosis OTHER ILL-DEFINED CONDITIONS (7998), |
| | DEFINED_CONDITIONS | count during F2: second blood test (weeks 16-19) |
| 834 | F1_clinic_5301_ESOPHAGITIS | Clinic diagnosis ESOPHAGITIS (5301), count during F1: first |
| | | blood test (weeks 6-12) |
| 835 | F1_clinic_8470_NECK_SPRAIN | Clinic diagnosis NECK SPRAIN (8470), count during F1: first |
| | | blood test (weeks 6-12) |
| 836 | glucose_change_coef_b | Estimated glucose at 26 weeks |
| 837 | F0_clinic_7998_OTHER_ILL_{—} | Clinic diagnosis OTHER ILL-DEFINED CONDITIONS (7998), |
| | DEFINED_CONDITIONS | count during F0: before and early pregnancy |
| 838 | F2_clinic_0549_HERPES_{—} | Clinic diagnosis HERPES SIMPLEX WITHOUT MENTION OF |
| | SIMPLEX_WITHOUT_{—} | COMPLICATION (0549), count during F2: second blood test |
| | MENTION_OF_{—} | (weeks 16-19) |
| | COMPLICATION | |
| 839 | F1_GOT_AST | Labtest GOT (AST), sampled during F1: first blood test |
| | | (weeks 6-12) |
| 840 | F2_pharma_R05CB01_{—} | Prescription of REOLIN EFFERV. TAB 200 MG 30 (R05CB01), |
| | REOLIN_EFFERV_TAB_{—} | count during F2: second blood test (weeks 16-19) |
| | 200MG_30 | |
| 841 | F1_clinic_V726_{—} | Clinic diagnosis LABORATORY EXAMINATION (V726), |
| | LABORATORY_{—} | count during F1: first blood test (weeks 6-12) |
| | EXAMINATION | |
| 842 | F1_hospital_Z65911_E_T | Hospital diagnosis E.T. (Z65911), count during F1: first blood |
| | | test (weeks 6-12) |
| 843 | F1_clinic_3671_MYOPIA | Clinic diagnosis MYOPIA (3671), count during F1: first blood |
| | | test (weeks 6-12) |
| 844 | F0_climc_V724_PREGNANCY_{—} | Clinic diagnosis PREGNANCY EXAMINATION OR TEST, |
| | EXAMINATION_OR_TEST_{—} | PREGNANCY UNCONFIRMED (V724), count during F0: |
| | PREGNANCY_UNCONFIRMED | before and early pregnancy |
| 845 | F0_BILIRUBIN_U_STRIP | Labtest BILIRUBIN- U STRIP, sampled during F0: before and |
| | | early pregnancy |
| 846 | F2_clinic_1_FILL_OUT_FORMS | Clinic diagnosis FILL OUT FORMS (1), count during F2: second |
| | | blood test (weeks 16-19) |
| 847 | F1_GLOM_FILTR_RATE | Labtest GLOM.FILTR.RATE, sampled during F1: first blood test |
| | | (weeks 6-12) |
| 848 | F1_clinic_5246_{—} | Clinic diagnosis TEMPOROMANDIBULAR JOINT |
| | TEMPOROMANDIBULAR_{—} | DISORDERS (5246), count during F1: first blood test |
| | JOINT_DISORDERS | (weeks 6-12) |
| 849 | F1_hospital_3051_{—} | Hospital diagnosis TOBACCO USE DISORDER (3051), count |
| | TOBACCO_USE_DISORDER | during F1: first blood test (weeks 6-12) |
| 850 | F1_pharma_M01AE01_{—} | Prescription of NUROFEN STRAWB.PED. SUS 100 mg/5 mL |
| | NUROFEN_STRAWB_PED_{—} | 100 mL (M01AE01), count during F1: first blood test |
| | SUS_100mg_OR_5mL_100mL | (weeks 6-12) |
| 851 | F1_APTT_R | Labtest APTT-R, sampled during F1: first blood test (weeks 6-12) |
| 852 | F0_climc_0_OTHER | Clinic diagnosis OTHER (0), count during F0: before and early |
| | | pregnancy |
| 853 | F1_clinic_07819_OTHER_{—} | Clinic diagnosis OTHER SPECIFIED VIRAL WARTS (07819), |
| | SPECIFIED_VIRAL_WARTS | count during F1: first blood test (weeks 6-12) |
| 854 | F1_HEMOGLOBIN_A | Labtest HEMOGLOBIN A, sampled during F1: first blood test |
| | | (weeks 6-12) |
| 855 | F0_height | Height sampled during F0: before and early pregnancy |
| 856 | F1_pharma_A11AA06_{—} | Prescription of PRENATAL PLUS CF TAB CF BOX 30 |
| | PRENATAL_PLUS_CF_{—} | (A11AA06), count during F1: first blood test (weeks 6-12) |
| | TAB_CF_BOX_30 | |
| 857 | F1_CREATININE_U_SAMPLE | Labtest CREATININE- U SAMPLE, sampled during F1: first |
| | | blood test (weeks 6-12) |
| 858 | F1_CHOLESTEROL_or_HDL | Labtest CHOLESTEROL/HDL, sampled during F1: first blood |
| | | test (weeks 6-12) |
| 859 | F0_pharma_H02AB07_{—} | Prescription of PREDNISONE TAB 5 MG 30 (H02AB07), count |
| | PREDNISONE_TAB_5MG_30 | during F0: before and early pregnancy |
| 860 | M4_BASO_abs | Labtest BASO abs, sampled during M4: year 4 before pregnancy |
| 861 | F0_clinic_7865_CHEST_PAIN | Clinic diagnosis CHEST PAIN (7865), count during F0: before |
| | | and early pregnancy |
| 862 | F2_PROT_S_ANTIGEN_FREE | Labtest PROT-S ANTIGEN (FREE, sampled during F2: second |
| | | blood test (weeks 16-19) |
| 863 | F1_FSH | Labtest FSH, sampled during F1: first blood test (weeks 6-12) |
| 864 | F0_NEUT_perc | Labtest NEUT %, sampled during F0: before and early pregnancy |
| 865 | F2_pharma_A04AA01_{—} | Prescription of ZOFRAN TAB 4 MG 10 (A04AA01), count during |
| | ZOFRAN_TAB_4MG_10 | F2: second blood test (weeks 16-19) |
| 866 | F0_clinic_677_LATE_EFFECT_{—} | Clinic diagnosis LATE EFFECT OF COMPLICATION OF |
| | OF_COMPLICATION_OF_{—} | PREGNANCY,CHILDBIRTH,PUERPERIUM (677), count |
| | PREGNANCY_CHILDBIRTH_{—} | during F0: before and early pregnancy |
| | PUERPERIUM | |
| 867 | F0_pharma_J01XE01_{—} | Prescription of MACRODANTIN 29/M CAP 100 MG 30 |
| | MACRODANTIN_29_OR_M_{—} | (J01XE01), count during F0: before and early pregnancy |
| | CAP_100MG_30 | |
| 868 | F0_EOS_abs | Labtest EOS.abs, sampled during F0: before and early pregnancy |
| 869 | F0_pharma_J01CE02_{—} | Prescription of PEN-RAFA VK CPL 500 mg 40 (J01CE02), count |
| | PEN_RAFA_VK_CPL_{—} | during F0: before and early pregnancy |
| | 500mg_40 | |
| 870 | F1_clinic_7820_{—} | Clinic diagnosis DISTURBANCE OF SKIN SENSATION |
| | DISTURBANCE_OF_{—} | (7820), count during F1: first blood test (weeks 6-12) |
| | SKIN_SENSATION | |
| 871 | F2_pharma_J01CA04_{—} | Prescription of MOXYVIT CAP 500 MG 20 (J01CA04), count |
| | MOXYVIT_CAP_500MG_20 | during F2: second blood test (weeks 16-19) |
| 872 | F1_pharma_D01AC01_{—} | Prescription of CLOTRIMAZOLE (CLOTRIVIT) CR 1% 20 g |
| | CLOTRIMAZOLE_CLOTRIVIT_{—} | (D01AC01), count during F1: first blood test (weeks 6-12) |
| | CR_1%_20g | |
| 873 | F1_PT_SEC | Labtest PT-SEC, sampled during F1: first blood test (weeks 6-12) |
| 874 | F0_pharma_H08 | Prescription of (H08), count during F0: before and early |
| | | pregnancy |
| 875 | F2_ICTERIC | Labtest ICTERIC, sampled during F2: second blood test |
| | | (weeks 16-19) |
| 876 | F0_PROTEIN_TOTAL | Labtest PROTEIN-TOTAL, sampled during F0: before and early |
| | | pregnancy |
| 877 | F2_clinic_7910_PROTEINURIA | Clinic diagnosis PROTEINURIA (7910), count during F2: second |
| | | blood test (weeks 16-19) |
| 878 | F0_PROTEIN_C_ACTIVITY | Labtest PROTEIN C ACTIVITY, sampled during F0: before and |
| | | early pregnancy |
| 879 | F0_clinic_268_VITAMIN_D_{—} | Clinic diagnosis VITAMIN D DEFICIENCY (268), count during |
| | DEFICIENCY | F0: before and early pregnancy |
| 880 | F0_MICRO_F | Labtest MICRO-F, sampled during F0: before and early |
| | | pregnancy |
| 881 | F1_RETICULOCYTES_COUNT_perc | Labtest RETICULOCYTES COUNT %, sampled during F1: first |
| | | blood test (weeks 6-12) |
| 882 | F0_clinic_477_ALLERGIC_{—} | Clinic diagnosis ALLERGIC RHINITIS (477), count during F0: |
| | RHINITIS | before and early pregnancy |
| 883 | F2_pharma_A06AX01_{—} | Prescription of GLYCERIN (SAM-ON) SUP 2 GM 20 |
| | GLYCERIN_SAM_ON_SUP_{—} | (A06AX01), count during F2: second blood test (weeks 16-19) |
| | 2GM_20 | |
| 884 | F2_CHOLESTEROL_or_HDL | Labtest CHOLESTEROL/HDL, sampled during F2: second |
| | | blood test (weeks 16-19) |
| 885 | F1_RHEUMATOID_FACTOR | Labtest RHEUMATOID FACTOR, sampled during F1: first |
| | | blood test (weeks 6-12) |
| 886 | F1_pharma_D01AC21_{—} | Prescription of HYDRO AGISTEN CR CF 15 GM (D01AC21), |
| | HYDROAGISTEN_CR_CF_{—} | count during F1: first blood test (weeks 6-12) |
| | 15GM | |
| 887 | prev_pregnancy_3_MCHC | Labtest MCHC, sampled during 3rd previous pregnancy |
| 888 | F2_HEPATITIS_Bs_Ab | Labtest HEPATITIS Bs Ab, sampled during F2: second blood test |
| | | (weeks 16-19) |
| 889 | F1_EBV_VCA_IgG | Labtest EBV VCA IgG, sampled during F1: first blood test |
| | | (weeks 6-12) |
| 890 | F1_pharma_M01AE01_{—} | Prescription of ADVIL LIQUI-GELS CAP 200 MG 40 |
| | ADVIL_LIQUI_GELS_CAP_{—} | (M01AE01), count during F1: first blood test (weeks 6-12) |
| | 200MG_40 | |
| 891 | F0_ANTI_CARDIOLIPIN_IgM | Labtest ANTI CARDIOLIPIN IgM, sampled during F0: before |
| | | and early pregnancy |
| 892 | F1_pharma_B03AD21_{—} | Prescription of FERRIFOL TAB CF BOX 30 (B03AD21), count |
| | FERRIFOL_TAB_CF_BOX_30 | during F1: first blood test (weeks 6-12) |
| 893 | F0_pharma_D01AC23_{—} | Prescription of TEVADERM CR CF 15 GM (D01AC23), count |
| | TEVADERM_CR_CF_15GM | during F0: before and early pregnancy |
| 894 | F0_T3_FREE | Labtest T3- FREE, sampled during F0: before and early |
| | | pregnancy |
| 895 | F0_hospital_V2689_{—} | Hospital diagnosis ENCOUNTER FOR OTHER SPECIFIED |
| | ENCOUNTER_FOR_OTHER_{—} | PROCREATIVE MANAGEMENT (V2689), count during F0: |
| | SPECIFIED_PROCREATIVE_{—} | before and early pregnancy |
| | MANAGEMENT | |
| 896 | F0_FOLIC_ACID | Labtest FOLIC ACID, sampled during F0: before and early |
| | | pregnancy |
| 897 | F2_clinic_7840_HEADACHE | Clinic diagnosis HEADACHE (7840), count during F2: second |
| | | blood test (weeks 16-19) |
| 898 | F2_pharma_N06AB10_{—} | Prescription of CIPRALEX (MUSHLAM) TAB 10 MG 28 |
| | CIPRALEX_MUSHLAM_TAB_{—} | (N06AB10), count during F2: second blood test (weeks 16-19) |
| | 10MG_28 | |
| 899 | F2_FIBRINOGEN_CALCU | Labtest FIBRINOGEN CALCU, sampled during F2: second |
| | | blood test (weeks 16-19) |
| 900 | F0_clinic_2449_{—} | Clinic diagnosis UNSPECIFIED ACQUIRED |
| | UNSPECIFIED_ACQUIRED_{—} | HYPOTHYROIDISM (2449), count during F0: before and early |
| | HYPOTHYROIDISM | pregnancy |
| 901 | F2_clinic_460_ACUTE_{—} | Clinic diagnosis ACUTE NASOPHARYNGITIS (COMMON |
| | NASOPHARYNGITIS_{—} | COLD) (460), count during F2: second blood test (weeks 16-19) |
| | COMMON_COLD | |
| 902 | F2_pharma_A11DB01_{—} | Prescription of TRIBEMIN TAB 20 (A11DB01), count during F2: |
| | TRIBEMIN_TAB_20 | second blood test (weeks 16-19) |
| 903 | F1_pharma_N02BE01_{—} | Prescription of ACAMOL CPL 500 MG 21 (N02BE01), count |
| | ACAMOL_CPL_500MG_21 | during F1: first blood test (weeks 6-12) |
| 904 | F2_clinic_U80_INJURIES | Clinic diagnosis INJURIES (U80), count during F2: second blood |
| | | test (weeks 16-19) |
| 905 | F2_clinic_487_INFLUENZA | Clinic diagnosis INFLUENZA (487), count during F2: second |
| | | blood test (weeks 16-19) |
| 906 | F0_A_B2_GLYCOPROTEI_IgG | Labtest A.B2 GLYCOPROTEI IgG, sampled during F0: before |
| | | and early pregnancy |
| 907 | F0_pharma_G03GA06_{—} | Prescription of PUREGON INJ 900IU (G03GA06), count during |
| | PUREGON_INJ_900IU | F0: before and early pregnancy |
| 908 | F2_hospital_Z9911_{—} | Hospital diagnosis INJECTION OF RH IMMUNE GLOBULIN |
| | INJECTION_OF_RH_{—} | (Z9911), count during F2: second blood test (weeks 16-19) |
| | IMMUNE_GLOBULIN | |
| 909 | F2_clinic_6465_{—} | Clinic diagnosis ASYMPTOMATIC BACTERIURIA IN |
| | ASYMPTOMATIC_{—} | PREGNANCY (6465), count during F2: second blood test |
| | BACTERIURIA_IN_{—} | (weeks 16-19) |
| | PREGNANCY | |
| 910 | F0_LDH | Labtest LDH, sampled during F0: before and early pregnancy |
| 911 | F1_clinic_268_VITAMIN_D_{—} | Clinic diagnosis VITAMIN D DEFICIENCY (268), count during |
| | DEFICIENCY | F1: first blood test (weeks 6-12) |
| 912 | F0_clinic_9599_OTHER_AND_{—} | Clinic diagnosis OTHER AND UNSPECIFIED INJURY TO |
| | UNSPECIFIED_INJURY_{—} | UNSPECIFIED SITE (9599), count during F0: before and early |
| | TO_UNSPECIFIED_SITE | pregnancy |
| 913 | F2_FIBRINOGEN | Labtest FIBRINOGEN, sampled during F2: second blood test |
| | | (weeks 16-19) |
| 914 | prev_pregnancy_3_BASO_abs | Labtest BASO abs, sampled during 3rd previous pregnancy |
| 915 | F0_clinic_9953_ALLERGY_{—} | Clinic diagnosis ALLERGY, UNSPECIFIED, NOT |
| | UNSPECIFIED_NOT_{—} | ELSEWHERE CLASSIFIED (9953), count during F0: before and |
| | ELSEWHERE_CLASSIFIED | early pregnancy |
| 916 | F0_PROTEIN_U_SAMPLE | Labtest PROTEIN- U SAMPLE, sampled during F0: before and |
| | | early pregnancy |
| 917 | F2_clinic_3540_CARPAL_{—} | Clinic diagnosis CARPAL TUNNEL SYNDROME (3540), count |
| | TUNNEL_SYNDROME | during F2: second blood test (weeks 16-19) |
| 918 | F2_PT_SEC | Labtest PT-SEC, sampled during F2: second blood test |
| | | (weeks 16-19) |
| 919 | F2_clinic_465_ACUTE_UPPER_{—} | Clinic diagnosis ACUTE UPPER RESPIRATORY INFECTIONS |
| | RESPIRATORY_INFECTIONS_{—} | OF MULTIPLE OR UNSP.SITES (465), count during F2: second |
| | OF_MULTIPLE_OR_UNSP_SITES | blood test (weeks 16-19) |
| 920 | F1_pharma_A03FA01_{—} | Prescription of PRAMIN SUP 20 MG 6 (A03FA01), count during |
| | PRAMIN_SUP_20MG_6 | F1: first blood test (weeks 6-12) |
| 921 | F0_clinic_799_OTHER_ILL_{—} | Clinic diagnosis OTHER ILL-DEFINED AND UNKNOWN |
| | DEFINED_AND_UNKNOWN_{—} | CAUSES OF MORBIDITY AND MORTALITY (799), count |
| | CAUSES_OF_MORBIDITY_{—} | during F0: before and early pregnancy |
| | AND_MORTALITY | |
| 922 | F2_clinic_7194_PAIN_IN_JOINT | Clinic diagnosis PAIN IN JOINT (7194), count during F2: second |
| | | blood test (weeks 16-19) |
| 923 | F1_LH | Labtest LH, sampled during F1: first blood test (weeks 6-12) |
| 924 | F0_CMV_IgG | Labtest CMV IgG, sampled during F0: before and early |
| | | pregnancy |
| 925 | F2_pharma_R01BA52_{—} | Prescription of CLARINASE REPETABS TAB 14 (R01BA52), |
| | CLARINASE_REPETABS_{—} | count during F2: second blood test (weeks 16-19) |
| | TAB_14 | |
| 926 | F2_clinic_2169_BENIGN_{—} | Clinic diagnosis BENIGN NEOPLASM OF SKIN, SITE |
| | NEOPLASM_OF_SKIN_{—} | UNSPECIFIED (2169), count during F2: second blood test |
| | SITE_UNSPECIFIED | (weeks 16-19) |
| 927 | F1_clinic_7242_LUMBAGO | Clinic diagnosis LUMBAGO (7242), count during F1: first blood |
| | | test (weeks 6-12) |
| 928 | F1_PROTEIN_URINE_24h | Labtest PROTEIN- URINE 24 h, sampled during F1: first blood |
| | | test (weeks 6-12) |
| 929 | F2_hospital_63791_{—} | Hospital diagnosis UNSPECIFIED |
| | UNSPECIFIED_ABORTION_{—} | ABORTION, INCOMPLETE, WITHOUT COMPLICATION |
| | INCOMPLETE_WITHOUT_{—} | (63791), count during F2: second blood test (weeks 16-19) |
| | COMPLICATION | |
| 930 | F2_hospital_7999_{—} | Hospital diagnosis UNKNOWN OR UNSPECIFIED CAUSE OF |
| | UNKNOWN_OR_{—} | MORBIDITY (DIAGNOSIS DEFERRED) (7999), count during |
| | UNSPECIFIED_CAUSE_{—} | F2: second blood test (weeks 16-19) |
| | OF_MORBIDITY_{—} | |
| | DIAGNOSIS_DEFERRED | |
| 931 | M5_MICRO_perc | Labtest MICRO %, sampled during M5: year 5 before pregnancy |
| 932 | F0_clinic_V700_ROUTINE_{—} | Clinic diagnosis ROUTINE GENERAL MEDICAL |
| | GENERAL_MEDICAL_{—} | EXAMINATION AT A HEALTH CARE FACILITY (V700), |
| | EXAMINATION_AT_A_{—} | count during F0: before and early pregnancy |
| | HEALTH_CARE_FACILITY | |
| 933 | F2_FOLIC_ACID | Labtest FOLIC ACID, sampled during F2: second blood test |
| | | (weeks 16-19) |
| 934 | F1_hospital_Z6591_{—} | Hospital diagnosis ASPIRATION OF OVARY (Z6591), count |
| | ASPIRATION_OF_OVARY | during F1: first blood test (weeks 6-12) |
| 935 | F1_clinic_5350_GASTRITIS | Clinic diagnosis GASTRITIS (5350), count during F1: first blood |
| | | test (weeks 6-12) |
| 936 | F1_CMV_IgG | Labtest CMV IgG, sampled during F1: first blood test |
| | | (weeks 6-12) |
| 937 | F0_pharma_J01CA04_{—} | Prescription of MOXYVIT CAP 500 MG 20 (J01CA04), count |
| | MOXYVIT_CAP_500MG_20 | during F0: before and early pregnancy |
| 938 | F1_pharma_Z07_AFTER_{—} | Prescription of AFTER 10 STICK KIT (Z07), count during F1: |
| | 10_STICK_KIT | first blood test (weeks 6-12) |
| 939 | F2_clinic_6463_HABITUAL_{—} | Clinic diagnosis HABITUAL ABORTER, CURRENTLY |
| | ABORTER_CURRENTLY_PREGNANT | PREGNANT (6463), count during F2: second blood test |
| | | (weeks 16-19) |
| 940 | F0_clinic_Z8936_MANUAL_{—} | Clinic diagnosis MANUAL EXAMINATION OF BREAST |
| | EXAMINATION_OF_BREAST | (Z8936), count during F0: before and early pregnancy |
| 941 | F0_clinic_7890_ABDOMINAL_PAIN | Clinic diagnosis ABDOMINAL PAIN (7890), count during F0: |
| | | before and early pregnancy |
| 942 | F2_ALBUMIN | Labtest ALBUMIN, sampled during F2: second blood test |
| | | (weeks 16-19) |
| 943 | F1_MAGNESIUM | Labtest MAGNESIUM, sampled during F1: first blood test |
| | | (weeks 6-12) |
| 944 | F0_clinic_7804_DIZZINESS_{—} | Clinic diagnosis DIZZINESS AND GIDDINESS (VERTIGO) |
| | AND_GIDDINESS_VERTIGO | (7804), count during F0: before and early pregnancy |
| 945 | F2_HEMOGLOBIN_S | Labtest HEMOGLOBIN S, sampled during F2: second blood test |
| | | (weeks 16-19) |
| 946 | F0_hospital_7840_HEADACHE | Hospital diagnosis HEADACHE (7840), count during F0: before |
| | | and early pregnancy |
| 947 | F2_pharma_M01AE01_ADVIL_{—} | Prescription of ADVIL LIQUI-GELS CAP 200 MG 40 |
| | LIQUI_GELS_CAP_200MG_40 | (M01AE01), count during F2: second blood test (weeks 16-19) |
| 948 | F2_ERYTHROCYTES_U_STRIP | Labtest ERYTHROCYTES-U STRIP, sampled during F2: second |
| | | blood test (weeks 16-19) |
| 949 | F0_pharma_B03BA01_VIT_{—} | Prescription of VIT B12(SOLGAR)SUBL TAB 1000 mcg 100 |
| | B12_SOLGAR_SUBL_TAB_{—} | (B03BA01), count during F0: before and early pregnancy |
| | 1000mcg_100 | |
| 950 | F2_pharma_J01CR02_{—} | Prescription of AUGMENTIN BID TAB 875 MG 14 (J01CR02), |
| | AUGMENTIN_BID_TAB_{—} | count during F2: second blood test (weeks 16-19) |
| | 875MG_14 | |
| 951 | F1_clinic_477_ALLERGIC_RHINITIS | Clinic diagnosis ALLERGIC RHINITIS (477), count during F1: |
| | | first blood test (weeks 6-12) |
| 952 | F1_clinic_7061_OTHER_ACNE | Clinic diagnosis OTHER ACNE (7061), count during F1: first |
| | | blood test (weeks 6-12) |
| 953 | M1_MACRO_perc | Labtest MACRO %, sampled during M1: year 1 before pregnancy |
| 954 | F1_pharma_R01BA55_{—} | Prescription of NUSSIDEX TAB CF 20 (R01BA55), count during |
| | NUSSIDEX_TAB_CF_20 | F1: first blood test (weeks 6-12) |
| 955 | F0_clinic_Y10_INFERTILITY_{—} | Clinic diagnosis INFERTILITY/SUBFERTILITY (Y10), count |
| | OR_SUBFERTILITY | during F0: before and early pregnancy |
| 956 | F0_HB_other | Labtest HB -other, sampled during F0: before and early pregnancy |
| 957 | F1_hospital_0_OTHER | Hospital diagnosis OTHER (0), count during F1: first blood test |
| | | (weeks 6-12) |
| 958 | F0_pharma_Unknown_ATC_{—} | Prescription of OTHER (Unknown_ATC), count during F0: |
| | OTHER | before and early pregnancy |
| 959 | F1_ANTITHROMBIN_III | Labtest ANTITHROMBIN-III, sampled during F1: first blood test |
| | | (weeks 6-12) |
| 960 | M2_EOS_perc | Labtest EOS %, sampled during M2: year 2 before pregnancy |
| 961 | F0_clinic_5301_ESOPHAGITIS | Clinic diagnosis ESOPHAGITIS (5301), count during F0: before |
| | | and early pregnancy |
| 962 | F0_pharma_R06AX26_{—} | Prescription of TELFAST TAB 180 MG 15 (R06AX26), count |
| | TELFAST_TAB_180MG_15 | during F0: before and early pregnancy |
| 963 | F0_hospital_7999_{—} | Hospital diagnosis UNKNOWN OR UNSPECIFIED CAUSE OF |
| | UNKNOWN_OR_{—} | MORBIDITY (DIAGNOSIS DEFERRED) (7999), count during |
| | UNSPECIFIED_CAUSE_{—} | F0: before and early pregnancy |
| | OF_MORBIDITY_{—} | |
| | DIAGNOSIS_DEFERRED | |
| 964 | F1_clinic_6482_ANEMIA_{—} | Clinic diagnosis ANEMIA COMPLICATING PREGNANCY, |
| | COMPLICATING_PREGNANCY_{—} | CHILDBIRTH, OR THE PUERPERIUM (6482), count during |
| | CHILDBIRTH_OR_THE_{—} | F1: first blood test (weeks 6-12) |
| | PUERPERIUM | |
| 965 | prev_pregnancy_3_PLT | Labtest PLT, sampled during 3rd previous pregnancy |
| 966 | F1_CHLORIDE | Labtest CHLORIDE, sampled during F1: first blood test |
| | | (weeks 6-12) |
| 967 | F2_IRON | Labtest IRON, sampled during F2: second blood test |
| | | (weeks 16-19) |
| 968 | F1_pharma_G01AA10_{—} | Prescription of DALACIN VAGINAL OVL 100 MG 3 |
| | DALACIN_VAGINAL_OVL_{—} | (G01AA10), count during F1: first blood test (weeks 6-12) |
| | 100MG_3 | |
| 969 | F2_hospital_64093_{—} | Hospital diagnosis UNSPECIFIED HEMORRHAGE IN EARLY |
| | UNSPECIFIED_HEMORRHAGE_{—} | PREGNANCY, ANTEPARTUM (64093), count during F2: |
| | IN_EARLY_PREGNANCY_{—} | second blood test (weeks 16-19) |
| | ANTEPARTUM | |
| 970 | F2_PROTEIN_C_ACTIVITY | Labtest PROTEIN C ACTIVITY, sampled during F2: second |
| | | blood test (weeks 16-19) |
| 971 | F0_pharma_B03AD02_{—} | Prescription of FORIC PREGNANCY TAB CF BOX 30 |
| | FORIC_PREGNANCY_TAB_{—} | (B03AD02), count during F0: before and early pregnancy |
| | CF_BOX_30 | |
| 972 | F1_pharma_G12A | Prescription of (G12A), count during F1: first blood test |
| | | (weeks 6-12) |
| 973 | F1_EBV_IgG_EBNA | Labtest EBV IgG-EBNA, sampled during F1: first blood test |
| | | (weeks 6-12) |
| 974 | F1_FREE_ANDROGEN_INDEX | Labtest FREE ANDROGEN INDEX, sampled during F1: first |
| | | blood test (weeks 6-12) |
| 975 | F2_TRANSFERRIN | Labtest TRANSFERRIN, sampled during F2: second blood test |
| | | (weeks 16-19) |
| 976 | F2_clinic_6510_TWIN_{—} | Clinic diagnosis TWIN PREGNANCY (6510), count during F2: |
| | PREGNANCY | second blood test (weeks 16-19) |
| 977 | F1_HEMOGLOBIN_H | Labtest HEMOGLOBIN H, sampled during F1: first blood test |
| | | (weeks 6-12) |
| 978 | F2_pharma_D01AC24_{—} | Prescription of POLYCUTAN CR 15GM (D01AC24), count |
| | POLYCUTAN_CR_15GM | during F2: second blood test (weeks 16-19) |
| 979 | F1_pharma_B01AB05_{—} | Prescription of CLEXANE INJ 40 mg/0.4 mL 2X0.4 mL |
| | CLEXANE_INJ_40mg_OR_{—} | (B01AB05), count during F1: first blood test (weeks 6-12) |
| | 0_4mL_2X0_4mL | |
| 980 | F1_hospital_Z6952_{—} | Hospital diagnosis ASPIRATION CURETTAGE FOLLOWING |
| | ASPIRATION_CURETTAGE_FO | DELIVERY OR ABORTION (Z6952), count during F1: first |
| | LLOWING_DELIVERY_{—} | blood test (weeks 6-12) |
| | OR_ABORTION | |
| 981 | M3_HEMOGLOBIN_A1C_perc | Labtest HEMOGLOBIN A1C %, sampled during M3: year 3 |
| | | before pregnancy |
| 982 | F0_pharma_G01AA10_{—} | Prescription of DALACIN VAGINAL OVL 100 MG 3 |
| | DALACIN_VAGINAL_OVL_{—} | (G01AA10), count during F0: before and early pregnancy |
| | 100MG_3 | |
| 983 | F2_pharma_B03BB01_{—} | Prescription of FOLI 5 (CTS) TAB 5 mg 30 (B03BB01), count |
| | FOLI_5_CTS_TAB_5mg_30 | during F2: second blood test (weeks 16-19) |
| 984 | F2_pharma_A11CC05_{—} | Prescription of BABY D3 DRP 200IU 10 ML (A11CC05), count |
| | BABY_D3_DRP_200IU_10ML | during F2: second blood test (weeks 16-19) |
| 985 | F1_ALBUMIN | Labtest ALBUMIN, sampled during F1: first blood test |
| | | (weeks 6-12) |
| 986 | F1_pharma_S02CA06_DEX_{—} | Prescription of DEX-OTIC AUR CF BOT 5 ML (S02CA06), |
| | OTIC_AUR_CF_BOT_5ML | count during F1: first blood test (weeks 6-12) |
| 987 | F1_hospital_V2389_{—} | Hospital diagnosis SUPERVISION OF OTHER HIGH-RISK |
| | SUPERVISION_OF_OTHER_{—} | PREGNANCY (V2389), count during F1: first blood test |
| | HIGH_RISK_PREGNANCY | (weeks 6-12) |
| 988 | F0_hospital_Z9649_OTHER_{—} | Hospital diagnosis OTHER GENITOURINARY INSTILLATION |
| | GENITOURINARY_INSTILLATION | (Z9649), count during F0: before and early pregnancy |
| 989 | F1_clinic_9249_CONTUSION_{—} | Clinic diagnosis CONTUSION OF UNSPECIFIED SITE (9249), |
| | OF_UNSPECIFIED_SITE | count during F1: first blood test (weeks 6-12) |
| 990 | F2_hospital_V2681_{—} | Hospital diagnosis ENCOUNTER FOR ASSISTED |
| | ENCOUNTER_FOR_ASSISTED_{—} | REPRODUCTIVE FERTILITY PROCEDURE CYCLE (V2681), |
| | REPRODUCTIVE_FERTILITY_{—} | count during F2: second blood test (weeks 16-19) |
| | PROCEDURE_CYCLE | |
| 991 | F2_pharma_Z07_YES_OR_NO_{—} | Prescription of YES OR NO DIRECT KIT 1 (Z07), count during |
| | DIRECT_KIT_1 | F2: second blood test (weeks 16-19) |
| 992 | F1_pharma_G03DA04_{—} | Prescription of UTROGESTAN ORAL/VAG CAP 100 mg 30 |
| | UTROGESTAN_ORAL_OR_VAG_{—} | (G03DA04), count during F1: first blood test (weeks 6-12) |
| | CAP_100mg_30 | |
| 993 | F1_clinic_799_OTHER_{—} | Clinic diagnosis OTHER ILL-DEFINED AND UNKNOWN |
| | ILL_DEFINED_AND_{—} | CAUSES OF MORBIDITY AND MORTALITY (799), count |
| | UNKNOWN_CAUSES_OF_{—} | during F1: first blood test (weeks 6-12) |
| | MORBIDITY_AND_MORTALITY | |
| 994 | F2_pharma_N02BB02_{—} | Prescription of OPTALGIN CPL 500 MG 21 (N02BB02), count |
| | OPTALGIN_CPL_500MG_21 | during F2: second blood test (weeks 16-19) |
| 995 | F1_pharma_J01CR02_{—} | Prescription of AUGMENTIN BID TAB 875 MG 14 (J01CR02), |
| | AUGMENTIN_BID_TAB_875MG_14 | count during F1: first blood test (weeks 6-12) |
| 996 | high_baseline_BP_count | Number of non pregnancy high BP tests recorded |
| 997 | F2_hospital_Z5123_{—} | Hospital diagnosis LAPAROSCOPIC |
| | LAPAROSCOPIC_{—} | CHOLECYSTECTOMY_1992 (Z5123), count during F2: |
| | CHOLECYSTECTOMY_1992 | second blood test (weeks 16-19) |
| 998 | F2_pharma_U2 | Prescription of (U2), count during F2: second blood test |
| | | (weeks 16-19) |
| 999 | F0_clinic_5246_{—} | Clinic diagnosis TEMPOROMANDIBULAR JOINT |
| | TEMPOROMANDIBULAR_{—} | DISORDERS (5246), count during F0: before and early |
| | JOINT_DISORDERS | pregnancy |
| 1000 | F2_pharma_B01AC06_{—} | Prescription of MICROPIRIN TAB 100 MG 28 (B01AC06), count |
| | MICROPIRIN_TAB_100MG_28 | during F2: second blood test (weeks 16-19) |
| 1001 | F2_HEMOGLOBIN_A | Labtest HEMOGLOBIN A. sampled during F2: second blood test |
| | | (weeks 16-19) |
| 1002 | F0_AMYLASE | Labtest AMYLASE, sampled during F0: before and early |
| | | pregnancy |
| 1003 | F2_RHEUMATOID_FACTOR | Labtest RHEUMATOID FACTOR, sampled during F2: second |
| | | blood test (weeks 16-19) |
| 1004 | F0_pharma_H03B | Prescription of (H03B), count during F0: before and early |
| | | pregnancy |
| 1005 | F0_pharma_G01AF02_{—} | Prescription of CLOTREE-TEVA VAG OVL 200 MG 3 |
| | CLOTREE_TEVA_VAG_OVL_{—} | (G01AF02), count during F0: before and early pregnancy |
| | 200MG_3 | |
| 1006 | F0_pharma_J01CA04_{—} | Prescription of MOXYPEN CAP 500 MG 10 (J01CA04), count |
| | MOXYPEN_CAP_500MG_10 | during F0: before and early pregnancy |
| 1007 | F0_pharma_L02AE04_{—} | Prescription of DECAPEPTYL INJ 0.1 mg/1 mL 7 (L02AE04), |
| | DECAPEPTYL_INJ_0_1mg_{—} | count during F0: before and early pregnancy |
| | OR_1mL_7 | |
| 1008 | F0_pharma_G03CA03_{—} | Prescription of PROGYNOVA TAB 2 MG 28 (G03CA03), count |
| | PROGYNOVA_TAB_2MG_28 | during F0: before and early pregnancy |
| 1009 | F0_pharma_R05DA21_{—} | Prescription of DEXAMOL COLD 30D + 20N CPL (R05DA21), |
| | DEXAMOL_COLD_30D+20N_CPL | count during F0: before and early pregnancy |
| 1010 | F0_pharma_J01CR02_{—} | Prescription of AUGMENTIN BID TAB 875 MG 14 (J01CR02), |
| | AUGMENTIN_BID_TAB_875MG_14 | count during F0: before and early pregnancy |
| 1011 | F0_pharma_D01AC21_{—} | Prescription of HYDROAGISTEN CR CF 15 GM (D01AC21), |
| | HYDROAGISTEN_CR_CF_{—} | count during F0: before and early pregnancy |
| | 15GM | |
| 1012 | F0_pharma_H03AA01_{—} | Prescription of ELTROXIN TAB 50 MCG 100 (H03AA01), count |
| | ELTROXIN_TAB_50MCG_100 | during F0: before and early pregnancy |
| 1013 | F0_pharma_B03AE02_IRON_{—} | Prescription of IRON BIS-GLYCINATE DH CAP 90 |
| | BIS_GLYCINATE_DH_CAP_90 | (B03AE02), count during F0: before and early pregnancy |
| 1014 | F0_pharma_C10AX06_{—} | Prescription of OMEGA-3 9 MONTHS (ALTMAN) CAP BOX |
| | OMEGA_3_9_MONTHS_{—} | 60 (C10AX06), count during F0: before and early pregnancy |
| | ALTMAN_CAP_BOX_60 | |
| 1015 | F0_pharma_A06AX01_{—} | Prescription of GLYCERIN (SAM-ON) SUP 2 GM 20 |
| | GLYCERIN_SAM_ON_SUP_{—} | (A06AX01), count during F0: before and early pregnancy |
| | 2GM_20 | |
| 1016 | F0_pharma_G03GA05_{—} | Prescription of GONAL-F-PEN INJ 900IU 1.5 ML (G03GA05), |
| | GONAL_F_PEN_INJ_{—} | count during F0: before and early pregnancy |
| | 900IU_1_5ML | |
| 1017 | F0_pharma_D01AC01_{—} | Prescription of CLOTRIMAZOLE (CLOTRIVIT) CR 1% 20 g |
| | CLOTRIMAZOLE_CLOTRIVIT_{—} | (D01AC01), count during F0: before and early pregnancy |
| | CR_1%_20g | |
| 1018 | F0_pharma_R05CB01_{—} | Prescription of REOLIN EFFERV. TAB 200 MG 30 (R05CB01), |
| | REOLIN_EFFERV_TAB_200MG_30 | count during F0: before and early pregnancy |
| 1019 | F0_pharma_G03DA04_{—} | Prescription of UTROGESTAN ORAL/VAG CAP 100 mg 30 |
| | UTROGESTAN_ORAL_OR_{—} | (G03DA04), count during F0: before and early pregnancy |
| | VAG_CAP_100mg_30 | |
| 1020 | F0_pharma_B03AD03_{—} | Prescription of FERROGRAD FOLIC TAB CF BOX 30 |
| | FERROGRAD_FOLIC_TAB_{—} | (B03AD03), count during F0: before and early pregnancy |
| | CF_BOX_30 | |
| 1021 | F0_pharma_R05CA20_{—} | Prescription of THYMI SYR BOT 100 ML (R05CA20), count |
| | THYMI_SYR_BOT_100ML | during F0: before and early pregnancy |
| 1022 | F0_pharma_G03DA03_{—} | Prescription of PROGESTERON RETARD 29/M INJ |
| | PROGESTERON_RETARD_{—} | 500 MG/2 ML 3 (G03DA03), count during F0: before and early |
| | 29_OR_M_INJ_500MG_OR_2ML_3 | pregnancy |
| 1023 | F0_pharma_H01CC02_{—} | Prescription of CETROTIDE VIA 0.25 mg 7 (H01CC02), count |
| | CETROTIDE_VIA_0_25mg_7 | during F0: before and early pregnancy |
| 1024 | F0_pharma_J01DB01_{—} | Prescription of CEFOVIT FORTE CAP 500 mg 20 (J01DB01), |
| | CEFOVIT_FORTE_CAP_500mg_20 | count during F0: before and early pregnancy |
| 1025 | F0_pharma_R01AX10_{—} | Prescription of OTRIMER SPR 0.9% 50 ML (R01AX10), count |
| | OTRIMER_SPR_0_9%_50ML | during F0: before and early pregnancy |
| 1026 | F0_pharma_A02BC01_{—} | Prescription of OMEPRADEX CPL 20 mg 30 (A02BC01), count |
| | OMEPRADEX_CPL_20mg_30 | during F0: before and early pregnancy |
| 1027 | F0_pharma_Z07_YES_OR_{—} | Prescription of YES OR NO DIRECT KIT 1 (Z07), count during |
| | NO_DIRECT_KIT_1 | F0: before and early pregnancy |
| 1028 | F0_pharma_B03BA01_{—} | Prescription of BEDODEKA INJ 1000 MCG/1 ML 100 |
| | BEDODEKA_INJ_1000MCG_{—} | (B03BA01), count during F0: before and early pregnancy |
| | OR_1ML_100 | |
| 1029 | F0_pharma_{——}STREP_ATEST_{—} | Prescription of STREP A TEST- QUICKVUE IN-LINE ( ), count |
| | QUICKVUE_IN_LINE | during F0: before and early pregnancy |
| 1030 | F0_pharma_G03GA02_{—} | Prescription of MENOPUR MULTIDOSE INJ 600 UNIT |
| | MENOPUR_MULTIDOSE_INJ_{—} | (G03GA02), count during F0: before and early pregnancy |
| | 600UNIT | |
| 1031 | F0_pharma_H03AA01_{—} | Prescription of EUTHYROX TAB 100 mcg 100 (H03AA01), |
| | EUTHYROX_TAB_100mcg_100 | count during F0: before and early pregnancy |
| 1032 | F0_pharma_D08AX08_{—} | Prescription of A1COTINT SPT 70% 100 ML (D08AX08), count |
| | A1COTINT_SPT_70%_100ML | during F0: before and early pregnancy |
| 1033 | F0_pharma_H03AA01_{—} | Prescription of EUTHYROX TAB 50 mcg 100 (H03AA01), count |
| | EUTHYROX_TAB_50mcg_100 | during F0: before and early pregnancy |
| 1034 | F0_pharma_M01AE01_{—} | Prescription of ADVIL LIQUI-GELS CAP 200 MG 16 |
| | ADVIL_LIQUI_GELS_CAP_200MG_16 | (M01AE01), count during F0: before and early pregnancy |
| 1035 | F0_pharma_D01AC22_{—} | Prescription of AGISTEN ALOEVERA CR 1% 20 GM |
| | AGISTEN_ALOEVERA_CR_{—} | (D01AC22), count during F0: before and early pregnancy |
| | 1%_20GM | |
| 1036 | F0_pharma_M01AE01_{—} | Prescription of NUROFEN STRAWB.CHILD SUS 100 MG/5 ML |
| | NUROFEN_STRAWB_CHILD_{—} | 150 ML (M01AE01), count during F0: before and early pregnancy |
| | SUS_100MG_OR_5ML_150ML | |
| 1037 | F0_pharma_R03AK07_{—} | Prescription of SYMBICORT TURBUHALER INH |
| | SYMBICORT_TURBUHALER_{—} | 160 mcg/4.5 mcg (R03AK07), count during F0: before and early |
| | INH_160mcg_OR_4_5mcg | pregnancy |
| 1038 | F0_pharma_A04AA01_{—} | Prescription of ZOFRAN TAB 8 MG 10 (A04AA01), count during |
| | ZOFRAN_TAB_8MG_10 | F0: before and early pregnancy |
| 1039 | F0_pharma_G01AF05_{—} | Prescription of GYNO-PEVARYL VAG TAB 150 MG 3 |
| | GYNO_PEVARYL_VAG_{—} | (G01AF05), count during F0: before and early pregnancy |
| | TAB_150MG_3 | |
| 1040 | F0_pharma_S02CA06_{—} | Prescription of DEX-OTIC AUR CF BOT 5 ML (S02CA06), |
| | DEX_OTIC_AUR_CF_BOT_5ML | count during F0: before and early pregnancy |
| 1041 | F0_pharma_J06BB01_{—} | Prescription of KAMRHO-D I.M. INJ 300 mcg 2 mL (J06BB01), |
| | KAMRHO_D_I_M_INJ_{—} | count during F0: before and early pregnancy |
| | 300mcg_2mL | |
| 1042 | F0_pharma_B01AB05_{—} | Prescription of CLEXANE INJ 40 mg/0.4 mL 2X0.4 mL |
| | CLEXANE_INJ_40mg_OR_{—} | (B01AB05), count during F0: before and early pregnancy |
| | 0_4mL_2X0_4mL | |
| 1043 | F0_pharma_B03AB10_{—} | Prescription of FERRIPEL-3 SYR 50 MG/5 ML 110 ML |
| | FERRIPEL_3_SYR_50MG_{—} | (B03AB10), count during F0: before and early pregnancy |
| | OR_5ML_110ML | |
| 1044 | F0_pharma_A11CC05_{—} | Prescription of VITAMIDYNE D DRP 200IU 10 ML (A11CC05), |
| | VITAMIDYNE_D_DRP_{—} | count during F0: before and early pregnancy |
| | 200IU_10ML | |
| 1045 | F0_pharma_{——}OTRIVONIM_{—} | Prescription of OTRIVONIM BABY WIP PKG 2X36U (), count |
| | BABY_WIP_PKG_2X36U | during F0: before and early pregnancy |
| 1046 | F0_pharma_R01AB22_{—} | Prescription of OTRIVIN MENTHOL M.D SPR 0.1% 10 ML |
| | OTRIVIN_MENTHOL_{—} | (R01AB22), count during F0: before and early pregnancy |
| | M_D_SPR_0_1%_10ML | |
| 1047 | F0_pharma_{——}PARO_{—} | Prescription of PARO TOOTHBRUSH MEDIC NO. 726 ( ), |
| | TOOTHBRUSH_MEDIC_{—} | count during F0: before and early pregnancy |
| | NO_726 | |
| 1048 | F0_pharma_B01AC06_{—} | Prescription of MICROPIRIN TAB 100 MG 28 (B01AC06), count |
| | MICROPIRIN_TAB_100MG_28 | during F0: before and early pregnancy |
| 1049 | F0_pharma_G04BX06_{—} | Prescription of SEDURAL TAB 100 MG 30 (G04BX06), count |
| | SEDURAL_TAB_100MG_30 | during F0: before and early pregnancy |
| 1050 | F0_pharma_C10AX06_{—} | Prescription of OMEGA D3 9 MONTHS (SUPH) S.GEL CAP 60 |
| | OMEGA_D3_9_MONTHS_{—} | (C10AX06), count during F0: before and early pregnancy |
| | SUPH_S_GEL_CAP_60 | |
| 1051 | F0_pharma_B03AE12_{—} | Prescription of IRON PLUS FEMINA (ALTMAN) TAB 100 |
| | IRON_PLUS_FEMINA_{—} | (B03AE12), count during F0: before and early pregnancy |
| | ALTMAN_TAB_100 | |
| 1052 | F0_pharma_B03AD02_{—} | Prescription of FOROL TAB 30 (B03AD02), count during F0: |
| | FOROL_TAB_30 | before and early pregnancy |
| 1053 | F0_pharma_G03DC02_{—} | Prescription of PRIMOLUT NOR TAB 5 MG 20 (G03DC02), |
| | PRIMOLUT_NORTAB_5MG_20 | count during F0: before and early pregnancy |
| 1054 | F0_pharma_J01CR02_{—} | Prescription of AUGMENTIN TAB 500 MG 20 (J01CR02), count |
| | AUGMENTIN_TAB_500MG_20 | during F0: before and early pregnancy |
| 1055 | F0_pharma_N01BB54_{—} | Prescription of EMLA CR 5% 30 GM (N01BB54), count during |
| | EMLA_CR_5%_30GM | F0: before and early pregnancy |
| 1056 | F0_pharma_R01AD08_{—} | Prescription of FLIXONASE NASAL SPR 0.05% (R01AD08), |
| | FLIXONASE_NASAL_SPR_0_05% | count during F0: before and early pregnancy |
| 1057 | F0_pharma_A11AA06_{—} | Prescription of PRE-GENTLY BADATZ (SUPHERB)TAB 60 |
| | PRE_GENTLY_BADATZ_{—} | (A11AA06), count during F0: before and early pregnancy |
| | SUPHERB_TAB_60 | |
| 1058 | F0_pharma_H01CC01_{—} | Prescription of ORGALUTRAN SYRI.<><> INJ 0.25 MG 0.5 ML |
| | ORGALUTRAN_SYRI_<><>_{—} | (H01CC01), count during F0: before and early pregnancy |
| | INJ_0_25MG_0_5ML | |
| 1059 | F0_pharma_A02AD01_{—} | Prescription of MAALOX SUS CF BOT 355 ML (A02AD01), |
| | MAALOX_SUS_CF_BOT_355ML | count during F0: before and early pregnancy |
| 1060 | F0_pharma_R03AC02_{—} | Prescription of VENTOLIN RESPIR. SOL 5 mg/1 mL 20 mL |
| | VENTOLIN_RESPIR_SOL_{—} | (R03AC02), count during F0: before and early pregnancy |
| | 5mg_OR_1mL_20mL | |
| 1061 | F0_pharma_R02AA03_{—} | Prescription of STREPSILS HONEY-LEMON LOZ CF 24 |
| | STREPSILS_HONEY_{—} | (R02AA03), count during F0: before and early pregnancy |
| | LEMON_LOZ_CF_24 | |
| 1062 | F0_pharma_M01AE01_{—} | Prescription of NUROFEN ORANGE CHILD SUS 100 MG/5 ML |
| | NUROFEN_ORANGE_{—} | 150 ML (M01AE01), count during F0: before and early pregnancy |
| | CHILD_SUS_100MG_{—} | |
| | OR_5ML_150ML | |
| 1063 | F0_pharma_B03BA01_{—} | Prescription of BEVITEX SUBLINGUAL TAB 1000 MCG 30 |
| | BEVITEX_SUBLINGUAL_{—} | (B03BA01), count during F0: before and early pregnancy |
| | TAB_1000MCG_30 | |
| 1064 | F0_pharma_D07CA03_{—} | Prescription of AFLUMYCIN CR CF 20 GM (D07CA03), count |
| | AFLUMYCIN_CR_CF_20GM | during F0: before and early pregnancy |
| 1065 | F0_pharma_R02AA20_{—} | Prescription of LEMOCIN LEMON SUGAR FREE LOZ CF 24 |
| | LEMOCIN_LEMON_SUGAR_{—} | (R02AA20), count during F0: before and early pregnancy |
| | FREE_LOZ_CF_24 | |
| 1066 | F0_pharma_J01AA02_{—} | Prescription of DOXYLIN TAB 100 MG 10 (J01AA02), count |
| | DOXYLIN_TAB_100MG_10 | during F0: before and early pregnancy |
| 1067 | F0_pharma_U0_LABON_TIME | Prescription of LABON TIME (U0), count during F0: before and |
| | | early pregnancy |
| 1068 | F0_pharma_A11CC05_{—} | Prescription of VITAMIN D3 D.H DRP 400 UNIT 15 ML |
| | VITAMIN_D3_D_H_DRP_{—} | (A11CC05), count during F0: before and early pregnancy |
| | 400UNIT_15ML | |
| 1069 | F0_pharma_H02AB07_{—} | Prescription of PREDNISONE TAB 20 MG 30 (H02AB07), count |
| | PREDNISONE_TAB_20MG_30 | during F0: before and early pregnancy |
| 1070 | F0_pharma_U1_CLEAR_TEST | Prescription of CLEAR TEST (U1), count during F0: before and |
| | | early pregnancy |
| 1071 | F0_pharma_B01AC06_{—} | Prescription of MICROPIRIN TAB 75 MG 28 (B01AC06), count |
| | MICROPIRIN_TAB_75MG_28 | during F0: before and early pregnancy |
| 1072 | F0_pharma_B01AC06_{—} | Prescription of MICROPIRIN TAB 75 mg 30 (B01AC06), count |
| | MICROPIRIN_TAB_75mg_30 | during F0: before and early pregnancy |
| 1073 | F0_pharma_R06AA59_{—} | Prescription of DICLECTIN DR TAB 10 mg/10 mg 100 |
| | DICLECTIN_DR_TAB_{—} | (R06AA59), count during F0: before and early pregnancy |
| | 10mg_OR_10mg_100 | |
| 1074 | F0_pharma_B03AA07_{—} | Prescription of AKTIFERRIN SOFT CAP 34 mg 30 (B03AA07), |
| | AKTIFERRIN_SOFT_CAP_{—} | count during F0: before and early pregnancy |
| | 34mg_30 | |
| 1075 | F0_pharma_G03GA05_{—} | Prescription of GONAL-F-PEN INJ 300IU 0.5 ML (G03GA05), |
| | GONAL_F_PEN_INJ_300IU_0_5ML | count during F0: before and early pregnancy |
| 1076 | F0_pharma_A12AX01_{—} | Prescription of VITA-CAL + D (NEW) TAB CF BOX 60 |
| | VITA_CAL+D_NEW_TAB_{—} | (A12AX01), count during F0: before and early pregnancy |
| | CF_BOX_60 | |
| 1077 | F0_pharma_G03GA05_{—} | Prescription of GONAL-F-PEN INJ 450IU 0.75 ML (G03GA05), |
| | GONAL_F_PEN_INJ_450IU_{—} | count during F0: before and early pregnancy |
| | 0_75ML | |
| 1078 | F0_pharma_A11DB01_{—} | Prescription of TRIBEMIN TAB 20 (A11DB01), count during F0 |
| | TRIBEMIN_TAB_20 | before and early pregnancy |
| 1079 | F0_pharma_U2 | Prescription of (U2), count during F0: before and early pregnancy |
| 1080 | F0_pharma_N02BE01_ACAMOLI_{—} | Prescription of ACAMOLI STRAW. S/F SYR 125 mg/5 mL |
| | STRAW_S_OR_F_SYR_125mg_OR_{—} | 100 mL (N02BE01), count during F0: before and early pregnancy |
| | 5mL_100mL | |
| 1081 | F0_pharma_B03AB10_TIPTIPOT_{—} | Prescription of TIPTIPOT FERRIPEL-3 DRP 50 mg/1 mL 15 mL |
| | FERRIPEL_3_DRP_50mg_OR_1mL_{—} | (B03AB10), count during F0: before and early pregnancy |
| | 15mL | |
| 1082 | F0_pharma_R06AX13_ALLERGYX_TAB_{—} | Prescription of ALLERGYX TAB 10 MG 10 (R06AX13), count |
| | 10MG_10 | during F0: before and early pregnancy |
| 1083 | F0_pharma_J01CA04_MOXYPEN_{—} | Prescription of MOXYPEN CAP 500 mg 20 (J01CA04), count |
| | CAP_500mg_20 | during F0: before and early pregnancy |
| 1084 | F0_pharma_J01CR02_AMOXICLAV_{—} | Prescription of AMOXICLAV TEVA TAB 875 mg 14 (J01CR02), |
| | TEVA_TAB_875mg_14 | count during F0: before and early pregnancy |
| 1085 | F0_pharma_G03GB02_IKACLOMIN_{—} | Prescription of IKACLOMIN TAB 50 MG 10 (G03GB02), count |
| | TAB_50MG_10 | during F0: before and early pregnancy |
| 1086 | F0_pharma_N02BE01_TIPT | Prescription of TIPTIPOT NOVIMOL SUS 100 mg/1 mL 15 mL |
| | IPOT_NOVIMOL_SUS_100mg_OR_{—} | (N02BE01), count during F0: before and early pregnancy |
| | 1mL_15mL | |
| 1087 | F0_pharma_N02BB02_OPTALGIN_{—} | Prescription of OPTALGIN TAB 500 MG 20 (N02BB02), count |
| | TAB_500MG_20 | during F0: before and early pregnancy |
| 1088 | F0_pharma_C10AX06_MAARAZ_9M_{—} | Prescription of MAARAZ 9M: OMEGA-3 + PRENATAL(ALT |
| | OMEGA_3+PRENATAL_ALT_KIT_120 | KIT 120 (C10AX06), count during F0: before and early |
| | | pregnancy |
| 1089 | F0_pharma_G01AF02_AGISTEN_1_{—} | Prescription of AGISTEN 1 VAGINAL TAB 500 MG |
| | VAGINAL_TAB_500MG | (G01AF02), count during F0: before and early pregnancy |
| 1090 | F0_pharma_M01AE01_ADVIL_LIQUI_{—} | Prescription of ADVIL LIQUI-GELS CAP 200 MG 40 |
| | GELS_CAP_200MG_40 | (M01 AE0 1), count during F0: before and early pregnancy |
| 1091 | F0_pharma_G12A | Prescription of (G12A), count during F0: before and early |
| | | pregnancy |
| 1092 | F0_pharma_B01AB05_CLEXANE_{—} | Prescription of CLEXANE SAFETY LOCK INJ 60 MG/0.6 ML 2 |
| | SAFETY_LOCK_INJ_60MG_OR_0_6ML_2 | (B01AB05), count during F0: before and early pregnancy |
| 1093 | F0_pharma_G03DB01_DUPHASTON_{—} | Prescription of DUPHASTON TAB 10 MG BOX 20 (G03DB01), |
| | TAB_10MG_BOX_20 | count during F0: before and early pregnancy |
| 1094 | F0_pharma_G01AF02_AGISTEN_{—} | Prescription of AGISTEN VAGINAL CR 2% 20 GM (G01AF02), |
| | VAGINAL_CR_2%_20GM | count during F0: before and early pregnancy |
| 1095 | F0_pharma_A04AA01_ZOFRAN_TAB_{—} | Prescription of ZOFRAN TAB 4 MG 10 (A04AA01), count during |
| | 4MG_10 | F0: before and early pregnancy |
| 1096 | F0_pharma_S01AA01_{—} | Prescription of SYNTHOMYCINE OCC 5% 3.6 GM (S01AA01), |
| | SYNTHOMYCINE_OCC_5%_3_6GM | count during F0: before and early pregnancy |
| 1097 | F0_pharma_A11CC05_BABY_D3_{—} | Prescription of BABY D3 DRP 200IU 10 ML (A11CC05), count |
| | DRP_200IU_10ML | during F0: before and early pregnancy |
| 1098 | F0_pharma_M02AA15_VOLTAREN_{—} | Prescription of VOLTAREN EMULGEL GEL 1% 100 GM |
| | EMULGEL_GEL_1%_100GM | (M02AA15), count during F0: before and early pregnancy |
| 1099 | F0_pharma_A03FA01_PRAMIN_INJ_{—} | Prescription of PRAMIN INJ 10 MG/2 ML 5 (A03FA01), count |
| | 10MG_OR_2ML_5 | during F0: before and early pregnancy |
| 1100 | F0_pharma_D07CB01_DER_{—} | Prescription of DERMACOMBIN CR CF 15 GM (D07CB01), |
| | MACOMBIN_CR_CF_15GM | count during F0: before and early pregnancy |
| 1101 | F0_pharma_R01BA55_NUSSIDEX_{—} | Prescription of NUSSIDEX TAB CF 20 (R01BA55), count during |
| | TAB_CF_20 | F0: before and early pregnancy |
| 1102 | F0_pharma_G01AF04_GYNO_{—} | Prescription of GYNO-DAKTARIN VAG. CAP 400 mg 3 |
| | DAKTARIN_VAG_CAP_400mg_3 | (G01AF04), count during F0: before and early pregnancy |
| 1103 | F0_pharma_B03AE02_GENTLE_IRON_{—} | Prescription of GENTLE IRON CAP 25 MG BOX 90 (B03AE02) |
| | CAP_25MG_BOX_90 | count during F0: before and early pregnancy |
| 1104 | F0_pharma_N02BE01_ACAMOLI_{—} | Prescription of ACAMOLI FRUIT S/F SYR 125 mg/5 mL 100 mL |
| | FRUIT_S_OR_F_SYR_125mg_OR_{—} | (N02BE01), count during F0: before and early pregnancy |
| | 5mL_100mL | |
| 1105 | F0_pharma_M01AE01_NUROFEN_{—} | Prescription of NUROFEN STRAWB.PED. SUS 100 mg/5 mL |
| | STRAWB_PED_SUS_100mg_OR_{—} | 100 mL (M01AE01), count during F0: before and early pregnancy |
| | 5mL_100mL | |
| 1106 | F0_pharma_C05AX05_PROCTO_{—} | Prescription of PROCTO-GLYVENOL CR CF TUB 30 GM |
| | GLYVENOL_CR_CF_TUB_30GM | (C05AX05), count during F0: before and early pregnancy |
| 1107 | F0_pharma_B03BA51_VIT_{—} | Prescription of VIT.B12 + FOLIC A.SUBLING. TAB CF 60 |
| | B12+FOLIC_A_SUBLING_TAB_CF_60 | (B03BA51), count during F0: before and early pregnancy |
| 1108 | F0_pharma_M01AE01_NUROFEN_{—} | Prescription of NUROFEN ORANGE CHILD SUS 100 MG/5 ML |
| | ORANGE_CHILD_SUS_100MG_OR_{—} | 100 ML (M01AE01), count during F0: before and early pregnancy |
| | 5ML_100ML | |
| 1109 | F0_pharma_D04AA13_FENISTIL_GEL_{—} | Prescription of FENISTIL GEL 0.1% 30 GM (D04AA13), count |
| | 0_1%_30GM | during F0: before and early pregnancy |
| 1110 | F0_pharma_A02BA03_FAMOTIDINE_{—} | Prescription of FAMOTIDINE-TEVA TAB 20 MG 30 |
| | TEVA_TAB_20MG_30 | (A02BA03), count during F0: before and early pregnancy |
| 1111 | F0_pharma_A03FA01_PRAMIN_SUP_{—} | Prescription of PRAMIN SUP 20 MG 6 (A03FA01), count during |
| | 20MG_6 | F0: before and early pregnancy |
| 1112 | F0_pharma_A11AA06_PRENATAL_{—} | Prescription of PRENATAL MULTIVITAMINS D.H TAB 100 |
| | MULTIVITAMINS_D_H_TAB_100 | (A11AA06), count during F0: before and early pregnancy |
| 1113 | F0_pharma_D01AC63_COMAGIS_CR_{—} | Prescription of COMAGIS CR CF 15 GM (D01AC63), count |
| | CF_15GM | during F0: before and early pregnancy |
| 1114 | F0_pharma_S02CA06_DESOREN_AUR_{—} | Prescription of DESOREN AUR CF BOT 5 ML (S02CA06), count |
| | CF_BOT_5ML | during F0: before and early pregnancy |
| 1115 | F0_pharma_B03BB01_FOLI_5_CTS_{—} | Prescription of FOLI 5 (CTS) TAB 5 mg 30 (B03BB01), count |
| | TAB_5mg_30 | during F0: before and early pregnancy |
| 1116 | F0_pharma_A03AD01_PAPAVERINE_{—} | Prescription of PAPAVERINE TAB 80 MG 30 (A03AD01), count |
| | TAB_80MG_30 | during F0: before and early pregnancy |
| 1117 | F0_pharma_S01CA20_DETHAMYCIN_{—} | Prescription of DETHAMYCIN EYE, EAR & NOSE DRP BOT |
| | EYE_EAR_&_NOSE_DRP_BOT_10mL | 10 mL (S01CA20), count during F0: before and early pregnancy |
| 1118 | F0_pharma_G03GA02_MENOGON_<><>_{—} | Prescription of MENOGON <><> INJ 75IU/75IU 10 (G03GA02), |
| | INJ_75IU_OR_75IU_10 | count during F0: before and early pregnancy |
| 1119 | F0_pharma_N02BE01_ACAMOLI_{—} | Prescription of ACAMOLI SUP 150 MG 12 (N02BE01), count |
| | SUP_150MG_12 | during F0: before and early pregnancy |
| 1120 | F0_pharma_{——}FEMINA_SOAP_LIQ_330ML | Prescription of FEMINA SOAP LIQ 330 ML ( ), count during F0: |
| | | before and early pregnancy |
| 1121 | F0_pharma_D01AC24_POLYCUTAN_{—} | Prescription of POLYCUTAN CR 15 GM (D01AC24), count |
| | CR_15GM | during F0: before and early pregnancy |
| 1122 | F0_clinic_V221_SUPERVISION_OF_{—} | Clinic diagnosis SUPERVISION OF OTHER NORMAL |
| | OTHER_NORMAL_PREGNANCY | PREGNANCY (V221), count during F0: before and early |
| | | pregnancy |
| 1123 | F0_pharma_R05CA20_THYMOLI_SYR_{—} | Prescription of THYMOLI SYR BOT 100 ML (R05CA20), count |
| | BOT_100ML | during F0: before and early pregnancy |
| 1124 | F2_pharma_A11CC05_VITAMIDYNE_{—} | Prescription of VITAMIDYNE D DRP 200IU 10 ML (A11CC05), |
| | D_DRP_200IU_10ML | count during F2: second blood test (weeks 16-19) |
| 1125 | F2_pharma_R06AA59_DICLECTIN_{—} | Prescription of DICLECTIN DR TAB 10 mg/10 mg 100 |
| | DR_TAB_10mg_OR_10mg_100 | (R06AA59), count during F2: second blood test (weeks 16-19) |
| 1126 | F2_pharma_A11CC05_VITAMIN_D3_{—} | Prescription of VITAMIN D3 D.H DRP 400 UNIT 15 ML |
| | D_H_DRP_400UNIT_15ML | (A11CC05), count during F2: second blood test (weeks 16-19) |
| 1127 | F2_pharma_H02AB07_PREDNISONE_{—} | Prescription of PREDNISONE TAB 5 MG 30 (H02AB07), count |
| | TAB_5MG_30 | during F2: second blood test (weeks 16-19) |
| 1128 | F2_pharma_R01AB22_OTRIVIN_{—} | Prescription of OTRIVIN MENTHOL M.D SPR 0.1% 10 ML |
| | MENTHOL_M_D_SPR_0_1%_10ML | (R01AB22), count during F2: second blood test (weeks 16-19) |
| 1129 | F2_pharma_N01BB54_EMLA_{—} | Prescription of EMLA CR 5% 30 GM (N01BB54), count during |
| | CR_5%_30GM | F2: second blood test (weeks 16-19) |
| 1130 | F2_pharma_R03AK07_SYMBICORT_{—} | Prescription of SYMBICORT TURBUHALER INH |
| | TURBUHALER_INH_160mcg_OR_{—} | 160 mcg/4.5 mcg (R03AK07), count during F2: second blood test |
| | 4_5mcg | (weeks 16-19) |
| 1131 | F2_pharma_A04AA01_ZOFRAN_{—} | Prescription of ZOFRAN TAB 8 MG 10 (A04AA01), count during |
| | TAB_8MG_10 | F2: second blood test (weeks 16-19) |
| 1132 | F2_pharma_B01AB05_CLEXANE_INJ_{—} | Prescription of CLEXANE INJ 40 mg/0.4 mL 2X0.4 mL |
| | 40mg_OR_0_4mL_2X0_4mL | (B01AB05), count during F2: second blood test (weeks 16-19) |
| 1133 | F2_pharma_B03AB10_FERRIPEL_3_{—} | Prescription of FERRIPEL-3 SYR 50 MG/5 ML 110 ML |
| | SYR_50MG_OR_5ML_110ML | (B03AB10), count during F2: second blood test (weeks 16-19) |
| 1134 | F2_pharma_M01AE01_NUROFEN_{—} | Prescription of NUROFEN STRAWB.CHILD SUS 100 MG/5 ML |
| | STRAWB_CHILD_SUS_100MG_OR_{—} | 150 ML (M01AE01), count during F2: second blood test |
| | 5ML_150ML | (weeks 16-19) |
| 1135 | F2_pharma_H02AB07_PREDNISONE_{—} | Prescription of PREDNISONE TAB 20 MG 30 (H02AB07), count |
| | TAB_20MG_30 | during F2: second blood test (weeks 16-19) |
| 1136 | F2_pharma_{——}OTRIVONIM_BABY_WIP_{—} | Prescription of OTRIVONIM BABY WIP PKG 2X36U ( ), count |
| | PKG_2X36U | during F2: second blood test (weeks 16-19) |
| 1137 | F2_pharma_G04BX06_SEDURAL_{—} | Prescription of SEDURAL TAB 100 MG 30 (G04BX06), count |
| | TAB_100MG_30 | during F2: second blood test (weeks 16-19) |
| 1138 | F2_pharma_C10AX06_OMEGA_D3_9_{—} | Prescription of OMEGA D3 9 MONTHS (SUPH) S.GEL CAP 60 |
| | MONTHS_SUPH_S_GEL_CAP_60 | (C10AX06), count during F2: second blood test (weeks 16-19) |
| 1139 | F2_pharma_B03AE12_IRON_PLUS_{—} | Prescription of IRON PLUS FEMINA (ALTMAN) TAB 100 |
| | FEMINA_ALTMAN_TAB_100 | (B03AE12), count during F2: second blood test (weeks 16-19) |
| 1140 | F2_pharma_G03DC02_PRIMOLUT_{—} | Prescription of PRIMOLUT NOR TAB 5 MG 20 (G03DC02), |
| | NOR_TAB_5MG_20 | count during F2: second blood test (weeks 16-19) |
| 1141 | F2_pharma_G03GA05_GONAL_F_PEN_{—} | Prescription of GONAL-F-PEN INJ 450IU 0.75 ML (G03GA05), |
| | INJ_450IU_0_75ML | count during F2: second blood test (weeks 16-19) |
| 1142 | F2_pharma_A11AA06_PREGENTLY_{—} | Prescription of PRE-GENTLY BADATZ (SUPHERB)TAB 60 |
| | BADATZ_SUPHERB_TAB_60 | (A11AA06), count during F2: second blood test (weeks 16-19) |
| 1143 | F2_pharma_R01BA55_NUSSIDEX_{—} | Prescription of NUSSIDEX TAB CF 20 (R01BA55), count during |
| | TAB_CF_20 | F2: second blood test (weeks 16-19) |
| 1144 | F2_pharma_B03AE02_GENTLE_{—} | Prescription of GENTLE IRON CAP 25 MG BOX 90 (B03AE02), |
| | IRON_CAP_25MG_BOX_90 | count during F2: second blood test (weeks 16-19) |
| 1145 | F2_pharma_U1_CLEAR_TEST | Prescription of CLEAR TEST (U1), count during F2: second |
| | | blood test (weeks 16-19) |
| 1146 | F2_pharma_U0_LABON_TIME | Prescription of LABON TIME (U0), count during F2: second |
| | | blood test (weeks 16-19) |
| 1147 | F2_pharma_B03BA51_VIT_B12+ | Prescription of VIT.B12 + FOLIC A.SUBLING. TAB CF 60 |
| | FOLIC_A_SUBLING_TAB_CF_60 | (B03BA51), count during F2: second blood test (weeks 16-19) |
| 1148 | F2_pharma_A11CC05_D_DROPS_{—} | Prescription of D-DROPS DRP 200 UNIT 10 mL (A11CC05), |
| | DRP_200UNIT_10mL | count during F2: second blood test (weeks 16-19) |
| 1149 | F2_pharma_{——}ELASTAN_TREAT_{—} | Prescription of ELASTAN TREAT CR 75 ML ( ), count during |
| | CR_75ML | F2: second blood test (weeks 16-19) |
| 1150 | F2_pharma_J06BB01_KAMRHO_{—} | Prescription of KAMRHO-D I.M. INJ 300 mcg 2 mL (J06BB01), |
| | D_I_M_INJ_300mcg_2mL | count during F2: second blood test (weeks 16-19) |
| 1151 | F2_pharma_S02CA06_DEX_OTIC_{—} | Prescription of DEX-OTIC AUR CF BOT 5 ML (S02CA06), |
| | AUR_CF_BOT_5ML | count during F2: second blood test (weeks 16-19) |
| 1152 | F2_pharma_G01AF05_GYNO_{—} | Prescription of GYNO-PEVARYL VAG TAB 150 MG 3 |
| | PEVARYL_VAG_TAB_150MG_3 | (G01AF05), count during F2: second blood test (weeks 16-19) |
| 1153 | F2_pharma_R01AD08_{—} | Prescription of FLIXONASE NASAL SPR 0.05% (R01AD08), |
| | FLIXONASE_NASAL_SPR_0_05% | count during F2: second blood test (weeks 16-19) |
| 1154 | F2_pharma_G03GA05_GONAL_{—} | Prescription of GONAL-F-PEN INJ 300IU 0.5 ML (G03GA05), |
| | F_PEN_INJ_300IU_0_5ML | count during F2: second blood test (weeks 16-19) |
| 1155 | F2_pharma_B03AA07_AKTIFERRIN_{—} | Prescription of AKTIFERRIN SOFT CAP 34 mg 30 (B03AA07), |
| | SOFT_CAP_34mg_30 | count during F2: second blood test (weeks 16-19) |
| 1156 | F2_pharma_S02DA30_OTIDIN_{—} | Prescription of OTIDIN AUR CF 10 GM (S02DA30), count |
| | AUR_CF_10GM | during F2: second blood test (weeks 16-19) |
| 1157 | F2_pharma_H01CC01_ORGALUTRAN_{—} | Prescription of ORGALUTRAN SYRI.<><> INJ 0.25 MG 0.5 ML |
| | SYRI_<><>_INJ_0_25MG_0_5ML | (H01CC01), count during F2: second blood test (weeks 16-19) |
| 1158 | F2_pharma_D07CA03_AFLUMYCIN_{—} | Prescription of AFLUMYCIN CR CF 20 GM (D07CA03), count |
| | CR_CF_20GM | during F2: second blood test (weeks 16-19) |
| 1159 | F2_pharma_J01AA02_DOXYLIN_{—} | Prescription of DOXYLIN TAB 100 MG 10 (J01AA02), count |
| | TAB_100MG_10 | during F2: second blood test (weeks 16-19) |
| 1160 | F2_pharma_B03BA01_BEVITEX_{—} | Prescription of BEVITEX SUBLINGUAL TAB 1000 MCG 30 |
| | SUBLINGUAL_TAB_1000MCG_30 | (B03BA01), count during F2: second blood test (weeks 16-19) |
| 1161 | F2_pharma_M01AE01_NUROFEN_{—} | Prescription of NUROFEN ORANGE CHILD SUS 100 MG/5 ML |
| | ORANGE_CHILD_SUS_100MG_OR_{—} | 150 ML (M01AE01), count during F2: second blood test |
| | 5ML_150ML | (weeks 16-19) |
| 1162 | F2_pharma_G03GA02_{—} | Prescription of MENOPUR VIA 75IU 10 (G03GA02), count |
| | MENOPUR_VIA_75IU_10 | during F2: second blood test (weeks 16-19) |
| 1163 | F2_pharma_R03AC02_VENTOLIN_{—} | Prescription of VENTOLIN RESPIR. SOL 5 mg/1 mL 20 mL |
| | RESPIR_SOL_5mg_OR_1mL_20mL | (R03AC02), count during F2: second blood test (weeks 16-19) |
| 1164 | F2_pharma_A02AD01_MAALOX_SUS_{—} | Prescription of MAALOX SUS CF BOT 355 ML (A02AD01), |
| | CF_BOT_355ML | count during F2: second blood test (weeks 16-19) |
| 1165 | F2_pharma_A03FA01_PRAMIN_{—} | Prescription of PRAMIN INJ 10 MG/2 ML 5 (A03FA01), count |
| | INJ_10MG_OR_2ML_5 | during F2: second blood test (weeks 16-19) |
| 1166 | F2_pharma_M01AE01_ADVIL_LIQUI_{—} | Prescription of ADVIL LIQUI-GELS CAP 200 MG 16 |
| | GELS_CAP_200MG_16 | (M01AE01), count during F2: second blood test (weeks 16-19) |
| 1167 | F2_pharma_R02AA03_STREPSILS_{—} | Prescription of STREPSILS HONEY-LEMON LOZ CF 24 |
| | HONEY_LEMON_LOZ_CF_24 | (R02AA03), count during F2: second blood test (weeks 16-19) |
| 1168 | F2_pharma_R02AA20_LEMOCIN_{—} | Prescription of LEMOCIN LEMON SUGAR FREE LOZ CF 24 |
| | LEMON_SUGAR_FREE_LOZ_CF_24 | (R02AA20), count during F2: second blood test (weeks 16-19) |
| 1169 | F2_pharma_C05AX05_PROCTO_{—} | Prescription of PROCTO-GLYVENOL CR CF TUB 30 GM |
| | GLYVENOL_CR_CF_TUB_30GM | (C05AX05), count during F2: second blood test (weeks 16-19) |
| 1170 | F2_pharma_R03AC02_VENTOLIN_CFC_{—} | Prescription of VENTOLIN CFC FREE INH 0.1 MG/INH |
| | FREE_INH_0_1MG_OR_INH | (R03AC02), count during F2: second blood test (weeks 16-19) |
| 1171 | F0_pharma_R01AA07_OTRIVIN_{—} | Prescription of OTRIVIN NASAL M.D. SPR 0.1% 10 ML |
| | NASAL_M_D_SPR_0_1%_10ML | (R01AA07), count during F0: before and early pregnancy |
| 1172 | F2_pharma_H03AA01_ELTROXIN_{—} | Prescription of ELTROXIN TAB 100 MCG 100 (H03AA01), |
| | TAB_100MCG_100 | count during F2: second blood test (weeks 16-19) |
| 1173 | F2_pharma_S01AA01_{—} | Prescription of SYNTHOMYCINE OCC 5% 3.6 GM (S01AA01), |
| | SYNTHOMYCINE_OCC_5%_3_6GM | count during F2: second blood test (weeks 16-19) |
| 1174 | F2_pharma_H03B | Prescription of (H03B), count during F2: second blood test |
| | | (weeks 16-19) |
| 1175 | F2_pharma_H01CC02_CETROTIDE_{—} | Prescription of CETROTIDE VIA 0.25 mg 7 (H01CC02), count |
| | VIA_0_25mg_7 | during F2: second blood test (weeks 16-19) |
| 1176 | F2_pharma_G03DA04_UTROGESTAN_{—} | Prescription of UTROGESTAN ORAL/VAG CAP 100 mg 30 |
| | ORAL_OR_VAG_CAP_100mg_30 | (G03DA04), count during F2: second blood test (weeks 16-19) |
| 1177 | F2_pharma_G03DA03_PROGESTERON_{—} | Prescription of PROGESTERON RETARD 29/M INJ |
| | RETARD_29_OR_M_INJ_500MG_OR_{—} | 500 MG/2 ML 3 (G03DA03), count during F2: second blood test |
| | 2ML_3 | (weeks 16-19) |
| 1178 | F2_pharma_J01CA04_MOXYPEN_{—} | Prescription of MOXYPEN CAP 500 MG 10 (J01CA04), count |
| | CAP_500MG_10 | during F2: second blood test (weeks 16-19) |
| 1179 | F2_pharma_G03CA03_PROGYNOVA_{—} | Prescription of PROGYNOVA TAB 2 MG 28 (G03CA03), count |
| | TAB_2MG_28 | during F2: second blood test (weeks 16-19) |
| 1180 | F2_pharma_L02AE04_DECAPEPTYL_{—} | Prescription of DECAPEPTYL INJ 0.1 mg/1 mL 7 (L02AE04), |
| | INJ_0_1mg_OR_1mL_7 | count during F2: second blood test (weeks 16-19) |
| 1181 | F2_pharma_J01DB01_CEFOVIT_{—} | Prescription of CEFOVIT FORTE CAP 500 mg 20 (J01DB01), |
| | FORTE_CAP_500mg_20 | count during F2: second blood test (weeks 16-19) |
| 1182 | F2_pharma_H03AA01_EUTHYROX_{—} | Prescription of EUTHYROX TAB 50 mcg 100 (H03AA01), count |
| | TAB_50mcg_100 | during F2: second blood test (weeks 16-19) |
| 1183 | F2_pharma_B01AB05_CLEXANE_{—} | Prescription of CLEXANE SAFETY LOCK INJ 60 MG/0.6 ML 2 |
| | SAFETY_LOCK_INJ_60MG_OR_0_6ML_2 | (B01AB05), count during F2: second blood test (weeks 16-19) |
| 1184 | F2_pharma_J01XE01_MACRODANTIN_{—} | Prescription of MACRODANTIN 29/M CAP 100 MG 30 |
| | 29_OR_M_CAP_100MG_30 | (J01XE01), count during F2: second blood test (weeks 16-19) |
| 1185 | F2_pharma_A02BC01_OMEPRADEX_{—} | Prescription of OMEPRADEX CPL 20 mg 30 (A02BC01), count |
| | CPL_20mg_30 | during F2: second blood test (weeks 16-19) |
| 1186 | F2_pharma_D01AC21_HYDROAGISTEN_{—} | Prescription of HYDRO AGISTEN CR CF 15 GM (D01AC21), |
| | CR_CF_15GM | count during F2: second blood test (weeks 16-19) |
| 1187 | F2_pharma_G03GA05_GONAL_F_{—} | Prescription of GONAL-F-PEN INJ 900IU 1.5 ML (G03GA05), |
| | PEN_INJ_900IU_1_5ML | count during F2: second blood test (weeks 16-19) |
| 1188 | F2_pharma_B03AE02_IRON_{—} | Prescription of IRON BIS-GLYCINATE DH CAP 90 |
| | BIS_GLYCINATE_DH_CAP_90 | (B03AE02), count during F2: second blood test (weeks 16-19) |
| 1189 | F2_pharma_J01CE02_PEN_RAFA_{—} | Prescription of PEN-RAFA VK CPL 500 mg 40 (J01CE02), count |
| | VK_CPL_500mg_40 | during F2: second blood test (weeks 16-19) |
| 1190 | F2_pharma_U3 | Prescription of (U3), count during F2: second blood test |
| | | (weeks 16-19) |
| 1191 | F2_pharma_Z07_AFTER_10_STICK_KIT | Prescription of AFTER 10 STICK KIT (Z07), count during F2: |
| | | second blood test (weeks 16-19) |
| 1192 | F2_pharma_G03CA03_ESTROFEM_{—} | Prescription of ESTROFEM TAB 2 MG 28 (G03CA03), count |
| | TAB_2MG_28 | during F2: second blood test (weeks 16-19) |
| 1193 | F2_pharma_G03DB01_DUPHASTON_{—} | Prescription of DUPHASTON TAB 10 MG BOX 20 (G03DB01), |
| | TAB_10MG_BOX_20 | count during F2: second blood test (weeks 16-19) |
| 1194 | F2_pharma_B03BA01_VIT_B12_{—} | Prescription of VIT B12(SOLGAR)SUBL TAB 1000 mcg 100 |
| | SOLGAR_SUBL_TAB_1000mcg_100 | (B03BA01), count during F2: second blood test (weeks 16-19) |
| 1195 | F2_pharma_M01AE01_NUROFEN_{—} | Prescription of NUROFEN ORANGE CHILD SUS 100 MG/5 ML |
| | ORANGE_CHILD_SUS_100MG_OR_{—} | 100 ML (M01AE01), count during F2: second blood test |
| | 5ML_100ML | (weeks 16-19) |
| 1196 | F2_pharma_G03DA04_GESTON_{—} | Prescription of GESTON INJ 50 mg/1 mL 1 mL (G03DA04), count |
| | INJ_50mg_OR_1mL_1mL | during F2: second blood test (weeks 16-19) |
| 1197 | F2_pharma_D04AA13_FENISTIL_{—} | Prescription of FENISTIL GEL 0.1% 30 GM (D04AA13), count |
| | GEL_0_1%_30GM | during F2: second blood test (weeks 16-19) |
| 1198 | F2_pharma_A02BA03_FAMOTIDINE_{—} | Prescription of FAMOTIDINE-TEVA TAB 20 MG 30 |
| | TEVA_TAB_20MG_30 | (A02BA03), count during F2: second blood test (weeks 16-19) |
| 1199 | F2_pharma_G01AF04_GYNO_{—} | Prescription of GYNO-DAKTARIN VAG. CAP 400 mg 3 |
| | DAKTARIN_VAG_CAP_400mg_3 | (G01AF04), count during F2: second blood test (weeks 16-19) |
| 1200 | F2_pharma_A03FA01_PRAMIN_{—} | Prescription of PRAMIN SUP 20 MG 6 (A03FA01), count during |
| | SUP_20MG_6 | F2: second blood test (weeks 16-19) |
| 1201 | F2_pharma_A03AD01_PAPAVERINE_{—} | Prescription of PAPAVERINE TAB 80 MG 30 (A03AD01), count |
| | TAB_80MG_30 | during F2: second blood test (weeks 16-19) |
| 1202 | F2_pharma_G03GA02_MENOGON_<><>_{—} | Prescription of MENOGON <><> INJ 75IU/75IU 10 (G03GA02), |
| | INJ_75IU_OR_75IU_10 | count during F2: second blood test (weeks 16-19) |
| 1203 | F2_pharma_R06AX26_TELFAST_{—} | Prescription of TELFAST TAB 180 MG 15 (R06AX26), count |
| | TAB_180MG_15 | during F2: second blood test (weeks 16-19) |
| 1204 | F2_pharma_N02BE01_ACAMOLI_{—} | Prescription of ACAMOLI SUP 150 MG 12 (N02BE01), count |
| | SUP_150MG_12 | during F2: second blood test (weeks 16-19) |
| 1205 | F2_pharma_{——}FEMINA_SOAP_LIQ_330ML | Prescription of FEMINA SOAP LIQ 330 ML ( ), count during F2: |
| | | second blood test (weeks 16-19) |
| 1206 | F2_pharma_A11AA06_PRENATAL_{—} | Prescription of PRENATAL MULTIVITAMINS D.H TAB 100 |
| | MULTIVITAMINS_D_H_TAB_100 | (A11AA06), count during F2: second blood test (weeks 16-19) |
| 1207 | F2_pharma_G01AF02_AGISTEN_1_{—} | Prescription of AGISTEN 1 VAGINAL TAB 500 MG |
| | VAGINAL_TAB_500MG | (G01AF02), count during F2: second blood test (weeks 16-19) |
| 1208 | F2_pharma_D07CB01_DERMACOMBIN_{—} | Prescription of DERMACOMBIN CR CF 15 GM (D07CB01), |
| | CR_CF_15GM | count during F2: second blood test (weeks 16-19) |
| 1209 | F2_pharma_B03AB10_TIPTIPOT_{—} | Prescription of TIPTIPOT FERRIPEL-3 DRP 50 mg/1 mL 15 mL |
| | FERRIPEL_3_DRP_50mg_OR_1mL_{—} | (B03AB10), count during F2: second blood test (weeks 16-19) |
| | 15mL | |
| 1210 | F2_pharma_R06AX13_ALLERGYX_{—} | Prescription of ALLERGYX TAB 10 MG 10 (R06AX13), count |
| | TAB_10MG_10 | during F2: second blood test (weeks 16-19) |
| 1211 | F2_pharma_J01CA04_MOXYPEN_{—} | Prescription of MOXYPEN CAP 500 mg 20 (J01CA04), count |
| | CAP_500mg_20 | during F2: second blood test (weeks 16-19) |
| 1212 | F2_pharma_J01CR02_AMOXICLAV_{—} | Prescription of AMOXICLAV TEVA TAB 875 mg 14 (J01CR02), |
| | TEVA_TAB_875mg_14 | count during F2: second blood test (weeks 16-19) |
| 1213 | F2_pharma_G03GB02_IKACLOMIN_{—} | Prescription of IKACLOMIN TAB 50 MG 10 (G03GB02), count |
| | TAB_50MG_10 | during F2: second blood test (weeks 16-19) |
| 1214 | F2_pharma_N02BE01_TIPTIPOT_{—} | Prescription of TIPTIPOT NOVIMOL SUS 100 mg/1 mL 15 mL |
| | NOVIMOL_SUS_100mg_OR_1mL_15mL | (N02BE01), count during F2: second blood test (weeks 16-19) |
| 1215 | F2_pharma_N02BB02_OPTALGIN_{—} | Prescription of OPTALGIN TAB 500 MG 20 (N02BB02), count |
| | TAB_500MG_20 | during F2: second blood test (weeks 16-19) |
| 1216 | F2_pharma_N02BE01_ACAMOLI_{—} | Prescription of ACAMOLI STRAW. S/F SYR 125 mg/5 mL |
| | STRAW_S_OR_F_SYR_125mg_{—} | 100 mL (N02BE01), count during F2: second blood test |
| | OR_5mL_100mL | (weeks 16-19) |
| 1217 | F2_pharma_{——}PARO_TOOTHBRUSH_{—} | Prescription of PARO TOOTHBRUSH MEDIC NO. 726 ( ), |
| | MEDIC_NO_726 | count during F2: second blood test (weeks 16-19) |
| 1218 | F2_pharma_B03AD02_FOROL_TAB_30 | Prescription of FOROL TAB 30 (B03AD02), count during F2: |
| | | second blood test (weeks 16-19) |
| 1219 | F2_pharma_J01CR02_AUGMENTIN_{—} | Prescription of AUGMENTIN TAB 500 MG 20 (J01CR02), count |
| | TAB_500MG_20 | during F2: second blood test (weeks 16-19) |
| 1220 | locality_type_Moshav_2000_to_4999_{—} | Lives in Moshav 2,000-4,999 residents |
| | residents | |
| 1221 | M5_HEMOGLOBIN_A1C_perc | Labtest HEMOGLOBIN A1C %, sampled during M5: year 5 |
| | | before pregnancy |
| 1222 | F0_clinic_0_UNKNOWN_CATEGORY | Clinic diagnosis UNKNOWN CATEGORY (0), count during F0: |
| | | before and early pregnancy |
| 1223 | M5_BASO_perc | Labtest BASO %, sampled during M5: year 5 before pregnancy |
| 1224 | main_religion_Other | Lives in locality with main religion Other |
| 1225 | main_religion_Muslims | Lives in locality with main religion Muslims |
| 1226 | main_religion_Jews | Lives in locality with main religion Jews |
| 1227 | main_religion_Druze | Lives in locality with main religion Druze |
| 1228 | locality_type_Non_Jewish_Locality_{—} | Lives in Non-Jewish Locality 5,000-9,999 residents |
| | 5000_to_9999_residents | |
| 1229 | locality_type_Non_Jewish_Locality_{—} | Lives in Non-Jewish Locality 2,000-4,999 residents |
| | 2000_to_4999_residents | |
| 1230 | locality_type_Jewish_Locality_{—} | Lives in Jewish Locality 50,000-99,999 residents |
| | 50000_to_99999_residents | |
| 1231 | M4_HYPO_perc | Labtest HYPO %, sampled during M4: year 4 before pregnancy |
| 1232 | locality_type_Jewish_Locality_{—} | Lives in Jewish Locality 5,000-9,999 residents |
| | 5000_to_9999_residents | |
| 1233 | locality_type_Collective_Moshav | Lives in Collective Moshav |
| 1234 | ethmcity_africa | Etnicity: Africa |
| 1235 | ethnicity_west_europe | Etnicity: West Europe |
| 1236 | ethnicity_mediterranean | Etnicity: Mediterranean |
| 1237 | ethnicity_latin_america | Etnicity: Latin America |
| 1238 | ethnicity_muslim_arab | Etnicity: Muslim Arab |
| 1239 | ethnicity_north_america | Etnicity: North America |
| 1240 | ethnicity_ethiopia | Etnicity: Ethiopia |
| 1241 | M5_Urine_culture | Labtest Urine culture, sampled during M5: year 5 before |
| | | pregnancy |
| 1242 | F0_clinic_V6801_ENCOUNTERS_{—} | Clinic diagnosis ENCOUNTERS FOR DISABILITY |
| | FOR_DISABILITY_EXAMINATION | EXAMINATION (V6801), count during F0: before and early |
| | | pregnancy |
| 1243 | F2_pharma_N02BE01_ACAMOLI_{—} | Prescription of ACAMOLI FRUIT S/F SYR 125 mg/5 mL 100 mL |
| | FRUIT_S_OR_F_SYR_{—} | (N02BE01), count during F2: second blood test (weeks 16-19) |
| | 125mg_OR_5mL_100mL | |
| 1244 | prev_pregnancy_3_MCH | Labtest MCH, sampled during 3rd previous pregnancy |
| 1245 | miscarriage_history | Recorded history of miscarriage |
| 1246 | PCOS_history | Recorded history of PCOS |
| 1247 | hypertension_history | Recorded history of hypertension |
| 1248 | heart_disease_history | Recorded history of heart disease |
| 1249 | prev_pregnancy_3_Urine_culture | Labtest Urine culture, sampled during 3rd previous pregnancy |
| 1250 | prev_pregnancy_3_HEMOGLOBIN_{—} | Labtest HEMOGLOBIN A1C %, sampled during 3rd previous |
| | A1C_perc | pregnancy |
| 1251 | prev_pregnancy_3_EOS_abs | Labtest EOS.abs, sampled during 3rd previous pregnancy |
| 1252 | prev_pregnancy_3_LYMP_abs | Labtest LYMP.abs, sampled during 3rd previous pregnancy |
| 1253 | prev_pregnancy_3_MACRO_perc | Labtest MACRO %, sampled during 3rd previous pregnancy |
| 1254 | prev_pregnancy_2_Urine_culture | Labtest Urine culture, sampled during 2nd previous pregnancy |
| 1255 | M4_HEMOGLOBIN_A1C_perc | Labtest HEMOGLOBIN A1C %, sampled during M4: year 4 |
| | | before pregnancy |
| 1256 | prev_pregnancy_2_BASO_perc | Labtest BASO %, sampled during 2nd previous pregnancy |
| 1257 | prev_pregnancy_2_HYPER_perc | Labtest HYPER %, sampled during 2nd previous pregnancy |
| 1258 | prev_pregnancy_1_Urine_culture | Labtest Urine culture, sampled during 1st previous pregnancy |
| 1259 | M2_HEMOGLOBIN_A1C_perc | Labtest HEMOGLOBIN A1C %, sampled during M2: year 2 |
| | | before pregnancy |
| 1260 | M2_MCHC | Labtest MCHC, sampled during M2: year 2 before pregnancy |
| 1261 | M3_Urine_culture | Labtest Urine culture, sampled during M3: year 3 before |
| | | pregnancy |
| 1262 | F0_clinic_V828_SCREENING_FOR_{—} | Clinic diagnosis SCREENING FOR OTHER SPECIFIED |
| | OTHER_SPECIFIED_CONDITIONS | CONDITIONS (V828), count during F0: before and early |
| | | pregnancy |
| 1263 | M3_MONO_abs | Labtest MONO.abs, sampled during M3: year 3 before pregnancy |
| 1264 | M3_MACRO_perc | Labtest MACRO %, sampled during M3: year 3 before pregnancy |
| 1265 | ethnicity_east_europe | Etnicity: East Europe |
| 1266 | ethnicity_iraq | Etnicity: Iraq |
| 1267 | F2_pharma_D07AC01_BETACORTEN_{—} | Prescription of BETACORTEN CR 0.1% 15 GM (D07AC01), |
| | CR_0_1%_15GM | count during F2: second blood test (weeks 16-19) |
| 1268 | F2_pharma_R05DA21_DEXAMOL_{—} | Prescription of DEXAMOL COLD 30D + 20N CPL (R05DA21), |
| | COLD_30D+20N_CPL | count during F2: second blood test (weeks 16-19) |
| 1269 | F2_pharma_D06BB03_ZOVIRAX_{—} | Prescription of ZOVIRAX CR 5% 2 GM (D06BB03), count |
| | CR_5%_2GM | during F2: second blood test (weeks 16-19) |
| 1270 | F2_pharma_R06AX13_LORATADINE_{—} | Prescription of LORATADINE (LORATADIM) TAB 10 mg 10 |
| | LORATADIM_TAB_10mg_10 | (R06AX13), count during F2: second blood test (weeks 16-19) |
| 1271 | F2_pharma_B03BA01_VIT_B12_{—} | Prescription of VIT B12 (FLORIS) SUBLING TAB 1000 MCG |
| | FLORIS_SUBLING_TAB_1000MCG_30 | 30 (B03BA01), count during F2: second blood test (weeks 16-19) |
| 1272 | F2_pharma_G03GA06_PUREGON_{—} | Prescription of PUREGON INJ 300IU 0.36 ML (G03GA06), count |
| | INJ_300IU_0_36ML | during F2: second blood test (weeks 16-19) |
| 1273 | F2_pharma_G03GA02_MENOPUR_{—} | Prescription of MENOPUR MULTIDOSE INJ 1200 UNIT |
| | MULTIDOSE_INJ_1200UNIT | (G03GA02), count during F2: second blood test (weeks 16-19) |
| 1274 | F2_pharma_R05CA20_THYMOLI_{—} | Prescription of THYMOLI SYR BOT 100 ML (R05CA20), count |
| | SYR_BOT_100ML | during F2: second blood test (weeks 16-19) |
| 1275 | F2_pharma_G03AA10_MELIANE_{—} | Prescription of MELIANE TAB 21 (G03AA10), count during F2: |
| | TAB_21 | second blood test (weeks 16-19) |
| 1276 | F2_pharma_D08AX08_A1COTINT_{—} | Prescription of A1COTINT SPT 70% 100 ML (D08AX08), count |
| | SPT_70%_100ML | during F2: second blood test (weeks 16-19) |
| 1277 | F2_pharma_R01AX10_OTRIMER_{—} | Prescription of OTRIMER SPR 0.9% 50 ML (R01AX10), count |
| | SPR_0_9%_50ML | during F2: second blood test (weeks 16-19) |
| 1278 | F2_pharma_R05CA20_THYMI_{—} | Prescription of THYMI SYR BOT 100 ML (R05CA20), count |
| | SYR_BOT_100ML | during F2: second blood test (weeks 16-19) |
| 1279 | F2_pharma_D06AX09_BACTROBAN_{—} | Prescription of BACTROBAN OIN 2% 15 g (D06AX09), count |
| | OIN_2%_15g | during F2: second blood test (weeks 16-19) |
| 1280 | F2_pharma_B03BA01_BEDODEKA_{—} | Prescription of BEDODEKA INJ 1000 MCG/1 ML 100 |
| | INJ_1000MCG_OR_1ML_100 | (B03BA01), count during F2: second blood test (weeks 16-19) |
| 1281 | F2_pharma_G03GA02_MENOPUR_{—} | Prescription of MENOPUR MULTIDOSE INJ 600 UNIT |
| | MULTIDOSE_INJ_600UNIT | (G03GA02), count during F2: second blood test (weeks 16-19) |
| 1282 | F2_pharma_D01AC22_AGISTEN_{—} | Prescription of AGISTEN ALOEVERA CR 1% 20 GM |
| | ALOEVERA_CR_1%_20GM | (D01AC22), count during F2: second blood test (weeks 16-19) |
| 1283 | F2_pharma_G01AA10_DALACIN_{—} | Prescription of DALACIN VAGINAL OVL 100 MG 3 |
| | VAGINAL_OVL_100MG_3 | (G01AA10), count during F2: second blood test (weeks 16-19) |
| 1284 | F2_pharma_M02AA15_VOLTAREN_{—} | Prescription of VOLTAREN EMULGEL GEL 1% 100 GM |
| | EMULGEL_GEL_1%_100GM | (M02AA15), count during F2: second blood test (weeks 16-19) |
| 1285 | F2_pharma_G12A | Prescription of (G12A), count during F2: second blood test |
| | | (weeks 16-19) |
| 1286 | F2_pharma_S01CA20_DETHAMYCIN_{—} | Prescription of DETHAMYCIN EYE, EAR & NOSE DRP BOT |
| | EYE_EAR_&_NOSE_DRP_BOT_10mL | 10 mL (S01CA20), count during F2: second blood test |
| | | (weeks 16-19) |
| 1287 | F2_pharma_G03GA06_PUREGON_{—} | Prescription of PUREGON INJ 900IU (G03GA06), count during |
| | INJ_900IU | F2: second blood test (weeks 16-19) |
| 1288 | F2_pharma_M01AE01_NUROFEN_{—} | Prescription of NUROFEN STRAWB.PED. SUS 100 mg/5 mL |
| | STRAWB_PED_SUS_100mg_OR_{—} | 100 mL (M01AE01), count during F2: second blood test |
| | 5mL_100mL | (weeks 16-19) |
| 1289 | F2_pharma_D01AC23_{—} | Prescription of TEVADERM CR CF 15 GM (D01AC23), count |
| | TEVADERM_CR_CF_15GM | during F2: second blood test (weeks 16-19) |
| 1290 | F2_pharma_A11CC05_D_TABS_{—} | Prescription of D-TABS TAB 400IU 90 (A11CC05), count during |
| | TAB_400IU_90 | F2: second blood test (weeks 16-19) |
| 1291 | F2_pharma_H08 | Prescription of (H08), count during F2: second blood test |
| | | (weeks 16-19) |
| 1292 | F2_pharma_D08AX08_ALCOHOL_{—} | Prescription of ALCOHOL SPT 70% 100 ML (D08AX08), count |
| | SPT_70%_100ML | during F2: second blood test (weeks 16-19) |
| 1293 | F2_pharma_A01AC21_{—} | Prescription of ORACORT E PAS TUB 5 g (A01AC21), count |
| | ORACORT_E_PAS_TUB_5g | during F2: second blood test (weeks 16-19) |
| 1294 | F2_pharma_R02AA20_LEMOCIN_{—} | Prescription of LEMOCIN CHERRY SUGAR FREE LOZ CF 24 |
| | CHERRY_SUGAR_FREE_LOZ_CF_24 | (R02AA20), count during F2: second blood test (weeks 16-19) |
| 1295 | F2_pharma_A07FA01_ACIDOPHILUS_{—} | Prescription of ACIDOPHILUS PROBIOTIC CAP BOX 30 |
| | PROBIOTIC_CAP_BOX_30 | (A07FA01), count during F2: second blood test (weeks 16-19) |
| 1296 | F2_pharma_S01AA32_PHENIMYXIN_{—} | Prescription of PHENIMYXIN COL 8 ML (S01AA32), count |
| | COL_8ML | during F2: second blood test (weeks 16-19) |
| 1297 | F2_pharma_N02BB02_OPTALGIN_{—} | Prescription of OPTALGIN CPL 500 mg 42 (N02BB02), count |
| | CPL_500mg_42 | during F2: second blood test (weeks 16-19) |
| 1298 | F2_pharma_M04AC01_COLCHICINE_{—} | Prescription of COLCHICINE TAB 0.5 MG 30 (M04AC01), |
| | TAB_0_5MG_30 | count during F2: second blood test (weeks 16-19) |
| 1299 | F2_pharma_R03BB01_AEROVENT_{—} | Prescription of AEROVENT SOL 0.25 mg/1 mL 20 mL |
| | SOL_0_25mg_OR_1mL_20mL | (R03BB01), count during F2: second blood test (weeks 16-19) |
| 1300 | F2_pharma_A01AD12_TEE | Prescription of TEEJEL GEL CF 10 GM (A01AD12), count |
| | JEL_GEL_CF_10GM | during F2: second blood test (weeks 16-19) |
| 1301 | F2_pharma_G01AF02_AGISTEN_V_{—} | Prescription of AGISTEN V VAG. TAB 200 MG 3 (G01AF02), |
| | VAG_TAB_200MG_3 | count during F2: second blood test (weeks 16-19) |
| 1302 | F2_pharma_R01AD05_NASOCORT_{—} | Prescription of NASOCORT AQUA NASAL SPR 64 MCG |
| | AQUA_NASAL_SPR_64MCG | (R01AD05), count during F2: second blood test (weeks 16-19) |
| 1303 | F2_pharma_R03AK06_SERETIDE_{—} | Prescription of SERETIDE DISKUS INH 50/250 MCG U |
| | DISKUS_INH_50_OR_250MCG_U | (R03AK06), count during F2: second blood test (weeks 16-19) |
| 1304 | F2_pharma_D01AC25_TRIDERM_{—} | Prescription of TRIDERM CR CF 15 GM (D01AC25), count |
| | CR_CF_15GM | during F2: second blood test (weeks 16-19) |
| 1305 | F2_pharma_A11AA06_PRENATAL_{—} | Prescription of PRENATAL MULTIVITAMINS D.H TAB 30 |
| | MULTIVITAMINS_D_H_TAB_30 | (A11AA06), count during F2: second blood test (weeks 16-19) |
| 1306 | F2_pharma_M01AE01_NUROFEN_{—} | Prescription of NUROFEN LIQUID CAP 200 MG BOX 40 |
| | LIQUID_CAP_200MG_BOX_40 | (M01AE01), count during F2: second blood test (weeks 16-19) |
| 1307 | F2_pharma_S02DA30_ANAESTHETIC_{—} | Prescription of ANAESTHETIC AUR 10 mL (S02DA30), count |
| | AUR_10mL | during F2: second blood test (weeks 16-19) |
| 1308 | F2_pharma_M01AE01_NUROFEN_{—} | Prescription of NUROFEN LIQUID CAP 200 MG BOX 20 |
| | LIQUID_CAP_200MG_BOX_20 | (M01AE01), count during F2: second blood test (weeks 16-19) |
| 1309 | F2_pharma_S01CA01_MAXITROL_{—} | Prescription of MAXITROL OCC CF TUB 3.5 GM (S01CA01), |
| | OCC_CF_TUB_3_5GM | count during F2: second blood test (weeks 16-19) |
| 1310 | F2_pharma_D07AC13_ELOCOM_{—} | Prescription of ELOCOM CR 0.1% 15 GM (D07AC13), count |
| | CR_0_1%_15GM | during F2: second blood test (weeks 16-19) |
| 1311 | F2_pharma_N02BB02_OPTALGIN_{—} | Prescription of OPTALGIN DRP 500 MG/1 ML 10 ML |
| | DRP_500MG_OR_1ML_10ML | (N02BB02), count during F2: second blood test (weeks 16-19) |
| 1312 | F2_pharma_B03AD02_FORIC_{—} | Prescription of FORIC PREGNANCY TAB CF BOX 30 |
| | PREGNANCY_TAB_CF_BOX_30 | (B03AD02), count during F2: second blood test (weeks 16-19) |
| 1313 | F2_pharma_G03GA08_OVITRELLE_{—} | Prescription of OVITRELLE PREFILLED INJ 250 mcg |
| | PREFILLED_INJ_250mcg | (G03GA08), count during F2: second blood test (weeks 16-19) |
| 1314 | F2_pharma_B03BB01_FOLIC_ACID_{—} | Prescription of FOLIC ACID (REKAH) TAB 5 MG 30 |
| | REKAH_TAB_5MG_30 | (B03BB01), count during F2: second blood test (weeks 16-19) |
| 1315 | F1_pharma_H03B | Prescription of (H03B), count during F1: first blood test |
| | | (weeks 6-12) |
| 1316 | F1_pharma_D01AC24POLYCUTAN_{—} | Prescription of POLYCUTAN CR 15 GM (D01AC24), count |
| | CR_15GM | during F1: first blood test (weeks 6-12) |
| 1317 | F1_pharma_G03GB02_IKACLOMIN_{—} | Prescription of IKACLOMIN TAB 50 MG 10 (G03GB02), count |
| | TAB_50MG_10 | during F1: first blood test (weeks 6-12) |
| 1318 | F1_pharma_N02BB02_OPTALGIN_{—} | Prescription of OPTALGIN TAB 500 MG 20 (N02BB02), count |
| | TAB_500MG_20 | during F1: first blood test (weeks 6-12) |
| 1319 | F1_pharma_C10AX06_MAARAZ_{—} | Prescription of MAARAZ 9M: OMEGA-3 + PRENATAL(ALT |
| | 9M_OMEGA_3+PRENATAL_{—} | KIT 120 (C10AX06), count during F1: first blood test |
| | ALT_KIT_120 | (weeks 6-12) |
| 1320 | F1_pharma_N02BE01_ACAMOLI_{—} | Prescription of ACAMOLI STRAW. S/F SYR 125 mg/5 mL |
| | STRAW_S_OR_F_SYR_125mg_OR_{—} | 100 mL (N02BE01), count during F1: first blood test (weeks 6-12) |
| | 5mL_100mL | |
| 1321 | F1_pharma_B03BA01_VIT_B12_{—} | Prescription of VIT B12(SOLGAR)SUBL TAB 100O mcg 100 |
| | SOLGAR_SUBL_TAB_1000mcg_100 | (B03BA01), count during F1: first blood test (weeks 6-12) |
| 1322 | F1_pharma_B01AB05_CLEXANE_{—} | Prescription of CLEXANE SAFETY LOCK INJ 60 MG/0.6 ML 2 |
| | SAFETY_LOCK_INJ_60MG_OR_0_6ML_2 | (B01AB05), count during F1: first blood test (weeks 6-12) |
| 1323 | F1_pharma_A04AA01_ZOFRAN_{—} | Prescription of ZOFRAN TAB 4 MG 10 (A04AA01), count during |
| | TAB_4MG_10 | F1: first blood test (weeks 6-12) |
| 1324 | F1_pharma_S01AA01_{—} | Prescription of SYNTHOMYCINE OCC 5% 3.6 GM (S01AA01), |
| | SYNTHOMYCINE_OCC_5%_3_6GM | count during F1: first blood test (weeks 6-12) |
| 1325 | F1_pharma_G01AF02_CLOTREE_{—} | Prescription of CLOTREE-TEVA VAG OVL 200 MG 3 |
| | TEVA_VAG_OVL_200MG_3 | (G01AF02), count during F1: first blood test (weeks 6-12) |
| 1326 | F1_pharma_J01CA04_MOXYPEN_{—} | Prescription of MOXYPEN CAP 500 mg 20 (J01CA04), count |
| | CAP_500mg_20 | during F1: first blood test (weeks 6-12) |
| 1327 | F1_pharma_H01CC02_CETROTIDE_{—} | Prescription of CETROTIDE VIA 0.25 mg 7 (H01CC02), count |
| | VIA_0_25mg_7 | during F1: first blood test (weeks 6-12) |
| 1328 | F1_pharma_G03DA03_PROGESTERON_{—} | Prescription of PROGESTERON RETARD 29/M INJ |
| | RETARD_29_OR_M_INJ_500MG_{—} | 500 MG/2 ML 3 (G03DA03), count during F1: first blood test |
| | OR_2ML_3 | (weeks 6-12) |
| 1329 | F1_pharma_J01CA04_MOXYPEN_{—} | Prescription of MOXYPEN CAP 500 MG 10 (J01CA04), count |
| | CAP_500MG_10 | during F1: first blood test (weeks 6-12) |
| 1330 | F1_pharma_N02BB02_{—} | Prescription of OPTALGIN CPL 500 MG 21 (N02BB02), count |
| | OPTALGIN_CPL_500MG_21 | during F1: first blood test (weeks 6-12) |
| 1331 | F1_pharma_G03CA03_PROGYNOVA_{—} | Prescription of PROGYNOVA TAB 2 MG 28 (G03CA03), count |
| | TAB_2MG_28 | during F1: first blood test (weeks 6-12) |
| 1332 | F1_pharma_L02AE04_DECAPEPTYL_{—} | Prescription of DECAPEPTYL INJ 0.1 mg/1 mL 7 (L02AE04), |
| | INJ_0_1mg_OR_1mL_7 | count during F1: first blood test (weeks 6-12) |
| 1333 | F1_pharma_H03AA01_ELTROXIN_{—} | Prescription of ELTROXIN TAB 100 MCG 100 (H03AA01), |
| | TAB_100MCG_100 | count during F1: first blood test (weeks 6-12) |
| 1334 | F1_pharma_H03AA01_EUTHYROX_{—} | Prescription of EUTHYROX TAB 50 mcg 100 (H03AA01), count |
| | TAB_50mcg_100 | during F1: first blood test (weeks 6-12) |
| 1335 | F1_pharma_A02BC01_OMEPRADEX_{—} | Prescription of OMEPRADEX CPL 20 mg 30 (A02BC01), count |
| | CPL_20mg_30 | during F1: first blood test (weeks 6-12) |
| 1336 | F1_pharma_J01CR02_AMOXICLAV_{—} | Prescription of AMOXICLAV TEVA TAB 875 mg 14 (J01CR02), |
| | TEVA_TAB_875mg_14 | count during F1: first blood test (weeks 6-12) |
| 1337 | F1_pharma_R06AX13_ALLERGYX_{—} | Prescription of ALLERGYX TAB 10 MG 10 (R06AX13), count |
| | TAB_10MG_10 | during F1: first blood test (weeks 6-12) |
| 1338 | F1_pharma_G03GA05_GONAL_F_{—} | Prescription of GONAL-F-PEN INJ 450IU 0.75 ML (G03GA05), |
| | PEN_INJ_450IU_0_75ML | count during F1: first blood test (weeks 6-12) |
| 1339 | F1_pharma_B03BB01_FOLI_5_{—} | Prescription of FOLI 5 (CTS) TAB 5 mg 30 (B03BB01), count |
| | CTS_TAB_5mg_30 | during F1: first blood test (weeks 6-12) |
| 1340 | F1_pharma_B03AE02_GENTLE_{—} | Prescription of GENTLE IRON CAP 25 MG BOX 90 (B03AE02), |
| | IRON_CAP_25MG_BOX_90 | count during F1: first blood test (weeks 6-12) |
| 1341 | F1_pharma_C05AX05_PROCTO_{—} | Prescription of PROCTO-GLYVENOL CR CF TUB 30 GM |
| | GLYVENOL_CR_CF_TUB_30GM | (C05AX05), count during F1: first blood test (weeks 6-12) |
| 1342 | F1_pharma_B03BA51_VIT_B12+ | Prescription of VIT.B12 + FOLIC A.SUBLING. TAB CF 60 |
| | FOLIC_A_SUBLING_TAB_CF_60 | (B03BA51), count during F1: first blood test (weeks 6-12) |
| 1343 | F1_pharma_R03AC02_VENTOLIN_{—} | Prescription of VENTOLIN CFC FREE INH 0.1 MG/INH |
| | CFC_FREE_INH_0_1MG_OR_INH | (R03AC02), count during F1: first blood test (weeks 6-12) |
| 1344 | F1_pharma_M01AE01_NUROFEN_{—} | Prescription of NUROFEN ORANGE CHILD SUS 100 MG/5 ML |
| | ORANGE_CHILD_SUS_100MG_{—} | 100 ML (M01AE01), count during F1: first blood test |
| | OR_5ML_100ML | (weeks 6-12) |
| 1345 | F1_pharma_D04AA13_FENISTIL_{—} | Prescription of FENISTIL GEL 0.1% 30 GM (D04AA13), count |
| | GEL_0_1%_30GM | during F1: first blood test (weeks 6-12) |
| 1346 | F1_pharma_D01AC24_TEVACUTAN_{—} | Prescription of TEVACUTAN CR 15 g (D01AC24), count during |
| | CR_15g | F1: first blood test (weeks 6-12) |
| 1347 | F1_pharma_G01AF04_GYNO_{—} | Prescription of GYNO-DAKTARIN VAG. CAP 400 mg 3 |
| | DAKTARIN_VAG_CAP_400mg_3 | (G01AF04), count during F1: first blood test (weeks 6-12) |
| 1348 | F1_pharma_S02CA06_DESOREN_{—} | Prescription of DESOREN AUR CF BOT 5 ML (S02CA06), count |
| | AUR_CF_BOT_5ML | during F1: first blood test (weeks 6-12) |
| 1349 | F1_pharma_A03AD01_PAPAVERINE_{—} | Prescription of PAPAVERINE TAB 80 MG 30 (A03AD01), count |
| | TAB_80MG_30 | during F1: first blood test (weeks 6-12) |
| 1350 | F1_pharma_B03AB10_TIPTIPOT_{—} | Prescription of TIPTIPOT FERRIPEL-3 DRP 50 mg/1 mL 15 mL |
| | FERRIPEL_3_DRP_50mg_OR_{—} | (B03AB10), count during F1: first blood test (weeks 6-12) |
| | 1mL_15mF | |
| 1351 | F1_pharma_G03GA02_{—} | Prescription of MENOGON <><> INJ 75IU/75IU 10 (G03GA02), |
| | MENOGON_<><>_INJ_75IU_{—} | count during F1: first blood test (weeks 6-12) |
| | OR_75IU_10 | |
| 1352 | F1_pharma_R06AX26_TELFAST_{—} | Prescription of TELFAST TAB 180 MG 15 (R06AX26), count |
| | TAB_180MG_15 | during F1: first blood test (weeks 6-12) |
| 1353 | F1_pharma_N06AB10_CIPRALEX_{—} | Prescription of CIPRALEX (MUSHLAM) TAB 10 MG 28 |
| | MUSHLAM_TAB_10MG_28 | (N06AB10), count during F1: first blood test (weeks 6-12) |
| 1354 | F1_pharma_N02BE01_ACAMOLI_{—} | Prescription of ACAMOLI SUP 150 MG 12 (N02BE01), count |
| | SUP_150MG_12 | during F1: first blood test (weeks 6-12) |
| 1355 | F1_pharma_{——}FEMINA_SOAP_{—} | Prescription of FEMINA SOAP LIQ 330 ML ( ), count during F1: |
| | LIQ_330ML | first blood test (weeks 6-12) |
| 1356 | F1_pharma_G03DA04_GESTON_{—} | Prescription of GESTON INJ 50 mg/1 mL 1 mL (G03DA04), count |
| | INJ_50mg_OR_1mL_1mL | during F1: first blood test (weeks 6-12) |
| 1357 | F1_pharma_A11AA06_PRENATAL_{—} | Prescription of PRENATAL MULTIVITAMINS D.H TAB 100 |
| | MULTIVITAMINS_D_H_TAB_100 | (A11AA06), count during F1: first blood test (weeks 6-12) |
| 1358 | F1_pharma_D07CB01_{—} | Prescription of DERMACOMBIN CR CF 15 GM (D07CB01), |
| | DERMACOMBIN_CR_CF_15GM | count during F1: first blood test (weeks 6-12) |
| 1359 | F1_pharma_U4_CLEAR_TEST | Prescription of CLEAR TEST (U4), count during F1: first blood |
| | | test (weeks 6-12) |
| 1360 | F1_pharma_Z07_YES_OR_NO_{—} | Prescription of YES OR NO DIRECT KIT 1 (Z07), count during |
| | DIRECT_KIT_1 | F1: first blood test (weeks 6-12) |
| 1361 | F1_pharma_G03GA05_GONAL_{—} | Prescription of GONAL-F-PEN INJ 900IU 1.5 ML (G03GA05), |
| | F_PEN_INJ_900IU_1_5ML | count during F1: first blood test (weeks 6-12) |
| 1362 | F1_pharma_B03AD03_FERROGRAD_{—} | Prescription of FERROGRAD FOLIC TAB CF BOX 30 |
| | FOLIC_TAB_CF_BOX_30 | (B03AD03), count during F1: first blood test (weeks 6-12) |
| 1363 | F0_pharma_M01AE01_NUROFEN_{—} | Prescription of NUROFEN LIQUID CAP 200 MG BOX 20 |
| | LIQUID_CAP_200MG_BOX_20 | (M01AE01), count during F0: before and early pregnancy |
| 1364 | F0_pharma_R01BA52_{—} | Prescription of CLARINASE REPETABS TAB 14 (R01BA52), |
| | CLARINASE_REPETABS_TAB_14 | count during F0: before and early pregnancy |
| 1365 | F0_pharma_R01AD08_{—} | Prescription of AVAMYS AQ. NASAL 120 INH SPR 27.5 MCG |
| | AVAMYS_AQ_NASAL_120_{—} | (R01AD08), count during F0: before and early pregnancy |
| | INH_SPR_27_5MCG | |
| 1366 | F0_pharma_A01AD12_TEEJEL_{—} | Prescription of TEEJEL GEL CF 10 GM (A01AD12), count |
| | G EL_CF_10GM | during F0: before and early pregnancy |
| 1367 | F0_pharma_G01AF02_AGISTEN_{—} | Prescription of AGISTEN V VAG. TAB 200 MG 3 (G01AF02), |
| | V_VAG_TAB_200MG_3 | count during F0: before and early pregnancy |
| 1368 | F0_pharma_R01AD05_{—} | Prescription of NASOCORT AQUA NASAL SPR 64 MCG |
| | NASOCORT_AQUA_NASAL_SPR_{—} | (R01AD05), count during F0: before and early pregnancy |
| | 64MCG | |
| 1369 | F0_pharma_R03AK06_{—} | Prescription of SERETIDE DISKUS INH 50/250 MCG U |
| | SERETIDE_DISKUS_INH_50_{—} | (R03AK06), count during F0: before and early pregnancy |
| | OR_250MCG_U | |
| 1370 | F0_pharma_D01AC25_{—} | Prescription of TRIDERM CR CF 15 GM (D01AC25), count |
| | TRIDERM_CR_CF_15GM | during F0: before and early pregnancy |
| 1371 | F0_pharma_M01AE01_{—} | Prescription of NUROFEN LIQUID CAP 200 MG BOX 40 |
| | NUROFEN_LIQUID_CAP_{—} | (M01AE01), count during F0: before and early pregnancy |
| | 200MG_BOX_40 | |
| 1372 | F0_pharma_S02DA30_{—} | Prescription of ANAESTHETIC AUR 10 mL (S02DA30), count |
| | ANAESTHETIC_AUR_10mL | during F0: before and early pregnancy |
| 1373 | F0_pharma_S01CA01_{—} | Prescription of MAXITROL OCC CF TUB 3.5 GM (S01CA01), |
| | MAXITROL_OCC_CF_TUB_3_5GM | count during F0: before and early pregnancy |
| 1374 | F1_pharma_B03AE02_IRON_{—} | Prescription of IRON BIS-GLYCINATE DH CAP 90 |
| | BIS_GLYCINATE_DH_CAP_90 | (B03AE02), count during F1: first blood test (weeks 6-12) |
| 1375 | F0_pharma_D07AC13_{—} | Prescription of ELOCOM CR 0.1% 15 GM (D07AC13), count |
| | ELOCOM_CR_0_1%_15GM | during F0: before and early pregnancy |
| 1376 | F0_pharma_N02BB02_OPTALGIN_{—} | Prescription of OPTALGIN DRP 500 MG/1 ML 10 ML |
| | DRP_500MG_OR_1ML_10ML | (N02BB02), count during F0: before and early pregnancy |
| 1377 | F0_pharma_D08AX08_ALCOHOL_{—} | Prescription of ALCOHOL SPT 70% 100 ML (D08AX08), count |
| | SPT_70%_100ML | during F0: before and early pregnancy |
| 1378 | F0_pharma_A11CC05_D_TABS_{—} | Prescription of D-TABS TAB 400IU 90 (A11CC05), count during |
| | TAB_400IU_90 | F0: before and early pregnancy |
| 1379 | F0_pharma_D06BB03_ZOVIRAX_{—} | Prescription of ZOVIRAX CR 5% 2 GM (D06BB03), count |
| | CR_5%_2GM | during F0: before and early pregnancy |
| 1380 | F0_pharma_R06AX13_{—} | Prescription of LORATADINE (LORATADIM) TAB 10 mg 10 |
| | LORATADINE_LORATADIM_{—} | (R06AX13), count during F0: before and early pregnancy |
| | TAB_10mg_10 | |
| 1381 | F0_pharma_B03BA01_VIT_B12_{—} | Prescription of VIT B12 (FLORIS) SUBLING TAB 1000 MCG |
| | FLORIS_SUBLING_TAB_{—} | 30 (B03BA01), count during F0: before and early pregnancy |
| | 1000MCG_30 | |
| 1382 | F0_pharma_G03GA06_{—} | Prescription of PUREGON INJ 300IU 0.36 ML (G03GA06), count |
| | PUREGON_INJ_300IU_0_{—} | during F0: before and early pregnancy |
| | 36ML | |
| 1383 | F0_pharma_G03GA02_{—} | Prescription of MENOPUR MULTIDOSE INJ 1200 UNIT |
| | MENOPUR_MULTIDOSE_{—} | (G03GA02), count during F0: before and early pregnancy |
| | INJ_1200UNIT | |
| 1384 | F0_pharma_R03BB01_{—} | Prescription of AEROVENT SOL 0.25 mg/1 mL 20 mL |
| | AEROVENT_SOL_0_25mg_{—} | (R03BB01), count during F0: before and early pregnancy |
| | OR_1mL_20mL | |
| 1385 | F0_pharma_M04AC01_{—} | Prescription of COLCHICINE TAB 0.5 MG 30 (M04AC01), |
| | COLCHICINE_TAB_0_5MG_30 | count during F0: before and early pregnancy |
| 1386 | F0_pharma_N02BB02_{—} | Prescription of OPTALGIN CPL 500 mg 42 (N02BB02), count |
| | OPTALGIN_CPL_500mg_42 | during F0: before and early pregnancy |
| 1387 | F0_pharma_S01AA32_{—} | Prescription of PHENIMYXIN COL 8 ML (S01AA32), count |
| | PHENIMYXIN_COL_8ML | during F0: before and early pregnancy |
| 1388 | F1_pharma_J01CE02_PEN_{—} | Prescription of PEN-RAFA VK CPL 500 mg 40 (J01CE02), count |
| | RAFA_VK_CPL_500mg_40 | during F1: first blood test (weeks 6-12) |
| 1389 | F1_pharma_U3 | Prescription of(U3), count during F1: first blood test (weeks 6-12) |
| 1390 | F1_pharma_N02BE01_{—} | Prescription of DEXAMOL CPL 500 MG 20 (N02BE01), count |
| | DEXAMOL_CPL_500MG_20 | during F1: first blood test (weeks 6-12) |
| 1391 | F1_pharma_B01AB05_{—} | Prescription of CLEXANE SAFETY LOCK INJ 40 MG/0.4 ML 2 |
| | CLEXANE_SAFETY_LOCK_{—} | (B01AB05), count during F1: first blood test (weeks 6-12) |
| | INJ_40MG_OR_0_4ML_2 | |
| 1392 | F1_pharma_B03AD02_{—} | Prescription of FORIC PREGNANCY TAB CF BOX 30 |
| | FORIC_PREGNANCY_TAB_{—} | (B03AD02), count during F1: first blood test (weeks 6-12) |
| | CF_BOX_30 | |
| 1393 | F1_pharma_G03GA08_{—} | Prescription of OVITRELLE PREFILLED INJ 250 mcg |
| | OVITRELLE_PREFILLED_{—} | (G03GA08), count during F1: first blood test (weeks 6-12) |
| | INJ_250mcg | |
| 1394 | F1_pharma_B03BB01_FOLIC_{—} | Prescription of FOLIC ACID (REKAH) TAB 5 MG 30 |
| | ACID_REKAH_TAB_5MG_30 | (B03BB01), count during F1: first blood test (weeks 6-12) |
| 1395 | F1_pharma_J01DC02_ZINNAT_{—} | Prescription of ZINNAT TAB 500 MG 10 (J01DC02), count |
| | TAB_500MG_10 | during F1: first blood test (weeks 6-12) |
| 1396 | F1_pharmna_J01CA04_MOXYVIT_{—} | Prescription of MOXYVIT CAP 500 MG 20 (J01CA04), count |
| | CAP_500MG_20 | during F1: first blood test (weeks 6-12) |
| 1397 | F1_pharma_G03DA04_{—} | Prescription of UTROGESTAN ORAL/VAG CAP 200 mg 15 |
| | UTROGESTAN_ORAL_OR_VAG_{—} | (G03DA04), count during F1: first blood test (weeks 6-12) |
| | CAP_200mg_15 | |
| 1398 | F1_pharma_J07BB02_VAXIGRIP_{—} | Prescription of VAXIGRIP VAC (J07BB02), count during F1: |
| | VAC | first blood test (weeks 6-12) |
| 1399 | F1_pharma_Unknown_ATC | Prescription of (Unknown_ATC), count during F1: first blood test |
| | | (weeks 6-12) |
| 1400 | F1_pharma_B03AD02_FOLEX_{—} | Prescription of FOLEX 400 TAB CF BOX 30 (B03AD02), count |
| | 400_TAB_CF_BOX_30 | during F1: first blood test (weeks 6-12) |
| 1401 | F1_pharma_B03BB01_FOLIC_{—} | Prescription of FOLIC ACID (NEW) D.H TAB 400 mcg 100 |
| | ACID_NEW_D_H_TAB_400mcg_100 | (B03BB01), count during F1: first blood test (weeks 6-12) |
| 1402 | F0_pharma_D07AC01_BETACORTEN_{—} | Prescription of BETACORTEN CR 0.1% 15 GM (D07AC01), |
| | CR_0_1%_15GM | count during F0: before and early pregnancy |
| 1403 | F0_pharma_D06AX09_BACTROBAN_{—} | Prescription of BACTROBAN OIN 2% 15 g (D06AX09), count |
| | OIN_2%_15g | during F0: before and early pregnancy |
| 1404 | F0_pharma_U0_LABON_TIME | Prescription of LABON TIME (U0), count during F0: before and |
| | | early pregnancy |
| 1405 | F0_pharma_A01AC21_ORACORT_{—} | Prescription of ORACORT E PAS TUB 5 g (A01AC21), count |
| | E_PAS_TUB_5g | during F0: before and early pregnancy |
| 1406 | F0_pharma_A07FA01_{—} | Prescription of ACIDOPHILUS PROBIOTIC CAP BOX 30 |
| | ACIDOPHILUS_PROBIOTIC_CAP_{—} | (A07FA01), count during F0: before and early pregnancy |
| | BOX_30 | |
| 1407 | F1_pharma_A11AA06_PRE_{—} | Prescription of PRE-GENTLY BADATZ (SUPHERB)TAB 60 |
| | GENTLY_BADATZ_SUPHERB_{—} | (A11AA06), count during F1: first blood test (weeks 6-12) |
| | TAB_60 | |
| 1408 | F1_pharma_G03DC02_{—} | Prescription of PRIMOLUT NOR TAB 5 MG 20 (G03DC02), |
| | PRIMOLUT_NOR_TAB_5MG_{—} | count during F1: first blood test (weeks 6-12) |
| | 20 | |
| 1409 | F2_pharma_A03FA01_{—} | Prescription of PRAMIN TAB 10 MG 30 (A03FA01), count |
| | PRAMIN_TAB_10MG_30 | during F2: second blood test (weeks 16-19) |
| 1410 | F1_pharma_D06BB03_{—} | Prescription of ZOVIRAX CR 5% 2 GM (D06BB03), count |
| | ZOVIRAX_CR_5%_2GM | during F1: first blood test (weeks 6-12) |
| 1411 | F1_pharma_A11AA06_{—} | Prescription of PRENATAL MULTIVITAMINS D.H TAB 30 |
| | PRENATAL_MULTIVITAMINS_{—} | (A11AA06), count during F1: first blood test (weeks 6-12) |
| | D_H_TAB_30 | |
| 1412 | F1_pharma_M01AE01_{—} | Prescription of NUROFEN LIQUID CAP 200 MG BOX 40 |
| | NUROFEN_LIQUID_CAP_{—} | (M01AE01), count during F1: first blood test (weeks 6-12) |
| | 200MG_BOX_40 | |
| 1413 | F1_pharma_S02DA30_{—} | Prescription of ANAESTHETIC AUR 10 mL (S02DA30), count |
| | ANAESTHETIC_AUR_10mL | during F1: first blood test (weeks 6-12) |
| 1414 | F1_pharma_S01CA01_{—} | Prescription of MAXITROL OCC CF TUB 3.5 GM (S01CA01), |
| | MAXITROL_OCC_CF_TUB_3_5GM | count during F1: first blood test (weeks 6-12) |
| 1415 | F1_pharma_D07AC13_{—} | Prescription of ELOCOM CR 0.1% 15 GM (D07AC13), count |
| | ELOCOM_CR_0_1%_15GM | during F1: first blood test (weeks 6-12) |
| 1416 | F1_pharma_N02BB02_{—} | Prescription of OPTALGIN DRP 500 MG/1 ML 10 ML |
| | OPTALGIN_DRP_500MG_{—} | (N02BB02), count during F1: first blood test (weeks 6-12) |
| | OR_1ML_10ML | |
| 1417 | F1_pharma_D08AX08_{—} | Prescription of ALCOHOL SPT 70% 100 ML (D08AX08), count |
| | ALCOHOL_SPT_70%_100ML | during F1: first blood test (weeks 6-12) |
| 1418 | F1_pharma_H08 | Prescription of (H08), count during F1: first blood test |
| | | (weeks 6-12) |
| 1419 | F1_pharma_D01AC23_{—} | Prescription of TEVADERM CR CF 15 GM (D01AC23), count |
| | TEVADERM_CR_CF_15GM | during F1: first blood test (weeks 6-12) |
| 1420 | F1_pharma_G03GA06_{—} | Prescription of PUREGON INJ 300IU 0.36 ML (G03GA06), count |
| | PUREGON_INJ_300IU_0_36ML | during F1: first blood test (weeks 6-12) |
| 1421 | F1_pharma_R03AK06_{—} | Prescription of SERETIDE DISKUS INH 50/250 MCG U |
| | SERETIDE_DISKUS_INH_50_{—} | (R03AK06), count during F1: first blood test (weeks 6-12) |
| | OR_250MCG_U | |
| 1422 | F1_pharma_G03GA02_{—} | Prescription of MENOPUR MULTIDOSE INJ 1200 UNIT |
| | MENOPUR_MULTIDOSE_{—} | (G03GA02), count during F1: first blood test (weeks 6-12) |
| | INJ_1200UNIT | |
| 1423 | F1_pharma_R01AA07_{—} | Prescription of OTRIVIN NASAL M.D. SPR 0.1% 10 ML |
| | TRIVIN_NASAL_M_D_{—} | (R01AA07), count during F1: first blood test (weeks 6-12) |
| | SPR_0_1%_10ML | |
| 1424 | F1_pharma_G03AA10_{—} | Prescription of MELIANE TAB 21 (G03AA10), count during F1: |
| | MELIANE_TAB_21 | first blood test (weeks 6-12) |
| 1425 | F1_pharma_R05CB01_{—} | Prescription of REOLIN EFFERV. TAB 200 MG 30 (R05CB01), |
| | REOLIN_EFFERV_TAB_200MG_30 | count during F1: first blood test (weeks 6-12) |
| 1426 | F1_pharma_D08AX08_{—} | Prescription of A1COTINT SPT 70% 100 ML (D08AX08), count |
| | ALCOTINT_SPT_70%_100ML | during F1: first blood test (weeks 6-12) |
| 1427 | F1_pharma_R01AX10_{—} | Prescription of OTRIMER SPR 0.9% 50 ML (R01AX10), count |
| | OTRIMER_SPR_0_9%_50ML | during F1: first blood test (weeks 6-12) |
| 1428 | F1_pharma_R05CA20_{—} | Prescription of THYMI SYR BOT 100 ML (R05CA20), count |
| | THYMI_SYR_BOT_100ML | during F1: first blood test (weeks 6-12) |
| 1429 | F1_pharma_B03BA01_{—} | Prescription of BEDODEKA INJ 1000 MCG/1 ML 100 |
| | BEDODEKA_INJ_1000MCG_{—} | (B03BA01), count during F1: first blood test (weeks 6-12) |
| | OR_1ML_100 | |
| 1430 | F1_pharma_G03GA02_{—} | Prescription of MENOPUR MULTIDOSE INJ 600 UNIT |
| | MENOPUR_MULTIDOSE_{—} | (G03GA02), count during F1: first blood test (weeks 6-12) |
| | INJ_600UNIT | |
| 1431 | F1_pharma_D01AC25_{—} | Prescription of TRIDERM CR CF 15 GM (D01AC25), count |
| | TRIDERM_CR_CF_15GM | during F1: first blood test (weeks 6-12) |
| 1432 | F1_pharma_R01AD05_{—} | Prescription of NASOCORT AQUA NASAL SPR 64 MCG |
| | NASOCORT_AQUA_NASAL_{—} | (R01AD05), count during F1: first blood test (weeks 6-12) |
| | SPR_64MCG | |
| 1433 | F1_pharma_B03AE12_{—} | Prescription of IRON PLUS FEMIN A (ALTMAN) TAB 100 |
| | IRON_PLUS_FEMINA_ALTMAN_{—} | (B03AE12), count during F1: first blood test (weeks 6-12) |
| | TAB_100 | |
| 1434 | F1_pharma_U0_LABON_TIME | Prescription of LABON TIME (U0), count during F1: first blood |
| | | test (weeks 6-12) |
| 1435 | F2_pharma_G03DA04_{—} | Prescription of UTROGESTAN ORAL/VAG CAP 200 mg 15 |
| | UTROGESTAN_ORAL_OR_{—} | (G03DA04), count during F2: second blood test (weeks 16-19) |
| | VAG_CAP_200mg_15 | |
| 1436 | F2_pharma_J07BB02_{—} | Prescription of VAXIGRIP VAC (J07BB02), count during F2: |
| | VAXIGRIP_VAC | second blood test (weeks 16-19) |
| 1437 | F2_pharma_Unknown_ATC | Prescription of (Unknown_ATC), count during F2: second blood |
| | | test (weeks 16-19) |
| 1438 | F2_pharma_G03DA04_{—} | Prescription of ENDOMETRIN VAGINAL TAB 100 MG BOX |
| | ENDOMETRIN_VAGINAL_{—} | 30 (G03DA04), count during F2: second blood test (weeks 16-19) |
| | TAB_100MG_BOX_30 | |
| 1439 | F2_pharma_B03AD02_{—} | Prescription of FOLEX 400 TAB CF BOX 30 (B03AD02), count |
| | FOLEX_400_TAB_CF_BOX_30 | during F2: second blood test (weeks 16-19) |
| 1440 | F2_pharma_B03BB01_{—} | Prescription of FOLIC ACID (NEW) D.H TAB 400 mcg 100 |
| | FOLIC_ACID_NEW_D_H_{—} | (B03BB01), count during F2: second blood test (weeks 16-19) |
| | TAB_400mcg_100 | |
| 1441 | F2_pharma_B03AD21_{—} | Prescription of FERRIFOL TAB CF BOX 30 (B03AD21), count |
| | FERRIFOL_TAB_CF_BOX_30 | during F2: second blood test (weeks 16-19) |
| 1442 | F1_pharma_D07ACO1_{—} | Prescription of BETACORTEN CR 0.1% 15 GM (D07AC01), |
| | BETACORTEN_CR_0_1%_15GM | count during F1: first blood test (weeks 6-12) |
| 1443 | F1_pharma_D06AX09_{—} | Prescription of BACTROBAN OIN 2% 15 g (D06AX09), count |
| | BACTROBAN_OIN_2%_15g | during F1: first blood test (weeks 6-12) |
| 1444 | F1_pharma_R02AA20_{—} | Prescription of LEMOCIN CHERRY SUGAR FREE LOZ CF 24 |
| | LEMOCIN_CHERRY_SUGAR_{—} | (R02AA20), count during F1: first blood test (weeks 6-12) |
| | FREE_LOZ_CF_24 | |
| 1445 | F1_pharma_G01AF02_{—} | Prescription of AGISTEN V VAG. TAB 200 MG 3 (G01AF02), |
| | AGISTEN_V_VAG_TAB_200MG_3 | count during F1: first blood test (weeks 6-12) |
| 1446 | F1_pharma_A07FA01_{—} | Prescription of ACIDOPHILUS PROBIOTIC CAP BOX 30 |
| | ACIDOPHILUS_PROBIOTIC_{—} | (A07FA01), count during F1: first blood test (weeks 6-12) |
| | CAP_BOX_30 | |
| 1447 | F1_pharma_S01AA32_{—} | Prescription of PHENIMYXIN COL 8 ML (S01AA32), count |
| | PHENIMYXIN_COL_8ML | during F1: first blood test (weeks 6-12) |
| 1448 | F1_pharma_M04AC01_{—} | Prescription of COLCHICINE TAB 0.5 MG 30 (M04AC01), |
| | COLCHICINE_TAB_0_5MG_30 | count during F1: first blood test (weeks 6-12) |
| 1449 | F1_pharma_R03BB01_{—} | Prescription of AEROVENT SOL 0.25 mg/1 mL 20 mL |
| | AEROVENT_SOL_0_25mg_{—} | (R03BB01), count during F1: first blood test (weeks 6-12) |
| | OR_1mL_20mL | |
| 1450 | F1_pharma_A06AD15_{—} | Prescription of NORMALAX PWD 240 GM (A06AD15), count |
| | NORMALAX_PWD_240GM | during F1: first blood test (weeks 6-12) |
| 1451 | F1_pharma_A02BC03_{—} | Prescription of LANTON CAP 30 MG BOX 28 (A02BC03), count |
| | LANTON_CAP_30MG_BOX_28 | during F1: first blood test (weeks 6-12) |
| 1452 | F1_pharma_R01BA52_{—} | Prescription of CLARINASE REPETABS TAB 14 (R01BA52), |
| | CLARINASE_REPETABS_TAB_14 | count during F1: first blood test (weeks 6-12) |
| 1453 | F1_pharma_R01AD08_{—} | Prescription of AVAMYS AQ. NASAL 120 INH SPR 27.5 MCG |
| | AVAMYS_AQ_NASAL_120_INH_{—} | (R01AD08), count during F1: first blood test (weeks 6-12) |
| | SPR_27_5MCG | |
| 1454 | F1_pharma_A01AD12_{—} | Prescription of TEEJEL GEL CF 10 GM (A01 AD 12), count |
| | TEEJEL_GEL_CF_10GM | during F1: first blood test (weeks 6-12) |
| 1455 | F1_pharma_D01AC22_{—} | Prescription of AGISTEN ALOEVERA CR 1% 20 GM |
| | AGISTEN_ALOEVERA_CR_1%_20GM | (D01AC22), count during F1: first blood test (weeks 6-12) |
| 1456 | F1_pharma_U2 | Prescription of(U2), count during F1: first blood test (weeks 6-12) |
| 1457 | F1_pharma_M02AA15_{—} | Prescription of VOLTAREN EMULGEL GEL 1% 100 GM |
| | VOLTAREN_EMULGEL_{—} | (M02AA15), count during F1: first blood test (weeks 6-12) |
| | GEL_1%_100GM | |
| 1458 | F1_pharma_R06AA59_{—} | Prescription of DICLECTIN DR TAB 10 mg/10 mg 100 |
| | DICLECTIN_DR_TAB_10mg_{—} | (R06AA59), count during F1: first blood test (weeks 6-12) |
| | OR_10mg_100 | |
| 1459 | F1_pharma_A02AD01_{—} | Prescription of MAALOX SUS CF BOT 355 ML (A02AD01), |
| | MAALOX_SUS_CF_BOT_355ML | count during F1: first blood test (weeks 6-12) |
| 1460 | F1_pharma_M01AE01_{—} | Prescription of ADVIL LIQUI-GELS CAP 200 MG 16 |
| | ADVIL_LIQUI_GELS_CAP_{—} | (M01AE01), count during F1: first blood test (weeks 6-12) |
| | 200MG_16 | |
| 1461 | F1_pharma_R02AA03_{—} | Prescription of STREPSILS HONEY-LEMON LOZ CF 24 |
| | STREPSILS_HONEY_LEMON_{—} | (R02AA03), count during F1: first blood test (weeks 6-12) |
| | LOZ_CF_24 | |
| 1462 | F1_pharma_R02AA20_{—} | Prescription of LEMOCIN LEMON SUGAR FREE LOZ CF 24 |
| | LEMOCIN_LEMON_SUGAR_{—} | (R02AA20), count during F1: first blood test (weeks 6-12) |
| | FREE_LOZ_CF_24 | |
| 1463 | F1_pharma_J01AA02_{—} | Prescription of DOXYLIN TAB 100 MG 10 (J01AA02), count |
| | DOXYLIN_TAB_100MG_10 | during F1: first blood test (weeks 6-12) |
| 1464 | F1_pharma_U0_LABON_TIME | Prescription of LABON TIME (U0), count during F1: first blood |
| | | test (weeks 6-12) |
| 1465 | F1_pharma_H02AB07_{—} | Prescription of PREDNISONE TAB 20 MG 30 (H02AB07), count |
| | PREDNISONE_TAB_20MG_30 | during F1: first blood test (weeks 6-12) |
| 1466 | F1_pharma_U4_CLEAR_TEST | Prescription of CLEAR TEST (U4), count during F1: first blood |
| | | test (weeks 6-12) |
| 1467 | F1_pharma_B01AC06_{—} | Prescription of MICROPIRIN TAB 75 MG 28 (B01AC06), count |
| | MICROPIRIN_TAB_75MG_28 | during F1: first blood test (weeks 6-12) |
| 1468 | F1_pharma_A11CC05_{—} | Prescription of VITAMIN D3 D.H DRP 400 UNIT 15 ML |
| | VITAMIN_D3_D_H_DRP_{—} | (A11CC05), count during F1: first blood test (weeks 6-12) |
| | 400UNIT_15ML | |
| 1469 | F1_pharma_A11CC05_{—} | Prescription of BABY D3 DRP 200IU 10 ML (A11CC05), count |
| | BABY_D3_DRP_200IU_10ML | during F1: first blood test (weeks 6-12) |
| 1470 | F1_pharma_R01AB22_{—} | Prescription of OTRIVIN MENTHOL M.D SPR 0.1% 10 ML |
| | OTRIVIN_MENTHOL_M_D_{—} | (R01AB22), count during F1: first blood test (weeks 6-12) |
| | SPR_0_1%_10ML | |
| 1471 | F1_pharma_N01BB54_{—} | Prescription of EMLA CR 5% 30 GM (N01BB54), count during |
| | EMLA_CR_5%_30GM | F1: first blood test (weeks 6-12) |
| 1472 | F1_pharma_R03AK07_{—} | Prescription of SYMBICORT TURBUHALER INH |
| | SYMBICORT_TURBUHALER_{—} | 160 mcg/4.5 mcg (R03AK07), count during F1: first blood test |
| | INH_160mcg_OR_4_5mcg | (weeks 6-12) |
| 1473 | F1_pharma_A04AA01_{—} | Prescription of ZOFRAN TAB 8 MG 10 (A04AA01), count during |
| | ZOFRAN_TAB_8MG_10 | F1: first blood test (weeks 6-12) |
| 1474 | F1_pharma_B03AB10_{—} | Prescription of FERRIPEL-3 SYR 50 MG/5 ML 110 ML |
| | FERRIPEL_3_SYR_50MG_{—} | (B03AB10), count during F1: first blood test (weeks 6-12) |
| | OR_5ML_110ML | |
| 1475 | F1_pharma_A11CC05_{—} | Prescription of VITAMIDYNE D DRP 200IU 10 ML (A11CC05), |
| | VITAMIDYNE_D_DRP_{—} | count during F1: first blood test (weeks 6-12) |
| | 200IU_10ML | |
| 1476 | F1_pharma_M01AE01_{—} | Prescription of NUROFEN STRAWB.CHILD SUS 100 MG/5 ML |
| | NUROFEN_STRAWB_CHILD_{—} | 150 ML (M01AE01), count during F1: first blood test |
| | SUS_100MG_OR_5ML_150ML | (weeks 6-12) |
| 1477 | F1_pharma_{——}OTRIVONIM_{—} | Prescription of OTRIVONIM BABY WIP PKG 2X36U ( ), count |
| | BABY_WIP_PKG_2X36U | during F1: first blood test (weeks 6-12) |
| 1478 | F1_pharma_G04BX06_{—} | Prescription of SEDURAL TAB 100 MG 30 (G04BX06), count |
| | SEDURAL_TAB_100MG_30 | during F1: first blood test (weeks 6-12) |
| 1479 | F1_pharma_R03AC02_{—} | Prescription of VENTOLIN RESPIR. SOL 5 mg/1 mL 20 mL |
| | VENTOLIN_RESPIR_SOL_{—} | (R03AC02), count during F1: first blood test (weeks 6-12) |
| | 5mg_OR_1mL_20mL | |
| 1480 | F1_pharma_G03GA02_{—} | Prescription of MENOPUR VIA 75IU 10 (G03GA02), count |
| | MENOPUR_VIA_75IU_10 | during F1: first blood test (weeks 6-12) |
| 1481 | F1_pharma_M01AE01_{—} | Prescription of NUROFEN ORANGE CHILD SUS 100 MG/5 ML |
| | NUROFEN_ORANGE_CHILD_{—} | 150 ML (M01AE01), count during F1: first blood test |
| | SUS_100MG_OR_5ML_150ML | (weeks 6-12) |
| 1482 | F1_pharma_B03BA01_{—} | Prescription of BEVITEX SUBLINGUAL TAB 1000 MCG 30 |
| | BEVITEX_SUBLINGUAL_{—} | (B03BA01), count during F1: first blood test (weeks 6-12) |
| | TAB_1000MCG_30 | |
| 1483 | F1_pharma_S01CA20_{—} | Prescription of DETHAMYCIN EYE, EAR & NOSE DRP BOT |
| | DETHAMYCIN_EYE_EAR_&_{—} | 10 mL (S01CA20), count during F1: first blood test (weeks 6-12) |
| | NOSE_DRP_BOT_10mL | |
| 1484 | F1_pharma_G03GA06_{—} | Prescription of PUREGON INJ 900IU (G03GA06), count during |
| | PUREGON_INJ_900IU | F1: first blood test (weeks 6-12) |
| 1485 | F1_pharma_D01AC63_{—} | Prescription of COMAGIS CR CF 15 GM (D01AC63), count |
| | COMAGIS_CR_CF_15GM | during F1: first blood test (weeks 6-12) |
| 1486 | F1_pharma_N02BE01_{—} | Prescription of ACAMOLI FRUIT S/F SYR 125 mg/5 mL 100 mL |
| | ACAMOLI_FRUIT_S_OR_F_{—} | (N02BE01), count during F1: first blood test (weeks 6-12) |
| | SYR_125mg_OR_5mL_100mL | |
| 1487 | F1_pharma_J01CR02_{—} | Prescription of AUGMENTIN TAB 500 MG 20 (J01CR02), count |
| | AUGMENTIN_TAB_500MG_20 | during F1: first blood test (weeks 6-12) |
| 1488 | F1_pharma_B03AD02_{—} | Prescription of FOROL TAB 30 (B03AD02), count during F1: |
| | FOROL_TAB_30 | first blood test (weeks 6-12) |
| 1489 | F1_pharma_B01AC06_{—} | Prescription of MICROPIRIN TAB 100 MG 28 (B01AC06), count |
| | MICROPIRIN_TAB_100MG_28 | during F1: first blood test (weeks 6-12) |
| 1490 | F1_pharma_{——}PARO_{—} | Prescription of PARO TOOTHBRUSH MEDIC NO. 726 ( ), |
| | TOOTHBRUSH_MEDIC_NO_726 | count during F1: first blood test (weeks 6-12) |
| 1491 | F1_pharma_{——}ELASTAN_{—} | Prescription of ELAST AN TREAT CR 75 ML ( ), count during |
| | TREAT_CR_75ML | F1: first blood test (weeks 6-12) |
| 1492 | F1_pharma_J06BB01_{—} | Prescription of KAMRHO-D I.M. INJ 300 mcg 2 mL (J06BB01), |
| | KAMRHO_D_I_M_INJ_{—} | count during F1: first blood test (weeks 6-12) |
| | 300mcg_2mL | |
| 1493 | F1_pharma_G01AF05_{—} | Prescription of GYNO-PEVARYL VAG TAB 150 MG 3 |
| | GYNO_PEVARYL_VAG_TAB_{—} | (G01AF05), count during F1: first blood test (weeks 6-12) |
| | 150MG_3 | |
| 1494 | F1_pharma_R01AD08_{—} | Prescription of FLIXONASE NASAL SPR 0.05% (R01AD08), |
| | FEIXONASE_NASAL_SPR_0_05% | count during F1: first blood test (weeks 6-12) |
| 1495 | F1_pharma_A12AX01_VITA_CAL+ | Prescription of VITA-CAL + D (NEW) TAB CF BOX 60 |
| | D_NEW_TAB_CF_BOX_60 | (A12AX01), count during F1: first blood test (weeks 6-12) |
| 1496 | F1_pharma_G03GA05_GONAL_{—} | Prescription of GONAL-F-PEN INJ 300IU 0.5 ML (G03GA05), |
| | F_PEN_INJ_300IU_0_5ML | count during F1: first blood test (weeks 6-12) |
| 1497 | F1_pharma_B03AA07_{—} | Prescription of AKTIFERRIN SOFT CAP 34 mg 30 (B03AA07), |
| | AKTIFERRIN_SOFT_CAP_34mg_30 | count during F1: first blood test (weeks 6-12) |
| 1498 | F1_pharma_S02DA30_{—} | Prescription of OTIDIN AUR CF 10 GM (S02DA30), count |
| | OTIDIN_AUR_CF_10GM | during F1: first blood test (weeks 6-12) |
| 1499 | F1_pharma_B01AC06_{—} | Prescription of MICROPIRIN TAB 75 mg 30 (B01AC06), count |
| | MICROPIRIN_TAB_75mg_30 | during F1: first blood test (weeks 6-12) |
| 1500 | F1_pharma_A11CC05_{—} | Prescription of D-DROPS DRP 200 UNIT 10 mL (A11CC05), |
| | D_DROPS_DRP_200UNIT_10mL | count during F1: first blood test (weeks 6-12) |
| 1501 | F1_pharma_D07CA03_{—} | Prescription of AFLUMYCIN CR CF 20 GM (D07CA03), count |
| | AFLUMYCIN_CR_CF_20GM | during F1: first blood test (weeks 6-12) |
| 1502 | F1_pharma_A06AX01_GLYCERIN_{—} | Prescription of GLYCERIN (SAM-ON) SUP 2 GM 20 |
| | SAM_ON_SUP_2GM_20 | (A06AX01), count during F1: first blood test (weeks 6-12) |
| 1503 | F1_A_B2_GLYCOPROTEI_IgM | Labtest A.B2 GLYCOPROTEI IgM, sampled during F1: first |
| | | blood test (weeks 6-12) |
| 1504 | F0_pharma_U3 | Prescription of (U3), count during F0: before and early pregnancy |
| 1505 | F2_clinic_L02_BACK_SYMPTOMS_{—} | Clinic diagnosis BACK SYMPTOMS/COMPLAINTS (L02), |
| | OR_COMPLAINTS | count during F2: second blood test (weeks 16-19) |
| 1506 | F2_clinic_7851_PALPITATIONS | Clinic diagnosis PALPITATIONS (7851), count during F2: |
| | | second blood test (weeks 16-19) |
| 1507 | F2_clinic_7860_DYSPNEA_AND_{—} | Clinic diagnosis DYSPNEA AND RESPIRATORY |
| | RESPIRATORY_ABNORMALITIES | ABNORMALITIES (7860), count during F2: second blood test |
| | | (weeks 16-19) |
| 1508 | F2_clinic_346_MIGRAINE | Clinic diagnosis MIGRAINE (346), count during F2: second |
| | | blood test (weeks 16-19) |
| 1509 | F2_clinic_V263_GENETIC_{—} | Clinic diagnosis GENETIC COUNSELING ON PROCREATIVE |
| | COUNSELING_ON_PROCREATIVE_{—} | MANAGEMENT (V263), count during F2: second blood test |
| | MANAGEMENT | (weeks 16-19) |
| 1510 | F2_clinic_242_THYROTOXICOSIS_{—} | Clinic diagnosis THYROTOXICOSIS WITH OR WITHOUT |
| | WITH_OR_WITHOUT_GOITER | GOITER (242), count during F2: second blood test (weeks 16-19) |
| 1511 | F2_clinic_525_DISEASES_AND_{—} | Clinic diagnosis DISEASES AND CONDITIONS OF THE |
| | CONDITIONS_OF_THE_TEETH_{—} | TEETH AND SUPPORT.STRUCTURES (525), count during F2: |
| | AND_SUPPORT_STRUCTURES | second blood test (weeks 16-19) |
| 1512 | F2_clinic_6235_LEUKORRHEA_{—} | Clinic diagnosis LEUKORRHEA, NOT SPECIFIED AS |
| | NOT_SPECIFIED_AS_INFECTIVE | INFECTIVE (6235), count during F2: second blood test |
| | | (weeks 16-19) |
| 1513 | F2_clinic_A97_NO_DISEASE | Clinic diagnosis NO DISEASE (A97), count during F2: second |
| | | blood test (weeks 16-19) |
| 1514 | F2_clinic_78791_DIARRHEA | Clinic diagnosis DIARRHEA (78791), count during F2: second |
| | | blood test (weeks 16-19) |
| 1515 | F2_clinic_2662_OTHER_B_{—} | Clinic diagnosis OTHER B-COMPLEX DEFICIENCIES (2662), |
| | COMPLEX_DEFICIENCIES | count during F2: second blood test (weeks 16-19) |
| 1516 | F2_clinic_7295_PAIN_IN_LIMB | Clinic diagnosis PAIN IN LIMB (7295), count during F2: second |
| | | blood test (weeks 16-19) |
| 1517 | F2_clinic_A03_FEVER | Clinic diagnosis FEVER (A03), count during F2: second blood |
| | | test (weeks 16-19) |
| 1518 | F2_clinic_4660_ACUTE_{—} | Clinic diagnosis ACUTE BRONCHITIS (4660), count during F2: |
| | BRONCHITIS | second blood test (weeks 16-19) |
| 1519 | F2_clinic_1121_CANDIDIASIS_{—} | Clinic diagnosis CANDIDIASIS OF VULVA AND VAGINA |
| | OF_VULVA_AND_VAGINA | (1121), count during F2: second blood test (weeks 16-19) |
| 1520 | F2_clinic_D09_NAUSEA | Clinic diagnosis NAUSEA (D09), count during F2: second blood |
| | | test (weeks 16-19) |
| 1521 | F2_clinic_V221_SUPERVISION_{—} | Clinic diagnosis SUPERVISION OF OTHER NORMAL |
| | OF_OTHER_NORMAL_PREGNANCY | PREGNANCY (V221), count during F2: second blood test (weeks |
| | | 16-19) |
| 1522 | F2_clinic_6260_ABSENCE_OF_{—} | Clinic diagnosis ABSENCE OF MENSTRUATION (6260), count |
| | MENSTRUATION | during F2: second blood test (weeks 16-19) |
| 1523 | F2_clinic_3720_ACUTE_{—} | Clinic diagnosis ACUTE CONJUNCTIVITIS (3720), count |
| | CONJUNCTIVITIS | during F2: second blood test (weeks 16-19) |
| 1524 | F2_clinic_A80_ACCIDENT_{—} | Clinic diagnosis ACCIDENT/INJURY; NOS (A80), count during |
| | OR_INJURY_NOS | F2: second blood test (weeks 16-19) |
| 1525 | F2_clinic_7881_DYSURIA | Clinic diagnosis DYSURIA (7881), count during F2: second |
| | | blood test (weeks 16-19) |
| 1526 | F2_clinic_2809_IRON_DEFICIENCY_{—} | Clinic diagnosis IRON DEFICIENCY ANEMIA, UNSPECIFIED |
| | ANEMIA_UNSPECIFIED | (2809), count during F2: second blood test (weeks 16-19) |
| 1527 | F2_clinic_6400_THREATENED_{—} | Clinic diagnosis THREATENED ABORTION (6400), count |
| | ABORTION | during F2: second blood test (weeks 16-19) |
| 1528 | F2_clinic_V67_FOLLOW_UP_{—} | Clinic diagnosis FOLLOW-UP EXAMINATION (V67), count |
| | EXAMINATION | during F2: second blood test (weeks 16-19) |
| 1529 | F2_clinic_477_ALLERGIC_RHINITIS | Clinic diagnosis ALLERGIC RHINITIS (477), count during F2: |
| | | second blood test (weeks 16-19) |
| 1530 | F2_clinic_493_ASTHMA | Clinic diagnosis ASTHMA (493), count during F2: second blood |
| | | test (weeks 16-19) |
| 1531 | F2_clinic_L08_SHOULDER_{—} | Clinic diagnosis SHOULDER SYMPTOMS/COMPLAINTS |
| | SYMPTOMS_OR_COMPLAINTS | (L08), count during F2: second blood test (weeks 16-19) |
| 1532 | F2_clinic_454_VARICOSE_{—} | Clinic diagnosis VARICOSE VEINS OF LOWER |
| | VEINS_OF_LOWER_EXTREMITIES | EXTREMITIES (454), count during F2: second blood test |
| | | (weeks 16-19) |
| 1533 | F2_clinic_5368_DYSPEPSIA_{—} | Clinic diagnosis DYSPEPSIA AND OTHER SPECIFIED |
| | AND_OTHER_SPECIFIED_{—} | DISORDERS OF FUNCTION OF STOMACH (5368), count |
| | DISORDERS_OF_FUNCTION_{—} | during F2: second blood test (weeks 16-19) |
| | OF_STOMACH | |
| 1534 | F2_clinic_7904_NONSPECIFIC_{—} | Clinic diagnosis NONSPECIFIC ELEVATION OF LEVELS OF |
| | ELEVATION_OF_LEVELS_{—} | TRANSAMINASE OR LDH (7904), count during F2: second |
| | OF_TRANSAMINASE_OR_LDH | blood test (weeks 16-19) |
| 1535 | F2_clinic_2564_POLYCYSTIC_{—} | Clinic diagnosis POLYCYSTIC OVARIES (2564), count during |
| | OVARIES | F2: second blood test (weeks 16-19) |
| 1536 | F2_clinic_Z8936_MANUAL_{—} | Clinic diagnosis MANUAL EXAMINATION OF BREAST |
| | EXAMINATION_OF_BREAST | (Z8936), count during F2: second blood test (weeks 16-19) |
| 1537 | F2_clinic_9599_OTHER_AND_{—} | Clinic diagnosis OTHER AND UNSPECIFIED INJURY TO |
| | UNSPECIFIED_INJURY_TO_{—} | UNSPECIFIED SITE (9599), count during F2: second blood test |
| | UNSPECIFIED_SITE | (weeks 16-19) |
| 1538 | F2_clinic_P027_ISSUE_OF_{—} | Clinic diagnosis ISSUE OF MEDICAL CERTIFICATE (P027), |
| | MEDICAL_CERTIFICATE | count during F2: second blood test (weeks 16-19) |
| 1539 | F2_clinic_6981_PRURITUS_{—} | Clinic diagnosis PRURITUS OF GENITAL ORGANS (6981), |
| | OF_GENITAL_ORGANS | count during F2: second blood test (weeks 16-19) |
| 1540 | F2_clinic_3671_MYOPIA | Clinic diagnosis MYOPIA (3671), count during F2: second blood |
| | | test (weeks 16-19) |
| 1541 | F2_clinic_455_HEMORRHOIDS | Clinic diagnosis HEMORRHOIDS (455), count during F2: second |
| | | blood test (weeks 16-19) |
| 1542 | F2_clinic_1101_{—} | Clinic diagnosis DERMATOPHYTOSIS OF NAIL (1101), count |
| | DERMATOPHYTOSIS_OF_NAIL | during F2: second blood test (weeks 16-19) |
| 1543 | F2_clinic_D10_VOMITING_{—} | Clinic diagnosis VOMITING (EXCL. PREG. W06) (D10), count |
| | EXCL_PREG_W06 | during F2: second blood test (weeks 16-19) |
| 1544 | F2_clinic_2765_VOLUME_{—} | Clinic diagnosis VOLUME DEPLETION DISORDER (2765), |
| | DEPLETION_DISORDER | count during F2: second blood test (weeks 16-19) |
| 1545 | F2_clinic_692_CONTACT_{—} | Clinic diagnosis CONTACT DERMATITIS AND OTHER |
| | DERMATITIS_AND_OTHER_{—} | ECZEMA (692), count during F2: second blood test |
| | ECZEMA | (weeks 16-19) |
| 1546 | F2_clinic_9953_ALLERGY_{—} | Clinic diagnosis ALLERGY, UNSPECIFIED, NOT |
| | UNSPECIFIED_NOT_{—} | ELSEWHERE CLASSIFIED (9953), count during F2: second |
| | ELSEWHERE_CLASSIFIED | blood test (weeks 16-19) |
| 1547 | F2_clinic_9249_CONTUSION_{—} | Clinic diagnosis CONTUSION OF UNSPECIFIED SITE (9249), |
| | OF_UNSPECIFIED_SITE | count during F2: second blood test (weeks 16-19) |
| 1548 | F2_clinic_3887_OTALGIA | Clinic diagnosis OTALGIA (3887), count during F2: second |
| | | blood test (weeks 16-19) |
| 1549 | F2_clinic_6117_SIGNS_{—} | Clinic diagnosis SIGNS AND SYMPTOMS IN BREAST (6117), |
| | AND_SYMPTOMS_IN_BREAST | count during F2: second blood test (weeks 16-19) |
| 1550 | F2_clinic_632_MISSED_{—} | Clinic diagnosis MISSED ABORTION (632), count during F2: |
| | ABORTION | second blood test (weeks 16-19) |
| 1551 | F2_clinic_3804_IMPACTED_{—} | Clinic diagnosis IMPACTED CERUMEN (3804), count during |
| | CERUMEN | F2: second blood test (weeks 16-19) |
| 1552 | F2_clinic_5589_OTHER_AND_{—} | Clinic diagnosis OTHER AND UNSPEC.NONINFECTIOUS |
| | UNSPEC_NONINFECTIOUS_{—} | GASTROENTERITIS AND COLITIS (5589), count during F2: |
| | GASTROENTERITIS_AND_{—} | second blood test (weeks 16-19) |
| | COLITIS | |
| 1553 | F2_clinic_268_VITAMIN_D_{—} | Clinic diagnosis VITAMIN D DEFICIENCY (268), count during |
| | DEFICIENCY | F2: second blood test (weeks 16-19) |
| 1554 | F2_clinic_6261_SCANTY_{—} | Clinic diagnosis SCANTY OR INFREQUENT |
| | OR_INFREQUENT_{—} | MENSTRUATION (6261), count during F2: second blood test |
| | MENSTRUATION | (weeks 16-19) |
| 1555 | F2_clinic_6200_FOLLICULAR_{—} | Clinic diagnosis FOLLICULAR CYST OF OVARY (6200), |
| | CYST_OF_OVARY | count during F2: second blood test (weeks 16-19) |
| 1556 | F2_clinic_7804_DIZZINESS_{—} | Clinic diagnosis DIZZINESS AND GIDDINESS (VERTIGO) |
| | AND_GIDDINESS_VERTIGO | (7804), count during F2: second blood test (weeks 16-19) |
| 1557 | F2_clinic_7862_COUGH | Clinic diagnosis COUGH (7862), count during F2: second blood |
| | | test (weeks 16-19) |
| 1558 | F2_clinic_461_ACUTE_SINUSITIS | Clinic diagnosis ACUTE SINUSITIS (461), count during F2: |
| | | second blood test (weeks 16-19) |
| 1559 | F2_clinic_6430_MILD_{—} | Clinic diagnosis MILD HYPEREMESIS GRAVIDARUM (6430), |
| | HYPEREMESIS_GRAVIDARUM | count during F2: second blood test (weeks 16-19) |
| 1560 | F1_clinic_3730_BLEPHARITIS | Clinic diagnosis BLEPHARITIS (3730), count during F1: first |
| | | blood test (weeks 6-12) |
| 1561 | F1_clinic_69010_SEBORRHEIC_{—} | Clinic diagnosis SEBORRHEIC DERMATITIS, UNSPECIFIED |
| | DERMATITIS_UNSPECIFIED | (69010), count during F1: first blood test (weeks 6-12) |
| 1562 | F1_clinic_9071_LATE_EFFECT_{—} | Clinic diagnosis LATE EFFECT OF INJURY TO CRANIAL |
| | OF_INJURY_TO_CRANIAL_NERVE | NERVE (9071), count during F1: first blood test (weeks 6-12) |
| 1563 | F1_clinic_X18_BREAST_PAIN | Clinic diagnosis BREAST PAIN (X18), count during F1: first |
| | | blood test (weeks 6-12) |
| 1564 | F1_clinic_3731_HORDEOLUM_{—} | Clinic diagnosis HORDEOLUM AND OTHER DEEP |
| | AND_OTHER_DEEP_{—} | INFLAMMATION OF EYELID (3731), count during F1: first |
| | INFLAMMATION_OF_EYELID | blood test (weeks 6-12) |
| 1565 | F1_clinic_28249_OTHER_{—} | Clinic diagnosis OTHER THALASSEMIA (28249), count during |
| | THALASSEMIA | F1: first blood test (weeks 6-12) |
| 1566 | F1_clinic_7243_SCIATICA | Clinic diagnosis SCIATICA (7243), count during F1: first blood |
| | | test (weeks 6-12) |
| 1567 | F1_clinic_5742_CALCULUS_{—} | Clinic diagnosis CALCULUS OF GALLBLADDER WITHOUT |
| | OF_GALLBLADDER_{—} | MENTION OF CHOLECYSTITIS (5742), count during F1: first |
| | WITHOUT_MENTION_OF_{—} | blood test (weeks 6-12) |
| | CHOLECYSTITIS | |
| 1568 | F1_clinic_L03_LOW_BACK_{—} | Clinic diagnosis LOW BACK COMPLT.W/O RADIATION |
| | COMPLT_W_OR_O_RADIATION | (L03), count during F1: first blood test (weeks 6-12) |
| 1569 | F1_clinic_606_INFERTILITY_{—} | Clinic diagnosis INFERTILITY, MALE (606), count during F1: |
| | MALE | first blood test (weeks 6-12) |
| 1570 | F1_clinic_3751_OTHER_{—} | Clinic diagnosis OTHER DISORDERS OF LACRIMAL GLAND |
| | DISORDERS_OF_LACRIMAL_{—} | (3751), count during F1: first blood test (weeks 6-12) |
| | GLAND | |
| 1571 | F1_clinic_W05_VOMITING_{—} | Clinic diagnosis VOMITING/NAUSEA OF PREGNANCY |
| | OR_NAUSEA_OF_PREGNANCY | (W05), count during F1: first blood test (weeks 6-12) |
| 1572 | F1_clinic_3732_CHALAZION | Clinic diagnosis CHALAZION (3732), count during F1: first |
| | | blood test (weeks 6-12) |
| 1573 | F1_clinic_V268_IN_VITRO_{—} | Clinic diagnosis IN VITRO FERTILIZATION OR TEST TUBE |
| | FERTILIZATION_OR_TEST_{—} | PREGNANCY (V268), count during F1: first blood test (weeks 6-12) |
| | TUBE_PREGNANCY | |
| 1574 | F1_clinic_7847_EPISTAXIS | Clinic diagnosis EPISTAXIS (7847), count during F1: first blood |
| | | test (weeks 6-12) |
| 1575 | F1_clinic_3540_CARPAL_{—} | Clinic diagnosis CARPAL TUNNEL SYNDROME (3540), count |
| | TUNNEL_SYNDROME | during F1: first blood test (weeks 6-12) |
| 1576 | F1_clinic_0549_HERPES_{—} | Clinic diagnosis HERPES SIMPLEX WITHOUT MENTION OF |
| | SIMPLEX_WITHOUT_{—} | COMPLICATION (0549), count during F1: first blood test |
| | MENTION_OF_COMPLICATION | (weeks 6-12) |
| 1577 | F1_clinic_B82_ANEMIA_{—} | Clinic diagnosis ANEMIA OTHER/UNSPECIFIED (B82), count |
| | OTHER_OR_UNSPECIFIED | during F1: first blood test (weeks 6-12) |
| 1578 | F1_clinic_5997_HEMATURIA | Clinic diagnosis HEMATURIA (5997), count during F1: first |
| | | blood test (weeks 6-12) |
| 1579 | F1_clinic_7871_HEARTBURN | Clinic diagnosis HEARTBURN (7871), count during F1: first |
| | | blood test (weeks 6-12) |
| 1580 | F1_clinic_5693_HEMORRHAGE_{—} | Clinic diagnosis HEMORRHAGE OF RECTUM AND ANUS |
| | OF_RECTUM_AND_ANUS | (5693), count during F1: first blood test (weeks 6-12) |
| 1581 | F1_clinic_R74_U_R_I_HEAD_COLD | Clinic diagnosis U.R.I. (HEAD COLD) (R74), count during F1: |
| | | first blood test (weeks 6-12) |
| 1582 | F1_clinic_V23_SUPERVISION_{—} | Clinic diagnosis SUPERVISION OF HIGH-RISK PREGNANCY |
| | OF_HIGH_RISK_PREGNANCY | (V23), count during F1: first blood test (weeks 6-12) |
| 1583 | F1_clinic_P760_ISSUE_OF_{—} | Clinic diagnosis ISSUE OF REPEAT PRESCRIPTION (P760), |
| | REPEAT_PRESCRIPTION | count during F1: first blood test (weeks 6-12) |
| 1584 | F1_clinic_5282_ORAL_APHTHAE | Clinic diagnosis ORAL APHTHAE (5282), count during F1: first |
| | | blood test (weeks 6-12) |
| 1585 | F1_clinic_7048_OTHER_{—} | Clinic diagnosis OTHER SPECIFIED DISEASES OF HAIR |
| | SPECIFIED_DISEASES_OF_{—} | AND HAIR FOLLICLES (7048), count during F1: first blood test |
| | HAIR_AND_HAIR_FOLLICLES | (weeks 6-12) |
| 1586 | F2_clinic_5990_URINARY_{—} | Clinic diagnosis URINARY TRACT INFECTION, SITE NOT |
| | TRACT_INFECTION_SITE_{—} | SPECIFIED (5990), count during F2: second blood test |
| | NOT_SPECIFIED | (weeks 16-19) |
| 1587 | F2_clinic_4_NOTHING_{—} | Clinic diagnosis NOTHING ABNORMAL FOUND (4), count |
| | ABNORMAL_FOUND | during F2: second blood test (weeks 16-19) |
| 1588 | F2_clinic_463_ACUTE_{—} | Clinic diagnosis ACUTE TONSILLITIS (463), count during F2: |
| | TONSILLITIS | second blood test (weeks 16-19) |
| 1589 | F2_clinic_Y10_INFERTILITY_{—} | Clinic diagnosis INFERTILITY/SUBFERTILITY (Y10), count |
| | OR_SUBFERTILITY | during F2: second blood test (weeks 16-19) |
| 1590 | F2_clinic_7870_NAUSEA_{—} | Clinic diagnosis NAUSEA AND VOMITING (7870), count |
| | AND_VOMITING | during F2: second blood test (weeks 16-19) |
| 1591 | F2_clinic_7242_LUMBAGO | Clinic diagnosis LUMBAGO (7242), count during F2: second |
| | | blood test (weeks 16-19) |
| 1592 | F2_clinic_W78_PREGNANCY_{—} | Clinic diagnosis PREGNANCY: CONFIRMED (W78), count |
| | CONFIRMED | during F2: second blood test (weeks 16-19) |
| 1593 | F2_clinic_0799_UNSP_{—} | Clinic diagnosis UNSPVIRAL INFECT.IN CONDITIONS |
| | VIRAL_INFECT_IN_{—} | CLASSIF.ELSEWHERE, UNSP.SITE (0799), count during F2: |
| | CONDITIONS_CLASSIF_{—} | second blood test (weeks 16-19) |
| | ELSEWHERE_UNSP_SITE | |
| 1594 | F2_clinic_4659_ACUTE_{—} | Clinic diagnosis ACUTE UPPER RESPIRATORY INFECTIONS |
| | UPPER_RESPIRATORY_{—} | OF UNSPECIFIED SITE (4659), count during F2: second blood |
| | INFECTIONS_OF_{—} | test (weeks 16-19) |
| | UNSPECIFIED_SITE | |
| 1595 | F2_clinic_Z8926_{—} | Clinic diagnosis GYNECOLOGICAL EXAMINATION (Z8926), |
| | GYNECOLOGICAL_{—} | count during F2: second blood test (weeks 16-19) |
| | EXAMINATION | |
| 1596 | F2_clinic_V726_LABORATORY_{—} | Clinic diagnosis LABORATORY EXAMINATION (V726), |
| | EXAMINATION | count during F2: second blood test (weeks 16-19) |
| 1597 | F2_clinic_628_INFERTILITY_FEMALE | Clinic diagnosis INFERTILITY, FEMALE (628), count during |
| | | F2: second blood test (weeks 16-19) |
| 1598 | F1_clinic_3000_ANXIETY_STATES | Clinic diagnosis ANXIETY STATES (3000), count during F1: |
| | | first blood test (weeks 6-12) |
| 1599 | F2_clinic_V828_SCREENING_{—} | Clinic diagnosis SCREENING FOR OTHER SPECIFIED |
| | FOR_OTHER_SPECIFIED_{—} | CONDITIONS (V828), count during F2: second blood test (weeks |
| | CONDITIONS | 16-19) |
| 1600 | F2_clinic_7890_ABDOMINAL_{—} | Clinic diagnosis ABDOMINAL PAIN (7890), count during F2: |
| | PAIN | second blood test (weeks 16-19) |
| 1601 | F2_clinic_0_OTHER | Clinic diagnosis OTHER (0), count during F2: second blood test |
| | | (weeks 16-19) |
| 1602 | F1_clinic_286_COAGULATION_{—} | Clinic diagnosis COAGULATION DEFECTS (286), count during |
| | DEFECTS | F1: first blood test (weeks 6-12) |
| 1603 | F1_clinic_8450_ANKLE_SPRAIN | Clinic diagnosis ANKLE SPRAIN (8450), count during F1: first |
| | | blood test (weeks 6-12) |
| 1604 | F1_clinic_5559_REGIONAL_{—} | Clinic diagnosis REGIONAL ENTERITIS OF UNSPECIFIED |
| | ENTERITIS_OF_UNSPECIFIED_{—} | SITE (5559), count during F1: first blood test (weeks 6-12) |
| | SITE | |
| 1605 | F1_clinic_218_UTERINE_{—} | Clinic diagnosis UTERINE LEIOMYOMA (218), count during |
| | LEIOMYOMA | F1: first blood test (weeks 6-12) |
| 1606 | F1_clinic_6465_ASYMPTOMATIC_{—} | Clinic diagnosis ASYMPTOMATIC BACTERIURIA IN |
| | BACTERIURIA_IN_PREGNANCY | PREGNANCY (6465), count during F1: first blood test |
| | | (weeks 6-12) |
| 1607 | F1_clinic_790_NONSPECIFIC_{—} | Clinic diagnosis NONSPECIFIC FINDINGS ON |
| | FINDINGS_ON_EXAMINATION_{—} | EXAMINATION OF BLOOD (790), count during F1: first blood |
| | OF_BLOOD | test (weeks 6-12) |
| 1608 | F1_clinic_S12_INSECT_BITE | Clinic diagnosis INSECT BITE (S12), count during F1: first |
| | | blood test (weeks 6-12) |
| 1609 | F2_clinic_7061_OTHER_ACNE | Clinic diagnosis OTHER ACNE (7061), count during F2: second |
| | | blood test (weeks 16-19) |
| 1610 | F2_clinic_799_OTHER_ILL_{—} | Clinic diagnosis OTHER ILL-DEFINED AND UNKNOWN |
| | DEFINED_AND_UNKNOWN_{—} | CAUSES OF MORBIDITY AND MORTALITY (799), count |
| | CAUSES_OF_MORBIDITY_{—} | during F2: second blood test (weeks 16-19) |
| | AND_MORTALITY | |
| 1611 | F0_pharma_G01AF02_{—} | Prescription of AGISTEN VAG TAB 200 mg 3 (G01AF02), count |
| | AGISTEN_VAG_TAB_200mg_3 | during F0: before and early pregnancy |
| 1612 | F0_hospital_6288_INFERTILITY_{—} | Hospital diagnosis INFERTILITY, FEMALE. OF OTHER |
| | FEMALE_OF_OTHER_{—} | SPECIFIED ORIGIN (6288), count during F0: before and early |
| | SPECIFIED_ORIGIN | pregnancy |
| 1613 | F0_hospital_Z6902_DILATION_{—} | Hospital diagnosis DILATION AND CURETTAGE |
| | AND_CURETTAGE_FOLLOWING_{—} | FOLLOWING DELIVERY OR ABORTION (Z6902), count |
| | DELIVERY_OR_ABORTION | during F0: before and early pregnancy |
| 1614 | F0_hospital_V221_SUPERVISION_{—} | Hospital diagnosis SUPERVISION OF OTHER NORMAL |
| | OF_OTHER_NORMAL_PREGNANCY | PREGNANCY (V221), count during F0: before and early |
| | | pregnancy |
| 1615 | F0_hospital_Z65910_I_V_F | Hospital diagnosis I.V.F. (Z65910), count during F0: before and |
| | | early pregnancy |
| 1616 | F0_hospital_78900_ABDOMINAL_{—} | Hospital diagnosis ABDOMINAL PAIN, UNSP. SITE (78900), |
| | PAIN_UNSP_SITE | count during F0: before and early pregnancy |
| 1617 | F0_hospital_632_MISSED_ABORTION | Hospital diagnosis MISSED ABORTION (632), count during F0: |
| | | before and early pregnancy |
| 1618 | F0_hospital_64303_MILD_{—} | Hospital diagnosis MILD HYPEREMESIS GRAVIDARUM, |
| | HYPEREMESIS_GRAVIDARUM_{—} | ANTEPARTUM (64303), count during F0: before and early |
| | ANTEPARTUM | pregnancy |
| 1619 | F0_hospital_V222_PREGNANT_{—} | Hospital diagnosis PREGNANT STATE, INCIDENTAL (V222), |
| | STATE_INCIDENTAL | count during F0: before and early pregnancy |
| 1620 | F0_hospital_0_OTHER | Hospital diagnosis OTHER (0), count during F0: before and early |
| | | pregnancy |
| 1621 | F2_clinic_07819_OTHER_{—} | Clinic diagnosis OTHER SPECIFIED VIRAL WARTS (07819), |
| | SPECIFIED_VIRAL_WARTS | count during F2: second blood test (weeks 16-19) |
| 1622 | F2_clinic_8450_ANKLE_SPRAIN | Clinic diagnosis ANKLE SPRAIN (8450), count during F2: |
| | | second blood test (weeks 16-19) |
| 1623 | F2_clinic_9223_CONTUSION_OF_BACK | Clinic diagnosis CONTUSION OF BACK (9223), count during |
| | | F2: second blood test (weeks 16-19) |
| 1624 | F2_clinic_5559_REGIONAL_{—} | Clinic diagnosis REGIONAL ENTERITIS OF UNSPECIFIED |
| | ENTERITIS_OF_UNSPECIFIED_SITE | SITE (5559), count during F2: second blood test (weeks 16-19) |
| 1625 | F2_clinic_218_UTERINE_{—} | Clinic diagnosis UTERINE LEIOMYOMA (218), count during |
| | LEIOMYOMA | F2: second blood test (weeks 16-19) |
| 1626 | F2_clinic_7880_RENAL_COLIC | Clinic diagnosis RENAL COLIC (7880), count during F2: second |
| | | blood test (weeks 16-19) |
| 1627 | F2_clinic_790_NONSPECIFIC_{—} | Clinic diagnosis NONSPECIFIC FINDINGS ON |
| | FINDINGS_ON_EXAMINATION_{—} | EXAMINATION OF BLOOD (790), count during F2: second |
| | OF_BLOOD | blood test (weeks 16-19) |
| 1628 | F2_clinic_S12_INSECT_BITE | Clinic diagnosis INSECT BITE (S12), count during F2: second |
| | | blood test (weeks 16-19) |
| 1629 | F2_clinic_7270_SYNOVITIS_AND_{—} | Clinic diagnosis SYNOVITIS AND TENOSYNOVITIS (7270), |
| | TENOSYNOVITIS | count during F2: second blood test (weeks 16-19) |
| 1630 | F2_clinic_3000_ANXIETY_STATES | Clinic diagnosis ANXIETY STATES (3000), count during F2: |
| | | second blood test (weeks 16-19) |
| 1631 | F2_clinic_7048_OTHER_{—} | Clinic diagnosis OTHER SPECIFIED DISEASES OF HAIR |
| | SPECIFIED_DISEASES_OF_{—} | AND HAIR FOLLICLES (7048), count during F2: second blood |
| | HAIR_AND_HAIR_FOLLICLES | test (weeks 16-19) |
| 1632 | F2_clinic_5282_ORAL_APHTHAE | Clinic diagnosis ORAL APHTHAE (5282), count during F2: |
| | | second blood test (weeks 16-19) |
| 1633 | F2_clinic_P760_ISSUE_OF_{—} | Clinic diagnosis ISSUE OF REPEAT PRESCRIPTION (P760), |
| | REPEAT_PRESCRIPTION | count during F2: second blood test (weeks 16-19) |
| 1634 | F2_clinic_3730_BLEPHARITIS | Clinic diagnosis BLEPHARITIS (3730), count during F2: second |
| | | blood test (weeks 16-19) |
| 1635 | F2_clinic_69010_SEBORRHEIC_{—} | Clinic diagnosis SEBORRHEIC DERMATITIS, UNSPECIFIED |
| | DERMATITIS_UNSPECIFIED | (69010), count during F2: second blood test (weeks 16-19) |
| 1636 | F0_hospital_64003_THREATENED_{—} | Hospital diagnosis THREATENED ABORTION, |
| | ABORTION_ANTEPARTUM | ANTEPARTUM (64003), count during F0: before and early |
| | | pregnancy |
| 1637 | F0_hospital_Z65911_E_T | Hospital diagnosis E.T. (Z65911), count during F0: before and |
| | | early pregnancy |
| 1638 | F2_clinic_L04_CHEST_SYMPTOMS_{—} | Clinic diagnosis CHEST SYMPTOMS/COMPLAINTS (L04), |
| | OR_COMPLAINTS | count during F2: second blood test (weeks 16-19) |
| 1639 | F0_hospital_Z96490_ABORTION_{—} | Hospital diagnosis ABORTION INDUCED BY MEDICATION |
| | INDUCED_BY_MEDICATION | (Z96490), count during F0: before and early pregnancy |
| 1640 | F0_hospital_V714_OBSERVATION_{—} | Hospital diagnosis OBSERVATION FOLLOWING OTHER |
| | FOLLOWING_OTHER_ACCIDENT | ACCIDENT (V714), count during F0: before and early pregnancy |
| 1641 | F0_hospital_0_UNKNOWN_CATEGORY | Hospital diagnosis UNKNOWN CATEGORY (0), count during |
| | | F0: before and early pregnancy |
| 1642 | F0_hospital_2449_UNSPECIFIED_{—} | Hospital diagnosis UNSPECIFIED ACQUIRED |
| | ACQUIRED_HYPOTHYROIDISM | HYPOTHYROIDISM (2449), count during F0: before and early |
| | | pregnancy |
| 1643 | F0_hospital_V220_SUPERVISION_{—} | Hospital diagnosis SUPERVISION OF NORMAL FIRST |
| | OF_NORMAL_FIRST_PREGNANCY | PREGNANCY (V220), count during F0: before and early |
| | | pregnancy |
| 1644 | F0_hospital_Z6992_ARTIFICIAL_{—} | Hospital diagnosis ARTIFICIAL INSEMINATION (Z6992), |
| | INSEMINATION | count during F0: before and early pregnancy |
| 1645 | F0_hospital_63491_SPONTANEOUS_{—} | Hospital diagnosis SPONTANEOUS |
| | ABORTION_INCOMPLETE_{—} | ABORTION, INCOMPLETE, WITHOUT COMPLICATION |
| | WITHOUT_COMPLICATION | (63491), count during F0: before and early pregnancy |
| 1646 | F0_hospital_Z6591_ASPIRATION_{—} | Hospital diagnosis ASPIRATION OF OVARY (Z6591), count |
| | OF_OVARY | during F0: before and early pregnancy |
| 1647 | F0_hospital_78903_ABDOMINAL_{—} | Hospital diagnosis ABDOMINAL PAIN, RT. LOWER |
| | PAIN_RT_LOWER_QUADRANT | QUADRANT (78903), count during F0: before and early |
| | | pregnancy |
| 1648 | F0_hospital_65103_TWIN_{—} | Hospital diagnosis TWIN PREGNANCY, ANTEPARTUM |
| | PREGNANCY_ANTEPARTUM_{—} | CONDITION OR COMPLICATION (65103), count during F0: |
| | CONDITION_OR_COMPLICATION | before and early pregnancy |
| 1649 | F0_hospital_7890_ABDOMINAL_PAIN | Hospital diagnosis ABDOMINAL PAIN (7890), count during F0: |
| | | before and early pregnancy |
| 1650 | F0_hospital_V2385_SUPERVISION_{—} | Hospital diagnosis SUPERVISION OF HIGH RISK |
| | OF_HIGH_RISK_PREGNANCY_{—} | PREGNANCY RESULTING FROM ASSISTED REPROD |
| | RESULTING_FROM_ASSISTED_{—} | UCTIVE TECHNOLOGY (V2385), count during F0: before and |
| | REPROD_UCTIVE_TECHNOLOGY | early pregnancy |
| 1651 | F0_hospital_65423_UTERINE_{—} | Hospital diagnosis UTERINE SCAR FROM |
| | SCAR_FROM_PREV_SURGERY_{—} | PREV.SURGERY, ANTEPARTUM CONDITION/COMPLIC. |
| | ANTEPARTUM_CONDITION_OR_{—} | (65423), count during F0: before and early pregnancy |
| | COMPLIC | |
| 1652 | F0_hospital_V140_PERSONAL_{—} | Hospital diagnosis PERSONAL HISTORY OF ALLERGY TO |
| | HISTORY_OF_ALLERGY_TO_{—} | PENICILLIN (V140), count during F0: before and early |
| | PENICILLIN | pregnancy |
| 1653 | F0_hospital_78909_ABDOMINAL_{—} | Hospital diagnosis ABDOMINAL PAIN, OTHER SPECIFIED |
| | PAIN_OTHER_SPECIFIED_SITE | SITE (78909), count during F0: before and early pregnancy |
| 1654 | F0_hospital_65453_CERVICAL_{—} | Hospital diagnosis CERVICAL INCOMPETENCE, |
| | INCOMPETENCE_ANTEPARTUM_{—} | ANTEPARTUM CONDITION OR COMPLICATION (65453), |
| | CONDITION_OR_COMPLICATION | count during F0: before and early pregnancy |
| 1655 | F0_hospital_64300_MILD_{—} | Hospital diagnosis MILD HYPEREMESIS GRAVIDARUM, |
| | HYPEREMESIS_GRAVIDARUM_{—} | UNSP.AS TO EPISODE OF CARE (64300), count during F0: |
| | UNSP_AS_TO_EPISODE_OF_CARE | before and early pregnancy |
| 1656 | F0_hospital_Z8878_DIAGNOSTIC_{—} | Hospital diagnosis DIAGNOSTIC ULTRASOUND OF GRAVID |
| | ULTRASOUND_OF_GRAVID_UTERUS | UTERUS (Z8878), count during F0: before and early pregnancy |
| 1657 | F0_hospital_5990_URINARY_{—} | Hospital diagnosis URINARY TRACT INFECTION, SITE NOT |
| | TRACT_INFECTION_SITE_NOT_{—} | SPECIFIED (5990), count during F0: before and early pregnancy |
| | SPECIFIED | |
| 1658 | F0_hospital_999999 | Hospital diagnosis (999999), count during F0: before and early |
| | | pregnancy |
| 1659 | F0_hospital_V22_NORMAL_{—} | Hospital diagnosis NORMAL PREGNANCY (V22), count during |
| | PREGNANCY | F0: before and early pregnancy |
| 1660 | F0_hospital_Z8879_OTHER_{—} | Hospital diagnosis OTHER DIAGNOSTIC ULTRASOUND |
| | DIAGNOSTIC_ULTRASOUND | (Z8879), count during F0: before and early pregnancy |
| 1661 | F0_hospital_Z6952_ASPIRATION_{—} | Hospital diagnosis ASPIRATION CURETTAGE FOLLOWING |
| | CURETTAGE_FOLLOWING_{—} | DELIVERY OR ABORTION (Z6952), count during F0: before |
| | DELIVERY_OR_ABORTION | and early pregnancy |
| 1662 | F0_hospital_64663_ANTEPARTUM_{—} | Hospital diagnosis ANTEPARTUM INFECTIONS OF |
| | INFECTIONS_OF_GENITOURINARY_{—} | GENITOURINARY TRACT (64663), count during F0: before |
| | TRACT | and early pregnancy |
| 1663 | F2_clinic_9071_LATE_EFFECT_{—} | Clinic diagnosis LATE EFFECT OF INJURY TO CRANIAL |
| | OF_INJURY_TO_CRANIAL_NERVE | NERVE (9071), count during F2: second blood test (weeks 16-19) |
| 1664 | F2_clinic_3731_HORDEOLUM_{—} | Clinic diagnosis HORDEOLUM AND OTHER DEEP |
| | AND_OTHER_DEEP_{—} | INFLAMMATION OF EYELID (3731), count during F2: second |
| | INFLAMMATION_OF_EYELID | blood test (weeks 16-19) |
| 1665 | F2_clinic_6221_DYSPLASIA_OF_{—} | Clinic diagnosis DYSPLASIA OF CERVIX (UTERI) (6221), |
| | CERVIX_UTERI | count during F2: second blood test (weeks 16-19) |
| 1666 | F2_clinic_28249_OTHER_{—} | Clinic diagnosis OTHER THALASSEMIA (28249), count during |
| | THALASSEMIA | F2: second blood test (weeks 16-19) |
| 1667 | F2_clinic_V700_ROUTINE_{—} | Clinic diagnosis ROUTINE GENERAL MEDICAL |
| | GENERAL_MEDICAL_{—} | EXAMINATION AT A HEALTH CARE FACILITY (V700), |
| | EXAMINATION_AT_A_{—} | count during F2: second blood test (weeks 16-19) |
| | HEALTH_CARE_FACILITY | |
| 1668 | F2_clinic_V724_PREGNANCY_{—} | Clinic diagnosis PREGNANCY EXAMINATION OR TEST, |
| | EXAMINATION_OR_TEST_{—} | PREGNANCY UNCONFIRMED (V724), count during F2: |
| | PREGNANCY_UNCONFIRMED | second blood test (weeks 16-19) |
| 1669 | F2_clinic_345_EPILEPSY | Clinic diagnosis EPILEPSY (345), count during F2: second blood |
| | | test (weeks 16-19) |
| 1670 | F2_clinic_595_CYSTITIS | Clinic diagnosis CYSTITIS (595), count during F2: second blood |
| | | test (weeks 16-19) |
| 1671 | F2_clinic_V7610_BREAST_{—} | Clinic diagnosis BREAST SCREENING FOR MALIGNANT |
| | SCREENING_FOR_{—} | NEOPLASMS, UNSP. (V7610), count during F2: second blood |
| | MALIGNANT_NEOPLASMS_{—} | test (weeks 16-19) |
| | UNSP | |
| 1672 | F2_clinic_7820_DISTURBANCE_{—} | Clinic diagnosis DISTURBANCE OF SKIN SENSATION |
| | OF_SKIN_SENSATION | (7820), count during F2: second blood test (weeks 16-19) |
| 1673 | F2_clinic_216_BENIGN_{—} | Clinic diagnosis BENIGN NEOPLASM OF SKIN (216), count |
| | NEOPLASM_OF_SKIN | during F2: second blood test (weeks 16-19) |
| 1674 | F2_clinic_7821_RASH_AND_{—} | Clinic diagnosis RASH AND OTHER NONSPECIFIC SKIN |
| | OTHER_NONSPECIFIC_SKIN_{—} | ERUPTION (7821), count during F2: second blood test |
| | ERUPTION | (weeks 16-19) |
| 1675 | F2_clinic_1104_DERMATOPHYTOSIS_{—} | Clinic diagnosis DERMATOPHYTOSIS OF FOOT (1104), count |
| | OF_FOOT | during F2: second blood test (weeks 16-19) |
| 1676 | F2_clinic_6264_IRREGULAR_{—} | Clinic diagnosis IRREGULAR MENSTRUAL CYCLE (6264), |
| | MENSTRUAL_CYCLE | count during F2: second blood test (weeks 16-19) |
| 1677 | F2_clinic_7856_ENLARGEMENT_{—} | Clinic diagnosis ENLARGEMENT OF LYMPH NODES (7856), |
| | OF_LYMPH_NODES | count during F2: second blood test (weeks 16-19) |
| 1678 | F2_clinic_244_ACQUIRED_{—} | Clinic diagnosis ACQUIRED HYPOTHYROIDISM (244), count |
| | HYPOTHYROIDISM | during F2: second blood test (weeks 16-19) |
| 1679 | F2_clinic_6220_EROSION_{—} | Clinic diagnosis EROSION AND ECTROPION OF CERVIX |
| | AND_ECTROPION_OF_CERVIX | (6220), count during F2: second blood test (weeks 16-19) |
| 1680 | F2_clinic_7040_ALOPECIA | Clinic diagnosis ALOPECIA (7040), count during F2: second |
| | | blood test (weeks 16-19) |
| 1681 | F2_clinic_V71_OBSERVATION_{—} | Clinic diagnosis OBSERVATION AND EVALUATION FOR |
| | AND_EVALUATION_FOR_{—} | SUSPECTED CONDITIONS (V71), count during F2: second |
| | SUSPECTED_CONDITIONS | blood test (weeks 16-19) |
| 1682 | F2_clinic_V25_CONTRACEPTIVE_{—} | Clinic diagnosis CONTRACEPTIVE MANAGEMENT (V25), |
| | MANAGEMENT | count during F2: second blood test (weeks 16-19) |
| 1683 | F2_clinic_634_SPONTANEOUS_{—} | Clinic diagnosis SPONTANEOUS ABORTION (634), count |
| | ABORTION | during F2: second blood test (weeks 16-19) |
| 1684 | F2_clinic_7090_DYSCHROMIA_{—} | Clinic diagnosis DYSCHROMIA, ANOMALOUS |
| | ANOMALOUS_PIGMENTATION | PIGMENTATION (7090), count during F2: second blood test |
| | | (weeks 16-19) |
| 1685 | F2_clinic_7291_MYALGIA_{—} | Clinic diagnosis MYALGIA AND MYOSITIS, UNSPECIFIED |
| | AND_MYOSITIS_UNSPECIFIED | (7291), count during F2: second blood test (weeks 16-19) |
| 1686 | F2_clinic_5350_GASTRITIS | Clinic diagnosis GASTRITIS (5350), count during F2: second |
| | | blood test (weeks 16-19) |
| 1687 | F2_clinic_S96_ACNE | Clinic diagnosis ACNE (S96), count during F2: second blood test |
| | | (weeks 16-19) |
| 1688 | F2_clinic_6409_UNSPECIFIED_{—} | Clinic diagnosis UNSPECIFIED HEMORRHAGE IN EARLY |
| | HEMORRHAGE_IN_EARLY_{—} | PREGNANCY (6409), count during F2: second blood test |
| | PREGNANCY | (weeks 16-19) |
| 1689 | F2_clinic_5301_ESOPHAGITIS | Clinic diagnosis ESOPHAGITIS (5301), count during F2: second |
| | | blood test (weeks 16-19) |
| 1690 | F2_clinic_5650_ANAL_FISSURE | Clinic diagnosis ANAL FISSURE (5650), count during F2: |
| | | second blood test (weeks 16-19) |
| 1691 | F2_clinic_Z74_CESAREAN_{—} | Clinic diagnosis CESAREAN SECTION AND REMOVAL OF |
| | SECTION_AND_REMOVAL_{—} | FETUS (Z74), count during F2: second blood test (weeks 16-19) |
| | OF_FETUS | |
| 1692 | F2_clinic_7062_SEBACEOUS_{—} | Clinic diagnosis SEBACEOUS CYST (7062), count during F2: |
| | CYST | second blood test (weeks 16-19) |
| 1693 | F2_clinic_7871_HEARTBURN | Clinic diagnosis HEARTBURN (7871), count during F2: second |
| | | blood test (weeks 16-19) |
| 1694 | F2_clinic_5742_CALCULUS_{—} | Clinic diagnosis CALCULUS OF GALLBLADDER WITHOUT |
| | OF_GALLBLADDER_WITHOUT_{—} | MENTION OF CHOLECYSTITIS (5742), count during F2: |
| | MENTION_OF_CHOLECYSTITIS | second blood test (weeks 16-19) |
| 1695 | F2_clinic_L03_LOW_BACK_{—} | Clinic diagnosis LOW BACK COMPLT.W/O RADIATION |
| | COMPLT_W_OR_O_RADIATION | (L03), count during F2: second blood test (weeks 16-19) |
| 1696 | F2_clinic_606_INFERTILITY_MALE | Clinic diagnosis INFERTILITY, MALE (606), count during F2: |
| | | second blood test (weeks 16-19) |
| 1697 | F2_clinic_3751_OTHER_{—} | Clinic diagnosis OTHER DISORDERS OF LACRIMAL GLAND |
| | DISORDERS_OF_LACRIMAL_{—} | (3751), count during F2: second blood test (weeks 16-19) |
| | GLAND | |
| 1698 | F2_clinic_W05_VOMITING_{—} | Clinic diagnosis VOMITING/NAUSEA OF PREGNANCY |
| | OR_NAUSEA_OF_PREGNANCY | (W05), count during F2: second blood test (weeks 16-19) |
| 1699 | F2_clinic_3732_CHALAZION | Clinic diagnosis CHALAZION (3732), count during F2: second |
| | | blood test (weeks 16-19) |
| 1700 | F2_clinic_708_URTICARIA | Clinic diagnosis URTICARIA (708), count during F2: second |
| | | blood test (weeks 16-19) |
| 1701 | F2_clinic_7019_UNSPECIFIED_{—} | Clinic diagnosis UNSPECIFIED HYPERTROPHIC AND |
| | HYPERTROPHIC_AND_{—} | ATROPHIC CONDITIONS OF SKIN (7019), count during F2: |
| | ATROPHIC_CONDITIONS_{—} | second blood test (weeks 16-19) |
| | OF_SKIN | |
| 1702 | F2_clinic_B82_ANEMIA_{—} | Clinic diagnosis ANEMIA OTHER/UNSPECIFIED (B82), count |
| | OTHER_OR_UNSPECIFIED | during F2: second blood test (weeks 16-19) |
| 1703 | F2_clinic_5997_HEMATURIA | Clinic diagnosis HEMATURIA (5997), count during F2: second |
| | | blood test (weeks 16-19) |
| 1704 | F2_clinic_5693_HEMORRHAGE_{—} | Clinic diagnosis HEMORRHAGE OF RECTUM AND ANUS |
| | OF_RECTUM_AND_ANUS | (5693), count during F2: second blood test (weeks 16-19) |
| 1705 | F2_clinic_1110_PITYRIASIS_{—} | Clinic diagnosis PITYRIASIS VERSICOLOR (1110), count |
| | VERSICOLOR | during F2: second blood test (weeks 16-19) |
| 1706 | F2_clinic_3_AFTERCARE_{—} | Clinic diagnosis AFTERCARE CHECK-UP NO DISEASE (3), |
| | CHECK_UP_NO_DISEASE | count during F2: second blood test (weeks 16-19) |
| 1707 | F2_clinic_R74_U_R_I_HEAD_{—} | Clinic diagnosis U.R.I. (HEAD COLD) (R74), count during F2: |
| | COLD | second blood test (weeks 16-19) |
| 1708 | F2_clinic_6918_OTHER_ATOPIC_{—} | Clinic diagnosis OTHER ATOPIC DERMATITIS AND |
| | DERMATITIS_AND_RELATED_{—} | RELATED CONDITIONS (6918), count during F2: second blood |
| | CONDITIONS | test (weeks 16-19) |
| 1709 | F2_clinic_V163_FAMILY_{—} | Clinic diagnosis FAMILY HISTORY OF MALIGNANT |
| | HISTORY_OF_MALIGNANT_{—} | NEOPLASM OF BREAST (V163), count during F2: second |
| | NEOPLASM_OF_BREAST | blood test (weeks 16-19) |
| 1710 | F2_clinic_678_OTHER_{—} | Clinic diagnosis OTHER FETAL CONDITIONS (678), count |
| | FETAL_CONDITIONS | during F2: second blood test (weeks 16-19) |
| 1711 | F2_clinic_6793_ORAL_{—} | Clinic diagnosis ORAL CONTRACEPTION PRESCRIPTION |
| | CONTRACEPTION_PRESCRIPTION | (6793), count during F2: second blood test (weeks 16-19) |
| 1712 | F2_clinic_6267_POSTCOITAL_{—} | Clinic diagnosis POSTCOITAL BLEEDING (6267), count during |
| | BLEEDING | F2: second blood test (weeks 16-19) |
| 1713 | F2_clinic_27800_OBESITY_{—} | Clinic diagnosis OBESITY, UNSPECIFIED (27800), count |
| | UNSPECIFIED | during F2: second blood test (weeks 16-19) |
| 1714 | F2_clinic_5246_{—} | Clinic diagnosis TEMPOROMANDIBULAR JOINT |
| | TEMPOROMANDIBULAR_{—} | DISORDERS (5246), count during F2: second blood test |
| | JOINT_DISORDERS | (weeks 16-19) |
| 1715 | F2_clinic_07810_VIRAL_{—} | Clinic diagnosis VIRAL WARTS, UNSPECIFIED (07810), count |
| | WARTS_UNSPECIFIED | during F2: second blood test (weeks 16-19) |
| 1716 | F1_clinic_2872_OTHER_{—} | Clinic diagnosis OTHER NONTHROMBOCYTOPENIC |
| | NONTHROMBOCYTOPENIC_{—} | PURPURAS (2872), count during F1: first blood test |
| | PURPURAS | (weeks 6-12) |
| 1717 | F1_clinic_V163_FAMILY_{—} | Clinic diagnosis FAMILY HISTORY OF MALIGNANT |
| | HISTORY_OF_MALIGNANT_{—} | NEOPLASM OF BREAST (V163), count during F1: first blood |
| | NEOPLASM_OF_BREAST | test (weeks 6-12) |
| 1718 | F1_clinic_2452_CHRONIC_{—} | Clinic diagnosis CHRONIC LYMPHOCYTIC THYROIDITIS |
| | LYMPHOCYTIC_THYROIDITIS | (2452), count during F1: first blood test (weeks 6-12) |
| 1719 | F0_clinic_7871_HEARTBURN | Clinic diagnosis HEARTBURN (7871), count during F0: before |
| | | and early pregnancy |
| 1720 | F0_clinic_3_AFTERCARE_{—} | Clinic diagnosis AFTERCARE CHECK-UP NO DISEASE (3), |
| | CHECK_UP_NO_DISEASE | count during F0: before and early pregnancy |
| 1721 | F0_clinic_6918_OTHER_{—} | Clinic diagnosis OTHER ATOPIC DERMATITIS AND |
| | ATOPIC_DERMATITIS_AND_{—} | RELATED CONDITIONS (6918), count during F0: before and |
| | RELATED_CONDITIONS | early pregnancy |
| 1722 | F0_clinic_V23_SUPERVISION_OF_{—} | Clinic diagnosis SUPERVISION OF HIGH-RISK PREGNANCY |
| | HIGH_RISK_PREGNANCY | (V23), count during F0: before and early pregnancy |
| 1723 | F0_clinic_2872_OTHER_{—} | Clinic diagnosis OTHER NONTHROMBOCYTOPENIC |
| | NONTHROMBOCYTOPENIC_{—} | PURPURAS (2872), count during F0: before and early pregnancy |
| | PURPURAS | |
| 1724 | F0_clinic_V163_FAMILY_{—} | Clinic diagnosis FAMILY HISTORY OF MALIGNANT |
| | HISTORY_OF_MALIGNANT_{—} | NEOPLASM OF BREAST (V163), count during F0: before and |
| | NEOPLASM_OF_BREAST | early pregnancy |
| 1725 | F0_clinic_6793_ORAL_{—} | Clinic diagnosis ORAL CONTRACEPTION PRESCRIPTION |
| | CONTRACEPTION_PRESCRIPTION | (6793), count during F0: before and early pregnancy |
| 1726 | F0_clinic_6267_POSTCOITAL_{—} | Clinic diagnosis POSTCOITAL BLEEDING (6267), count during |
| | BLEEDING | F0: before and early pregnancy |
| 1727 | F0_clinic_6829_CELLULITIS_{—} | Clinic diagnosis CELLULITIS AND ABSCESS OF |
| | AND_ABSCESS_OF_{—} | UNSPECIFIED SITES (6829), count during F0: before and early |
| | UNSPECIFIED_SITES | pregnancy |
| 1728 | F0_clinic_27800_OBESITY_{—} | Clinic diagnosis OBESITY, UNSPECIFIED (27800), count |
| | UNSPECIFIED | during F0: before and early pregnancy |
| 1729 | F0_clinic_07810_VIRAL_{—} | Clinic diagnosis VIRAL WARTS, UNSPECIFIED (07810), count |
| | WARTS_UNSPECIFIED | during F0: before and early pregnancy |
| 1730 | F0_clinic_1110_PITYRIASIS_{—} | Clinic diagnosis PITYRIASIS VERSICOLOR (1110), count |
| | VERSICOLOR | during F0: before and early pregnancy |
| 1731 | F0_clinic_7844_VOICE_{—} | Clinic diagnosis VOICE DISTURBANCE (7844), count during |
| | DISTURBANCE | F0: before and early pregnancy |
| 1732 | F0_clinic_Z74_CESAREAN_{—} | Clinic diagnosis CESAREAN SECTION AND REMOVAL OF |
| | SECTION_AND_REMOVAL_OF_FETUS | FETUS (Z74), count during F0: before and early pregnancy |
| 1733 | F0_clinic_6929_CONTACT_{—} | Clinic diagnosis CONTACT DERMATITIS AND OTHER |
| | DERMATITIS_AND_OTHER_{—} | ECZEMA, UNSPECIFIED CAUSE (6929), count during F0: |
| | ECZEMA_UNSPECIFIED_CAUSE | before and early pregnancy |
| 1734 | F0_clinic_5650_ANAL_FISSURE | Clinic diagnosis ANAL FISSURE (5650), count during F0: before |
| | | and early pregnancy |
| 1735 | F0_clinic_345_EPILEPSY | Clinic diagnosis EPILEPSY (345), count during F0: before and |
| | | early pregnancy |
| 1736 | F0_clinic_595_CYSTITIS | Clinic diagnosis CYSTITIS (595), count during F0: before and |
| | | early pregnancy |
| 1737 | F0_clinic_V7610_BREAST_{—} | Clinic diagnosis BREAST SCREENING FOR MALIGNANT |
| | SCREENING_FOR_MALIGNANT_{—} | NEOPLASMS, UNSP. (V7610), count during F0: before and |
| | NEOPLASMS_UNSP | early pregnancy |
| 1738 | F0_clinic_7820_DISTURBANCE_{—} | Clinic diagnosis DISTURBANCE OF SKIN SENSATION |
| | OF_SKIN_SENSATION | (7820), count during F0: before and early pregnancy |
| 1739 | F0_clinic_7821_RASH_AND_{—} | Clinic diagnosis RASH AND OTHER NONSPECIFIC SKIN |
| | OTHER_NONSPECIFIC_SKIN_{—} | ERUPTION (7821), count din ing F0: before and early pregnancy |
| | ERUPTION | |
| 1740 | F0_clinic_1104_DERMATOPHYTOSIS_{—} | Clinic diagnosis DERMATOPHYTOSIS OF FOOT (1104), count |
| | OF_FOOT | during F0: before and early pregnancy |
| 1741 | F0_clinic_6264_IRREGULAR_{—} | Clinic diagnosis IRREGULAR MENSTRUAL CYCLE (6264), |
| | MENSTRUAL_CYCLE | count during F0: before and early pregnancy |
| 1742 | F0_clinic_7856_ENLARGEMENT_{—} | Clinic diagnosis ENLARGEMENT OF LYMPH NODES (7856), |
| | OF_LYMPH_NODES | count during F0: before and early pregnancy |
| 1743 | F0_clinic_5693_HEMORRHAGE_{—} | Clinic diagnosis HEMORRHAGE OF RECTUM AND ANUS |
| | OF_RECTUM_AND_ANUS | (5693), count during F0: before and early pregnancy |
| 1744 | F0_clinic_U80_INJURIES | Clinic diagnosis INJURIES (U80), count during F0: before and |
| | | early pregnancy |
| 1745 | F1_clinic_6793_ORAL_{—} | Clinic diagnosis ORAL CONTRACEPTION PRESCRIPTION |
| | CONTRACEPTION_PRESCRIPTION | (6793), count during F1: first blood test (weeks 6-12) |
| 1746 | F0_clinic_5997_HEMATURIA | Clinic diagnosis HEMATURIA (5997), count during F0: before |
| | | and early pregnancy |
| 1747 | F0_clinic_3000_ANXIETY_STATES | Clinic diagnosis ANXIETY STATES (3000), count during F0: |
| | | before and early pregnancy |
| 1748 | F0_clinic_7048_OTHER_{—} | Clinic diagnosis OTHER SPECIFIED DISEASES OF HAIR |
| | SPECIFIED_DISEASES_OF_{—} | AND HAIR FOLLICLES (7048), count during F0: before and |
| | HAIR_AND_HAIR_FOLLICLES | early pregnancy |
| 1749 | F0_clinic_5282_ORAL_APHTHAE | Clinic diagnosis ORAL APHTHAE (5282), count during F0: |
| | | before and early pregnancy |
| 1750 | F0_clinic_P760_ISSUE_OF_{—} | Clinic diagnosis ISSUE OF REPEAT PRESCRIPTION (P760), |
| | REPEAT_PRESCRIPTION | count during F0: before and early pregnancy |
| 1751 | F0_clinic_3730_BLEPHARITIS | Clinic diagnosis BLEPHARITIS (3730), count during F0: before |
| | | and early pregnancy |
| 1752 | F0_clinic_9071_LATE_EFFECT_{—} | Clinic diagnosis LATE EFFECT OF INJURY TO CRANIAL |
| | OF_INJURY_TO_CRANIAL_NERVE | NERVE (9071), count during F0: before and early pregnancy |
| 1753 | F0_clinic_X18_BREAST_PAIN | Clinic diagnosis BREAST PAIN (X18), count during F0: before |
| | | and early pregnancy |
| 1754 | F0_clinic_3731_HORDEOLUM_{—} | Clinic diagnosis HORDEOLUM AND OTHER DEEP |
| | AND_OTHER_DEEP_{—} | INFLAMMATION OF EYELID (3731), count during F0: before |
| | INFLAMMATION_OF_EYELID | and early pregnancy |
| 1755 | F0_clinic_6221_DYSPLASIA_{—} | Clinic diagnosis DYSPLASIA OF CERVIX (UTERI) (6221), |
| | OF_CERVIX_UTERI | count during F0: before and early pregnancy |
| 1756 | F0_clinic_28249_OTHER_{—} | Clinic diagnosis OTHER THALASSEMIA (28249), count during |
| | THALASSEMIA | F0: before and early pregnancy |
| 1757 | F0_clinic_7243_SCIATICA | Clinic diagnosis SCIATICA (7243), count during F0: before and |
| | | early pregnancy |
| 1758 | F0_clinic_5742_CALCULUS_{—} | Clinic diagnosis CALCULUS OF GALLBLADDER WITHOUT |
| | OF_GALLBLADDER_{—} | MENTION OF CHOLECYSTITIS (5742), count during F0: |
| | WITHOUT_MENTION_OF_{—} | before and early pregnancy |
| | CHOLECYSTITIS | |
| 1759 | F0_clinic_L03_LOW_BACK_{—} | Clinic diagnosis LOW BACK COMPLT.W/O RADIATION |
| | COMPLT_W_OR_O_RADIATION | (L03), count during F0: before and early pregnancy |
| 1760 | F0_clinic_606_INFERTILITY_{—} | Clinic diagnosis INFERTILITY, MALE (606), count during F0: |
| | MALE | before and early pregnancy |
| 1761 | F0_clinic_3751_OTHER_{—} | Clinic diagnosis OTHER DISORDERS OF LACRIMAL GLANE |
| | DISORDERS_OF_LACRIMAL_{—} | (3751), count din ing F0: before and early pregnancy |
| | GLAND | |
| 1762 | F0_clinic_W05_VOMITING_{—} | Clinic diagnosis VOMITING/NAUSEA OF PREGNANCY |
| | OR_NAUSEA_OF_PREGNANCY | (W05), count during F0: before and early pregnancy |
| 1763 | F0_clinic_3732_CHALAZION | Clinic diagnosis CHALAZION (3732), count during F0: before |
| | | and early pregnancy |
| 1764 | F0_clinic_V268_IN_VITRO_{—} | Clinic diagnosis IN VITRO FERTILIZATION OR TEST TUBE |
| | FERTILIZATION_OR_TEST_{—} | PREGNANCY (V268), count during F0: before and early |
| | TUBE_PREGNANCY | pregnancy |
| 1765 | F0_clinic_7847_EPISTAXIS | Clinic diagnosis EPISTAXIS (7847), count during F0: before and |
| | | early pregnancy |
| 1766 | F0_clinic_3540_CARPAL_{—} | Clinic diagnosis CARPAL TUNNEL SYNDROME (3540), count |
| | TUNNEL_SYNDROME | during F0: before and early pregnancy |
| 1767 | F0_clinic_0549_HERPES_{—} | Clinic diagnosis HERPES SIMPLEX WITHOUT MENTION OF |
| | SIMPLEX_WITHOUT_{—} | COMPLICATION (0549), count during F0: before and early |
| | MENTION_OF_COMPLICATION | pregnancy |
| 1768 | F0_clinic_7019_UNSPECIFIED_{—} | Clinic diagnosis UNSPECIFIED HYPERTROPHIC AND |
| | HYPERTROPHIC_AND_{—} | ATROPHIC CONDITIONS OF SKIN (7019), count during F0: |
| | ATROPHIC_CONDITIONS_{—} | before and early pregnancy |
| | OF_SKIN | |
| 1769 | F0_clinic_B82_ANEMIA_{—} | Clinic diagnosis ANEMIA OTHER/UNSPECIFIED (B82), count |
| | OTHER_OR_UNSPECIFIED | during F0: before and early pregnancy |
| 1770 | F0_clinic_244_ACQUIRED_{—} | Clinic diagnosis ACQUIRED HYPOTHYROIDISM (244), count |
| | HYPOTHYROIDISM | during F0: before and early pregnancy |
| 1771 | F0_clinic_6220_EROSION_{—} | Clinic diagnosis EROSION AND ECTROPION OF CERVIX |
| | AND_ECTROPION_OF_CERVIX | (6220), count during F0: before and early pregnancy |
| 1772 | F0_clinic_7040_ALOPECIA | Clinic diagnosis ALOPECIA (7040), count during F0: before and |
| | | early pregnancy |
| 1773 | F0_clinic_V71_OBSERVATION_{—} | Clinic diagnosis OBSERVATION AND EVALUATION FOR |
| | AND_EVALUATION_FOR_{—} | SUSPECTED CONDITIONS (V71), count during F0: before and |
| | SUSPECTED_CONDITIONS | early pregnancy |
| 1774 | F0_clinic_3810_ACUTE_{—} | Clinic diagnosis ACUTE NONSUPPURATIVE OTITIS MEDIA |
| | NONSUPPURATIVE_OTITIS_{—} | (3810), count during F0: before and early pregnancy |
| | MEDIA | |
| 1775 | F0_clinic_3804_IMPACTED_{—} | Clinic diagnosis IMPACTED CERUMEN (3804), count during |
| | CERUMEN | F0: before and early pregnancy |
| 1776 | F0_clinic_6261_SCANTY_OR_{—} | Clinic diagnosis SCANTY OR INFREQUENT |
| | INFREQUENT_MENSTRUATION | MENSTRUATION (6261), count during F0: before and early |
| | | pregnancy |
| 1777 | F0_clinic_6200_FOLLICULAR_{—} | Clinic diagnosis FOLLICULAR CYST OF OVARY (6200), |
| | CYST_OF_OVARY | count during F0: before and early pregnancy |
| 1778 | F0_clinic_5640_CONSTIPATION | Clinic diagnosis CONSTIPATION (5640), count during F0: |
| | | before and early pregnancy |
| 1779 | F0_clinic_454_VARICOSE_{—} | Clinic diagnosis VARICOSE VEINS OF LOWER |
| | VEINS_OF_LOWER_EXTREMITIES | EXTREMITIES (454), count during F0: before and early |
| | | pregnancy |
| 1780 | F0_clinic_493_ASTHMA | Clinic diagnosis ASTHMA (493), count during F0: before and |
| | | early pregnancy |
| 1781 | F0_clinic_L02_BACK_{—} | Clinic diagnosis BACK SYMPTOMS/COMPLAINTS (L02), |
| | SYMPTOMS_OR_COMPLAINTS | count during F0: before and early pregnancy |
| 1782 | F0_clinic_643_EXCESSIVE_{—} | Clinic diagnosis EXCESSIVE VOMITING IN PREGNANCY |
| | VOMITING_IN_PREGNANCY | (643), count during F0: before and early pregnancy |
| 1783 | F0_clinic_6792_ORAL_{—} | Clinic diagnosis ORAL CONTRACEPTION COUNSELING |
| | CONTRACEPTION_COUNSELING | (6792), count during F0: before and early pregnancy |
| 1784 | F0_clinic_7860_DYSPNEA_{—} | Clinic diagnosis DYSPNEA AND RESPIRATORY |
| | AND_RESPIRATORY_{—} | ABNORMALITIES (7860), count during F0: before and early |
| | ABNORMALITIES | pregnancy |
| 1785 | F0_clinic_V263_GENETIC_{—} | Clinic diagnosis GENETIC COUNSELING ON PROCREATIVE |
| | COUNSELING_ON_PROCREATIVE_{—} | MANAGEMENT (V263), count during F0: before and early |
| | MANAGEMENT | pregnancy |
| 1786 | F0_clinic_242_THYROTOXICOSIS_{—} | Clinic diagnosis THYROTOXICOSIS WITH OR WITHOUT |
| | WITH_OR_WITHOUT_GOITER | GOITER (242), count during F0: before and early pregnancy |
| 1787 | F0_clinic_525_DISEASES_AND_{—} | Clinic diagnosis DISEASES AND CONDITIONS OF THE |
| | CONDITIONS_OF_THE_TEETH_{—} | TEETH AND SUPPORT.STRUCTURES (525), count during F0: |
| | AND_SUPPORT_STRUCTURES | before and early pregnancy |
| 1788 | F0_clinic_A97_NO_DlSEASE | Clinic diagnosis NO DISEASE (A97), count during F0: before |
| | | and early pregnancy |
| 1789 | F0_clinic_78791_DIARRHEA | Clinic diagnosis DIARRHEA (78791), count during F0: before |
| | | and early pregnancy |
| 1790 | F0_clinic_7231_CERVICALGIA | Clinic diagnosis CERVICALGIA (7231), count during F0: before |
| | | and early pregnancy |
| 1791 | F0_clinic_6466_INFECTIONS_{—} | Clinic diagnosis INFECTIONS OF GENITOURINARY TRACT |
| | OF_GENITOURINARY_TRACT_{—} | IN PREGNANCY (6466), count during F0: before and early |
| | IN_PREGNANCY | pregnancy |
| 1792 | F0_clinic_7295_PAIN_IN_LIMB | Clinic diagnosis PAIN IN LIMB (7295), count during F0: before |
| | | and early pregnancy |
| 1793 | F0_clinic_6510_TWIN_PREGNANCY | Clinic diagnosis TWIN PREGNANCY (6510), count during F0: |
| | | before and early pregnancy |
| 1794 | F0_clinic_2169_BENIGN_{—} | Clinic diagnosis BENIGN NEOPLASM OF SKIN, SITE |
| | NEOPLASM_OF_SKIN_SITE_{—} | UNSPECIFIED (2169), count during F0: before and early |
| | UNSPECIFIED | pregnancy |
| 1795 | F0_clinic_4660_ACUTE_BRONCHITIS | Clinic diagnosis ACUTE BRONCHITIS (4660), count during F0: |
| | | before and early pregnancy |
| 1796 | F0_clinic_6161_VAGINITIS_{—} | Clinic diagnosis VAGINITIS AND VULVOVAGINITIS (6161), |
| | AND_VULVOVAGINITIS | count during F0: before and early pregnancy |
| 1797 | F0_clinic_6117_SIGNS_AND_{—} | Clinic diagnosis SIGNS AND SYMPTOMS IN BREAST (6117), |
| | SYMPTOMS_IN_BREAST | count during F0: before and early pregnancy |
| 1798 | F0_clinic_3887_OTALGIA | Clinic diagnosis OTALGIA (3887), count during F0: before and |
| | | early pregnancy |
| 1799 | F0_clinic_L05_FLANK_{—} | Clinic diagnosis FLANK SYMPTOMS/COMPLAINTS (L05), |
| | SYMPTOMS_OR_COMPLAINTS | count during F0: before and early pregnancy |
| 1800 | F0_clinic_2564_POLYCYSTIC_{—} | Clinic diagnosis POLYCYSTIC OVARIES (2564), count during |
| | OVARIES | F0: before and early pregnancy |
| 1801 | F0_clinic_634_SPONTANEOUS_{—} | Clinic diagnosis SPONTANEOUS ABORTION (634), count |
| | ABORTION | during F0: before and early pregnancy |
| 1802 | F0_clinic_7090_DYSCHROMIA_{—} | Clinic diagnosis DYSCHROMIA, ANOMALOUS |
| | ANOMALOUS_PIGMENTATION | PIGMENTATION (7090), count during F0: before and early |
| | | pregnancy |
| 1803 | F0_clinic_7291_MYALGIA_AND_{—} | Clinic diagnosis MYALGIA AND MYOSITIS, UNSPECIFIED |
| | MYOSITIS_UNSPECIFIED | (7291), count during F0: before and early pregnancy |
| 1804 | F0_clinic_5350_GASTRITIS | Clinic diagnosis GASTRITIS (5350), count during F0: before and |
| | | early pregnancy |
| 1805 | F0_clinic_S96_ACNE | Clinic diagnosis ACNE (S96), count during F0: before and early |
| | | pregnancy |
| 1806 | F0_clinic_6409_UNSPECIFIED_{—} | Clinic diagnosis UNSPECIFIED HEMORRHAGE IN EARLY |
| | HEMORRHAGE_IN_EARLY_{—} | PREGNANCY (6409), count during F0: before and early |
| | PREGNANCY | pregnancy |
| 1807 | F0_clinic_L04_CHEST_{—} | Clinic diagnosis CHEST SYMPTOMS/COMPLAINTS (L04), |
| | SYMPTOMS_OR_COMPLAINTS | count during F0: before and early pregnancy |
| 1808 | F0_clinic_L08_SHOULDER_{—} | Clinic diagnosis SHOULDER SYMPTOMS/COMPLAINTS |
| | SYMPTOMS_OR_COMPLAINTS | (L08), count during F0: before and early pregnancy |
| 1809 | F0_clinic_7061_OTHER_ACNE | Clinic diagnosis OTHER ACNE (7061), count during F0: before |
| | | and early pregnancy |
| 1810 | F0_clinic_7904_NONSPECIFIC_{—} | Clinic diagnosis NONSPECIFIC ELEVATION OF LEVELS OF |
| | ELEVATION_OF_LEVELS_{—} | TRANSAMINASE OR LDH (7904), count during F0: before and |
| | OF_TRANSAMINASE_OR_LDH | early pregnancy |
| 1811 | F0_clinic_0092_INFECTIOUS_{—} | Clinic diagnosis INFECTIOUS DIARRHEA (0092), count during |
| | DIARRHEA | F0: before and early pregnancy |
| 1812 | F0_clinic_692_CONTACT_{—} | Clinic diagnosis CONTACT DERMATITIS AND OTHER |
| | DERMATITIS_AND_OTHER_{—} | ECZEMA (692), count during F0: before and early pregnancy |
| | ECZEMA | |
| 1813 | F0_clinic_P027_ISSUE_OF_{—} | Clinic diagnosis ISSUE OF MEDICAL CERTIFICATE (P027), |
| | MEDICAL_CERTIFICATE | count during F0: before and early pregnancy |
| 1814 | F0_clinic_6981_PRURITUS_{—} | Clinic diagnosis PRURITUS OF GENITAL ORGANS (6981), |
| | OF_GENITAL_ORGANS | count during F0: before and early pregnancy |
| 1815 | F0_clinic_Z8909_CONSULTATION_{—} | Clinic diagnosis CONSULTATION, NOT OTHERWISE |
| | NOT_OTHERWISE_SPECIFIED | SPECIFIED (Z8909), count during F0: before and early |
| | | pregnancy |
| 1816 | F0_clinic_3671_MYOPIA | Clinic diagnosis MYOPIA (3671), count during F0: before and |
| | | early pregnancy |
| 1817 | F0_clinic_455_HEMORRHOIDS | Clinic diagnosis HEMORRHOIDS (455), count during F0: before |
| | | and early pregnancy |
| 1818 | F0_clinic_7194_PAIN_IN_JOINT | Clinic diagnosis PAIN IN JOINT (7194), count during F0: before |
| | | and early pregnancy |
| 1819 | F0_clinic_28709_OBESITY_BMI_>30 | Clinic diagnosis OBESITY (BMI >30) (28709), count during F0: |
| | | before and early pregnancy |
| 1820 | F0_clinic_D10_VOMITING_{—} | Clinic diagnosis VOMITING (EXCL.PREG. W06) (D10), count |
| | EXCL_PREG_W06 | during F0: before and early pregnancy |
| 1821 | F0_clinic_6463_HABITUAL_{—} | Clinic diagnosis HABITUAL ABORTER, CURRENTLY |
| | ABORTER_CURRENTLY_{—} | PREGNANT (6463), count during F0: before and early pregnancy |
| | PREGNANT | |
| 1822 | F0_clinic_2765_VOLUME_{—} | Clinic diagnosis VOLUME DEPLETION DISORDER (2765), |
| | DEPLETION_DISORDER | count during F0: before and early pregnancy |
| 1823 | F0_clinic_7270_SYNOVITIS_{—} | Clinic diagnosis SYNOVITIS AND TENOSYNOVITIS (7270), |
| | AND_TENOSYNOVITIS | count during F0: before and early pregnancy |
| 1824 | F0_clinic_S12_INSECT_BITE | Clinic diagnosis INSECT BITE (S12), count during F0: before |
| | | and early pregnancy |
| 1825 | F0_clinic_790_NONSPECIFIC_{—} | Clinic diagnosis NONSPECIFIC FINDINGS ON |
| | FINDINGS_ON_EXAMINATION_{—} | EXAMINATION OF BLOOD (790), count during F0: before and |
| | OF_BLOOD | early pregnancy |
| 1826 | F1_clinic_L02_BACK_SYMPTOMS_{—} | Clinic diagnosis BACK SYMPTOMS/COMPLAINTS (L02), |
| | OR_COMPLAINTS | count during F1: first blood test (weeks 6-12) |
| 1827 | F1_clinic_2564_POLYCYSTIC_{—} | Clinic diagnosis POLYCYSTIC OVARIES (2564), count during |
| | OVARIES | F1: first blood test (weeks 6-12) |
| 1828 | F1_clinic_Z8936_MANUAL_{—} | Clinic diagnosis MANUAL EXAMINATION OF BREAST |
| | EXAMINATION_OF_BREAST | (Z8936), count during F1: first blood test (weeks 6-12) |
| 1829 | F1_clinic_9599_OTHER_AND_{—} | Clinic diagnosis OTHER AND UNSPECIFIED INJURY TO |
| | UNSPECIFIED_INJURY_TO_{—} | UNSPECIFIED SITE (9599), count during F1: first blood test |
| | UNSPECIFIED_SITE | (weeks 6-12) |
| 1830 | F1_clinic_0092_INFECTIOUS_{—} | Clinic diagnosis INFECTIOUS DIARRHEA (0092), count during |
| | DIARRHEA | F1: first blood test (weeks 6-12) |
| 1831 | F1_clinic_Z8909_CONSULTATION_{—} | Clinic diagnosis CONSULTATION, NOT OTHERWISE |
| | NOT_OTHERWISE_SPECIFIED | SPECIFIED (Z8909), count during F1: first blood test |
| | | (weeks 6-12) |
| 1832 | F1_climc_455_HEMORRHOIDS | Clinic diagnosis HEMORRHOIDS (455), count during F1: first |
| | | blood test (weeks 6-12) |
| 1833 | F1_clinic_7194_PAIN_IN_JOINT | Clinic diagnosis PAIN IN JOINT (7194), count during F1: first |
| | | blood test (weeks 6-12) |
| 1834 | F1_clinic_28709_OBESITY_BMI_>30 | Clinic diagnosis OBESITY (BMI >30) (28709), count during F1: |
| | | first blood test (weeks 6-12) |
| 1835 | F1_clinic_L15_KNEE_SYMPTOMS_{—} | Clinic diagnosis KNEE SYMPTOMS/COMPLAINTS (L15), |
| | OR_COMPLAINTS | count during F1: first blood test (weeks 6-12) |
| 1836 | F1_clinic_D10_VOMITING_EXCL_{—} | Clinic diagnosis VOMITING (EXCL.PREG. W06) (D10), count |
| | PREG_W06 | during F1: first blood test (weeks 6-12) |
| 1837 | F1_clinic_6463_HABITUAL_ABORTER_{—} | Clinic diagnosis HABITUAL ABORTER, CURRENTLY |
| | CURRENTLY_PREGNANT | PREGNANT (6463), count during F1: first blood test (weeks 6-12) |
| 1838 | F1_clinic_2765_VOLUME_{—} | Clinic diagnosis VOLUME DEPLETION DISORDER (2765), |
| | DEPLETION_DISORDER | count during F1: first blood test (weeks 6-12) |
| 1839 | F1_clinic_9953_ALLERGY_{—} | Clinic diagnosis ALLERGY, UNSPECIFIED, NOT |
| | UNSPECIFIED_NOT_{—} | ELSEWHERE CLASSIFIED (9953), count during F1: first blood |
| | ELSEWHERE_CLASSIFIED | test (weeks 6-12) |
| 1840 | F1_clinic_3887_OTALGIA | Clinic diagnosis OTALGIA (3887), count during F1: first blood |
| | | test (weeks 6-12) |
| 1841 | F1_clinic_6117_SIGNS_AND_{—} | Clinic diagnosis SIGNS AND SYMPTOMS IN BREAST (6117), |
| | SYMPTOMS_IN_BREAST | count during F1: first blood test (weeks 6-12) |
| 1842 | F1_clinic_3810_ACUTE_{—} | Clinic diagnosis ACUTE NONSUPPURATIVE OTITIS MEDIA |
| | NONSUPPURATIVE_OTITIS_MEDIA | (3810), count during F1: first blood test (weeks 6-12) |
| 1843 | F1_clinic_632_MISSED_ABORTION | Clinic diagnosis MISSED ABORTION (632), count during F1: |
| | | first blood test (weeks 6-12) |
| 1844 | F1_clinic_3804_IMPACTED_CERUMEN | Clinic diagnosis IMPACTED CERUMEN (3804), count during |
| | | F1: first blood test (weeks 6-12) |
| 1845 | F1_clinic_5589_OTHER_AND_{—} | Clinic diagnosis OTHER AND UNSPEC.NONINFECTIOUS |
| | UNSPEC_NONINFECTIOUS_{—} | GASTROENTERITIS AND COLITIS (5589), count during F1: |
| | GASTROENTERITIS_AND_COLITIS | first blood test (weeks 6-12) |
| 1846 | F1_clinic_6261_SCANTY_OR_{—} | Clinic diagnosis SCANTY OR INFREQUENT |
| | INFREQUENT_MENSTRUATION | MENSTRUATION (6261), count during F1: first blood test |
| | | (weeks 6-12) |
| 1847 | F1_clinic_6200_FOLLICULAR_{—} | Clinic diagnosis FOLLICULAR CYST OF OVARY (6200), |
| | CYST_OF_OVARY | count during F1: first blood test (weeks 6-12) |
| 1848 | F1_clinic_5640_CONSTIPATION | Clinic diagnosis CONSTIPATION (5640), count during F1: first |
| | | blood test (weeks 6-12) |
| 1849 | F1_clinic_454_VARICOSE_VEINS_{—} | Clinic diagnosis VARICOSE VEINS OF LOWER |
| | OF_LOWER_EXTREMITIES | EXTREMITIES (454), count during F1: first blood test (weeks 6-12) |
| 1850 | F1_clinic_7904_NONSPEClFIC_{—} | Clinic diagnosis NONSPECIFIC ELEVATION OF LEVELS OF |
| | ELEVATION_OF_LEVELS_OF_{—} | TRANSAMINASE OR LDH (7904), count during F1: first blood |
| | TRANSAMINASE_OR_LDH | test (weeks 6-12) |
| 1851 | F1_clinic_5368_DYSPEPSIA_AND_{—} | Clinic diagnosis DYSPEPSIA AND OTHER SPECIFIED |
| | OTHER_SPECIFIED_DISORDERS_{—} | DISORDERS OF FUNCTION OF STOMACH (5368), count |
| | OF_FUNCTION_OF_STOMACH | during F1: first blood test (weeks 6-12) |
| 1852 | F1_clinic_401_ESSENTIAL_{—} | Clinic diagnosis ESSENTIAL HYPERTENSION (401), count |
| | HYPERTENSION | during F1: first blood test (weeks 6-12) |
| 1853 | F1_clinic_216_BENIGN_{—} | Clinic diagnosis BENIGN NEOPLASM OF SKIN (216), count |
| | NEOPLASM_OF_SKIN | during F1: first blood test (weeks 6-12) |
| 1854 | F1_clinic_6267_POSTCOITAL_{—} | Clinic diagnosis POSTCOITAL BLEEDING (6267), count during |
| | BLEEDING | F1: first blood test (weeks 6-12) |
| 1855 | F1_clinic_6829_CELLULITIS_{—} | Clinic diagnosis CELLULITIS AND ABSCESS OF |
| | AND_ABSCESS_OF_UNSPECIFIED_SITES | UNSPECIFIED SITES (6829), count during F1: first blood test |
| | | (weeks 6-12) |
| 1856 | F1_clinic_27800_OBESITY_{—} | Clinic diagnosis OBESITY, UNSPECIFIED (27800), count |
| | UNSPECIFIED | during F1: first blood test (weeks 6-12) |
| 1857 | F1_clinic_1110_PITYRIASIS_{—} | Clinic diagnosis PITYRIASIS VERSICOLOR (1110), count |
| | VERSICOLOR | during F1: first blood test (weeks 6-12) |
| 1858 | F1_clinic_7844_VOICE_DISTURBANCE | Clinic diagnosis VOICE DISTURBANCE (7844), count during |
| | | F1: first blood test (weeks 6-12) |
| 1859 | F1_clinic_Z74_CESAREAN_{—} | Clinic diagnosis CESAREAN SECTION AND REMOVAL OF |
| | SECTION_AND_REMOVAL_OF_FETUS | FETUS (Z74), count during F1: first blood test (weeks 6-12) |
| 1860 | F1_clinic_5650_ANAL_FISSURE | Clinic diagnosis ANAL FISSURE (5650), count during F1: first |
| | | blood test (weeks 6-12) |
| 1861 | F1_clinic_V700_ROUTINE_{—} | Clinic diagnosis ROUTINE GENERAL MEDICAL |
| | GENERAL_MEDICAL_{—} | EXAMINATION AT A HEALTH CARE FACILITY (V700), |
| | EXAMINATION_AT_A_{—} | count during F1: first blood test (weeks 6-12) |
| | HEALTH_CARE_FACILITY | |
| 1862 | F1_clinic_345_EPILEPSY | Clinic diagnosis EPILEPSY (345), count during F1: first blood |
| | | test (weeks 6-12) |
| 1863 | F1_clinic_V7610_BREAST_{—} | Clinic diagnosis BREAST SCREENING FOR MALIGNANT |
| | SCREENING_FOR_{—} | NEOPLASMS, UNSP. (V7610), count during F1: first blood test |
| | MALIGNANT_NEOPLASMS_{—} | (weeks 6-12) |
| | UNSP | |
| 1864 | F1_clinic_7821_RASH_AND_{—} | Clinic diagnosis RASH AND OTHER NONSPECIFIC SKIN |
| | OTHER_NONSPECIFIC_SKIN_{—} | ERUPTION (7821), count during F1: first blood test (weeks 6-12) |
| | ERUPTION | |
| 1865 | F1_clinic_L08_SHOULDER_{—} | Clinic diagnosis SHOULDER SYMPTOMS/COMPLAINTS |
| | SYMPTOMS_OR_COMPLAINTS | (L08), count during F1: first blood test (weeks 6-12) |
| 1866 | F1_clinic_7856_ENLARGEMENT_{—} | Clinic diagnosis ENLARGEMENT OF LYMPH NODES (7856), |
| | OF_LYMPH_NODES | count during F1: first blood test (weeks 6-12) |
| 1867 | F1_clinic_244_ACQUIRED_{—} | Clinic diagnosis ACQUIRED HYPOTHYROIDISM (244), count |
| | HYPOTHYROIDISM | during F1: first blood test (weeks 6-12) |
| 1868 | F1_clinic_6220_EROSION_AND_{—} | Clinic diagnosis EROSION AND ECTROPION OF CERVIX |
| | ECTROPION_OF_CERVIX | (6220), count during F1: first blood test (weeks 6-12) |
| 1869 | F1_clinic_7040_ALOPECIA | Clinic diagnosis ALOPECIA (7040), count during F1: first blood |
| | | test (weeks 6-12) |
| 1870 | F1_clinic_634_SPONTANEOUS_{—} | Clinic diagnosis SPONTANEOUS ABORTION (634), count |
| | ABORTION | during F1: first blood test (weeks 6-12) |
| 1871 | F1_clinic_7090_DYSCHROMIA_{—} | Clinic diagnosis DYSCHROMIA, ANOMALOUS |
| | ANOMALOUS_PIGMENTATION | PIGMENTATION (7090), count during F1: first blood test |
| | | (weeks 6-12) |
| 1872 | F1_clinic_S96_ACNE | Clinic diagnosis ACNE (S96), count during F1: first blood test |
| | | (weeks 6-12) |
| 1873 | F1_clinic_7865_CHEST_PAIN | Clinic diagnosis CHEST PAIN (7865), count during F1: first |
| | | blood test (weeks 6-12) |
| 1874 | F1_clinic_6409_UNSPECIFIED_{—} | Clinic diagnosis UNSPECIFIED HEMORRHAGE IN EARLY |
| | HEMORRHAGE_IN_EARLY_{—} | PREGNANCY (6409), count during F1: first blood test (weeks 6-12) |
| | PREGNANCY | |
| 1875 | F1_clinic_L04_CHEST_SYMPTOMS_{—} | Clinic diagnosis CHEST SYMPTOMS/COMPLAINTS (L04), |
| | OR_COMPLAINTS | count during F1: first blood test (weeks 6-12) |
| 1876 | F1_clinic_493_ASTHMA | Clinic diagnosis ASTHMA (493), count during F1: first blood test |
| | | (weeks 6-12) |
| 1877 | F1_clinic_7851_PALPITATIONS | Clinic diagnosis PALPITATIONS (7851), count during F1: first |
| | | blood test (weeks 6-12) |
| 1878 | F0_clinic_6465_ASYMPTOMATIC_{—} | Clinic diagnosis ASYMPTOMATIC BACTERIURIA IN |
| | BACTERIURIA_IN_PREGNANCY | PREGNANCY (6465), count during F0: before and early |
| | | pregnancy |
| 1879 | F1_clinic_3801_INFECTIVE_{—} | Clinic diagnosis INFECTIVE OTITIS EXTERNA (3801), count |
| | OTITIS_EXTERNA | during F1: first blood test (weeks 6-12) |
| 1880 | F1_clinic_78079_OTHER_MALAISE_{—} | Clinic diagnosis OTHER MALAISE AND FATIGUE (78079), |
| | AND_FATIGUE | count during F1: first blood test (weeks 6-12) |
| 1881 | F1_clinic_7862_COUGH | Clinic diagnosis COUGH (7862), count during F1: first blood test |
| | | (weeks 6-12) |
| 1882 | F1_clinic_463_ACUTE_TONSILLITIS | Clinic diagnosis ACUTE TONSILLITIS (463), count during F1: |
| | | first blood test (weeks 6-12) |
| 1883 | F1_clinic_7870_NAUSEA_AND_{—} | Clinic diagnosis NAUSEA AND VOMITING (7870), count |
| | VOMITING | during F1: first blood test (weeks 6-12) |
| 1884 | F1_clinic_2449_UNSPECIFIED_{—} | Clinic diagnosis UNSPECIFIED ACQUIRED |
| | ACQUIRED_HYPOTHYROIDISM | HYPOTHYROIDISM (2449), count during F1: first blood test |
| | | (weeks 6-12) |
| 1885 | F1_clinic_462_ACUTE_PHARYNGITIS | Clinic diagnosis ACUTE PHARYNGITIS (462), count during F1: |
| | | first blood test (weeks 6-12) |
| 1886 | F1_clinic_W84_PREGNANCY_{—} | Clinic diagnosis PREGNANCY HIGH RISK (W84), count during |
| | HIGH_RISK | F1: first blood test (weeks 6-12) |
| 1887 | F1_clinic_7840_HEADACHE | Clinic diagnosis HEADACHE (7840), count during F1: first blood |
| | | test (weeks 6-12) |
| 1888 | F1_clinic_W78_PREGNANCY_{—} | Clinic diagnosis PREGNANCY: CONFIRMED (W78), count |
| | CONFIRMED | during F1: first blood test (weeks 6-12) |
| 1889 | F1_clinic_4659_ACUTE_UPPER_{—} | Clinic diagnosis ACUTE UPPER RESPIRATORY INFECTIONS |
| | RESPIRATORY_INFECTIONS_OF_{—} | OF UNSPECIFIED SITE (4659), count during F1: first blood test |
| | UNSPECIFIED_SITE | (weeks 6-12) |
| 1890 | F1_clinic_Z8926_GYNECOLOGICAL_{—} | Clinic diagnosis GYNECOLOGICAL EXAMINATION (Z8926), |
| | EXAMINATION | count during F1: first blood test (weeks 6-12) |
| 1891 | F1_clinic_465_ACUTE_UPPER_{—} | Clinic diagnosis ACUTE UPPER RESPIRATORY INFECTIONS |
| | RESPIRATORY_INFECTIONS_OF_{—} | OF MULTIPLE OR UNSP.SITES (465), count during F1: first |
| | MULTIPLE_OR_UNSP_SITES | blood test (weeks 6-12) |
| 1892 | F1_clinic_7890_ABDOMINAL_PAIN | Clinic diagnosis ABDOMINAL PAIN (7890), count during F1: |
| | | first blood test (weeks 6-12) |
| 1893 | F0_clinic_6260_ABSENCE_OF_{—} | Clinic diagnosis ABSENCE OF MENSTRUATION (6260), count |
| | MENSTRUATION | during F0: before and early pregnancy |
| 1894 | F1_clinic_1_FILL_OUT_FORMS | Clinic diagnosis FILL OUT FORMS (1), count during F1: first |
| | | blood test (weeks 6-12) |
| 1895 | F0_clinic_07819_OTHER_SPECIFIED_{—} | Clinic diagnosis OTHER SPECIFIED VIRAL WARTS (07819), |
| | VIRAL_WARTS | count during F0: before and early pregnancy |
| 1896 | F0_clinic_286_COAGULATION_DEFECTS | Clinic diagnosis COAGULATION DEFECTS (286), count during |
| | | F0: before and early pregnancy |
| 1897 | F0_clinic_7910_PROTEINURIA | Clinic diagnosis PROTEINURIA (7910), count during F0: before |
| | | and early pregnancy |
| 1898 | F0_clinic_9223_CONTUSION_OF_BACK | Clinic diagnosis CONTUSION OF BACK (9223), count during |
| | | F0: before and early pregnancy |
| 1899 | F0_clinic_5559_REGIONAL_{—} | Clinic diagnosis REGIONAL ENTERITIS OF UNSPECIFIED |
| | ENTERITIS_OF_UNSPECIFIED_SITE | SITE (5559), count during F0: before and early pregnancy |
| 1900 | F0_clinic_218_UTERINE_{—} | Clinic diagnosis UTERINE LEIOMYOMA (218), count during |
| | LEIOMYOMA | F0: before and early pregnancy |
| 1901 | F0_clinic_7880_RENAL_COLIC | Clinic diagnosis RENAL COLIC (7880), count during F0: before |
| | | and early pregnancy |
| 1902 | F0_clinic_7030_INGROWING_NAIL | Clinic diagnosis INGROWING NAIL (7030), count during F0: |
| | | before and early pregnancy |
| 1903 | F1_clinic_7804_DIZZINESS_AND_{—} | Clinic diagnosis DIZZINESS AND GIDDINESS (VERTIGO) |
| | GIDDINESS_VERTIGO | (7804), count during F1: first blood test (weeks 6-12) |
| 1904 | F1_clinic_V67_FOLLOW_UP_{—} | Clinic diagnosis FOLLOW-UP EXAMINATION (V67), count |
| | EXAMINATION | during F1: first blood test (weeks 6-12) |
| 1905 | F1_clinic_6400_THREATENED_{—} | Clinic diagnosis THREATENED ABORTION (6400), count |
| | ABORTION | during F1: first blood test (weeks 6-12) |
| 1906 | F1_clinic_1121_CANDIDIASIS_OF_{—} | Clinic diagnosis CANDIDIASIS OF VULVA AND VAGINA |
| | VULVA_AND_VAGINA | (1121), count during F1: first blood test (weeks 6-12) |
| 1907 | F1_clinic_6792_ORAL_{—} | Clinic diagnosis ORAL CONTRACEPTION COUNSELING |
| | CONTRACEPTION_COUNSELING | (6792), count during F1: first blood test (weeks 6-12) |
| 1908 | F1_clinic_7860_DYSPNEA_AND_{—} | Clinic diagnosis DYSPNEA AND RESPIRATORY |
| | RESPIRATORY_ABNORMALITIES | ABNORMALITIES (7860), count during F1: first blood test |
| | | (weeks 6-12) |
| 1909 | F1_clinic_6235_LEUKORRHEA_NOT_{—} | Clinic diagnosis LEUKORRHEA, NOT SPECIFIED AS |
| | SPECIFIED_AS_INFECTIVE | INFECTIVE (6235), count during F1: first blood test (weeks 6-12) |
| 1910 | F1_clinic_7231_CERVICALGIA | Clinic diagnosis CERVICALGIA (7231), count during F1: first |
| | | blood test (weeks 6-12) |
| 1911 | F1_clinic_6466_INFECTIONS_OF_{—} | Clinic diagnosis INFECTIONS OF GENITOURINARY TRACT |
| | GENITOURINARY_TRACT_IN_PREGNANCY | IN PREGNANCY (6466), count during F1: first blood test (weeks 6-12) |
| 1912 | F1_clinic_2662_OTHER_B_COMPLEX_{—} | Clinic diagnosis OTHER B-COMPLEX DEFICIENCIES (2662), |
| | DEFICIENCIES | count during F1: first blood test (weeks 6-12) |
| 1913 | F1_climc_7295_PAIN_IN_LIMB | Clinic diagnosis PAIN IN LIMB (7295), count during F1: first |
| | | blood test (weeks 6-12) |
| 1914 | F1_clinic_A03_FEVER | Clinic diagnosis FEVER (A03), count during F1: first blood test |
| | | (weeks 6-12) |
| 1915 | F1_clinic_2169_BENIGN_NEOPLASM_{—} | Clinic diagnosis BENIGN NEOPLASM OF SKIN, SITE |
| | OF_SKIN_SITE_UNSPECIFIED | UNSPECIFIED (2169), count during F1: first blood test (weeks 6-12) |
| 1916 | F1_clinic_4660_ACUTE_BRONCHITIS | Clinic diagnosis ACUTE BRONCHITIS (4660), count during F1: |
| | | first blood test (weeks 6-12) |
| 1917 | F1_clinic_6161_VAGINITIS_AND_{—} | Clinic diagnosis VAGINITIS AND VULVOVAGINITIS (6161), |
| | VULVOVAGINITIS | count during F1: first blood test (weeks 6-12) |
| 1918 | F1_clinic_0091_COLITIS_ENTERITIS_{—} | Clinic diagnosis COLITIS,ENTERITIS,GASTROENTERITIS |
| | GASTROENTERITIS_PRESUMED_{—} | PRESUMED INFECTIOUS ORIGIN (0091), count during F1: |
| | INFECTIOUS_ORIGIN | first blood test (weeks 6-12) |
| 1919 | F1_clinic_D09_NAUSEA | Clinic diagnosis NAUSEA (D09), count during F1: first blood test |
| | | (weeks 6-12) |
| 1920 | F1_clinic_V221_SUPERVISION_OF_{—} | Clinic diagnosis SUPERVISION OF OTHER NORMAL |
| | OTHER_NORMAL_PREGNANCY | PREGNANCY (V221), count during F1: first blood test (weeks 6-12) |
| 1921 | F1_climc_6260_ABSENCE_OF_{—} | Clinic diagnosis ABSENCE OF MENSTRUATION (6260), count |
| | MENSTRUATION | during F1: first blood test (weeks 6-12) |
| 1922 | F1_climc_7998_OTHER_ILL_DEFINED_{—} | Clinic diagnosis OTHER ILL-DEFINED CONDITIONS (7998), |
| | CONDITIONS | count during F1: first blood test (weeks 6-12) |
| 1923 | F1_clinic_7993_DEBILITY_{—} | Clinic diagnosis DEBILITY, UNSPECIFIED (7993), count |
| | UNSPECIFIED | during F1: first blood test (weeks 6-12) |
| 1924 | F1_clinic_3720_ACUTE_{—} | Clinic diagnosis ACUTE CONJUNCTIVITIS (3720), count |
| | CONJUNCTIVITIS | during F1: first blood test (weeks 6-12) |
| 1925 | F1_clinic_460_ACUTE_{—} | Clinic diagnosis ACUTE NASOPHARYNGITIS (COMMON |
| | NASOPHARYNGITIS_COMMON_COLD | COLD) (460), count during F1: first blood test (weeks 6-12) |
| 1926 | F1_clinic_A80_ACCIDENT_OR_{—} | Clinic diagnosis ACCIDENT/INJURY; NOS (A80), count during |
| | INJURY_NOS | F1: first blood test (weeks 6-12) |
| 1927 | F1_clinic_7881_DYSURIA | Clinic diagnosis DYSURIA (7881), count during F1: first blood |
| | | test (weeks 6-12) |
| 1928 | F1_clinic_2809_IRON_DEFICIENCY_{—} | Clinic diagnosis IRON DEFICIENCY ANEMIA, UNSPECIFIED |
| | ANEMIA_UNSPECIFIED | (2809), count during F1: first blood test (weeks 6-12) |
| 1929 | F0_hospital_Z6812_HYSTEROSCOPY | Hospital diagnosis HYSTEROSCOPY (Z6812), count during F0: |
| | | before and early pregnancy |
| 1930 | F0_hospital_6289 | Hospital diagnosis INFERTILITY. FEMALE. SECONDARY |
| | 2_INFERTILITY_{—} | (6289 2), count during F0: before and early pregnancy |
| | FEMALE_SECONDARY | |
| 1931 | F0_hospital_59080_PYELONEPHRITIS_{—} | Hospital diagnosis PYELONEPHRITIS, UNSPECIFIED (59080) |
| | UNSPECIFIED | count during F0: before and early pregnancy |
| 1932 | F1_MICROALBUMIN_U_SAMP | Labtest MICROALBUMIN-U SAMP, sampled during F1: first |
| | | blood test (weeks 6-12) |
| 1933 | F1_HEMOLYTIC | Labtest HEMOLYTIC, sampled during F1: first blood test (weeks 6-12) |
| 1934 | F1_ICTERIC | Labtest ICTERIC, sampled during F1: first blood test (weeks 6-12) |
| 1935 | F1_MICROALBUMIN_or_CREAT | Labtest MICROALBUMIN/CREAT, sampled during F1: first |
| | | blood test (weeks 6-12) |
| 1936 | F0_PROLACTIN | Labtest PROLACTIN, sampled during F0: before and early |
| | | pregnancy |
| 1937 | F0_17_OH_PROGESTERONE | Labtest 17-OH-PROGESTERONE, sampled during F0: before |
| | | and early pregnancy |
| 1938 | F0_clinic_7245_BACKACHE_{—} | Clinic diagnosis BACKACHE, UNSPECIFIED (7245), count |
| | UNSPECIFIED | during F0: before and early pregnancy |
| 1939 | F0_ANTI_BODY_SCREEN_I | Labtest ANTI BODY SCREEN I, sampled during F0: before and |
| | | early pregnancy |
| 1940 | F0_FIBRINOGEN_CALCU | Labtest FIBRINOGEN CALCU, sampled during F0: before and |
| | | early pregnancy |
| 1941 | F0_EPITHELIAL_SED | Labtest EPITHELIAL-SED, sampled during F0: before and early |
| | | pregnancy |
| 1942 | F0_LEUCOCYTES_SED | Labtest LEUCOCYTES-SED, sampled during F0: before and |
| | | early pregnancy |
| 1943 | F0_UROBILINOGEN_U_STRIP | Labtest UROBILINOGEN-U STRIP, sampled during F0: before |
| | | and early pregnancy |
| 1944 | F0_NITRITE_U_STRIP | Labtest NITRITE- U STRIP, sampled during F0: before and early |
| | | pregnancy |
| 1945 | F0_PROTEIN_U_STRIP | Labtest PROTEIN- U STRIP, sampled during F0: before and |
| | | early pregnancy |
| 1946 | F0_LIPASE | Labtest LIPASE, sampled during F0: before and early pregnancy |
| 1947 | F0_CK_CREAT_KINASE_CPK | Labtest CK-CREAT.KINASE(CPK), sampled during F0: before |
| | | and early pregnancy |
| 1948 | F0_clinic_7993_DEBILITY_{—} | Clinic diagnosis DEBILITY, UNSPECIFIED (7993), count |
| | UNSPECIFIED | during F0: before and early pregnancy |
| 1949 | F0_PHOSPHORUS | Labtest PHOSPHORUS, sampled during F0: before and early |
| | | pregnancy |
| 1950 | F0_POTASSIUM | Labtest POTASSIUM, sampled during F0: before and early |
| | | pregnancy |
| 1951 | F0_SODIUM | Labtest SODIUM, sampled during F0: before and early pregnancy |
| 1952 | F0_UREA | Labtest UREA, sampled during F0: before and early pregnancy |
| 1953 | F0_clinic_6482_ANEMIA_{—} | Clinic diagnosis ANEMIA COMPLICATING PREGNANCY, |
| | COMPLICATING_PREGNANCY_{—} | CHILDBIRTH, OR THE PUERPERIUM (6482), count during |
| | CHILDBIRTH_OR_THE_PUERPERIUM | F0: before and early pregnancy |
| 1954 | F0_CONTROL_PTT | Labtest CONTROL PTT, sampled during F0: before and early |
| | | pregnancy |
| 1955 | F0_CMV_AVIDITY | Labtest CMV AVIDITY, sampled during F0: before and early |
| | | pregnancy |
| 1956 | F1_LIPEMIC | Labtest LIPEMIC, sampled during F1: first blood test (weeks 6-12) |
| 1957 | F1_ANTINUCLEAR_Ab_{——}ANA | Labtest ANTINUCLEAR Ab_(ANA), sampled during F1: first |
| | | blood test (weeks 6-12) |
| 1958 | F2_hospital_78060_FEVER_{—} | Hospital diagnosis FEVER, UNSPECIFIED (78060), count |
| | UNSPECIFIED | during F2: second blood test (weeks 16-19) |
| 1959 | F1_ANTI_CARDIOLIPIN_IgM | Labtest ANTI CARDIOLIPIN IgM, sampled during F1: first |
| | | blood test (weeks 6-12) |
| 1960 | F0_clinic_78079_OTHER_MALAISE_{—} | Clinic diagnosis OTHER MALAISE AND FATIGUE (78079), |
| | AND_FATIGUE | count during F0: before and early pregnancy |
| 1961 | F1_LEFT_SHIFT | Labtest LEFT SHIFT, sampled during F1: first blood test |
| | | (weeks 6-12) |
| 1962 | F1_BLAST_F | Labtest BLAST-F, sampled during F1: first blood test |
| | | (weeks 6-12) |
| 1963 | F1_MICRO_F | Labtest MICRO-F, sampled during F1: first blood test |
| | | (weeks 6-12) |
| 1964 | F0_clinic_V67_FOLLOW_UP_{—} | Clinic diagnosis FOLLOW-UP EXAMINATION (V67), count |
| | EXAMINATION | during F0: before and early pregnancy |
| 1965 | F1_PCT | Labtest PCT, sampled during F1: first blood test (weeks 6-12) |
| 1966 | F0_clinic_6400_THREATENED_{—} | Clinic diagnosis THREATENED ABORTION (6400), count |
| | ABORTION | during F0: before and early pregnancy |
| 1967 | F0_clinic_0091_COLITIS_ENTERITIS_{—} | Clinic diagnosis COLITIS, ENTERITIS, GASTROENTERITIS |
| | GASTROENTERITIS_PRESUMED_{—} | PRESUMED INFECTIOUS ORIGIN (0091), count during F0: |
| | INFECTIOUS_ORIGIN | before and early pregnancy |
| 1968 | F0_clinic_2809_IRON_DEFICIENCY_{—} | Clinic diagnosis IRON DEFICIENCY ANEMIA, UNSPECIFIED |
| | ANEMIA_UNSPECIFIED | (2809), count during F0: before and early pregnancy |
| 1969 | F0_clinic_7881_DYSURIA | Clinic diagnosis DYSURIA (7881), count during F0: before and |
| | | early pregnancy |
| 1970 | F1_RAPID_PL_REAGIN_VDRL | Labtest RAPID PL.REAGIN-VDRL, sampled during F1: first |
| | | blood test (weeks 6-12) |
| 1971 | F1_CMV_IgG_Add_VIDAS | Labtest CMV IgG (Add VIDAS), sampled during F1: first blood |
| | | test (weeks 6-12) |
| 1972 | F1_CMV_IgM_Add_VIDAS | Labtest CMV IgM(Add.-VIDAS), sampled during F1: first blood |
| | | test (weeks 6-12) |
| 1973 | F1_VARICELLA_ZOSTER_IgM | Labtest VARICELLA ZOSTER IgM, sampled during F1: first |
| | | blood test (weeks 6-12) |
| 1974 | F0_clinic_A80_ACCIDENT_OR_{—} | Clinic diagnosis ACCIDENT/INJURY; NOS (A80), count during |
| | INJURY_NOS | F0: before and early pregnancy |
| 1975 | F1_EBV_VCA_IgM | Labtest EBV VCA IgM, sampled during F1: first blood test |
| | | (weeks 6-12) |
| 1976 | F1_CMV_IgM | Labtest CMV IgM, sampled during F1: first blood test |
| | | (weeks 6-12) |
| 1977 | F1_HEPATITIS_Be_Ab | Labtest HEPATITIS Be Ab, sampled during F1: first blood test |
| | | (weeks 6-12) |
| 1978 | F1_HEPATITIS_Bs_Ab | Labtest HEPATITIS Bs Ab, sampled during F1: first blood test |
| | | (weeks 6-12) |
| 1979 | F1_HEPATITIS_Bs_Ag | Labtest HEPATITIS Bs Ag, sampled during F1: first blood test |
| | | (weeks 6-12) |
| 1980 | F1_TOXOPLASMA_IgM | Labtest TOXOPLASMA IgM, sampled during F1: first blood test |
| | | (weeks 6-12) |
| 1981 | F1_TOXOPLASMA_IgG | Labtest TOXOPLASMA IgG, sampled during F1: first blood test |
| | | (weeks 6-12) |
| 1982 | F1_DNA_ds_Ab | Labtest DNA (ds) Ab, sampled during F1: first blood test |
| | | (weeks 6-12) |
| 1983 | F0_HEMOGLOBIN_S | Labtest HEMOGLOBIN S, sampled during F0: before and early |
| | | pregnancy |
| 1984 | F0_HEMOGLOBIN_H | Labtest HEMOGLOBIN H, sampled during F0: before and early |
| | | pregnancy |
| 1985 | F0_HEMOGLOBIN_O | Labtest HEMOGLOBIN O, sampled during F0: before and early |
| | | pregnancy |
| 1986 | F0_HEMOGLOBIN_C | Labtest HEMOGLOBIN C, sampled during F0: before and early |
| | | pregnancy |
| 1987 | F0_RHEUMATOID_FACTOR | Labtest RHEUMATOID FACTOR, sampled during F0: before |
| | | and early pregnancy |
| 1988 | F0_LIPEMIC | Labtest LIPEMIC. sampled during F0: before and early pregnancy |
| 1989 | F0_HEMOLYTIC | Labtest HEMOLYTIC, sampled during F0: before and early |
| | | pregnancy |
| 1990 | F0_ICTERIC | Labtest ICTERIC, sampled during F0: before and early pregnancy |
| 1991 | F0_MICROALBUMIN_or_CREAT | Labtest MICROALBUMIN/CREAT, sampled during F0: before |
| | | and early pregnancy |
| 1992 | F2_hospital_Z9649_OTHER_{—} | Hospital diagnosis OTHER GENITOURINARY INSTILLATION |
| | GENITOURINARY_INSTILLATION | (Z9649), count during F2: second blood test (weeks 16-19) |
| 1993 | F2_hospital_Z6595_RELEASE_OF_{—} | Hospital diagnosis RELEASE OF TORSION OF OVARY |
| | TORSION_OF_OVARY | (Z6595), count during F2: second blood test (weeks 16-19) |
| 1994 | F2_hospital_6201_CORPUS_LUTEUM_{—} | Hospital diagnosis CORPUS LUTEUM CYST OR HEMATOMA |
| | CYST_OR_HEMATOMA | (6201), count during F2: second blood test (weeks 16-19) |
| 1995 | F2_hospital_Z8875_DIAGNOSTIC_{—} | Hospital diagnosis DIAGNOSTIC ULTRASOUND OF |
| | ULTRASOUND_OF_URINARY_SYSTEM | URINARY SYSTEM (Z8875), count during F2: second blood test |
| | | (weeks 16-19) |
| 1996 | F2_hospital_64973_CERVICAL_{—} | Hospital diagnosis CERVICAL SHORTENING |
| | SHORTENING_COMPLICATING_{—} | COMPLICATING PREGNANCY. ANTEPARTUM |
| | PREGNANCY_ANTEPARTUM_{—} | CONDITION OR COMPLICATION (64973), count during F2: |
| | CONDITION_OR_COMPLICATION | second blood test (weeks 16-19) |
| 1997 | F2_hospital_591_HYDRONEPHROSIS | Hospital diagnosis HYDRONEPHROSIS (591), count during F2: |
| | | second blood test (weeks 16-19) |
| 1998 | F2_hospital_6400_THREATENED_{—} | Hospital diagnosis THREATENED ABORTION (6400), count |
| | ABORTION | during F2: second blood test (weeks 16-19) |
| 1999 | F2_hospital_V180_Family_HISTORY_{—} | Hospital diagnosis FAMILY HISTROY OF DIABETES |
| | OF_DIABETES_MELLITUS | MELLITUS (V180), count during F2: second blood text |
| | | (weeks 16-19) |
| 2000 | F2_hostpital_V270_MOTHER_WITH_{—} | Hospital diagnosis MOTHER WITH SINGLE LIVEBORN |
| | SINGLE_LIVEBORN | (V270), count during F2: second blood test (weeks 16-19) |
| 2001 | F2_hospital_V2389_SUPERVISION_{—} | Hospital diagnosis SUPERVISION OF OTHER HIGH-RISK |
| | OF_OTHER_HIGH_RISK_PREGNANCY | PREGNANCY (V2389), count during F2: second blood test |
| | | (weeks 16-19) |
| 2002 | F2_hospital_78609_OTHER_DYSPNEA_{—} | Hospital diagnosis OTHER DYSPNEA AND RESPIRATORY |
| | AND_RESPIRATORY_ABNORMALITY | ABNORMALITY (78609), count during F2: second blood test |
| | | (weeks 16-19) |
| 2003 | F2_hospital_7880_RENAL_COLIC | Hospital diagnosis RENAL COLIC (7880), count during F2: |
| | | second blood test (weeks 16-19) |
| 2004 | F2_hospital_6238_OTHER_SPECIFIED_{—} | Hospital diagnosis OTHER SPECIFIED |
| | NONINFLAMMATORY_DISORDERS_{—} | NONINFLAMMATORY DISORDERS OF VAGINA (6238), |
| | OF_VAGINA | count during F2: second blood test (weeks 16-19) |
| 2005 | F2_hospital_64313_HYPEREMESIS_{—} | Hospital diagnosis HYPEREMESIS GRAVIDARUM WITH |
| | GRAVIDARUM_WITH_METABOLIC_{—} | METABOLIC DISTURBANCE, ANTEPARTUM (64313), count |
| | DISTURBANCE_ANTEPARTUM | during F2: second blood test (weeks 16-19) |
| 2006 | F2_hospital_5589_OTHER_AND_{—} | Hospital diagnosis OTHER AND UNSPEC.NONINFECTIOUS |
| | UNSPEC_NONINFECTIOUS_{—} | GASTROENTERITIS AND COLITIS (5589), count during F2: |
| | GASTROENTERITIS_AND_COLITIS | second blood test (weeks 16-19) |
| 2007 | F2_hospital_6289 | Hospital diagnosis INFERTILITY, FEMALE, PRIMARY (6289 |
| | 1_INFERTILITY_{—} | 1), count during F2: second blood test (weeks 16-19) |
| | FEMALE_PRIMARY | |
| 2008 | F2_hospital_64813_ANTEPARTUM_{—} | Hospital diagnosis ANTEPARTUM THYROID DYSFUNCTION |
| | THYROID_DYSFUNCTION | (64813), count during F2: second blood test (weeks 16-19) |
| 2009 | F2_hospital_57420_CALCUL_OF_{—} | Hospital diagnosis CALCUL.OF GALLBLADDER WITHOUT |
| | GALLBLADDER_WITHOUT_{—} | CHOLECYSTITIS,WITHOUT OBSTR. (57420), count during |
| | CHOLECYSTITIS_WITHOUT_OBSTR | F2: second blood test (weeks 16-19) |
| 2010 | F0_ANTINUCLEAR_Ab_{——}ANA | Labtest ANTINUCLEAR Ab_(ANA), sampled during F0: before |
| | | and early pregnancy |
| 2011 | F0_DNA_ds_Ab | Labtest DNA (ds) Ab, sampled during F0: before and early |
| | | pregnancy |
| 2012 | F0_TOXOPLASMA_IgG | Labtest TOXOPLASMA IgG, sampled during F0: before and |
| | | early pregnancy |
| 2013 | F0_HCT_or_HGB_Ratio | Labtest HCT/HGB Ratio, sampled during F0: before and early |
| | | pregnancy |
| 2014 | F0_HbA2 | Labtest HbA2, sampled during F0: before and early pregnancy |
| 2015 | F0_NORMOBLAST_perc | Labtest NORMOBLAST. %, sampled during F0: before and early |
| | | pregnancy |
| 2016 | F0_NORMOBLAST_abs | Labtest NORMOBLAST.abs, sampled during F0: before and early |
| | | pregnancy |
| 2017 | F0_PLATLATE_CLUMPS | Labtest PLATLATE CLUMPS, sampled during F0: before and |
| | | early pregnancy |
| 2018 | F0_LIC | Labtest LIC, sampled during F0: before and early pregnancy |
| 2019 | F0_LIC_perc | Labtest LIC %, sampled during F0: before and early pregnancy |
| 2020 | F0_ALY | Labtest ALY, sampled during F0: before and early pregnancy |
| 2021 | F0_ALY_perc | Labtest ALY %, sampled during F0: before and early pregnancy |
| 2022 | F0_RETICUL_COUNT_abs | Labtest RETICUL, COUNT abs, sampled during F0: before and |
| | | early pregnancy |
| 2023 | F0_RDW_SD | Labtest RDW-SD, sampled during F0: before and early pregnancy |
| 2024 | F0_ANISO_F | Labtest ANISO-F, sampled during F0: before and early pregnancy |
| 2025 | F0_TOXOPLASMA_IgM | Labtest TOXOPLASMA IgM, sampled during F0: before and |
| | | early pregnancy |
| 2026 | F0_LEFT_SHIFT | Labtest LEFT SHIFT, sampled during F0: before and early |
| | | pregnancy |
| 2027 | F0_BLAST_F | Labtest BLAST-F, sampled during F0: before and early pregnancy |
| 2028 | F0_PLT | Labtest PLT, sampled during F0: before and early pregnancy |
| 2029 | F0_CMV_IgM_Add_VIDAS | Labtest CMV IgM(Add.-VIDAS), sampled during F0: before and |
| | | early pregnancy |
| 2030 | F0_BLOOD_TYPE | Labtest BLOOD TYPE, sampled during F0: before and early |
| | | pregnancy |
| 2031 | F0_ANTI_THYROID_PEROXID | Labtest ANTI THYROID PEROXID, sampled during F0: before |
| | | and early pregnancy |
| 2032 | F0_EBV_IgG_EBNA | Labtest EBV IgG-EBNA, sampled during F0: before and early |
| | | pregnancy |
| 2033 | F0_EBV_VCA_IgG | Labtest EBV VCA_IgG, sampled during F0: before and early |
| | | pregnancy |
| 2034 | F0_CMV_IgM | Labtest CMV IgM, sampled during F0: before and early |
| | | pregnancy |
| 2035 | F0_HEPATITIS_Bs_Ab | Labtest HEPATITIS Bs Ab, sampled during F0: before and early |
| | | pregnancy |
| 2036 | F1_ANISO_F | Labtest ANISO-F, sampled during F1: first blood test |
| | | (weeks 6-12) |
| 2037 | F0_clinic_6236_VAGINAL_HEMATOMA | Clinic diagnosis VAGINAL HEMATOMA (6236), count during |
| | | F0: before and early pregnancy |
| 2038 | F0_clinic_7862_COUGH | Clinic diagnosis COUGH (7862), count during F0: before and |
| | | early pregnancy |
| 2039 | F0_clinic_4659_ACUTE_UPPER_{—} | Clinic diagnosis ACUTE UPPER RESPIRATORY INFECTIONS |
| | RESPIRATORY_INFECTIONS_OF_{—} | OF UNSPECIFIED SITE (4659), count during F0: before and |
| | UNSPECIFIED_SITE | early pregnancy |
| 2040 | F2_PHOSPHORUS | Labtest PHOSPHORUS, sampled during F2: second blood test |
| | | (weeks 16-19) |
| 2041 | F2_POTASSIUM | Labtest POTASSIUM, sampled during F2: second blood test |
| | | (weeks 16-19) |
| 2042 | F2_SODIUM | Labtest SODIUM, sampled during F2: second blood test |
| | | (weeks 16-19) |
| 2043 | F0_clinic_465_ACUTE_UPPER_{—} | Clinic diagnosis ACUTE UPPER RESPIRATORY INFECTIONS |
| | RESPIRATORY_INF | OF MULTIPLE OR UNSP.SITES (465), count during F0: before |
| | ECTIONS_OF_MULTIPLE_OR_UNSP_{—} | and early pregnancy |
| | SITES | |
| 2044 | F2_CONTROL_PTT | Labtest CONTROL PTT, sampled during F2: second blood test |
| | | (weeks 16-19) |
| 2045 | F2_CMV_AVIDITY | Labtest CMV AVIDITY, sampled during F2: second blood test |
| | | (weeks 16-19) |
| 2046 | F2_HEMOGLOBIN_H | Labtest HEMOGLOBIN H, sampled during F2: second blood test |
| | | (weeks 16-19) |
| 2047 | F2_HEMOGLOBIN_O | Labtest HEMOGLOBIN O, sampled during F2: second blood test |
| | | (weeks 16-19) |
| 2048 | F2_HEMOGLOBIN_C | Labtest HEMOGLOBIN C, sampled during F2: second blood test |
| | | (weeks 16-19) |
| 2049 | F2_HB_other | Labtest HB -other, sampled during F2: second blood test |
| | | (weeks 16-19) |
| 2050 | F2_HbA2 | Labtest HbA2, sampled during F2: second blood test |
| | | (weeks 16-19) |
| 2051 | F2_NORMOBLAST_perc | Labtest NORMOBLAST. %, sampled during F2: second blood test |
| | | (weeks 16-19) |
| 2052 | F0_clinic_5990_URINARY_TRACT_{—} | Clinic diagnosis URINARY TRACT INFECTION, SITE NOT |
| | INFECTION_SITE_NOT_SPECIFIED | SPECIFIED (5990), count during F0: before and early pregnancy |
| 2053 | F2_PLATLATE_CLUMPS | Labtest PLATLATE CLUMPS, sampled during F2: second blood |
| | | test (weeks 16-19) |
| 2054 | F2_CH | Labtest CH, sampled during F2: second blood test (weeks 16-19) |
| 2055 | F2_LIC | Labtest LIC, sampled during F2: second blood test (weeks 16-19) |
| 2056 | F2_LIC_perc | Labtest LIC %, sampled during F2: second blood test |
| | | (weeks 16-19) |
| 2057 | F2_ALY | Labtest ALY, sampled during F2: second blood test |
| | | (weeks 16-19) |
| 2058 | F2_ALY_perc | Labtest ALY %, sampled during F2: second blood test |
| | | (weeks 16-19) |
| 2059 | F2_HCT_or_HGB_Ratio | Labtest HCT/HGB Ratio, sampled during F2: second blood test |
| | | (weeks 16-19) |
| 2060 | F2_BASO_abs | Labtest BASO abs, sampled during F2: second blood test |
| | | (weeks 16-19) |
| 2061 | F2_MONO_perc | Labtest MONO %, sampled during F2: second blood test |
| | | (weeks 16-19) |
| 2062 | F0_clinic_Z8926_GYNECOLOGICAL_{—} | Clinic diagnosis GYNECOLOGICAL EXAMINATION (Z8926), |
| | EXAMINATION | count during F0: before and early pregnancy |
| 2063 | F2_BILIRUBIN_DIRECT | Labtest BILIRUBIN-DIRECT, sampled during F2: second blood |
| | | test (weeks 16-19) |
| 2064 | F2_LIPASE | Labtest LIPASE, sampled during F2: second blood test |
| | | (weeks 16-19) |
| 2065 | F2_SHBG | Labtest SHBG, sampled during F2: second blood test |
| | | (weeks 16-19) |
| 2066 | F0_pharma_B03AD02_FOLEX_400_{—} | Prescription of FOLEX 400 TAB CF BOX 30 (B03AD02), count |
| | TAB_CF_BOX_30 | during F0: before and early pregnancy |
| 2067 | F0_pharma_N02BE01_DEXAMOL_{—} | Prescription of DEXAMOL CPL 500 MG 20 (N02BE01), count |
| | CPL_500MG_20 | during F0: before and early pregnancy |
| 2068 | F0_pharma_A11AA06_PRENATAL_{—} | Prescription of PRENATAL 9 MONTHS (ALTMAN) CPL 60 |
| | 9_MONTHS_ALTMAN_CPL_60 | (A11AA06), count during F0: before and early pregnancy |
| 2069 | F0_pharma_G03CA03_ESTROFEM_{—} | Prescription of ESTROFEM TAB 2 MG 28 (G03CA03), count |
| | TAB_2MG_28 | during F0: before and early pregnancy |
| 2070 | F0_pharma_B01AB05_CLEXANE_{—} | Prescription of CLEXANE SAFETY LOCK INJ 40 MG/0.4 ML 2 |
| | SAFETY_LOCK_INJ_40MG_OR_0_4ML_2 | (B01AB05), count during F0: before and early pregnancy |
| 2071 | F0_pharma_B03BB01_FOLIC_ACID_{—} | Prescription of FOLIC ACID (REKAH) TAB 5 MG 30 |
| | REKAH_TAB_5MG_30 | (B03BB01), count during F0: before and early pregnancy |
| 2072 | F0_pharma_A03FA01_PRAACAMOL1 | Prescription of PRAMIN TAB 10 MG 30 (A03FA01), count |
| | | during F0: before and early pregnancy |
| 2073 | F0_pharma_G03DA04_UTROGESTAN_{—} | Prescription of UTROGESTAN ORAL/VAG CAP 200 mg 15 |
| | ORAL_OR_VAG_CAP_200mg_15 | (G03DA04), count during F0: before and early pregnancy |
| 2074 | F0_pharma_J07BB02_VAXIGRIP_{—} | Prescription of VAXIGRIP VAC (J07BB02), count during F0: |
| | VAC | before and early pregnancy |
| 2075 | F0_pharma_Unknown_ATC | Prescription of (Unknown_ATC), count during F0: before and |
| | | early pregnancy |
| 2076 | F0_pharma_G03DA04_ENDOMETRIN_{—} | Prescription of ENDOMETRIN VAGINAL TAB 100 MG BOX |
| | VAGINAL_TAB_100MG_BOX_30 | 30 (G03DA04), count during F0: before and early pregnancy |
| 2077 | F0_pharma_B03AD21_FERRIFOL_{—} | Prescription of FERRIFOL TAB CF BOX 30 (B03AD21), count |
| | TAB_CF_BOX_30 | during F0: before and early pregnancy |
| 2078 | F2_FREE_ANDROGEN_INDEX | Labtest FREE ANDROGEN INDEX, sampled during F2: second |
| | | blood test (weeks 16-19) |
| 2079 | F0_pharma_A11AA06_PRENATAL_{—} | Prescription of PRENATAL PLUS CF TAB CF BOX 30 |
| | PLUS_CF_TAB_CF_BOX_30 | (A11AA06), count during F0: before and early pregnancy |
| 2080 | F2_Urine_culture | Labtest Urine culture, sampled during F2: second blood test |
| | | (weeks 16-19) |
| 2081 | F2_DHEA_SULPHATE | Labtest DHEA SULPHATE, sampled during F2: second blood |
| | | test (weeks 16-19) |
| 2082 | F2_PROLACTIN | Labtest PROLACTIN, sampled during F2: second blood test |
| | | (weeks 16-19) |
| 2083 | F2_FSH | Labtest FSH. sampled during F2: second blood test (weeks 16-19) |
| 2084 | F2_ANTI_BODY_SCREEN_I | Labtest ANTI BODY SCREEN I, sampled during F2: second |
| | | blood test (weeks 16-19) |
| 2085 | F2_ERYTHROCYTES_SED | Labtest ERYTHROCYTES-SED, sampled during F2: second |
| | | blood test (weeks 16-19) |
| 2086 | F2_UROBILINOGEN_U_STRIP | Labtest UROBILINOGEN-U STRIP, sampled during F2: second |
| | | blood test (weeks 16-19) |
| 2087 | F2_NITRITE_U_STRIP | Labtest NITRITE- U STRIP, sampled during F2: second blood |
| | | test (weeks 16-19) |
| 2088 | F2_BILIRUBIN_U_STRIP | Labtest BILIRUBIN- U STRIP, sampled during F2: second blood |
| | | test (weeks 16-19) |
| 2089 | F2_ANISO_F | Labtest ANISO-F, sampled during F2: second blood test |
| | | (weeks 16-19) |
| 2090 | F2_LEFT_SHIFT | Labtest LEFT SHIFT, sampled during F2: second blood test |
| | | (weeks 16-19) |
| 2091 | F0_clinic_461_ACUTE_SINUSITIS | Clinic diagnosis ACUTE SINUSITIS (461), count during F0: |
| | | before and early pregnancy |
| 2092 | F2_BLAST_F | Labtest BLAST-F, sampled during F2: second blood test |
| | | (weeks 16-19) |
| 2093 | F1_NITRITE_U_STRIP | Labtest NITRITE- U STRIP, sampled during F1: first blood test |
| | | (weeks 6-12) |
| 2094 | F1_PROTEIN_U_STRIP | Labtest PROTEIN- U STRIP, sampled during F1: first blood test |
| | | (weeks 6-12) |
| 2095 | F1_BILIRUBIN_U_STRIP | Labtest BILIRUBIN- U STRIP, sampled during F1: first blood |
| | | test (weeks 6-12) |
| 2096 | F1_SHBG | Labtest SHBG, sampled during F1: first blood test (weeks 6-12) |
| 2097 | F1_LIPASE | Labtest LIPASE, sampled during F1: first blood test (weeks 6-12) |
| 2098 | F0_clinic_462_ACUTE_PHARYNGITIS | Clinic diagnosis ACUTE PHARYNGITIS (462), count during F0 |
| | | before and early pregnancy |
| 2099 | F1_POTASSIUM | Labtest POTASSIUM, sampled during F1: first blood test |
| | | (weeks 6-12) |
| 2100 | F1_SODIUM | Labtest SODIUM, sampled during F1: first blood test |
| | | (weeks 6-12) |
| 2101 | F0_clinic_7870_NAUSEA_{—} | Clinic diagnosis NAUSEA AND VOMITING (7870), count |
| | AND_VOMITING | during F0: before and early pregnancy |
| 2102 | F0_clinic_463_ACUTE_TONSILLITIS | Clinic diagnosis ACUTE TONSILLITIS (463), count during F0: |
| | | before and early pregnancy |
| 2103 | F1_HEMOGLOBIN_O | Labtest HEMOGLOBIN O, sampled during F1: first blood test |
| | | (weeks 6-12) |
| 2104 | F1_HEMOGLOBIN_C | Labtest HEMOGLOBIN C, sampled during F1: first blood test |
| | | (weeks 6-12) |
| 2105 | F1_HB_other | Labtest HB -other, sampled during F1: first blood test |
| | | (weeks 6-12) |
| 2106 | F1_NORMOBLAST_perc | Labtest NORMOBLAST. %, sampled during F1: first blood test |
| | | (weeks 6-12) |
| 2107 | F1_NORMOBLAST_abs | Labtest NORMOBLAST.abs, sampled during F1: first blood test |
| | | (weeks 6-12) |
| 2108 | F0_clinic_4_NOTHING_ABNORMAL_{—} | Clinic diagnosis NOTHING ABNORMAL FOUND (4), count |
| | FOUND | during F0: before and early pregnancy |
| 2109 | F1_PLATLATE_CLUMPS | Labtest PLATLATE CLUMPS, sampled during F1: first blood |
| | | test (weeks 6-12) |
| 2110 | F0_clinic_6430_MILD_HYPEREMESIS_{—} | Clinic diagnosis MILD HYPEREMESIS GRAVIDARUM (6430), |
| | GRAVIDARUM | count during F0: before and early pregnancy |
| 2111 | F1_LIC | Labtest LIC, sampled during F1: first blood test (weeks 6-12) |
| 2112 | F1_LIC_perc | Labtest LIC %, sampled during F1: first blood test (weeks 6-12) |
| 2113 | F1_ALY | Labtest ALY, sampled during F1: first blood test (weeks 6-12) |
| 2114 | F1_ALY_perc | Labtest ALY %, sampled during F1: first blood test (weeks 6-12) |
| 2115 | F1_RDW_CV | Labtest RDW-CV, sampled during F1: first blood test |
| | | (weeks 6-12) |
| 2116 | F1_LEUCOCYTES_SED | Labtest LEUCOCYTES-SED, sampled during F1: first blood test |
| | | (weeks 6-12) |
| 2117 | F1_ANTI_BODY_SCREEN_I | Labtest ANTI BODY SCREEN I, sampled during F1: first blood |
| | | test (weeks 6-12) |
| 2118 | F0_clinic_7840_HEADACHE | Clinic diagnosis HEADACHE (7840), count during F0: before |
| | | and early pregnancy |
| 2119 | F2_HEPATITIS_C_Ab | Labtest HEPATITIS C Ab, sampled during F2: second blood test |
| | | (weeks 16-19) |
| 2120 | F2_MICRO_F | Labtest MICRO-F, sampled during F2: second blood test |
| | | (weeks 16-19) |
| 2121 | F0_clinic_W78_PREGNANCY_{—} | Clinic diagnosis PREGNANCY: CONFIRMED (W78), count |
| | CONFIRMED | during F0: before and early pregnancy |
| 2122 | F2_CMV_IgG_Add_VIDAS | Labtest CMV IgG (Add VIDAS), sampled during F2: second |
| | | blood test (weeks 16-19) |
| 2123 | F2_CMV_IgM_Add_VIDAS | Labtest CMV IgM(Add.-VIDAS), sampled during F2: second |
| | | blood test (weeks 16-19) |
| 2124 | F2_BLOOD_TYPE | Labtest BLOOD TYPE, sampled during F2: second blood test |
| | | (weeks 16-19) |
| 2125 | F2_ANTI_CARDIOLIPIN_IgG | Labtest ANTI CARDIOLIPIN IgG, sampled during F2: second |
| | | blood test (weeks 16-19) |
| 2126 | F2_EBV_VCA_IgM | Labtest EBV VCA IgM, sampled during F2: second blood test |
| | | (weeks 16-19) |
| 2127 | F2_EBV_IgG_EBNA | Labtest EBV IgG-EBNA, sampled during F2: second blood test |
| | | (weeks 16-19) |
| 2128 | F2_EBV_VCA_IgG | Labtest EBV VCA_IgG, sampled during F2: second blood test |
| | | (weeks 16-19) |
| 2129 | F2_CMV_IgM | Labtest CMV IgM, sampled during F2: second blood test |
| | | (weeks 16-19) |
| 2130 | F2_HEPATITIS_Bs_Ag | Labtest HEPATITIS Bs Ag, sampled during F2: second blood test |
| | | (weeks 16-19) |
| 2131 | F1_Urine_culture | Labtest Urine culture, sampled during F1: first blood test |
| | | (weeks 6-12) |
| 2132 | F2_TOXOPLASMA_IgM | Labtest TOXOPLASMA IgM, sampled during F2: second blood |
| | | test (weeks 16-19) |
| 2133 | F2_TOXOPLASMA_IgG | Labtest TOXOPLASMA IgG, sampled during F2: second blood |
| | | test (weeks 16-19) |
| 2134 | F2_COMPLEMENT_C4 | Labtest COMPLEMENT C4, sampled during F2: second blood |
| | | test (weeks 16-19) |
| 2135 | F2_DNA_ds_Ab | Labtest DNA (ds) Ab, sampled during F2: second blood test |
| | | (weeks 16-19) |
| 2136 | F2_ANTI_CARDIOLIPIN_IgG | Labtest ANTI CARDIOLIPIN IgM, sampled during F2: second |
| | | blood test (weeks 16-19) |
| 2137 | F2_ANTINUCLEAR_Ab_{——}ANA | Labtest ANTINUCLEAR Ab_(ANA), sampled during F2: second |
| | | blood test (weeks 16-19) |
| 2138 | F2_MICROALBUMIN_U_SAMP | Labtest MICROALBUMIN-U SAMP, sampled during F2: second |
| | | blood test (weeks 16-19) |
| 2139 | F2_LIPEMIC | Labtest LIPEMIC, sampled during F2: second blood test |
| | | (weeks 16-19) |
| 2140 | F2_HEMOLYTIC | Labtest HEMOLYTIC, sampled during F2: second blood test |
| | | (weeks 16-19) |
| 2141 | F1_First_isolate | Labtest First isolate, sampled during F1: first blood test |
| | | (weeks 6-12) |
| 2142 | F2_hospital_65420_UTERINE_{—} | Hospital diagnosis UTERINE SCAR FROM |
| | SCAR_FROM_PREV_{—} | PREV.SURGERY, UNSP.EPISODE OF CARE, IN PREGN. |
| | SURGERY_UNSP_EPISODE_OF_{—} | (65420), count during F2: second blood test (weeks 16-19) |
| | CARE_IN_PREGN | |
| 2143 | F2_hospital_3051_TOBACCO_USE_{—} | Hospital diagnosis TOBACCO USE DISORDER (3051), count |
| | DISORDER | during F2: second blood test (weeks 16-19) |
| 2144 | F0_hospital_6202_OTHER_AND_{—} | Hospital diagnosis OTHER AND UNSPECIFIED OVARIAN |
| | UNSPECIFIED_OVARIAN_CYST | CYST (6202), count during F0: before and early pregnancy |
| 2145 | F1_hospital_65103_TWIN_PREGNANCY_{—} | Hospital diagnosis TWIN PREGNANCY, ANTEPARTUM |
| | ANTEPARTUM_CONDITION_OR_{—} | CONDITION OR COMPLICATION (65103), count during F1: |
| | COMPLICATION | first blood test (weeks 6-12) |
| 2146 | F1_hospital_V2385_SUPERVISION_OF_{—} | Hospital diagnosis SUPERVISION OF HIGH RISK |
| | HIGH_RISK_PREGNANCY_RESULTING_{—} | PREGNANCY RESULTING FROM ASSISTED REPROD |
| | FROM_ASSISTED_REPROD_UCTIVE_{—} | UCTIVE TECHNOLOGY (V2385), count during F1: first blood |
| | TECHNOLOGY | test (weeks 6-12) |
| 2147 | F1_hospital_65423_UTERINE_SCAR_{—} | Hospital diagnosis UTERINE SCAR FROM |
| | FROM_PREV_SURGERY_ANTEPARTUM_{—} | PREV.SURGERY, ANTEPARTUM CONDITION/COMPLIC. |
| | CONDITION_OR_COMPLIC | (65423), count during F1: first blood test (weeks 6-12) |
| 2148 | F1_hospital_V140_PERSONAL_{—} | Hospital diagnosis PERSONAL HISTORY OF ALLERGY TO |
| | HISTORY_OF_ALLERGY_TO_{—} | PENICILLIN (V140), count during F1: first blood test |
| | PENICILLIN | (weeks 6-12) |
| 2149 | F1_hospital_78909_ABDOMINAL_{—} | Hospital diagnosis ABDOMINAL PAIN, OTHER SPECIFIED |
| | PAIN_OTHER_SPECIFIED_SITE | SITE (78909), count during F1: first blood test (weeks 6-12) |
| 2150 | F1_hospital_65453_CERVICAL_{—} | Hospital diagnosis CERVICAL INCOMPETENCE, |
| | INCOMPETENCE_ANTEPARTUM_{—} | ANTEPARTUM CONDITION OR COMPLICATION (65453), |
| | CONDITION_OR_COMPLICATION | count during F1: first blood test (weeks 6-12) |
| 2151 | F1_hospital_64300_MILD_{—} | Hospital diagnosis MILD HYPEREMESIS GRAVIDARUM, |
| | HYPEREMESIS_GRAVIDARUM_UNSP_{—} | UNSP.AS TO EPISODE OF CARE (64300), count during F1: |
| | AS_TO_EPISODE_OF_CARE | first blood test (weeks 6-12) |
| 2152 | F1_hospital_Z8878_DIAGNOSTIC_{—} | Hospital diagnosis DIAGNOSTIC ULTRASOUND OF GRAVID |
| | ULTRASOUND_OF_GRAVID_UTERUS | UTERUS (Z8878), count during F1: first blood test (weeks 6-12) |
| 2153 | F1_hospital_Z9921_INJECTION_OF_{—} | Hospital diagnosis INJECTION OF ANTIBIOTIC (Z9921), count |
| | ANTIBIOTIC | during F1: first blood test (weeks 6-12) |
| 2154 | F1_hospital_5990_URINARY_TRACT_{—} | Hospital diagnosis URINARY TRACT INFECTION, SITE NOT |
| | INFECTION_SITE_NOT_SPECIFIED | SPECIFIED (5990), count during F1: first blood test (weeks 6-12) |
| 2155 | F1_hospital_999999 | Hospital diagnosis (999999), count during F1: first blood test |
| | | (weeks 6-12) |
| 2156 | F1_hospital_V22_NORMAL_PREGNANCY | Hospital diagnosis NORMAL PREGNANCY (V22), count during |
| | | F1: first blood test (weeks 6-12) |
| 2157 | F1_hospital_Z8879_OTHER_{—} | Hospital diagnosis OTHER DIAGNOSTIC ULTRASOUND |
| | DIAGNOSTIC_ULTRASOUND | (Z8879), count during F1: first blood test (weeks 6-12) |
| 2158 | F1_hospital_64663_ANTEPARTUM_{—} | Hospital diagnosis ANTEPARTUM INFECTIONS OF |
| | INFECTIONS_OF_GENITOURINARY_{—} | GENITOURINARY TRACT (64663), count during F1: first |
| | TRACT | blood test (weeks 6-12) |
| 2159 | F1_hospital_Z96490_ABORTION_{—} | Hospital diagnosis ABORTION INDUCED BY MEDICATION |
| | INDUCED_BY_MEDICATION | (Z96490), count during F1: first blood test (weeks 6-12) |
| 2160 | F1_hospital_6288_INFERTILITY_{—} | Hospital diagnosis INFERTILITY, FEMALE, OF OTHER |
| | FEMALE_OF_OTHER_SPECIFIED_{—} | SPECIFIED ORIGIN (6288), count during F1: first blood test |
| | ORIGIN | (weeks 6-12) |
| 2161 | F1_hospital_64003_THREATENED_{—} | Hospital diagnosis THREATENED ABORTION, |
| | ABORTION_ANTEPARTUM | ANTEPARTUM (64003), count during F1: first blood test |
| | | (weeks 6-12) |
| 2162 | F1_hospital_Z6902_DILATION_AND_{—} | Hospital diagnosis DILATION AND CURETTAGE |
| | CURETTAGE_FOLLOWING_DELIVERY_{—} | FOLLOWING DELIVERY OR ABORTION (Z6902), count |
| | OR_ABORTION | during F1: first blood test (weeks 6-12) |
| 2163 | F1_hospital_V221_SUPERVISION_OF_{—} | Hospital diagnosis SUPERVISION OF OTHER NORMAL |
| | OTHER_NORMAL_PREGNANCY | PREGNANCY (V221), count during F1: first blood test |
| | | (weeks 6-12) |
| 2164 | F1_hospital_Z65910_I_V_F | Hospital diagnosis I.V.F. (Z65910), count during F1: first blood |
| | | test (weeks 6-12) |
| 2165 | F1_hospital_78900_ABDOMINAL_{—} | Hospital diagnosis ABDOMINAL PAIN, UNSP. SITE (78900), |
| | PAIN_UNSP_SITE | count during F1: first blood test (weeks 6-12) |
| 2166 | F1_hospital_V2681_ENCOUNTER_{—} | Hospital diagnosis ENCOUNTER FOR ASSISTED |
| | FOR_ASSISTED_REPRODUCTIVE_{—} | REPRODUCTIVE FERTILITY PROCEDURE CYCLE (V2681), |
| | FERTILITY_PROCEDURE_CYCLE | count during F1: first blood test (weeks 6-12) |
| 2167 | F1_hospital_V222_PREGNANT_STATE_{—} | Hospital diagnosis PREGNANT STATE. INCIDENTAL (V222), |
| | INCIDENTAL | count during F1: first blood test (weeks 6-12) |
| 2168 | F0_hospital_Z6595_RELEASE_OF_{—} | Hospital diagnosis RELEASE OF TORSION OF OVARY |
| | TORSION_OF_OVARY | (Z6595), count during F0: before and early pregnancy |
| 2169 | F1_hospital_7890_ABDOMINAL_PAIN | Hospital diagnosis ABDOMINAL PAIN (7890), count during F1: |
| | | first blood test (weeks 6-12) |
| 2170 | F1_hospital_78903_ABDOMINAL_{—} | Hospital diagnosis ABDOMINAL PAIN, RT. LOWER |
| | PAIN_RT_LOWER_QUADRANT | QUADRANT (78903), count during F1: first blood test |
| | | (weeks 6-12) |
| 2171 | F2_hospital_63490_SPONTANEOUS_{—} | Hospital diagnosis SPONTANEOUS |
| | ABORTION_UNSPECIFIED_WITHOUT_{—} | ABORTION, UNSPECIFIED, WITHOUT COMPLICATION |
| | COMPLICATION | (63490), count during F2: second blood test (weeks 16-19) |
| 2172 | F1_hospital_63491_SPONTANEOUS_{—} | Hospital diagnosis SPONTANEOUS |
| | ABORTION_INCOMPLETE_WITHOUT_{—} | ABORTION, INCOMPLETE, WITHOUT COMPLICATION |
| | COMPLICATION | (63491), count during F1: first blood test (weeks 6-12) |
| 2173 | F1_hospital_6280 | Hospital diagnosis INFERTILITY, FEMALE, PRIMARY (6280 1), |
| | 1_INFERTILITY_{—} | count during F1: first blood test (weeks 6-12) |
| | FEMALE_PRIMARY | |
| 2174 | F1_hospital_Z8876_DIAGNOSTIC_{—} | Hospital diagnosis DIAGNOSTIC ULTRASOUND OF |
| | ULTRASOUND_OF_ABDOMEN_AND_{—} | ABDOMEN AND RETROPERITONEUM (Z8876), count during |
| | RETROPERITONEUM | F1: first blood test (weeks 6-12) |
| 2175 | F1_hospital_6289_INFERTILITY_{—} | Hospital diagnosis INFERTILITY, FEMALE, OF UNSPECIFIED |
| | FEMALE_OF_UNSPECIFIED_ORIGIN | ORIGIN (6289), count during F1: first blood test (weeks 6-12) |
| 2176 | F1_hospital_V141_PERSONAL_{—} | Hospital diagnosis PERSONAL HISTORY OF ALLERGY TO |
| | HISTORY_OF_ALLERGY_TO_OTHER_{—} | OTHER ANTIBIOTIC AGENT (V141), count during F1: first |
| | ANTIBIOTIC_AGENT | blood test (weeks 6-12) |
| 2177 | F1_hospital_64000_THREATENED_{—} | Hospital diagnosis THREATENED ABORTION, UNSPECIFIED |
| | ABORTION_UNSPECIFIED_AS_TO_{—} | AS TO EPISODE OF CARE (64000), count during F1: first blood |
| | EPISODE_OF_CARE | test (weeks 6-12) |
| 2178 | F1_hospital_64823_ANTEPARTUM_{—} | Hospital diagnosis ANTEPARTUM ANEMIA (64823), count |
| | ANEMIA | during F1: first blood test (weeks 6-12) |
| 2179 | F1_hospital_63791_UNSPECIFIED_{—} | Hospital diagnosis UNSPECIFIED |
| | ABORTION_INCOMPLETE_WITHOUT_{—} | ABORTION, INCOMPLETE, WITHOUT COMPLICATION |
| | COMPLICATION | (63791), count during F1: first blood test (weeks 6-12) |
| 2180 | F1_hospital_Z6759_CERCLAGE_{—} | Hospital diagnosis CERCLAGE-UNSPECIFIED |
| | UNSPECIFIED_CERVIX_ISTHMUS_{—} | (CERVIX)(ISTHMUS UTERI) (Z6759), count during F1: first |
| | UTERI | blood test (weeks 6-12) |
| 2181 | F1_hospital_V232_SUPERVISION_OF_{—} | Hospital diagnosis SUPERVISION OF HIGH-RISK |
| | HIGH_RISK_PREGNANCY_WITH_{—} | PREGNANCY WITH HISTORY OF ABORTION (V232), count |
| | HISTORY_OF_ABORTION | during F1: first blood test (weeks 6-12) |
| 2182 | F1_hospital_7840_HEADACHE | Hospital diagnosis HEADACHE (7840), count during F1: first |
| | | blood test (weeks 6-12) |
| 2183 | F1_hospital_Z9929_INJECT_OR_{—} | Hospital diagnosis INJECTOR INFUSION OF OTHER |
| | INFUSION_OF_OTHER_THERAPEUTIC_{—} | THERAPEUTIC OR PROPHYLACTIC SUBS. (Z9929), count |
| | OR_PROPHYLACTIC_SUBS | during F1: first blood test (weeks 6-12) |
| 2184 | F1_hospital_V148_PERSONAL_{—} | Hospital diagnosis PERSONAL HISTORY OF ALLERGY TO |
| | HISTORY_OF_ALLERGY_TO_{—} | SPECIFIED MEDICINAL AGENTS (V148), count during F1: |
| | SPECIFIED_MEDICINAL_AGENTS | first blood test (weeks 6-12) |
| 2185 | F1_hospital_6280 | Hospital diagnosis INFERTILITY, FEMALE, SECONDARY |
| | 2_INFERTILITY_{—} | (6280 2), count during F1: first blood test (weeks 6-12) |
| | FEMALE_SECONDARY | |
| 2186 | F1_hospital_64393_UNSPECIFIED_{—} | Hospital diagnosis UNSPECIFIED VOMITING OF |
| | VOMITING_OF_PREGNANCY_{—} | PREGNANCY, ANTEPARTUM (64393), count during F1: first |
| | ANTEPARTUM | blood test (weeks 6-12) |
| 2187 | F1_hospital_2561_OTHER_OVARIAN_{—} | Hospital diagnosis OTHER OVARIAN HYPERFUNCTION |
| | HYPERFUNCTION | (2561), count during F1: first blood test (weeks 6-12) |
| 2188 | F1_hospital_6202_OTHER_AND_{—} | Hospital diagnosis OTHER AND UNSPECIFIED OVARIAN |
| | UNSPECIFIED_OVARIAN_CYST | CYST (6202), count during F1: first blood test (weeks 6-12) |
| 2189 | F1_hospital_59080_PYELONEPHRITIS_{—} | Hospital diagnosis PYELONEPHRITIS, UNSPECIFIED (59080), |
| | UNSPECIFIED | count during F1: first blood test (weeks 6-12) |
| 2190 | F1_hospital_6289 | Hospital diagnosis INFERTILITY, FEMALE, SECONDARY |
| | 2_INFERTILITY_{—} | (6289 2), count during F1: first blood test (weeks 6-12) |
| | FEMALE_SECONDARY | |
| 2191 | F1_hospital_Z6812_HYSTEROSCOPY | Hospital diagnosis HYSTEROSCOPY (Z6812), count during F1: |
| | | first blood test (weeks 6-12) |
| 2192 | F1_hospital_V714_OBSERVATION_{—} | Hospital diagnosis OBSERVATION FOLLOWING OTHER |
| | FOLLOWING_OTHER_ACCIDENT | ACCIDENT (V714), count during F1: first blood test |
| | | (weeks 6-12) |
| 2193 | F1_hospital_0_UNKNOWN_CATEGORY | Hospital diagnosis UNKNOWN CATEGORY (0), count during |
| | | F1: first blood test (weeks 6-12) |
| 2194 | F1_hospital_7999_UNKNOWN_OR_{—} | Hospital diagnosis UNKNOWN OR UNSPECIFIED CAUSE OF |
| | UNSPECIFIED_CAUSE_OF_MORBIDITY_{—} | MORBIDITY (DIAGNOSIS DEFERRED) (7999), count during |
| | DIAGNOSIS_DEFERRED | F1: first blood test (weeks 6-12) |
| 2195 | F1_hospital_2449_UNSPECIFIED_{—} | Hospital diagnosis UNSPECIFIED ACQUIRED |
| | ACQUIRED_HYPOTHYROIDISM | HYPOTHYROIDISM (2449), count during F1: first blood test |
| | | (weeks 6-12) |
| 2196 | F0_hospital_6201_CORPUS_LUTEUM_{—} | Hospital diagnosis CORPUS LUTEUM CYST OR HEMATOMA |
| | CYST_OR_HEMATOMA | (6201), count during F0: before and early pregnancy |
| 2197 | F0_hospital_Z8875_DIAGNOSTIC_{—} | Hospital diagnosis DIAGNOSTIC ULTRASOUND OF |
| | ULTRASOUND_OF_URINARY_SYSTEM | URINARY SYSTEM (Z8875), count during F0: before and early |
| | | pregnancy |
| 2198 | F0_hospital_64973_CERVICAL_{—} | Hospital diagnosis CERVICAL SHORTENING |
| | SHORTENING_COMPLICATING_{—} | COMPLICATING PREGNANCY, ANTEPARTUM |
| | PREGNANCY_ANTEPARTUM_{—} | CONDITION OR COMPLICATION (64973), count during F0: |
| | CONDITION_OR_COMPLICATION | before and early pregnancy |
| 2199 | F0_hospital_591_HYDRONEPHROSIS | Hospital diagnosis HYDRONEPHROSIS (591), count during F0: |
| | | before and early pregnancy |
| 2200 | F0_hospital_Z5421_LAPAROSCOPY | Hospital diagnosis LAPAROSCOPY (Z5421), count during F0: |
| | | before and early pregnancy |
| 2201 | F0_hospital_5409_ACUTE_{—} | Hospital diagnosis ACUTE APPENDICITIS WITHOUT |
| | APPENDICITIS_WITHOUT_MENTION_{—} | MENTION OF PERITONITIS (5409), count during F0: before |
| | OF_PERITONITIS | and early pregnancy |
| 2202 | F0_hospital_63590_LEG_INDUCED_{—} | Hospital diagnosis LEG.INDUCED |
| | ABORTION_UNSPEC_WITHOUT_{—} | ABORTION, UNSPEC., WITHOUT MENTION OF |
| | MENTION_OF_COMPLICATION | COMPLICATION (63590), count during F0: before and early |
| | | pregnancy |
| 2203 | F0_hospital_V719_OBSERVATION_{—} | Hospital diagnosis OBSERVATION FOR UNSPECIFIED |
| | FOR_UNSPECIFIED_SUSPECTED_{—} | SUSPECTED CONDITION (V719), count during F0: before and |
| | CONDITION | early pregnancy |
| 2204 | F0_hospital_49390_ASTHMA_{—} | Hospital diagnosis ASTHMA, UNSPECIFIED TYPE, WITHOUT |
| | UNSPECIFIED_TYPE_WITHOUT_{—} | MENTION OF STATUS ASTHMATICUS (49390), count during |
| | MENTION_OF_STATUS_ASTHMATICUS | F0: before and early pregnancy |
| 2205 | F0_hospital_6205_TORSION_OF_{—} | Hospital diagnosis TORSION OF OVARY, OVARIAN |
| | OVARY_OVARIAN_PEDICLE_OR_{—} | PEDICLE, OR FALLOPIAN TUBE (6205), count during F0: |
| | FALLOPIAN_TUBE | before and early pregnancy |
| 2206 | F0_hospital_64403_THREATENED_{—} | Hospital diagnosis THREATENED PREMATURE LABOR, |
| | PREMATURE_LABOR_ANTEPARTUM | ANTEPARTUM (64403), count during F0: before and early |
| | | pregnancy |
| 2207 | F0_hospital_Z67591_CERCLAGE_MC_{—} | Hospital diagnosis CERCLAGE-MC DONALD |
| | DONALD_ENCIRCLEMENT_SUTURE_{—} | (ENCIRCLEMENT SUTURE, CERVIX) (Z67591), count during |
| | CERVIX | F0: before and early pregnancy |
| 2208 | F0_hospital_2859_ANEMIA_{—} | Hospital diagnosis ANEMIA, UNSPECIFIED (2859), count |
| | UNSPECIFIED | during F0: before and early pregnancy |
| 2209 | F0_hospital_6280 | Hospital diagnosis INFERTILITY, FEMALE, PRIMARY (6280 |
| | 1_INFERTILITY_{—} | 1), count during F0: before and early pregnancy |
| | FEMALE_PRIMARY | |
| 2210 | F0_hospital_Z8876_DIAGNOSTIC_{—} | Hospital diagnosis DIAGNOSTIC ULTRASOUND OF |
| | ULTRASOUND_OF_ABDOMEN_AND_{—} | ABDOMEN AND RETROPERITONEUM (Z8876), count during |
| | RETROPERITONEUM | F0: before and early pregnancy |
| 2211 | F0_hospital_6289_INFERTILITY_{—} | Hospital diagnosis INFERTILITY, FEMALE, OF UNSPECIFIED |
| | FEMALE_OF_UNSPECIFIED_ORIGIN | ORIGIN (6289), count during F0: before and early pregnancy |
| 2212 | F0_hospital_V141_PERSONAL_{—} | Hospital diagnosis PERSONAL HISTORY OF ALLERGY TO |
| | HISTORY_OF_ALLERGY_TO_OTHER_{—} | OTHER ANTIBIOTIC AGENT (V141), count during F0: before |
| | ANTIBIOTIC_AGENT | and early pregnancy |
| 2213 | F0_hospital_64000_THREATENED_{—} | Hospital diagnosis THREATENED ABORTION, UNSPECIFIED |
| | ABORTION_UNSPECIFIED_AS_TO_{—} | AS TO EPISODE OF CARE (64000), count during F0: before |
| | EPISODE_OF_CARE | and early pregnancy |
| 2214 | F0_hospital_64823_ANTEPARTUM_{—} | Hospital diagnosis ANTEPARTUM ANEMIA (64823), count |
| | ANEMIA | during F0: before and early pregnancy |
| 2215 | F0_hospital_63791_UNSPECIFIED_{—} | Hospital diagnosis UNSPECIFIED |
| | ABORTION_INCOMPLETE_WITHOUT_{—} | ABORTION, INCOMPLETE, WITHOUT COMPLICATION |
| | COMPLICATION | (63791), count during F0: before and early pregnancy |
| 2216 | F0_hospital_Z6759_CERCLAGE_{—} | Hospital diagnosis CERCLAGE-UNSPECIFIED |
| | UNSPECIFIED_CERVIX_ISTHMUS_UTERI | (CERVIX)(ISTHMUS UTERI) (Z6759), count during F0: before |
| | | and early pregnancy |
| 2217 | F0_hospital_V232_SUPERVISION_OF_{—} | Hospital diagnosis SUPERVISION OF HIGH-RISK |
| | HIGH_RISK_PREGNANCY_WITH_{—} | PREGNANCY WITH HISTORY OF ABORTION (V232), count |
| | HISTORY_OF_ABORTION | during F0: before and early pregnancy |
| 2218 | F0_hospital_Z9929_INJECT_OR_{—} | Hospital diagnosis INJECTOR INFUSION OF OTHER |
| | INFUSION_OF_OTHER_THERAPEUTIC_{—} | THERAPEUTIC OR PROPHYLACTIC SUBS. (Z9929), count |
| | OR_PROPHYLACTIC_SUBS | during F0: before and early pregnancy |
| 2219 | F0_hospital_V148_PERSONAL_{—} | Hospital diagnosis PERSONAL HISTORY OF ALLERGY TO |
| | HISTORY_OF_ALLERGY_TO_{—} | SPECIFIED MEDICINAL AGENTS (V148), count during F0: |
| | SPECIFIED_MEDICINAL_AGENTS | before and early pregnancy |
| 2220 | F0_hospital_62802_INFERTILITY_{—} | Hospital diagnosis INFERTILITY, FEMALE, SECONDARY |
| | FEMALE_SECONDARY | (6280 2), count during F0: before and early pregnancy |
| 2221 | F0_hospital_64393_UNSPECIFIED_{—} | Hospital diagnosis UNSPECIFIED VOMITING OF |
| | VOMITING_OF_PREGNANCY_{—} | PREGNANCY, ANTEPARTUM (64393), count during F0: |
| | ANTEPARTUM | before and early pregnancy |
| 2222 | F0_hospital_2561_OTHER_OVARIAN_{—} | Hospital diagnosis OTHER OVARIAN HYPERFUNCTION |
| | HYPERFUNCTION | (2561), count during F0: before and early pregnancy |
| 2223 | F0_hospital_7802_SYNCOPE_AND_{—} | Hospital diagnosis SYNCOPE AND COLLAPSE (7802), count |
| | COLLAPSE | during F0: before and early pregnancy |
| 2224 | F0_hospital_Z4701_{—} | Hospital diagnosis LAPAROSCOPIC APPENDECTOMY |
| | LAPAROSCOPIC_APPENDECTOMY | (Z4701), count during F0: before and early pregnancy |
| 2225 | F0_hospital_04149_OTHER_AND_{—} | Hospital diagnosis OTHER AND UNSPECIFIED |
| | UNSPECIFIED_ESCHERICHIA_{—}COLI_{—} | ESCHERICHIA COLI (E. COLI) (04149), count during F0: |
| | E_{—}COLI | before and early pregnancy |
| 2226 | F0_hospital_6238_OTHER_{—} | Hospital diagnosis OTHER SPECIFIED |
| | SPECIFIED_NONINFLAMMATORY_{—} | NONINFLAMMATORY DISORDERS OF VAGINA (6238), |
| | DISORDERS_OF_VAGINA | count during F0: before and early pregnancy |
| 2227 | F0_hospital_64193_UNSPECIFIED_{—} | Hospital diagnosis UNSPECIFIED ANTEPARTUM |
| | ANTEPARTUM_HEMORRHAGE | HEMORRHAGE (64193), count during F0: before and early |
| | | pregnancy |
| 2228 | F0_hospital_6400_THREATENED_{—} | Hospital diagnosis THREATENED ABORTION (6400), count |
| | ABORTION | during F0: before and early pregnancy |
| 2229 | F0_hospital_V180_FAMILY_{—} | Hospital diagnosis FAMILY HISTORY OF DIABETES |
| | HISTORY_OF_DIABETES_MELLITUS | MELLITUS (V180), count during F0: before and early pregnancy |
| 2230 | F0_hospital_Z5123_LAPAROSCOPIC_{—} | Hospital diagnosis LAPAROSCOPIC |
| | CHOLECYSTECTOMY_1992 | CHOLECYSTECTOMY_1992 (Z5123), count during F0: before |
| | | and early pregnancy |
| 2231 | F0_hospital_V270_MOTHER_WITH_{—} | Hospital diagnosis MOTHER WITH SINGLE LIVEBORN |
| | SINGLE_LIVEBORN | (V270), count during F0: before and early pregnancy |
| 2232 | F0_hospital_V2389_SUPERVISION_{—} | Hospital diagnosis SUPERVISION OF OTHER HIGH-RISK |
| | OF_OTHER_HIGH_RISK_PREGNANCY | PREGNANCY (V2389), count during F0: before and early |
| | | pregnancy |
| 2233 | F0_hospital_64093_UNSPECIFIED_{—} | Hospital diagnosis UNSPECIFIED HEMORRHAGE IN EARLY |
| | HEMORRHAGE_IN_EARLY_{—} | PREGNANCY, ANTEPARTUM (64093), count during F0: |
| | PREGNANCY_ANTEPARTUM | before and early pregnancy |
| 2234 | F0_hospital_Z9911_INJECTION_OF_{—} | Hospital diagnosis INJECTION OF RH IMMUNE GLOBULIN |
| | RH_IMMUNE_GLOBULIN | (Z9911), count during F0: before and early pregnancy |
| 2235 | F0_hospital_78609_OTHER_DYSPNEA_{—} | Hospital diagnosis OTHER DYSPNEA AND RESPIRATORY |
| | AND_RESPIRATORY_ABNORMALITY | ABNORMALITY (78609), count during F0: before and early |
| | | pregnancy |
| 2236 | F0_hospital_7880_RENAL_COLIC | Hospital diagnosis RENAL COLIC (7880), count during F0: |
| | | before and early pregnancy |
| 2237 | F0_hospital_64313_HYPEREMESIS_{—} | Hospital diagnosis HYPEREMESIS GRAVIDARUM WITH |
| | GRAVIDARUM_WITH_METABOLIC_{—} | METABOLIC DISTURBANCE, ANTEPARTUM (64313), count |
| | DISTURBANCE_ANTEPARTUM | during F0: before and early pregnancy |
| 2238 | F0_hospital_V2341_PREGNANCY_{—} | Hospital diagnosis PREGNANCY WITH HISTORY OF PRE- |
| | WITH_HISTORY_OF_PRE_TERM_LABOR | TERM LABOR (V2341), count during F0: before and early |
| | | pregnancy |
| 2239 | F0_hospital_5589_OTHER_AND_{—} | Hospital diagnosis OTHER AND UNSPEC.NONINFECTIOUS |
| | UNSPEC_NONINFECTIOUS_{—} | GASTROENTERITIS AND COLITIS (5589), count during F0: |
| | GASTROENTERITIS_AND_COLITIS | before and early pregnancy |
| 2240 | F0_hospital_6289 | Hospital diagnosis INFERTILITY, FEMALE, PRIMARY (6289 |
| | 1_INFERTILITY_{—} | 1), count during F0: before and early pregnancy |
| | FEMALE_PRIMARY | |
| 2241 | F0_hospital_64813_ANTEPARTUM_{—} | Hospital diagnosis ANTEPARTUM THYROID DYSFUNCTION |
| | THYROID_DYSFUNCTION | (64813), count during F0: before and early pregnancy |
| 2242 | F0_hospital_57420_CALCUL_OF_{—} | Hospital diagnosis CALCUL.OF GALLBLADDER WITHOUT |
| | GALLBLADDER_WITHOUT_{—} | CHOLECYSTITIS, WITHOUT OBSTR. (57420), count during |
| | CHOLECYSTITIS_WITHOUT_OBSTR | F0: before and early pregnancy |
| 2243 | F0_hospital_V234_SUPERVISION_OF_{—} | Hospital diagnosis SUPERVISION OF HIGH-RISK |
| | HIGH_RISK_PREGN_WITH_POOR_{—} | PREGN.WITH POOR OBSTETRIC HISTORY (V234), count |
| | OBSTETRIC_HISTORY | during F0: before and early pregnancy |
| 2244 | F0_hospital_65420_UTERINE_SCAR_{—} | Hospital diagnosis UTERINE SCAR FROM |
| | FROM_PREV_SURGERY_UNSP_{—} | PREV.SURGERY, UNSP.EPISODE OF CARE, IN PREGN. |
| | EPISODE_OF_CARE_IN_PREGN | (65420), count during F0: before and early pregnancy |
| 2245 | F0_hospital_3051_TOBACCO_USE_{—} | Hospital diagnosis TOBACCO USE DISORDER (3051), count |
| | DISORDER | during F0: before and early pregnancy |
| 2246 | F0_hospital_63490_SPONTANEOUS_{—} | Hospital diagnosis SPONTANEOUS |
| | ABORTION_UNSPECIFIED_WITHOUT_{—} | ABORTION, UNSPECIFIED, WITHOUT COMPLICATION |
| | COMPLICATION | (63490), count during F0: before and early pregnancy |
| 2247 | F0_hospital_65963_OTHER_{—} | Hospital diagnosis OTHER ADVANCED MATERNAL AGE, |
| | ADVANCED_MATERNAL_AGE_{—} | ANTEPARTUM CONDITION/COMPLICATION (65963), count |
| | ANTEPARTUM_CONDITION_OR_{—} | during F0: before and early pregnancy |
| | COMPLICATION | |
| 2248 | F0_hospital_6318_OTHER_ABNORMAL_{—} | Hospital diagnosis OTHER ABNORMAL PRODUCTS OF |
| | PRODUCTS_OF_CONCEPTION | CONCEPTION (6318), count during F0: before and early |
| | | pregnancy |
| 2249 | F1_hospital_2859_ANEMIA_{—} | Hospital diagnosis ANEMIA, UNSPECIFIED (2859), count |
| | UNSPECIFIED | during F1: first blood test (weeks 6-12) |
| 2250 | F1_hospital_64403_THREATENED_{—} | Hospital diagnosis THREATENED PREMATURE LABOR, |
| | PREMATURE_LABOR_ANTEPARTUM | ANTEPARTUM (64403), count during F1: first blood test |
| | | (weeks 6-12) |
| 2251 | F1_hospital_6205_TORSION_OF_{—} | Hospital diagnosis TORSION OF OVARY, OVARIAN |
| | OVARY_OVARIAN_PEDICLE_OR_{—} | PEDICLE, OR FALLOPIAN TUBE (6205), count during F1: firs |
| | FALLOPIAN_TUBE | blood test (weeks 6-12) |
| 2252 | F2_hospital_999999 | Hospital diagnosis (999999), count during F2: second blood test |
| | | (weeks 16-19) |
| 2253 | F2_hospital_V148_PERSONAL_{—} | Hospital diagnosis PERSONAL HISTORY OF ALLERGY TO |
| | HISTORY_OF_ALLERGY_TO_{—} | SPECIFIED MEDICINAL AGENTS (V148), count during F2: |
| | SPECIFIED_MEDICINAL_AGENTS | second blood test (weeks 16-19) |
| 2254 | F2_hospital_62802_INFERTILITY_{—} | Hospital diagnosis INFERTILITY, FEMALE, SECONDARY |
| | FEMALE_SECONDARY | (6280 2), count during F2: second blood test (weeks 16-19) |
| 2255 | F2_hospital_64393_UNSPECIFIED_{—} | Hospital diagnosis UNSPECIFIED VOMITING OF |
| | VOMITING_OF_PREGNANCY_{—} | PREGNANCY, ANTEPARTUM (64393), count during F2: |
| | ANTEPARTUM | second blood test (weeks 16-19) |
| 2256 | F2_hospital_2561_OTHER_OVARIAN_{—} | Hospital diagnosis OTHER OVARIAN HYPERFUNCTION |
| | HYPERFUNCTION | (2561), count during F2: second blood test (weeks 16-19) |
| 2257 | F2_hospital_6202_OTHER_AND_{—} | Hospital diagnosis OTHER AND UNSPECIFIED OVARIAN |
| | UNSPECIFIED_OVARIAN_CYST | CYST (6202), count during F2: second blood test (weeks 16-19) |
| 2258 | F2_hospital_59080_PYELONEPHRITIS_{—} | Hospital diagnosis PYELONEPHRITIS, UNSPECIFIED (59080), |
| | UNSPECIFIED | count during F2: second blood test (weeks 16-19) |
| 2259 | F2_hospital_62892_INFERTILITY_{—} | Hospital diagnosis INFERTILITY, FEMALE, SECONDARY |
| | FEMALE_SECONDARY | (6289 2), count during F2: second blood test (weeks 16-19) |
| 2260 | F2_hospital_V714_OBSERVATION_{—} | Hospital diagnosis OBSERVATION FOLLOWING OTHER |
| | FOLLOWING_OTHER_ACCIDENT | ACCIDENT (V714), count during F2: second blood test |
| | | (weeks 16-19) |
| 2261 | F2_hospital_0_UNKNOWN_CATEGORY | Hospital diagnosis UNKNOWN CATEGORY (0), count during |
| | | F2: second blood test (weeks 16-19) |
| 2262 | F2_hospital_2449_UNSPECIFIED_{—} | Hospital diagnosis UNSPECIFIED ACQUIRED |
| | ACQUIRED_HYPOTHYROIDISM | HYPOTHYROIDISM (2449), count during F2: second blood test |
| | | (weeks 16-19) |
| 2263 | F2_hospital_V220_SUPERVISION_{—} | Hospital diagnosis SUPERVISION OF NORMAL FIRST |
| | OF_NORMAL_FIRST_PREGNANCY | PREGNANCY (V220), count during F2: second blood test |
| | | (weeks 16-19) |
| 2264 | F2_hospital_Z6992_ARTIFICIAL_{—} | Hospital diagnosis ARTIFICIAL INSEMINATION (Z6992), |
| | INSEMINATION | count during F2: second blood test (weeks 16-19) |
| 2265 | F2_hospital_63491_SPONTANEOUS_{—} | Hospital diagnosis SPONTANEOUS |
| | ABORTION_INCOMPLETE_WITHOUT_{—} | ABORTION, INCOMPLETE, WITHOUT COMPLICATION |
| | COMPLICATION | (63491), count during F2: second blood test (weeks 16-19) |
| 2266 | F2_hospital_Z6591_ASPIRATION_{—} | Hospital diagnosis ASPIRATION OF OVARY (Z6591), count |
| | OF_OVARY | during F2: second blood test (weeks 16-19) |
| 2267 | F2_hospital_65103_TWIN_{—} | Hospital diagnosis TWIN PREGNANCY, ANTEPARTUM |
| | PREGNANCY_ANTEPARTUM_{—} | CONDITION OR COMPLICATION (65103), count during F2: |
| | CONDITION_OR_COIMPLICATION | second blood test (weeks 16-19) |
| 2268 | F2_hospital_7890_ABDOMINAL_PAIN | Hospital diagnosis ABDOMINAL PAIN (7890), count during F2: |
| | | second blood test (weeks 16-19) |
| 2269 | F2_hospital_65423_UTERINE_SCAR_{—} | Hospital diagnosis UTERINE SCAR FROM |
| | FROM_PREV_SURGERY_{—} | PREV.SURGERY, ANTEPARTUM CONDITION/COMPLIC. |
| | ANTEPARTUM_CONDITION_OR_{—} | (65423), count during F2: second blood test (weeks 16-19) |
| | COMPLIC | |
| 2270 | F2_hospital_V140_PERSONAL_{—} | Hospital diagnosis PERSONAL HISTORY OF ALLERGY TO |
| | HISTORY_OF_ALLERGY_TO_PENICILLIN | PENICILLIN (V140), count during F2: second blood test |
| | | (weeks 16-19) |
| 2271 | F2_hospital_78909_ABDOMINAL_{—} | Hospital diagnosis ABDOMINAL PAIN, OTHER SPECIFIED |
| | PAIN_OTHER_SPECIFIED_SITE | SITE (78909), count during F2: second blood test (weeks 16-19) |
| 2272 | F2_hospital_65453_CERVICAL_{—} | Hospital diagnosis CERVICAL INCOMPETENCE, |
| | INCOMPETENCE_ANTEPARTUM_{—} | ANTEPARTUM CONDITION OR COMPLICATION (65453), |
| | CONDITION_OR_COMPLICATION | count during F2: second blood test (weeks 16-19) |
| 2273 | F2_hospital_64300_MILD_{—} | Hospital diagnosis MILD HYPEREMESIS GRAVIDARUM, |
| | HYPEREMESIS_GRAVIDARUM_UNSP_{—} | UNSP.AS TO EPISODE OF CARE (64300), count during F2: |
| | AS_TO_EPISODE_OF_CARE | second blood test (weeks 16-19) |
| 2274 | F2_hospital_Z8878_DIAGNOSTIC_{—} | Hospital diagnosis DIAGNOSTIC ULTRASOUND OF GRAVID |
| | ULTRASOUND_OF_GRAVID_UTERUS | UTERUS (Z8878), count during F2: second blood test |
| | | (weeks 16-19) |
| 2275 | F2_hospital_Z9921_INJECTION_OF_{—} | Hospital diagnosis INJECTION OF ANTIBIOTIC (Z9921), count |
| | ANTIBIOTIC | during F2: second blood test (weeks 16-19) |
| 2276 | F2_hospital_Z9929_INJECT_OR_{—} | Hospital diagnosis INJECTOR INFUSION OF OTHER |
| | INFUSION_OF_OTHER_THERAPEUTIC_{—} | THERAPEUTIC OR PROPHYLACTIC SUBS. (Z9929), count |
| | OR_PROPHYLACTIC_SUBS | during F2: second blood test (weeks 16-19) |
| 2277 | F2_hospital_7840_HEADACHE | Hospital diagnosis HEADACHE (7840), count during F2: second |
| | | blood test (weeks 16-19) |
| 2278 | F2_hospital_V232_SUPERVISION_{—} | Hospital diagnosis SUPERVISION OF HIGH-RISK |
| | OF_HIGH_RISK_PREGNANCY_WITH_{—} | PREGNANCY WITH HISTORY OF ABORTION (V232), count |
| | HISTORY_OF_ABORTION | during F2: second blood test (weeks 16-19) |
| 2279 | F2_hospital_V2689_ENCOUNTER_{—} | Hospital diagnosis ENCOUNTER FOR OTHER SPECIFIED |
| | FOR_OTHER_SPECIFIED_{—} | PROCREATIVE MANAGEMENT (V2689), count during F2: |
| | PROCREATIVE_MANAGEMENT | second blood test (weeks 16-19) |
| 2280 | F2_hospital_65963_OTHER_{—} | Hospital diagnosis OTHER ADVANCED MATERNAL AGE, |
| | ADVANCED_MATERNAL_AGE_{—} | ANTEPARTUM CONDITION/COMPLICATION (65963), count |
| | ANTEPARTUM_CONDITION_OR_{—} | during F2: second blood test (weeks 16-19) |
| | COMPLICATION | |
| 2281 | F2_hospital_6318_OTHER_{—} | Hospital diagnosis OTHER ABNORMAL PRODUCTS OF |
| | ABNORMAL_PRODUCTS_OF_{—} | CONCEPTION (6318), count during F2: second blood test |
| | CONCEPTION | (weeks 16-19) |
| 2282 | F2_hospital_V2341_PREGNANCY_{—} | Hospital diagnosis PREGNANCY WITH HISTORY OF PRE- |
| | WITH_HISTORY_OF_PRE_TERM_{—} | TERM LABOR (V2341), count during F2: second blood test |
| | LABOR | (weeks 16-19) |
| 2283 | F2_hospital_04149_OTHER_AND_{—} | Hospital diagnosis OTHER AND UNSPECIFIED |
| | UNSPECIFIED_ESCHERICHIA_{—}COLI_{—} | ESCHERICHIA COLI (E. COLI) (04149), count during F2: |
| | E_{—}COLI | second blood test (weeks 16-19) |
| 2284 | F2_hospital_Z4701_LAPAROSCOPIC_{—} | Hospital diagnosis LAPAROSCOPIC APPENDECTOMY |
| | APPENDECTOMY | (Z4701), count during F2: second blood test (weeks 16-19) |
| 2285 | F2_hospital_7802_SYNCOPE_AND_{—} | Hospital diagnosis SYNCOPE AND COLLAPSE (7802), count |
| | COLLAPSE | during F2: second blood test (weeks 16-19) |
| 2286 | F2_hospital_Z5421_LAPAROSCOPY | Hospital diagnosis LAPAROSCOPY (Z5421), count during F2: |
| | | second blood test (weeks 16-19) |
| 2287 | F2_hospital_5409_ACUTE_{—} | Hospital diagnosis ACUTE APPENDICITIS WITHOUT |
| | APPENDICITIS_WITHOUT_{—} | MENTION OF PERITONITIS (5409), count during F2: second |
| | MENTION_OF_PERITONITIS | blood test (weeks 16-19) |
| 2288 | F2_hospital_63590_LEG_INDUCED_{—} | Hospital diagnosis LEG.INDUCED |
| | ABORTION_UNSPEC_WITHOUT_{—} | ABORTION, UNSPEC., WITHOUT MENTION OF |
| | MENTION_OF_COMPLICATION | COMPLICATION (63590), count during F2: second blood test |
| | | (weeks 16-19) |
| 2289 | F2_hospital_V719_OBSERVATION_{—} | Hospital diagnosis OBSERVATION FOR UNSPECIFIED |
| | FOR_UNSPECIFIED_SUSPECTED_{—} | SUSPECTED CONDITION (V719), count during F2: second |
| | CONDITION | blood test (weeks 16-19) |
| 2290 | F2_hospital_49390_ASTHMA_{—} | Hospital diagnosis ASTHMA, UNSPECIFIED TYPE, WITHOUT |
| | UNSPECIFIED_TYPE_WITHOUT_{—} | MENTION OF STATUS ASTHMATICUS (49390), count during |
| | MENTION_OF_STATUS_ASTHMATICUS | F2: second blood test (weeks 16-19) |
| 2291 | F2_hospital_Z6759_CERCLAGE_{—} | Hospital diagnosis CERCLAGE-UNSPECIFIED |
| | UNSPECIFIED_CERVIX_ISTHMUS_{—} | (CERVIX)(ISTHMUS UTERI) (Z6759), count during F2: second |
| | UTERI | blood test (weeks 16-19) |
| 2292 | F2_hospital_6205_TORSION_OF_{—} | Hospital diagnosis TORSION OF OVARY, OVARIAN |
| | OVARY_OVARIAN_PEDICLE_OR_{—} | PEDICLE, OR FALLOPIAN TUBE (6205), count during F2: |
| | FALLOPIAN_TUBE | second blood test (weeks 16-19) |
| 2293 | F2_hospital_64403_THREATENED_{—} | Hospital diagnosis THREATENED PREMATURE LABOR, |
| | PREMATURE_LABOR_ANTEPARTUM | ANTEPARTUM (64403), count during F2: second blood test |
| | | (weeks 16-19) |
| 2294 | F2_hospital_Z67591_CERCLAGE_MC_{—} | Hospital diagnosis CERCLAGE-MC DONALD |
| | DONALD_ENCIRCLEMENT_SUTURE_{—} | (ENCIRCLEMENT SUTURE, CERVIX) (Z67591), count during |
| | CERVIX | F2: second blood test (weeks 16-19) |
| 2295 | F2_hospital_2859_ANEMIA_{—} | Hospital diagnosis ANEMIA, UNSPECIFIED (2859), count |
| | UNSPECIFIED | during F2: second blood test (weeks 16-19) |
| 2296 | F2_hospital_6280 | Hospital diagnosis INFERTILITY, FEMALE, PRIMARY (6280 |
| | 1_INFERTILITY_{—} | 1), count during F2: second blood test (weeks 16-19) |
| | FEMALE_PRIMARY | |
| 2297 | F2_hospital_Z8876_DIAGNOSTIC_{—} | Hospital diagnosis DIAGNOSTIC ULTRASOUND OF |
| | ULTRASOUND_OF_ABDOMEN_AND_{—} | ABDOMEN AND RETROPERITONEUM (Z8876), count during |
| | RETROPERITONEUM | F2: second blood test (weeks 16-19) |
| 2298 | F2_hospital_6289_INFERTILITY_{—} | Hospital diagnosis INFERTILITY, FEMALE, OF UNSPECIFIED |
| | FEMALE_OF_UNSPECIFIED_ORIGIN | ORIGIN (6289), count during F2: second blood test |
| | | (weeks 16-19) |
| 2299 | F2_hospital_V141_PERSONAL_{—} | Hospital diagnosis PERSONAL HISTORY OF ALLERGY TO |
| | HISTORY_OF_ALLERGY_TO_OTHER_{—} | OTHER ANTIBIOTIC AGENT (V141), count during F2: second |
| | ANTIBIOTIC_AGENT | blood test (weeks 16-19) |
| 2300 | F2_hospital_64000_THREATENED_{—} | Hospital diagnosis THREATENED ABORTION, UNSPECIFIED |
| | ABORTION_UNSPECIFIED_AS_TO_{—} | AS TO EPISODE OF CARE (64000), count during F2: second |
| | EPISODE_OF_CARE | blood test (weeks 16-19) |
| 2301 | F2_hospital_64823_ANTEPARTUM_{—} | Hospital diagnosis ANTEPARTUM ANEMIA (64823), count |
| | ANEMIA | during F2: second blood test (weeks 16-19) |
| 2302 | F2_hospital_5990_URINARY_{—} | Hospital diagnosis URINARY TRACT INFECTION, SITE NOT |
| | TRACT_INFECTION_SITE_NOT_{—} | SPECIFIED (5990), count during F2: second blood test |
| | SPECIFIED | (weeks 16-19) |
| 2303 | F2_hospital_V22_NORMAL_{—} | Hospital diagnosis NORMAL PREGNANCY (V22), count during |
| | PREGNANCY | F2: second blood test (weeks 16-19) |
| 2304 | F1_hospital_49390_ASTHMA_{—} | Hospital diagnosis ASTHMA, UNSPECIFIED TYPE, WITHOUT |
| | UNSPECIFIED_TYPE_WITHOUT_{—} | MENTION OF STATUS ASTHMATICUS (49390), count during |
| | MENTION_OF_STATUS_ASTHMATICUS | F1: first blood test (weeks 6-12) |
| 2305 | F2_hospital_Z8879_OTHER_{—} | Hospital diagnosis OTHER DIAGNOSTIC ULTRASOUND |
| | DIAGNOSTIC_ULTRASOUND | (Z8879), count during F2: second blood test (weeks 16-19) |
| 2306 | F1_hospital_78609_OTHER_{—} | Hospital diagnosis OTHER DYSPNEA AND RESPIRATORY |
| | DYSPNEA_AND_RESPIRATORY_{—} | ABNORMALITY (78609), count during F1: first blood test |
| | ABNORMALITY | (weeks 6-12) |
| 2307 | F1_hospital_7880_RENAL_COLIC | Hospital diagnosis RENAL COLIC (7880), count during F1: first |
| | | blood test (weeks 6-12) |
| 2308 | F1_hospital_6238_OTHER_{—} | Hospital diagnosis OTHER SPECIFIED |
| | SPECIFIED_NONINFLAMMATORY_{—} | NONINFLAMMATORY DISORDERS OF VAGINA (6238), |
| | DISORDERS_OF_VAGINA | count during F1: first blood test (weeks 6-12) |
| 2309 | F1_hospital_64313_HYPEREMESIS_{—} | Hospital diagnosis HYPEREMESIS GRAVIDARUM WITH |
| | GRAVIDARUM_WITH_METABOLIC_{—} | METABOLIC DISTURBANCE, ANTEPARTUM (64313), count |
| | DISTURBANCE_ANTEPARTUM | during F1: first blood test (weeks 6-12) |
| 2310 | F1_hospital_5589_OTHER_AND_{—} | Hospital diagnosis OTHER AND UNSPEC.NONINFECTIOUS |
| | UNSPEC_NONINFECTIOUS_{—} | GASTROENTERITIS AND COLITIS (5589), count during F1: |
| | GASTROENTERITIS_AND_COLITIS | first blood test (weeks 6-12) |
| 2311 | F1_hospital_6289 | Hospital diagnosis INFERTILITY, FEMALE, PRIMARY (6289 |
| | 1_INFERTILITY_{—} | 1), count during F1: first blood test (weeks 6-12) |
| | FEMALE_PRIMARY | |
| 2312 | F1_hospital_64813_ANTEPARTUM_{—} | Hospital diagnosis ANTEPARTUM THYROID DYSFUNCTION |
| | THYROID_DYSFUNCTION | (64813), count during F1: first blood test (weeks 6-12) |
| 2313 | F1_hospital_57420_CALCUL_OF_{—} | Hospital diagnosis CALCUL.OF GALLBLADDER WITHOUT |
| | GALLBLADDER_WITHOUT_{—} | CHOLECYSTITIS, WITHOUT OBSTR. (57420), count during |
| | CHOLECYSTITIS_WITHOUT_OBSTR | F1: first blood test (weeks 6-12) |
| 2314 | F1_hospital_V234_SUPERVISION_OF_{—} | Hospital diagnosis SUPERVISION OF HIGH-RISK |
| | HIGH_RISK_PREGN_WITH_POOR_{—} | PREGN.WITH POOR OBSTETRIC HISTORY (V234), count |
| | OBSTETRIC_HISTORY | during F1: first blood test (weeks 6-12) |
| 2315 | F1_hospital_V146_PERSONAL_{—} | Hospital diagnosis PERSONAL HISTORY OF ALLERGY TO |
| | HISTORY_OF_ALLERGY_TO_{—} | ANALGESIC AGENT (V146), count during F1: first blood test |
| | ANALGESIC_AGENT | (weeks 6-12) |
| 2316 | F1_hospital_78060_FEVER_{—} | Hospital diagnosis FEVER, UNSPECIFIED (78060), count |
| | UNSPECIFIED | during F1: first blood test (weeks 6-12) |
| 2317 | F1_hospital_63490_SPONTANEOUS_{—} | Hospital diagnosis SPONTANEOUS |
| | ABORTION_UNSPECIFIED_WITHOUT_{—} | ABORTION, UNSPECIFIED, WITHOUT COMPLICATION |
| | COMPLICATION | (63490), count during F1: first blood test (weeks 6-12) |
| 2318 | F1_hospital_65963_OTHER_{—} | Hospital diagnosis OTHER ADVANCED MATERNAL AGE, |
| | ADVANCED_MATERNAL_AGE_{—} | ANTEPARTUM CONDITION/COMPLICATION (65963), count |
| | ANTEPARTUM_CONDITION_OR_{—} | during F1: first blood test (weeks 6-12) |
| | COMPLICATION | |
| 2319 | F1_hospital_6318_OTHER_{—} | Hospital diagnosis OTHER ABNORMAL PRODUCTS OF |
| | ABNORMAL_PRODUCTS_OF_{—} | CONCEPTION (6318), count during F1: first blood test |
| | CONCEPTION | (weeks 6-12) |
| 2320 | F1_hospital_V2341_PREGNANCY_{—} | Hospital diagnosis PREGNANCY WITH HISTORY OF PRE- |
| | WITH_HISTORY_OF_PRE_TERM_{—} | TERM LABOR (V2341), count during F1: first blood test |
| | LABOR | (weeks 6-12) |
| 2321 | F1_hospital_04149_OTHER_AND_{—} | Hospital diagnosis OTHER AND UNSPECIFIED |
| | UNSPECIFIED_ESCHERICHIA_{—}COLI_{—} | ESCHERICHIA COLI (E. COLI) (04149), count during F1: first |
| | E_{—}COLI | blood test (weeks 6-12) |
| 2322 | F1_hospital_Z4701_LAPAROSCOPIC_{—} | Hospital diagnosis LAPAROSCOPIC APPENDECTOMY |
| | APPENDECTOMY | (Z4701), count during F1: first blood test (weeks 6-12) |
| 2323 | F1_hospital_7802_SYNCOPE_AND_{—} | Hospital diagnosis SYNCOPE AND COLLAPSE (7802), count |
| | COLLAPSE | during F1: first blood test (weeks 6-12) |
| 2324 | F1_hospital_Z5421_LAPAROSCOPY | Hospital diagnosis LAPAROSCOPY (Z5421), count during F1: |
| | | first blood test (weeks 6-12) |
| 2325 | F1_hospital_5409_ACUTE_{—} | Hospital diagnosis ACUTE APPENDICITIS WITHOUT |
| | APPENDICITIS_WITHOUT_{—} | MENTION OF PERITONITIS (5409), count during F1: first |
| | MENTION_OF_PERITONITIS | blood test (weeks 6-12) |
| 2326 | F1_hospital_63590_LEG_INDUCED_{—} | Hospital diagnosis LEG.INDUCED |
| | ABORTION_UNSPEC_WITHOUT_{—} | ABORTION, UNSPEC., WITHOUT MENTION OF |
| | MENTION_OF_COMPLICATION | COMPLICATION (63590), count during F1: first blood test |
| | | (weeks 6-12) |
| 2327 | F1_hospital_V719_OBSERVATION_{—} | Hospital diagnosis OBSERVATION FOR UNSPECIFIED |
| | FOR_UNSPECIFIED_{—} | SUSPECTED CONDITION (V719), count during F1: first blood |
| | SUSPECTED_CONDITION | test (weeks 6-12) |
| 2328 | F1_hospital_V2689_ENCOUNTER_{—} | Hospital diagnosis ENCOUNTER FOR OTHER SPECIFIED |
| | FOR_OTHER_SPECIFIED_{—} | PROCREATIVE MANAGEMENT (V2689), count during F1: |
| | PROCREATIVE_MANAGEMENT | first blood test (weeks 6-12) |
| 2329 | F1_hospital_Z9911_INJECTION_OF_{—} | Hospital diagnosis INJECTION OF RH IMMUNE GLOBULIN |
| | RH_IMMUNE_GLOBULIN | (Z9911), count during F1: first blood test (weeks 6-12) |
| 2330 | F1_hospital_64093_UNSPECIFIED_{—} | Hospital diagnosis UNSPECIFIED HEMORRHAGE IN EARLY |
| | HEMORRHAGE_IN_EARLY_{—} | PREGNANCY, ANTEPARTUM (64093), count during F1: first |
| | PREGNANCY_ANTEPARTUM | blood test (weeks 6-12) |
| 2331 | F1_hospital_V270_MOTHER_WITH_{—} | Hospital diagnosis MOTHER WITH SINGLE LIVEBORN |
| | SINGLE_LIVEBORN | (V270), count during F1: first blood test (weeks 6-12) |
| 2332 | F2_hospital_632_MISSED_ABORTION | Hospital diagnosis MISSED ABORTION (632), count during F2: |
| | | second blood test (weeks 16-19) |
| 2333 | F2_hospital_Z6952_ASPIRATION_{—} | Hospital diagnosis ASPIRATION CURETTAGE FOLLOWING |
| | CURETTAGE_FOLLOWING_{—} | DELIVERY OR ABORTION (Z6952), count during F2: second |
| | DELIVERY_OR_ABORTION | blood test (weeks 16-19) |
| 2334 | F2_hospital_64663_ANTEPARTUM_{—} | Hospital diagnosis ANTEPARTUM INFECTIONS OF |
| | INFECTIONS_OF_GENITOURINARY_{—} | GENITOURINARY TRACT (64663), count during F2: second |
| | TRACT | blood test (weeks 16-19) |
| 2335 | F2_hospital_Z96490_ABORTION_{—} | Hospital diagnosis ABORTION INDUCED BY MEDICATION |
| | INDUCED_BY_MEDICATION | (Z96490), count during F2: second blood test (weeks 16-19) |
| 2336 | F2_hospital_Z65911_E_T | Hospital diagnosis E.T. (Z65911), count during F2: second blood |
| | | test (weeks 16-19) |
| 2337 | F2_hospital_6288_INFERTILITY_{—} | Hospital diagnosis INFERTILITY, FEMALE, OF OTHER |
| | FEMALE_OF_OTHER_SPECIFIED_ORIGIN | SPECIFIED ORIGIN (6288), count during F2: second blood test |
| | | (weeks 16-19) |
| 2338 | F2_hospital_64003_THREATENED_{—} | Hospital diagnosis THREATENED ABORTION, |
| | ABORTION_ANTEPARTUM | ANTEPARTUM (64003), count during F2: second blood test |
| | | (weeks 16-19) |
| 2339 | F2_hospital_Z6902_DILATION_{—} | Hospital diagnosis DILATION AND CURETTAGE |
| | AND_CURETTAGE_FOLLOWING_{—} | FOLLOWING DELIVERY OR ABORTION (Z6902), count |
| | DELIVERY_OR_ABORTION | during F2: second blood test (weeks 16-19) |
| 2340 | F2_hospital_V221_SUPERVISION_{—} | Hospital diagnosis SUPERVISION OF OTHER NORMAL |
| | OF_OTHER_NORMAL_PREGNANCY | PREGNANCY (V221), count during F2: second blood test |
| | | (weeks 16-19) |
| 2341 | F2_hospital_Z65910_I_V_F | Hospital diagnosis I.V.F. (Z65910), count during F2: second |
| | | blood test (weeks 16-19) |
| 2342 | F2_hospital_78900_ABDOMINAL_{—} | Hospital diagnosis ABDOMINAL PAIN, UNSP. SITE (78900), |
| | PAIN_UNSP_SITE | count during F2: second blood test (weeks 16-19) |
| 2343 | F2_hospital_64303_MILD_{—} | Hospital diagnosis MILD HYPEREMESIS GRAVIDARUM, |
| | HYPEREMESIS_GRAVIDARUM_{—} | ANTEPARTUM (64303), count during F2: second blood test |
| | ANTEPARTUM | (weeks 16-19) |
| 2344 | F1_hospital_Z5123_LAPAROSCOPIC_{—} | Hospital diagnosis LAPAROSCOPIC |
| | CHOLECYSTECTOMY_1992 | CHOLECYSTECTOMY_1992 (Z5123), count during F1: first |
| | | blood test (weeks 6-12) |
| 2345 | F2_hospital_V222_PREGNANT_{—} | Hospital diagnosis PREGNANT STATE, INCIDENTAL (V222), |
| | STATE_INCIDENTAL | count during F2: second blood test (weeks 16-19) |
| 2346 | F2_hospital_0_OTHER | Hospital diagnosis OTHER (0), count during F2: second blood |
| | | test (weeks 16-19) |
| 2347 | F1_hospital_Z6595_RELEASE_OF_{—} | Hospital diagnosis RELEASE OF TORSION OF OVARY |
| | TORSION_OF_OVARY | (Z6595), count during F1: first blood test (weeks 6-12) |
| 2348 | F1_hospital_6201_CORPUS_{—} | Hospital diagnosis CORPUS LUTEUM CYST OR HEMATOMA |
| | LUTEUM_CYST_OR_HEMATOMA | (6201), count during F1: first blood test (weeks 6-12) |
| 2349 | F1_hospital_Z8875_DIAGNOSTIC_{—} | Hospital diagnosis DIAGNOSTIC ULTRASOUND OF |
| | ULTRASOUND_OF_URINARY_SYSTEM | URINARY SYSTEM (Z8875), count during F1: first blood test |
| | | (weeks 6-12) |
| 2350 | F1_hospital_64973_CERVICAL_{—} | Hospital diagnosis CERVICAL SHORTENING |
| | SHORTENING_COMPLICATING_{—} | COMPLICATING PREGNANCY, ANTEPARTUM |
| | PREGNANCY_ANTEPARTUM_{—} | CONDITION OR COMPLICATION (64973), count during F1: |
| | CONDITION_OR_COMPLICATION | first blood test (weeks 6-12) |
| 2351 | F1_hospital_591_HYDRONEPHROSIS | Hospital diagnosis HYDRONEPHROSIS (591), count during F1: |
| | | first blood test (weeks 6-12) |
| 2352 | F1_hospital_64193_UNSPECIFIED_{—} | Hospital diagnosis UNSPECIFIED ANTEPARTUM |
| | ANTEPARTUM_HEMORRHAGE | HEMORRHAGE (64193), count during F1: first blood test |
| | | (weeks 6-12) |
| 2353 | F1_hospital_6400_THREATENED_{—} | Hospital diagnosis THREATENED ABORTION (6400), count |
| | ABORTION | during F1: first blood test (weeks 6-12) |
| 2354 | F1_hospital_V180_FAMILY_{—} | Hospital diagnosis FAMILY HISTORY OF DIABETES |
| | HISTORY_OF_DIABETES_MELLITUS | MELLITUS (V180), count during F1: first blood test |
| | | (weeks 6-12) |
| 2355 | prev_pregnancy_3_is_GDM | GDM positive at 3rd previous pregnancy |

### DISCUSSION

In the above Examples, the ability to utilize EHRs for predicting GDM in early stages of pregnancy was examined. The results show that EHRs can be used to produce accurate predictions of GDM risk, performing significantly better than a baseline model based on commonly assessed risk factors. The analysis presented in the above Examples demonstrate that accurate prediction of GDM is feasible even at pregnancy initiation, with an auROC of 0.836, close to the performance of a predictor constructed later on in pregnancy, which reaches an auROC of 0.854.

The predictor of the present embodiments may be used to identify and recruit a high risk cohort with risk of up to 70% for GDM development. The study presented in the above Examples thus allows early prediction and detection of GDM and prevention interventions.

Other than well known risk factors for GDM development such as maternal age and family history of diabetes, the analysis presented in the above Examples reveals factors that were not previously described to be highly predictive of gestational diabetes. The main risk factors identified were results of the 50 g GTT tests in previous pregnancies. While the medical system already addresses women with a history of GDM in previous pregnancies as being at increased risk for GDM in the current pregnancy, the experiments performed by the inventors according to some embodiments of the present invention show that the 50 g GTT result is far more predictive. Thus, according to some embodiments of the present invention explicit GTT value can be used as a risk assessment guideline with more weight than, and more preferably instead of, GDM diagnosis.

Although maximal prediction accuracy requires using the patient's entire EHR, the above Examples demonstrate that 9 simple questions that can be answered by the patient herself still enable accurate prediction (auROC of 0.801). This allows patients to get accurate GDM risk estimation by web- or smartphone-based self assessment tools.

Currently, in Israel, a 50 g GTT is used as a universal screening test for GDM, followed by the 100 g GTT if needed. The results presented in the above Examples show that the use of prediction model, which is more cost-effective and efficient, allows the identification of low risk women who can therefore avoid the 50 g GTTs entirely. Thus, the present embodiments comprise a selective screening method, wherein women for which the prediction is low are screened out of the 50 g GTT. Avoiding 50% of the 50 g GTTs of patients who previously did a GTT would result in only 5% miss rate when diagnosing GDM according to the two steps approach guidelines. Accurate selective screening is highly desirable, as it can both reduces costs and physical inconvenience for women at low risk for GDM development.

Although the predictor presented in the above Examples is based on retrospective EHR which have inherent biases and are influenced by the interaction of the patient with the health system, these biases are reduced since the data contains information originating from a non-governmental, non-profit organization which includes the majority of the Israeli population, and since the outcome of the model is based on routine pregnancy tests. Other than the data used in the above Examples, some embodiments of the present invention contemplate use of additional types of information for predicting the likelihood for GDM. These types of information include, without limitation, information regarding lifestyle habits. Although the predictor was trained and validated on Israeli population data, the size of the dataset, the validation process, and the fact that the analysis validated the utility of established risk factors for GDM development, supports the ability of the method of the present embodiments to provide prediction also for other populations.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

[1] Donovan, P. J. & McIntyre, H. D. Drugs for gestational diabetes. Aust. Prescr. 33, 141-144 (2010).
[2] American Diabetes Association. 2. Classification and Diagnosis of Diabetes: Standards of Medical Care in Diabetes-2018. Diabetes Care 41, S13-S27 (2018).
[3] Lowe, L. P. et al. Hyperglycemia and Adverse Pregnancy Outcome (HAPO) Study: associations of maternal A1C and glucose with pregnancy outcomes. Diabetes Care 35, 574-580 (2012).
[4] Lowe, W. L. et al. Hyperglycemia and Adverse Pregnancy Outcome Follow-up Study (HAPO FUS): Maternal Gestational Diabetes Mellitus and Childhood Glucose Metabolism. Diabetes Care 42, 372-380 (2019).
[5] Lowe, W. L. et al. Association of gestational diabetes with maternal disorders of glucose metabolism and childhood adiposity. JAMA 320, 1005-1016 (2018).
[6] Zhao, P. et al. Maternal gestational diabetes and childhood obesity at age 9-11: results of a multinational study. Diabetologia 59, 2339-2348 (2016).
[7] Hunt, K. J. & Schuller, K. L. The increasing prevalence of diabetes in pregnancy. Obstet. Gynecol. Clin. North Am. 34, 173-99, vii (2007).
[8] Bain, E. et al. Diet and exercise interventions for preventing gestational diabetes mellitus. Cochrane Database Syst. Rev. CD010443 (2015). doi: 10.1002/14651858.CD010443.pub2
[9] Koivusalo, S. B. et al. Gestational diabetes mellitus can be prevented by lifestyle intervention: the finnish gestational diabetes prevention study (RADIEL): A randomized controlled trial. Diabetes Care 39, 24-30 (2016).
[10] Wang, C. et al. A randomized clinical trial of exercise during pregnancy to prevent gestational diabetes mellitus and improve pregnancy outcome in overweight and obese pregnant women. Am. J. Obstet. Gynecol. 216, 340-351 (2017).
[11] Avati, A. et al. Improving palliative care with deep learning. in 2017 IEEE International Conference on Bioinformatics and Biomedicine (BIBM) 311-316 (IEEE, 2017). doi:10.1109/BIBM.2017.8217669
[12] Silva, I., Moody, G., Scott, D. J., Celi, L. A. & Mark, R. G. Predicting In-Hospital Mortality of ICU Patients: The PhysioNet/Computing in Cardiology Challenge 2012. Comput Cardiol (2010) 39, 245-248 (2012).
[13] Razavian, N., Marcus, J. & Sontag, D. Multi-task Prediction of Disease Onsets from Longitudinal Lab Tests. arXiv (2016).
[14] Oh, J. et al. A Generalizable, Data-Driven Approach to Predict Daily Risk of Clostridium difficile Infection at Two Large Academic Health Centers. Infect. Control Hosp. Epidemiol. 39, 425-433 (2018).
[15] Miotto, R., Li, L., Kidd, B. A. & Dudley, J. T. Deep Patient: An Unsupervised Representation to Predict the Future of Patients from the Electronic Health Records. Sci. Rep. 6, 26094 (2016).
[16] Danilenko-Dixon, D. R., Van Winter, J. T., Nelson, R. L. & Ogburn, P. L. Universal versus selective gestational diabetes screening: application of 1997 American Diabetes Association recommendations. Am. J. Obstet. Gynecol. 181, 798-802 (1999).
[17] Data—Clalit Research Institute. at www(dot)clalitresearchDOTorg/about-us/our-data/[18]
[18] Vandorsten, J. P. et al. NIH consensus development conference: diagnosing gestational diabetes mellitus. NIH Consens. State Sci. Statements 29, 1-31 (2013).
[19] Monitoring of Pregnancy and Medical Examinations During Pregnancy, Ministry of Health. at www(dot)healthDOTgovDOTil/English/Topics/Pregnancy/during/examination/Pages/permanent(dot)aspx
[20] Arlot, S. & Celisse, A. A survey of cross-validation procedures for model selection. Stat. Surv. 4, 40-79 (2010).
[21] U.S. DEPARTMENT OF HEALTH AND HUMAN SERVICES, National Institutes of Health, Eunice Kennedy Shriver National Institute of Child Health and Human Development. Am I at risk for gestational diabetes? at www(dot)nichdDOTnihDOTgov/sites/default/files/publications/pubs/Documents/gestational_diabetes_2012(dot)pdf
[22] Mining, D. et al. The Elements of Statistical Learning.
[23] Fernández-Delgado, M., Cernadas, E., Barro, S. & Amorim, D. Do we Need Hundreds of Classifiers to Solve Real World Classification Problems? Journal of Machine Learning Research (2014).
[24] Omar, K. XGBoost and LGBM for Porto Seguro's Kaggle challenge: A comparison Semester Project. (2018).
[25] biendata competitions. KDD Cup 2018—Winners List. at www(dot)biendataDOTcom/competition/kdd_2018/winners/[26]
[26] Lundberg, S. & Lee, S.-I. A Unified Approach to Interpreting Model Predictions. arXiv (2017).
[27] Lundberg, S. M. et al. Explainable machine-learning predictions for the prevention of hypoxaemia during surgery. Nat. Biomed. Eng. 2, 749-760 (2018).
[28] Steyerberg, E. W. et al. Assessing the performance of prediction models: a framework for traditional and novel measures. Epidemiology 21, 128-138 (2010).
[29] Chu, S. Y. et al. Maternal obesity and risk of gestational diabetes mellitus. Diabetes Care 30, 2070-2076 (2007).
[30] Williams, M. A., Qiu, C., Dempsey, J. C. & Luthy, D. A. Familial aggregation of type 2 diabetes and chronic hypertension in women with gestational diabetes mellitus. J. Reprod. Med. 48, 955-962 (2003).
[31] Lamain-de Ruiter, M. et al. External validation of prognostic models to predict risk of gestational diabetes mellitus in one Dutch cohort: prospective multicentre cohort study. BMJ 354, i4338 (2016).
[32] Shepherd, E. et al. Combined diet and exercise interventions for preventing gestational diabetes mellitus. Cochrane Database Syst. Rev. 11, CD010443 (2017).
[33] Lao, T. T., Ho, L.-F., Chan, B. C. P. & Leung, W.-C. Maternal age and prevalence of gestational diabetes mellitus. Diabetes Care 29, 948-949 (2006).
[34] Di Cianni, G. et al. Prevalence and risk factors for gestational diabetes assessed by universal screening. Diabetes Res. Clin. Pract. 62, 131-137 (2003).
[35] Teh, W. T. et al. Risk factors for gestational diabetes mellitus: implications for the application of screening guidelines. Aust. N. Z. J. Obstet. Gynaecol. 51, 26-30 (2011).
[36] Davey, R. X. Selective versus universal screening for gestational diabetes mellitus: an evaluation of predictive risk factors | The Medical Journal of Australia. The Medical Journal of Australia (2001).
[37] Kalter-Leibovici, O. et al. Screening and diagnosis of gestational diabetes mellitus: critical appraisal of the new International Association of Diabetes in Pregnancy Study Group recommendations on a national level. Diabetes Care 35, 1894-1896 (2012).
[38] Phelan, M., Bhaysar, N. A. & Goldstein, B. A. Illustrating Informed Presence Bias in Electronic Health Records Data: How Patient Interactions with a Health System Can Impact Inference. EGEMS (Wash. D.C.) 5, 22 (2017).
[39] Zhang, C. & Ning, Y. Effect of dietary and lifestyle factors on the risk of gestational diabetes: review of epidemiologic evidence. Am. J. Clin. Nutr. 94, 1975S-1979S (2011).
[40] CBS Site. at www(dot)cbsDOTgovDOTil/en/pages/default(dot)aspx
[41] Ke, G. et al. LightGBM: A Highly Efficient Gradient Boosting Decision Tree. undefined (2017).

1. A method of predicting likelihood for gestational diabetes, comprising:
obtaining a plurality of parameters characterizing a female subject, said plurality of parameters comprising at least 10 of the parameters that are listed at lines 1-40 of Table 6.1;
accessing a computer readable medium storing a machine learning procedure trained for predicting likelihoods for gestational diabetes;
feeding said procedure with said plurality of parameters; and
receiving from said procedure an output indicative of a likelihood that said subject has, or expected to develop, gestational diabetes, wherein said output indicative is related non-linearly to said parameters.

2. (canceled)

3. The method according to claim 1, comprising presenting to said subject, by a user interface, a questionnaire and a set of questionnaire controls, receiving a set of response parameters entered by the subject using said questionnaire controls, wherein said plurality of parameters comprises said response parameters.

4. The method according to claim 1, wherein said plurality of parameters comprises at least one parameter extracted from a body liquid test applied to said subject.

5. The method according to claim 1, wherein said plurality of parameters comprises at least one parameter extracted from a diagnosis previously recorded for said subject.

6. The method according to claim 1, wherein said plurality of parameters comprises at least one parameter indicative of a pharmaceutical prescribed for said subject.

7. The method according to claim 1, wherein said female subject is pregnant.

8. The method of claim 7, wherein said pregnant subject is at less than 12 weeks gestation.

9. The method of claim 7, wherein said pregnant subject is at less than 8 weeks gestation.

10. (canceled)

11. The method according to claim 1, wherein said plurality of parameters comprises a result of a blood glucose test applied to said subject.

12. The method according to claim 1, wherein said plurality of parameters comprises an absolute neutrophil count (NEUT.abs) obtained from a blood of said subject.

13. The method according to claim 1, wherein said plurality of parameters comprises white blood cells count WBC, obtained from a blood of said subject.

14. The method according to claim 1, wherein said plurality of parameters comprises a result of a blood glucose test applied to said subject within about 1 year before said pregnancy.

15. The method according to claim 1, wherein said female subject is not pregnant.

16-17. (canceled)

18. The method according to claim 1, wherein said plurality of parameters comprises at least one parameter indicative of a glucose tolerance test applied to said subject.

19. The method according to claim 1, wherein said subject has been previously pregnant, and wherein said plurality of parameters comprises a result of a glucose tolerance test applied to said subject during the previous pregnancy.

20. The method according to claim 1, wherein said subject has been previously pregnant, and wherein said plurality of parameters comprises a result of a blood glucose test applied to said subject during the previous pregnancy.

21. The method according to claim 19, wherein said previous pregnancy is a most recent previous pregnancy.

22. The method according to claim 19, wherein said previous pregnancy is a next-to-most recent previous pregnancy.

23. (canceled)

24. The method according to claim 1, wherein said plurality of parameters comprises at least 12 or at least 14 or at least 16 of the parameters that are listed at lines 1-40 more preferably lines 1-30 more preferably lines 1-20 of Table 6.1.

25-26. (canceled)

27. A method of predicting likelihood for gestational diabetes, comprising:
presenting on a user interface a questionnaire and a set of questionnaire controls, and receiving from said user interface a set of response parameters entered using said questionnaire controls, wherein said set of response parameters characterizes a female subject, said parameters comprising anthropometric parameters, a parameter indicative of an age of the female subject, one or more parameters indicative of history of diabetes for the female subject and for a first-degree relative of the female subject, one or more parameters indicative of diagnoses of the female subject, one or more parameters indicative of blood test results of the female subject, and pregnancy history of the female subject;
accessing a computer readable medium storing a machine learning procedure trained for predicting likelihoods for gestational diabetes;
feeding said procedure with said set of parameters; and
receiving from said procedure an output indicative of a likelihood that said female subject has, or expected to develop, gestational diabetes, wherein said output indicative is related non-linearly to said parameters.

28. (canceled)
